(19) United States
(12) Patent Application Publication
(10) Pub. No.: US 2006/0141575 A1
(43) Pub. Date: Jun. 29, 2006

(54) Secreted proteins and nucleic acids encoding them

(75) Inventor: Thomas M. Barnes, Boston, MA (US)

Correspondence Address: MILLENNIUM PHARMACEUTICALS, INC., 40 Landsdowne Street, CAMBRIDGE, MA, 02139, US

(73) Assignee: Millennium Pharmaceuticals, Inc.

(21) Appl. No.: 11/354,653

(22) Filed: Feb. 15, 2006

Related U.S. Application Data
(63) Continuation of application No. 10/116,512, filed on Apr. 3, 2002, now abandoned.
Application No. 10/116,512 is a continuation of application No. 09/409,920, filed on Sep. 30, 1999, now abandoned.
Application No. 09/409,920 is a continuation-in-part of application No. 09/164,169, filed on Oct. 2, 1998, now abandoned.
Application No. 09/409,920 is a continuation-in-part of application No. 09/164,220, filed on Sep. 30, 1998, now abandoned.

(51) Int. Cl. C07K 14/715; C07K 14/52; C07H 21/04; C12P 21/06

(52) U.S. Cl. 435/69.1; 435/320.1; 435/325; 530/350; 530/351; 536/23.5

(57) ABSTRACT

The invention concerns cDNA molecules encoding TANGO 180, TANGO 181, TANGO 182, TANGO 183, TANGO 184, TANGO 185, TANGO 186, TANGO 187, TANGO 188, TANGO 189, and TANGO 215, all of which are either secreted or transmembrane proteins. The nucleic acids and polypeptides of the present invention are useful as modulating agents in regulating a variety of cellular processes. Accordingly, in one aspect, this invention provides isolated nucleic acid molecules encoding a polypeptide of the invention or biologically active portion thereof. The present invention also provides nucleic acid molecules which are suitable as primers or hybridization probes for the detection of nucleic acids encoding a polypeptide of the invention.

### RELATED APPLICATION INFORMATION

This application is a continuation-in-part of application Ser. No. 09/164,169, filed Oct. 2, 1998, which is a continuation-in-part of application Ser. No. 09/164,220, filed Sep. 30, 1998.

### BACKGROUND OF THE INVENTION

Many secreted proteins, for example, cytokines and cytokine receptors, play a vital role in the regulation of cell growth, cell differentiation, and a variety of specific cellular responses. A number of medically useful proteins, including erythropoietin, granulocyte-macrophage colony stimulating factor, human growth hormone, and various interleukins, are secreted proteins. Thus, an important goal in the design and development of new therapies is the identification and characterization of secreted and transmembrane proteins and the genes which encode them.

Many secreted proteins are receptors which bind a ligand and transduce an intracellular signal, leading to a variety of cellular responses. The identification and characterization of such a receptor enables one to identify both the ligands which bind to the receptor and the intracellular molecules and signal transduction pathways associated with the receptor, permitting one to identify or design modulators of receptor activity, e.g., receptor agonists or antagonists and modulators of signal transduction.

### SUMMARY OF THE INVENTION

The present invention is based, at least in part, on the discovery of cDNA molecules encoding TANGO 180, TANGO 181, TANGO 182, TANGO 183, TANGO 184, TANGO 185, TANGO 186, TANGO 187, TANGO 188, TANGO 189, and TANGO 215, all of which are predicted to be either wholly secreted or transmembrane proteins. These proteins, fragments, derivatives, and variants thereof are collectively referred to as “polypeptides of the invention” or “proteins of the invention.” Nucleic acid molecules encoding polypeptides of the invention are collectively referred to as “nucleic acids of the invention.”

The nucleic acids and polypeptides of the present invention are useful as modulating agents in regulating a variety of cellular processes. Accordingly, in one aspect, the present invention provides isolated nucleic acid molecules encoding a polypeptide of the invention or a biologically active portion thereof. The present invention also provides nucleic acid molecules which are suitable as primers or hybridization probes for the detection of nucleic acids encoding a polypeptide of the invention.

The invention features nucleic acid molecules which are at least 45% (or 55%, 65%, 75%, 85%, 95%, or 98%) identical to the nucleotide sequence of any of SEQ ID Nos:1-22, 34-43 and ______-______ or the nucleotide sequence of the cDNA of a clone deposited with ATCC as any of Accession Numbers 98899, 98900 and 98901 (the “cDNA of a clone deposited as any of ATCC 98899, 98900, and 989001”), or a complement thereof.

The invention features nucleic acid molecules which include a fragment of at least 300 (325, 350, 375, 400, 425, 450, 500, 550, 600, 650, 700, 800, 900, 1000, or 1200) nucleotides of the nucleotide sequence of any of SEQ ID Nos:1-22, 34-43 and ______-______ or the nucleotide sequence of the cDNA of a clone deposited as any of ATCC 98899, 98900, and 989001, or a complement thereof.

The invention also features nucleic acid molecules which include a nucleotide sequence encoding a protein having an amino acid sequence that is at least 45% (or 55%, 65%, 75%, 85%, 95%, or 98%) identical to the amino acid sequence of any of SEQ ID Nos:23-33, 54-63, and ______-______ or the amino acid sequence encoded by the cDNA of a clone deposited as any of ATCC 98899, 98900, and 989001, or a complement thereof.

In preferred embodiments, the nucleic acid molecules have the nucleotide sequence of any of SEQ ID NOs:1-22, 34-43 and ______-______ or the nucleotide sequence of the cDNA of a clone deposited as any of ATCC 98899, 98900, and 989001.

Also within the invention are nucleic acid molecules which encode a fragment of a polypeptide having the amino acid sequence of any of SEQ ID Nos:23-33, 54-63, and ______-______ the fragment including at least 15 (25, 30, 50, 100, 150, 300, or 400) contiguous amino acids of any of SEQ ID Nos:23-33, 54-63, and ______-______ or the polypeptide encoded by the cDNA of a clone deposited as any of ATCC 98899, 98900, and 989001.

The invention includes nucleic acid molecules which encode a naturally occurring allelic variant of a polypeptide comprising the amino acid sequence of any of SEQ ID Nos:23-33, 54-63, and ______-______ or an amino acid sequence encoded by the cDNA of a clone deposited as any of ATCC 98899, 98900, and 989001, wherein the nucleic acid molecule hybridizes under stringent conditions to a nucleic acid molecule having a nucleic acid sequence encoding any of SEQ ID NOs:22-33, 54-63, and ______-______, or a complement thereof.

Also within the invention are: isolated polypeptides or proteins having an amino acid sequence that is at least about 65%, preferably 75%, 85%, 95%, or 98% identical to the amino acid sequence of any of SEQ ID NOs: 22-33, 54-63, and ______-______.

Also within the invention are: isolated polypeptides or proteins which are encoded by a nucleic acid molecule having a nucleotide sequence that is at least about 65%, preferably 75%, 85%, or 95% identical the nucleic acid sequence encoding any of SEQ ID Nos:22-33, 54-63, and ______-______ and isolated polypeptides or proteins which are encoded by a nucleic acid molecule having a nucleotide sequence which hybridizes under stringent hybridization conditions to a nucleic acid molecule having the sequence of any of SEQ ID NOs:1-22, 34-43, and ______-______, and a complement thereof or the non-coding strand of the cDNA of a clone deposited as any of ATCC 98899, 98900, and 989001.

Also within the invention are polypeptides which are naturally occurring allelic variants of a polypeptide that includes the amino acid sequence of any of SEQ ID NOs:22-33, 54-63, and ______-______ or an amino acid sequence encoded by the cDNA of a clone deposited as any of ATCC 98899, 98900, and 989001, wherein the polypeptide is encoded by a nucleic acid molecule which hybridizes under stringent conditions to a nucleic acid molecule having the sequence of any of SEQ ID NOs:1-22, 34-43, and ______-______, or a complement thereof.

The invention also features nucleic acid molecules that hybridize under stringent conditions to a nucleic acid molecule comprising the nucleotide sequence of any of SEQ ID NOs:1-22, 34-43, and ______-______, of the cDNA of a clone deposited as any of ATCC 98899, 98900, and 989001, or a complement thereof. In other embodiments, the nucleic acid molecules are at least 300 (325, 350, 375, 400, 425, 450, 500, 550, 600, 650, 700, 800, 900, 1000, or 1290) nucleotides in length and hybridize under stringent conditions to a nucleic acid molecule comprising the nucleotide sequence of any of SEQ ID NOs:1-22, 34-43, and ______-______ of the cDNA of a clone deposited as any of ATCC 98899, 98900, and 989001, or a complement thereof. In preferred embodiments, the isolated nucleic acid molecules encode a cytoplasmic, transmembrane, or extracellular domain of a polypeptide of the invention. In another embodiment, the invention provides an isolated nucleic acid molecule which is antisense to the coding strand of a nucleic acid of the invention.

Another aspect of the invention provides vectors, e.g., recombinant expression vectors, comprising a nucleic acid molecule of the invention. In another embodiment the invention provides host cells containing such a vector. The invention also provides methods for producing a polypeptide of the invention by culturing, in a suitable medium, a host cell of the invention containing a recombinant expression vector encoding a polypeptide of the invention such that the polypeptide of the invention is produced.

Another aspect of this invention features isolated or recombinant proteins and polypeptides of the invention. Preferred proteins and polypeptides possess at least one biological activity possessed by the corresponding naturally-occurring human polypeptide. An activity, a biological activity, and a functional activity of a polypeptide of the invention refers to an activity exerted by a protein or polypeptide of the invention on a responsive cell as determined in vivo, or in vitro, according to standard techniques. Such activities can be a direct activity, such as an association with or an enzymatic activity on a second protein or an indirect activity, such as a cellular signaling activity mediated by interaction of the protein with a second protein. Thus, such activities include, e.g., (1) the ability to form protein-protein interactions with proteins in the signaling pathway of the naturally-occurring polypeptide; (2) the ability to bind a ligand of the naturally-occurring polypeptide; (3) the ability to bind to an intracellular target of the naturally-occurring polypeptide. Other activities include: (1) the ability to modulate cellular proliferation; (2) the ability to modulate cellular differentiation; and (3) the ability to modulate cell death.

In one embodiment, a polypeptide of the invention has an amino acid sequence sufficiently identical to an identified domain of a polypeptide of the invention. As used herein, the term “sufficiently identical” refers to a first amino acid or nucleotide sequence which contains a sufficient or minimum number of identical or equivalent (e.g., with a similar side chain) amino acid residues or nucleotides to a second amino acid or nucleotide sequence such that the first and second amino acid or nucleotide sequences have a common structural domain and/or common functional activity. For example, amino acid or nucleotide sequences which contain a common structural domain having about 65% identity, preferably 75% identity, more preferably 85%, 95%, or 98% identity are defined herein as sufficiently identical.

In one embodiment, the isolated polypeptide of the invention lacks both a transmembrane and a cytoplasmic domain. In another embodiment, the polypeptide lacks both a transmembrane domain and a cytoplasmic domain and is soluble under physiological conditions.

The polypeptides of the present invention, or biologically active portions thereof, can be operably linked to a heterologous amino acid sequence to form fusion proteins. The invention further features antibodies that specifically bind a polypeptide of the invention such as monoclonal or polyclonal antibodies. In addition, the polypeptides of the invention or biologically active portions thereof can be incorporated into pharmaceutical compositions, which optionally include pharmaceutically acceptable carriers.

In another aspect, the present invention provides methods for detecting the presence of the activity or expression of a polypeptide of the invention in a biological sample by contacting the biological sample with an agent capable of detecting an indicator of activity such that the presence of activity is detected in the biological sample.

In another aspect, the invention provides methods for modulating activity of a polypeptide of the invention comprising contacting a cell with an agent that modulates (inhibits or stimulates) the activity or expression of a polypeptide of the invention such that activity or expression in the cell is modulated. In one embodiment, the agent is an antibody that specifically binds to a polypeptide of the invention.

In another embodiment, the agent modulates expression of a polypeptide of the invention by modulating transcription, splicing, or translation of an mRNA encoding a polypeptide of the invention. In yet another embodiment, the agent is a nucleic acid molecule having a nucleotide sequence that is antisense to the coding strand of an mRNA encoding a polypeptide of the invention.

The present invention also provides methods to treat a subject having a disorder characterized by aberrant activity of a polypeptide of the invention or aberrant expression of a nucleic acid of the invention by administering an agent which is a modulator of the activity of a polypeptide of the invention or a modulator of the expression of a nucleic acid of the invention to the subject. In one embodiment, the modulator is a protein of the invention. In another embodiment, the modulator is a nucleic acid of the invention. In other embodiments, the modulator is a peptide, peptidomimetic, or other small molecule.

The present invention also provides diagnostic assays for identifying the presence or absence of a genetic lesion or mutation characterized by at least one of: (i) aberrant modification or mutation of a gene encoding a polypeptide of the invention, (ii) mis-regulation of a gene encoding a polypeptide of the invention, and (iii) aberrant post-translational modification of a polypeptide of the invention wherein a wild-type form of the gene encodes a polypeptide having the activity of the polypeptide of the invention.

In another aspect, the invention provides a method for identifying a compound that binds to or modulates the activity of a polypeptide of the invention. In general, such methods entail measuring a biological activity of the polypeptide in the presence and absence of a test compound and identifying those compounds which alter the activity of the polypeptide.

The invention also features methods for identifying a compound which modulates the expression of a polypeptide or nucleic acid of the invention by measuring the expression of the polypeptide or nucleic acid in the presence and absence of the compound.

Other features and advantages of the invention will be apparent from the following detailed description and claims.

### BRIEF DESCRIPTION OF THE DRAWINGS

FIG. 1 depicts the cDNA sequence (SEQ ID NO:1) and predicted amino acid sequence (SEQ ID NO:23) of human TANGO 180.

FIG. 2 depicts the cDNA sequence (SEQ ID NO:34) and predicted amino acid sequence (SEQ ID NO:54) of murine TANGO 180.

FIG. 3 depicts the cDNA sequence (SEQ ID NO:2) and predicted amino acid sequence (SEQ ID NO:24) of human TANGO 181.

FIG. 4 depicts the partial cDNA sequence (SEQ ID NO:35; partial) and predicted amino acid sequence (SEQ ID NO:55; partial) of murine TANGO 181.

FIG. 5 depicts the cDNA sequence (SEQ ID NO:3) and predicted amino acid sequence (SEQ ID NO:25) of human TANGO 182.

FIG. 6 depicts the partial cDNA sequence (SEQ ID NO:36; partial) and predicted amino acid sequence (SEQ ID NO:56; partial) of murine TANGO 182.

FIG. 7 depicts the cDNA sequence (SEQ ID NO:4) and predicted amino acid sequence (SEQ ID NO:26) of human TANGO 183.

FIG. 8 depicts the cDNA sequence (SEQ ID NO:37) and predicted amino acid sequence (SEQ ID NO:57) of murine TANGO 183.

FIG. 9 depicts the cDNA sequence (SEQ ID NO:5) and predicted amino acid sequence (SEQ ID NO:27) of human TANGO 184.

FIG. 10 depicts the cDNA sequence (SEQ ID NO:38) and predicted amino acid sequence (SEQ ID NO:58) of murine TANGO 184.

FIG. 11 depicts the cDNA sequence (SEQ ID NO:6) and predicted amino acid sequence (SEQ ID NO:28) of human TANGO 185.

FIG. 12 depicts the cDNA sequence (SEQ ID NO:39) and predicted amino acid sequence (SEQ ID NO:59) of murine TANGO 185.

FIG. 13 depicts the cDNA sequence (SEQ ID NO:7) and predicted amino acid sequence (SEQ ID NO:29) of human TANGO 186.

FIG. 14 depicts the cDNA sequence (SEQ ID NO:40) and predicted amino acid sequence (SEQ ID NO:60) of murine TANGO 186.

FIG. 15 depicts the cDNA sequence (SEQ ID NO:8) and predicted amino acid sequence (SEQ ID NO:30) of human TANGO 188.

FIG. 16 depicts the cDNA sequence (SEQ ID NO:41) and predicted amino acid sequence (SEQ ID NO:61) of murine TANGO 188.

FIG. 17 depicts the cDNA sequence (SEQ ID NO:9) and predicted amino acid sequence (SEQ ID NO:31) of human TANGO 189.

FIG. 18 depicts the cDNA sequence (SEQ ID NO:42) and predicted amino acid sequence (SEQ ID NO:62) of murine TANGO 189.

FIG. 19 depicts the cDNA sequence (SEQ ID NO:10) and predicted amino acid sequence (SEQ ID NO:32) of human TANGO 215.

FIG. 20 depicts the cDNA sequence (SEQ ID NO:11) and predicted amino sequence of human TANGO 187-1/3 (SEQ ID NO:22).

FIG. 21 depicts the cDNA sequence (SEQ ID NO:43; partial) and predicted amino acid sequence of murine TANGO 187 (SEQ ID NO:63; partial).

FIG. 22 depicts an alignment of the predicted amino acid sequences of human (SEQ ID NO:23) and murine (SEQ ID NO:54) TANGO 180.

FIG. 23 depicts an alignment of the predicted amino acid sequences of human (SEQ ID NO:24) and murine (SEQ ID NO:55; partial) TANGO 181.

FIG. 24 depicts an alignment of the predicted amino acid sequences of human (SEQ ID NO:25) and murine (SEQ ID NO:5; partial). TANGO 182.

FIG. 25 depicts an alignment of the predicted amino acid sequences of human (SEQ ID NO:26) and murine (SEQ ID NO:57) TANGO 183.

FIG. 26 depicts an alignment of the predicted amino acid sequences of human (SEQ ID NO:27) and murine (SEQ ID NO:58) TANGO 184.

FIG. 27 depicts an alignment of the predicted amino acid sequences of human (SEQ ID NO:28) and murine (SEQ ID NO:59) TANGO 185.

FIG. 28 depicts an alignment of the predicted amino acid sequences of human (SEQ ID NO:29) and murine (SEQ ID NO:60) TANGO 186.

FIG. 29 depicts an alignment of the predicted amino acid sequences of human (SEQ ID NO:30) and murine (SEQ ID NO:61) TANGO 188.

FIG. 30 depicts an alignment of the predicted amino acid sequences of human (SEQ ID NO:31) and murine (SEQ ID NO:62) TANGO 189.

FIG. 31 depicts an alignment of the predicted amino acid sequences of human (SEQ ID NO:33) and murine (SEQ ID NO:63; partial) TANGO 187.

FIG. 32 depicts an alignment of the cDNA sequences of human (SEQ ID NO:1) and murine (SEQ ID NO:34) TANGO 180.

FIG. 33 depicts an alignment of the cDNA sequences of human (SEQ ID NO:2) and murine (SEQ ID NO:35; partial) TANGO 181.

FIG. 34 depicts an alignment of the cDNA sequences of human (SEQ ID NO:3) and murine (SEQ ID NO:36; partial) TANGO 182.

FIG. 35 depicts an alignment of the cDNA sequences of human (SEQ ID NO:4) and murine (SEQ ID NO:37) TANGO 183.

FIG. 36 depicts an alignment of the cDNA sequences of human (SEQ ID NO:5) and murine (SEQ ID NO:38) TANGO 184.

FIG. 37 depicts an alignment of the cDNA sequences of human (SEQ ID NO:6) and murine (SEQ ID NO:39) TANGO 185.

FIG. 38 depicts an alignment of the cDNA sequences of human (SEQ ID NO:7) and murine (SEQ ID NO:40) TANGO 186.

FIG. 39 depicts an alignment of the cDNA sequences of human (SEQ ID NO:8) and murine (SEQ ID NO:41) TANGO 188.

FIG. 40 depicts an alignment of the cDNA sequences of human (SEQ ID NO:9) and murine (SEQ ID NO:42) TANGO 189.

FIG. 41 depicts an alignment of the cDNA sequences of human (SEQ ID NO:11) and murine (SEQ ID NO:43; partial) TANGO 187.

FIG. 42 depicts an alignment of the amino acid sequences of human TANGO 181 (SEQ ID NO:24), murine TANGO 181 (SEQ ID NO:55; partial), human TANGO 182 (SEQ ID NO:25), and murine TANGO 182 (SEQ ID NO:56; partial).

FIG. 43 depicts an alignment of the amino acid sequences of human TANGO 184 (SEQ ID NO:27) and human TANGO 183 (SEQ ID NO:26).

FIG. 44 depicts an alignment of the amino acid sequences of murine TANGO 184 (SEQ ID NO:58) and murine TANGO 183 (SEQ ID NO:57).

FIG. 45 depicts and alignment of the amino acid sequences of human TANGO 180 (SEQ ID NO:23), murine TANGO 180 (SEQ ID NO:54), agkistrodon PLA2 (SQ ID NO:109), acanthahis PLA2 (SEQ ID NO:110), and bovine PLA2 (SEQ ID NO:111).

FIG. 46 depicts the cDNA sequence (SEQ ID NO:______) and predicted amino acid sequence (SEQ ID NO:______) of TANGO 187-1.

FIG. 47 depicts the cDNA sequence (SEQ ID NO:______) and predicted amino acid sequence (SEQ ID NO:______) of TANGO 187-2/3.

FIG. 48 depicts the cDNA sequence (SEQ ID NO:______) and predicted amino acid sequence (SEQ ID NO:______) of TANGO 187-1/2/3.

FIG. 49 depicts the cDNA sequence (SEQ ID NO:______) and predicted amino acid sequence (SEQ ID NO:______) of TANGO 187-1/2.

FIG. 50 depicts the cDNA sequence (SEQ ID NO:______) and predicted amino acid sequence (SEQ ID NO:______) of TANGO 187-2.

FIG. 51 depicts the cDNA sequence (SEQ ID NO:______) and predicted amino acid sequence (SEQ ID NO:______) of TANGO 187-3.

FIG. 52 depicts the cDNA sequence (SEQ ID NO:______) and predicted amino acid sequence (SEQ ID NO:______) of TANGO 187.

FIG. 53 depicts a complete cDNA sequence (SEQ ID NO:______) and predicted amino acid sequence (SEQ ID NO:______) of murine TANGO 181.

FIG. 54 depicts a complete cDNA sequence (SEQ ID NO:______) and predicted amino acid sequence (SEQ ID NO:______) of murine TANGO 182.

FIG. 55 depicts a complete cDNA sequence (SEQ ID NO:______) and predicted amino acid sequence (SEQ ID NO:______) of murine TANGO 187.

FIG. 56 depicts a complete cDNA sequence (SEQ ID NO:______) and predicted amino acid sequence (SEQ ID NO:______) of murine TANGO 215.

### DETAILED DESCRIPTION OF THE INVENTION

The present invention is based on the discovery of cDNA molecules encoding TANGO 180, TANGO 181, TANGO 182, TANGO 183, TANGO 184, TANGO 185, TANGO 186, TANGO 188, TANGO 189, TANGO 215, and TANGO 187, all of which are predicted to be either wholly secreted or transmembrane proteins.

TANGO 180

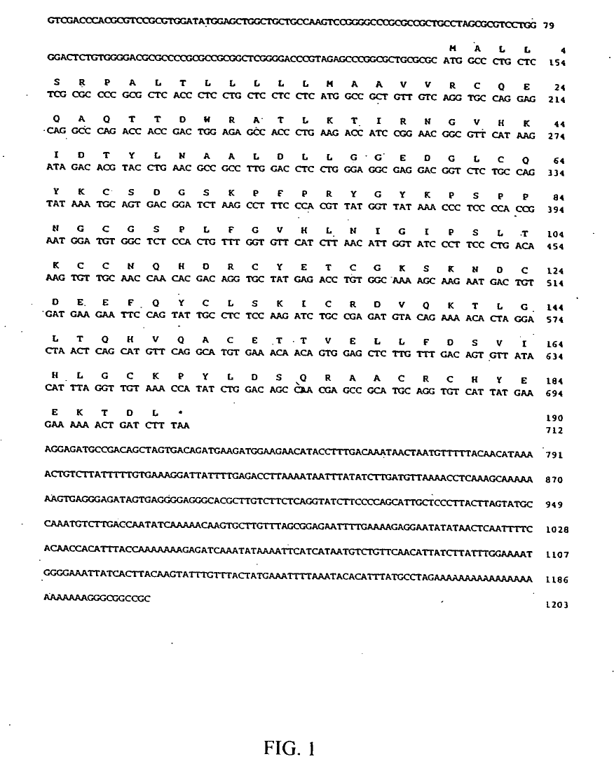

The human TANGO 180 cDNA of SEQ ID NO:1 has a 567 nucleotide open reading frame (SEQ ID NO:12) encoding a 189 amino acid protein (SEQ ID NO:23). The cDNA and protein sequences of human TANGO 180 are shown in FIG. 1.

Human TANGO 180 is predicted to be a wholly secreted protein having a 22 amino acid signal sequence (amino acids 1-22 of SEQ ID NO:23; SEQ ID NO:64) followed by a 167 amino acid mature protein (amino acids 23-189 of SEQ ID NO:23; SEQ ID NO:76). TANGO 180 is predicted to have a molecular weight of 21.0 kDa prior to cleavage of its signal peptide and a molecular weight of 18.5 kDa subsequent to cleavage of its signal peptide.

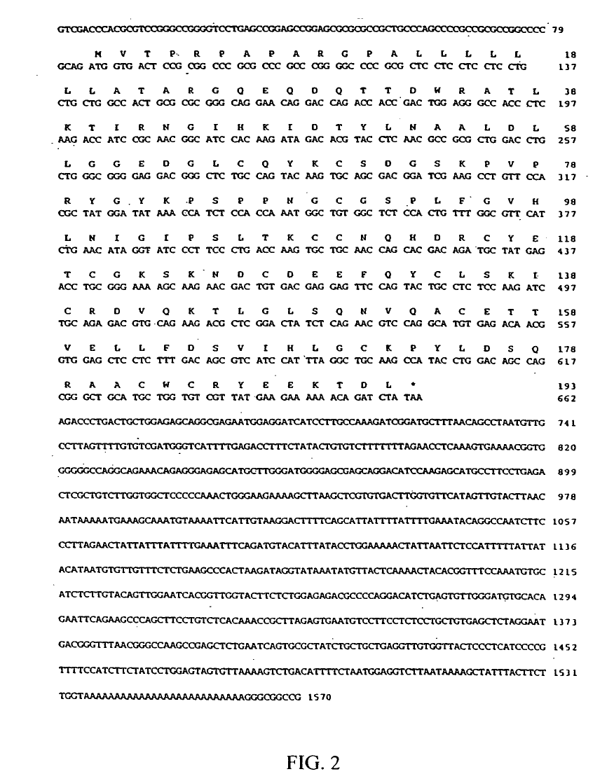

The murine TANGO 180 of SEQ ID NO:34 has a 576 nucleotide open reading frame (SEQ ID NO:44) encoding a 192 amino acid protein (SEQ ID NO:54). The cDNA and protein sequences of murine TANGO 180 are shown in FIG. 2.

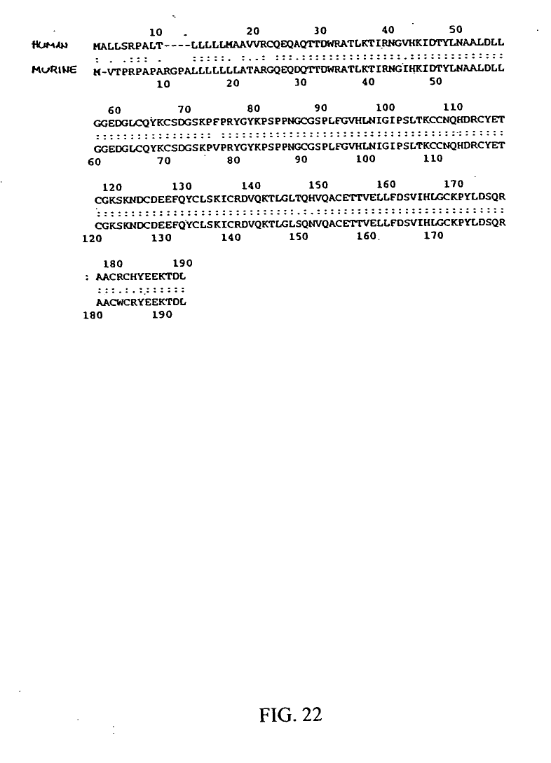

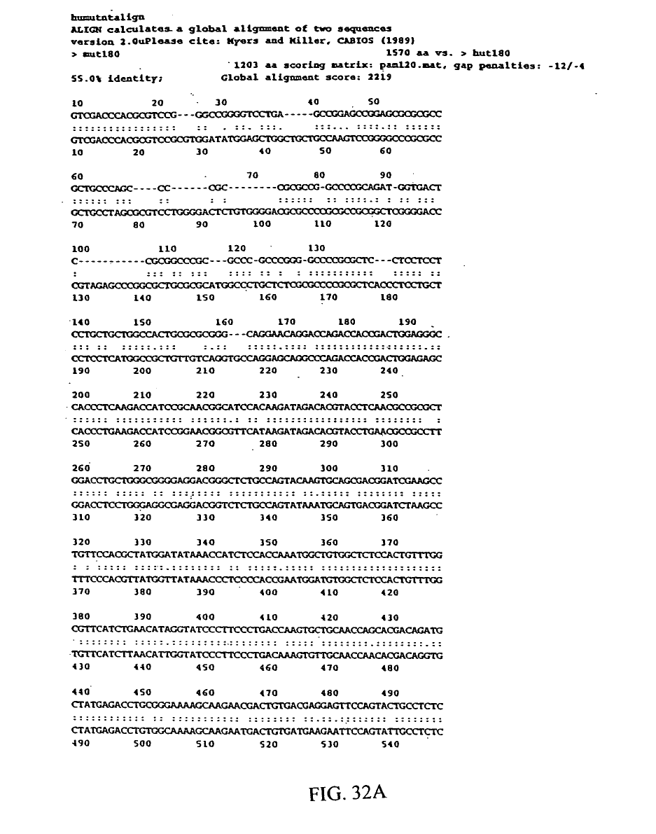

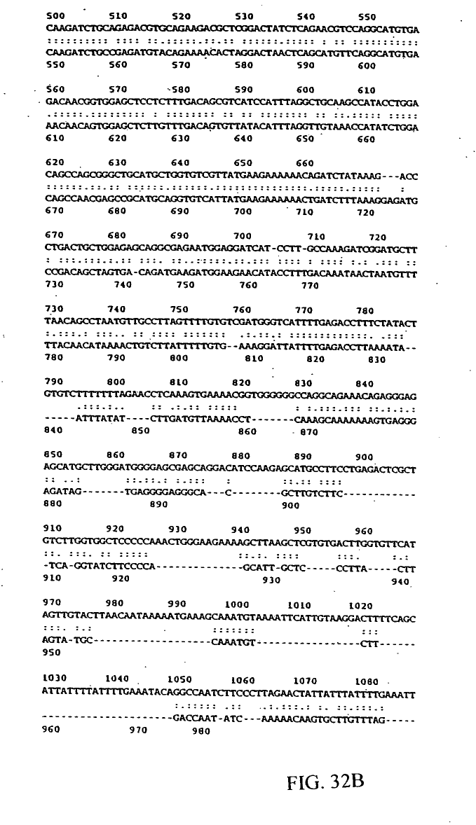

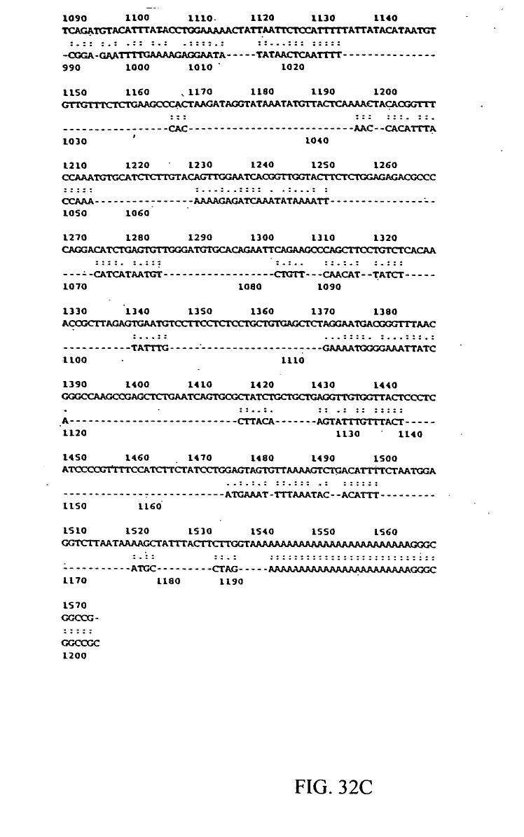

FIG. 22 depicts an alignment of the predicted amino acids sequences of human (SEQ ID NO:23) and murine (SEQ ID NO:54) TANGO 180 (88.7% identity). FIG. 32 depicts an alignment of the cDNA sequences of human (SEQ ID NO:1) and murine (SEQ ID NO:34) TANGO 180 (55% identity).

Northern analysis of human TANGO 180 mRNA expression revealed the presence of two major transcripts (1.3 and 5.25 kb) and three minor transcripts (0.95, 1.8, and 4.15 kb). This analysis also revealed that all five transcripts are expressed at a low level in placenta, lung, and liver; that the 1.3 and the 5.25 kb transcripts are expressed at a moderate level in brain and kidney; that the 5.25 kb transcript is expressed at a moderate level in heart, skeletal muscle, and pancreas; and that the 1.3 kb transcript is expressed at a high level in heart, skeletal muscle, and pancreas.

In situ expression analysis of TANGO 180 in adult murine tissue revealed no significant expression in bladder, pancreas, heart, thymus, kidney, brain, colon, placenta, eye, liver, spleen, lung, skeletal muscle/diaphram, or small intestine. In situ expression analysis of murine embryonic tissue revealed expression in the liver at E13.5 through E16.5. Liver expression was also observed, although at a lower level, at E17.5 and P1.5.

TANGO 180 maps to human chromosome location 4q25.

TANGO 180 is predicted to have a phospholipase A2 histidine active site domain at amino acids 106-113 of SEQ ID NO:23 and a phospholipase A2 aspartic acid active site-like domain at amino acids 124-131 of SEQ ID NO:23.

An apparent genomic sequence of TANGO 180 appears at GenBank Accession Number AC004067.

Human TANGO 180 bears some similarity to a number of C. elegans proteins.

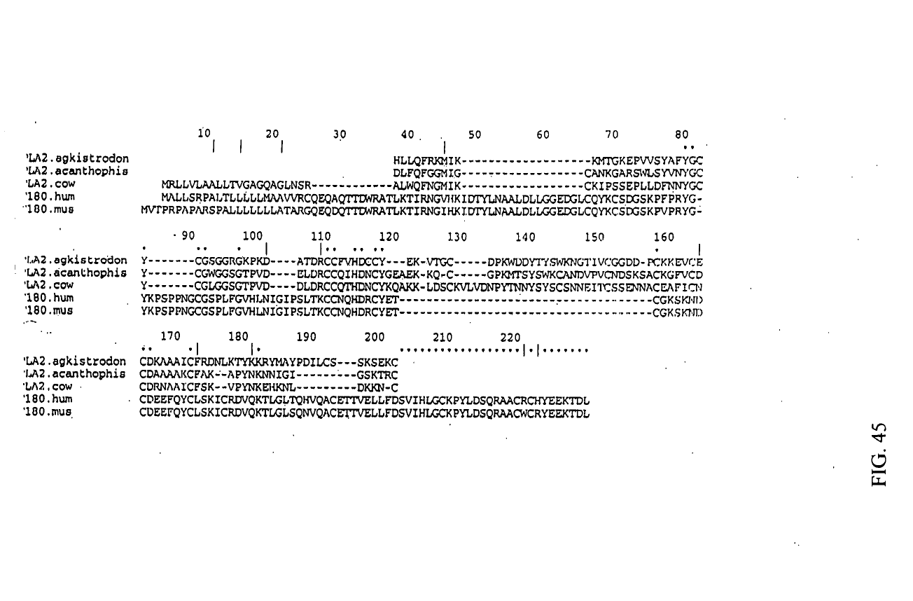

TANGO 180 bears some similarity to a number of known phospholipase A2 (PLA2) proteins (Lambeau et al. (1994) J. Biol. Chem. 269:1575-78; Lambeau et al. (1995) J. Biol. Chem. 270:5534-40). TANGO 180 may play a role similar to that of a phospholipase A2. FIG. 45 depicts and alignment of the amino acid sequences of human TANGO 180 (SEQ ID NO:23), murine TANGO 180 (SEQ ID NO:54), agkistrodon PLA2 (SQ ID NO:109), acanthahis PLA2 (SEQ ID NO:110), and bovine PLA2 (SEQ ID NO:111). There are thought to be at least two important regions within many PLA2's: CCXXHCCX (hisitidine at active site) and LIVMACLIVMFYWPCSTCDXXXXXC (aspratic acid active site). Various phospholipase A2 proteins are thought to be involved in inflammation. Moreover, it appears that the expression and synthesis of at least some phospholipase A2 proteins are induced by pro-inflammatory modulators such as interleukin-1, interleukin-6, and tumor necrosis factor. Thus, TANGO 180 may be involved in inflammation, e.g., arthritis, endotoxic shock, peritonitis, psoriasis, acute pancreatitis, and respiratory distress syndrome. Accordingly, TANGO 180 nucleic acid molecules and polypeptides as well as anti-TANGO 180 antibodies and modulators of TANGO 180 expression or activity may be useful in the treatment of such disorders. Moreover, PLA2's have been implicates in digestion, airway contraction, smooth muslce contraction, fertilization, and cell proliferation. Thus, TANGO 180 nucleic acid molecules and polypeptides as well as anti-TANGO 180 antibodies and modulators of TANGO 180 expression or activity may be useful in the treatment of disorders of digestion, airway contraction, smooth muslce contraction, fertilization, and cell proliferation.

TANGO 181

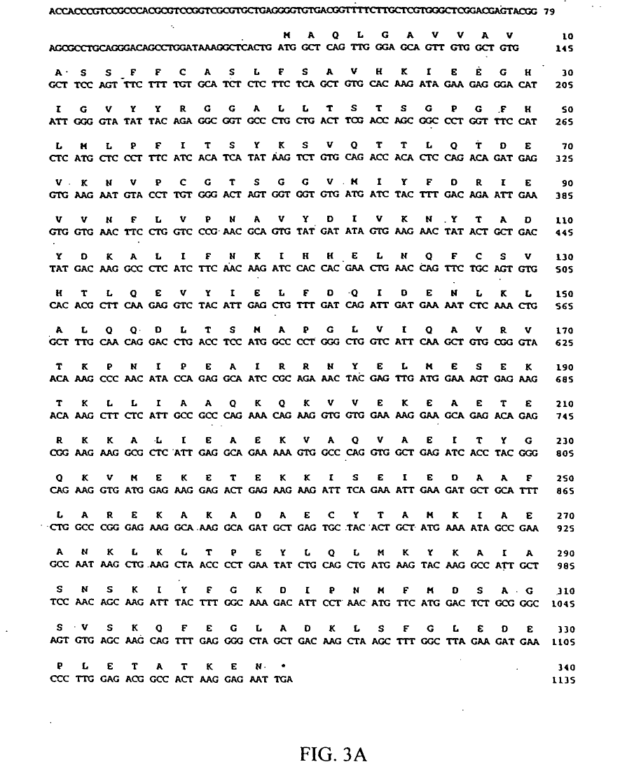

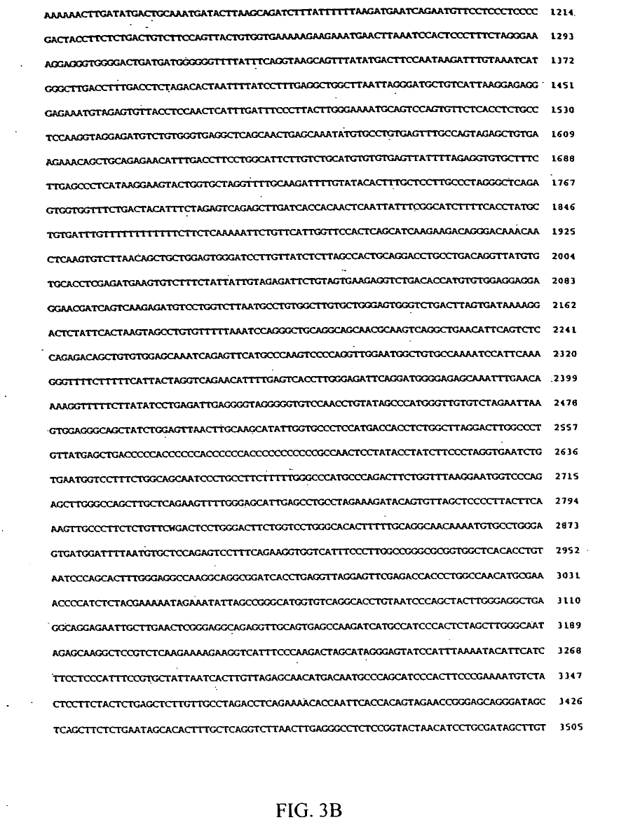

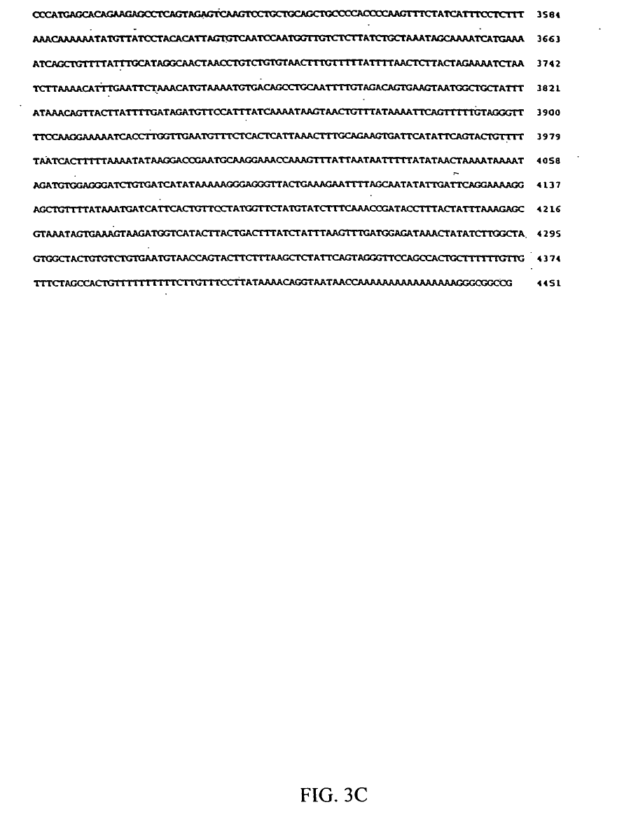

The human TANGO 181 cDNA of SEQ ID NO:2 has a 1017 nucleotide open reading frame (SEQ ID NO:12) encoding a 339 amino acid protein (SEQ ID NO:23). The cDNA and protein sequences of human TANGO 181 are shown in FIG. 3.

Human TANGO 181 is predicted to be a secreted protein having a 22 amino acid signal sequence (amino acids 1-22 of SEQ ID NO:24; SEQ ID NO:65) followed by a 317 amino acid mature protein (amino acids 23-339 of SEQ ID NO:24; SEQ ID NO:77). TANGO 181 is predicted to have a molecular weight of 37.8 kDa prior to cleavage of its signal peptide and a molecular weight of 35.2 subsequent to cleavage of its signal peptide.

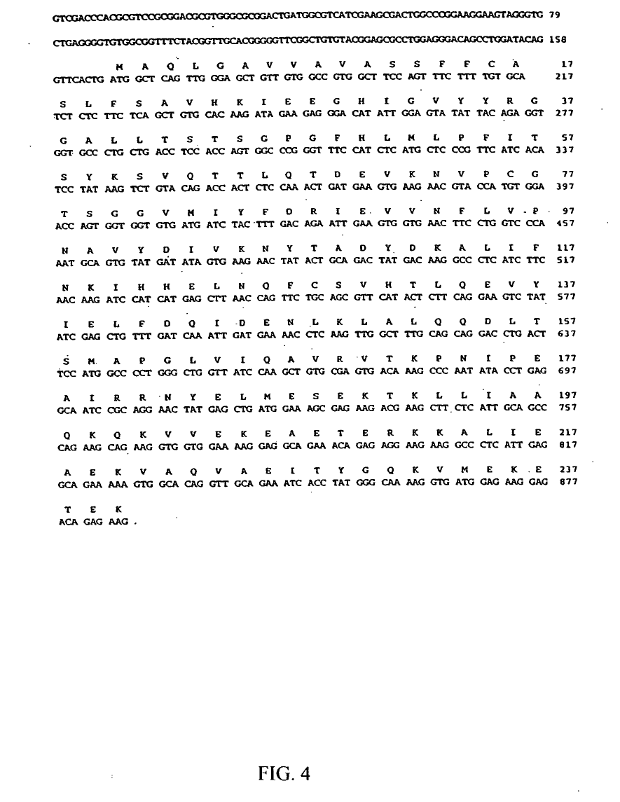

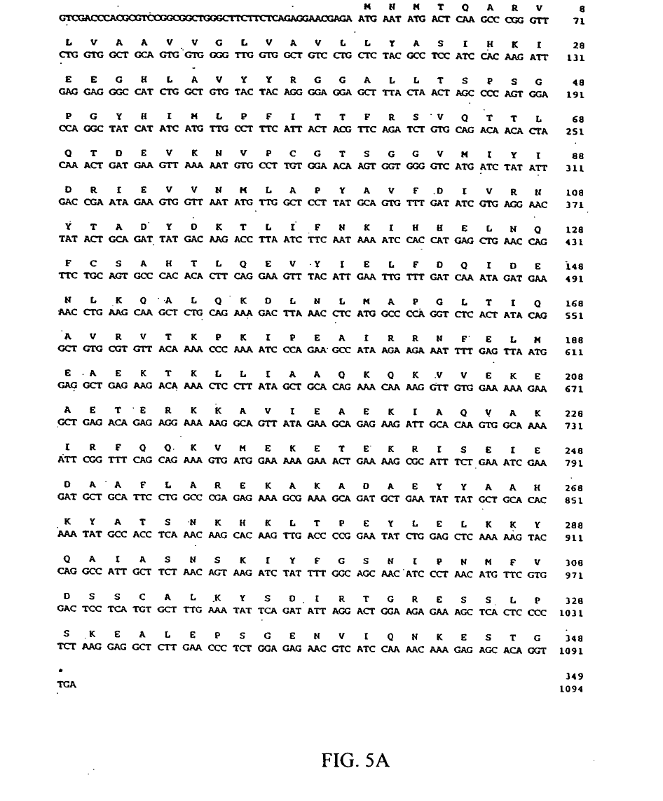

The murine TANGO 181 partial cDNA of SEQ ID NO:35 has a 747 nucleotide open reading frame (SEQ ID NO:45) encoding a 249 amino acid protein (SEQ ID NO:55). The partial cDNA and protein sequences of murine TANGO 181 are shown in FIG. 4.

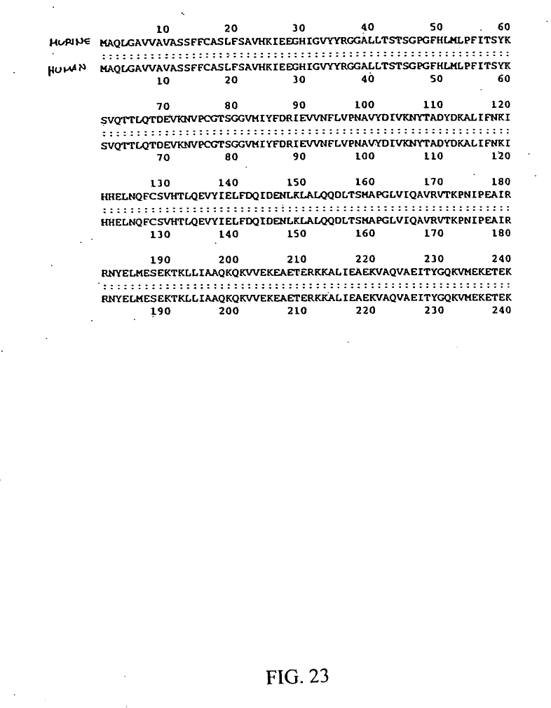

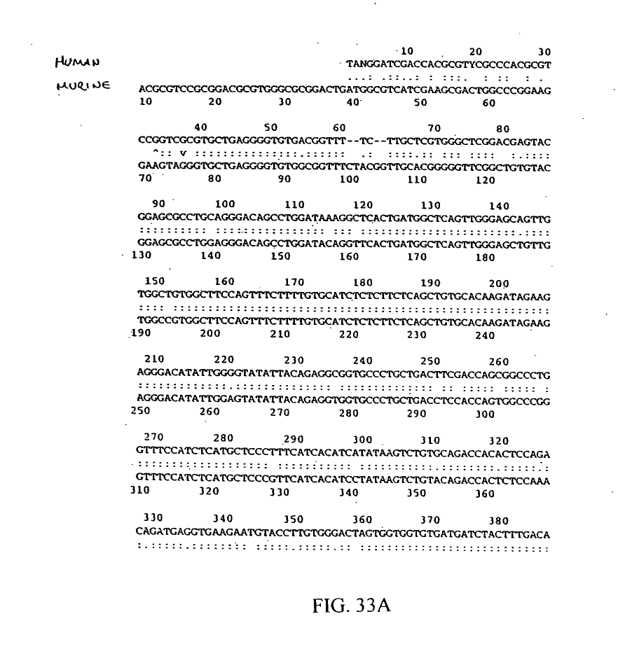

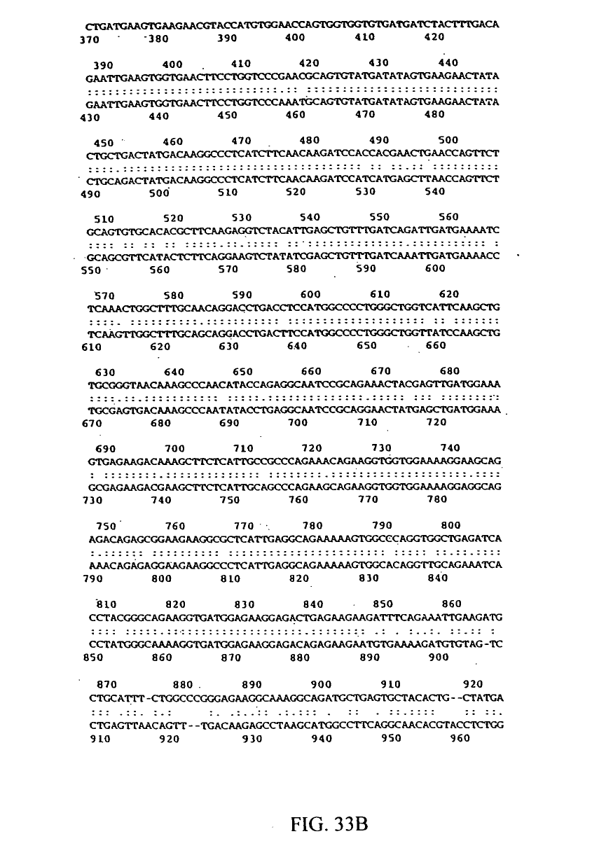

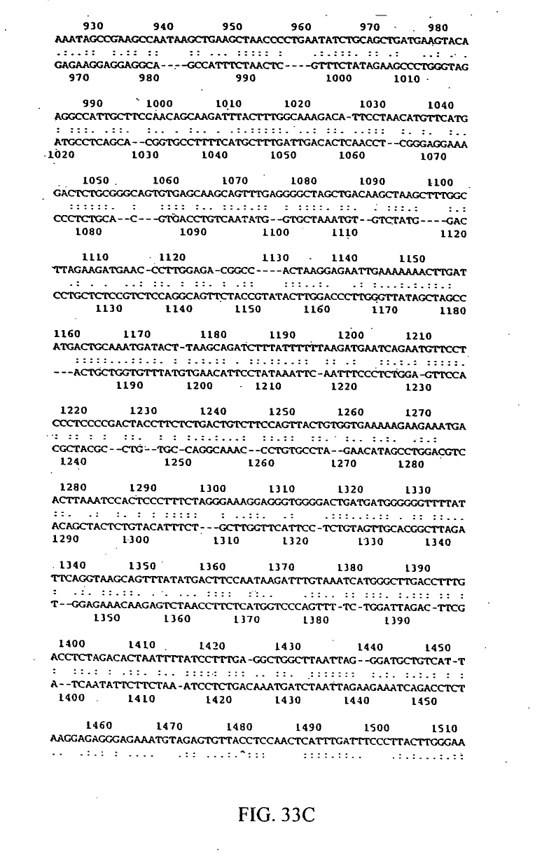

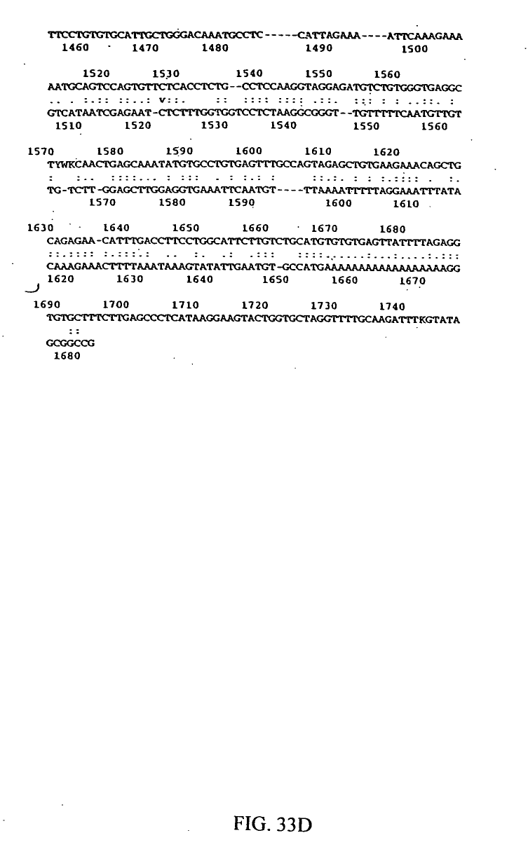

FIG. 23 depicts an alignment of the predicted amino acids sequences of human (SEQ ID NO:24) and murine (SEQ ID NO:55; partial) TANGO 181 (72.1% identity). FIG. 33 depicts an alignment of the cDNA sequences of human (SEQ ID NO:2) and murine (SEQ ID NO:35; partial) TANGO 181 (65.4% identity). The pair of cysteines at amino acids 76 and 129 might be important for disulfide bond formation. The single cysteine at amino acid 262 might enable TANGO 181 to form homodimers (or heterodimers with TANGO 182).

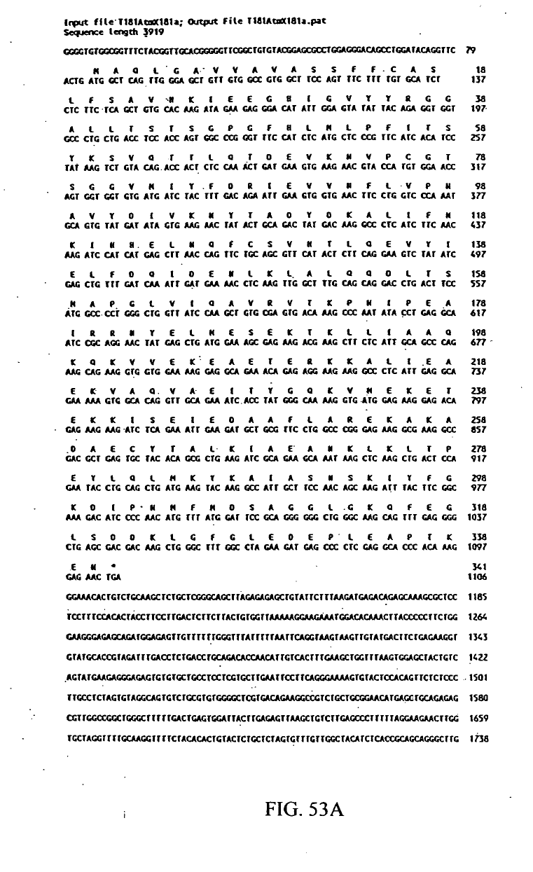

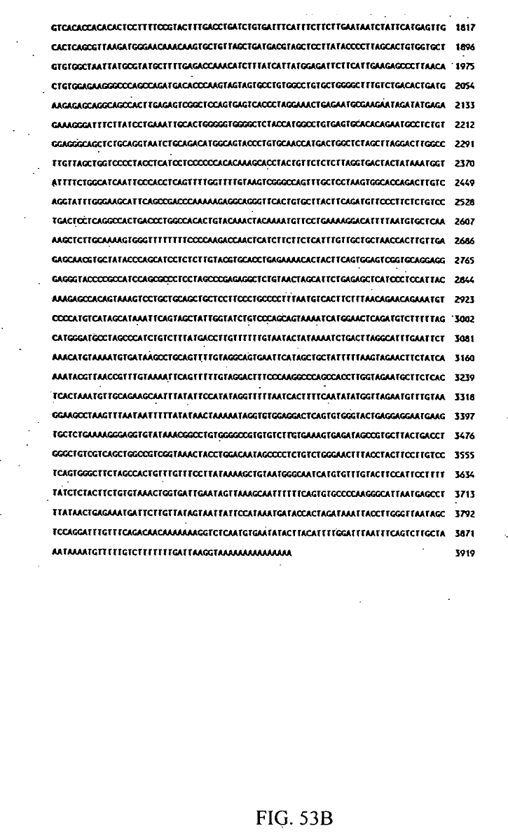

The cDNA sequence (SEQ ID NO:______) and predicted amino acid sequence (SEQ ID NO:______) of a full-length murine TANGO 181 clone are shown in FIG. 53.

Northern analysis of human TANGO 181 mRNA expression revealed the presence of two transcripts (4.3 and 4.5 kb) expressed at a low level in heart, brain, placenta, lung, liver, skeletal muscle, kidney, and pancreas, with the level of expression in the pancreas being higher than in the other tissues.

Murine in situ expression analysis revealed that TANGO 181 is weakly expressed in adult brain (choroid plexus and olfactory bulb). This analysis also revealed TANGO 180 expression in the liver and kidney (medulla). High level TANGO 180 expression was observed in testis. This analysis detected little or no expression of TANGO 181 in adult liver, ovary, heart, lung, spleen, fat, muscle, skin, stomach, duodenum, colon, pancreas, thymus, pituitary, and eye. In situ expression analysis of embryos revealed that TANGO 181 is ubiquitously expressed at stages E12.5, E13.5, and E14.5.

TANGO 181 maps to human chromosome location 8p12. WI-5768 and AFMB057WG5 are markers which flank TANGO 181. Nearby loci include WRN (Werner Syndrome) and SPG5A (Spastic Paraplegia 5A), and nearby known genes include FGFR1 (fibroblast growth factor receptor), STAR (Steroidogenic acute regulatory protein), ANK1 (abkyrin 1), CALB1 (calbindin 1), CHRNB3 (cholinergic receptor, nicotinic). The human chromosomal location corresponds to a position on mouse chromosome 8 near fgfri (fibroblast growth factor receptor), cyrn (cyritesin 1), tissue plasminogen activator, and ank (ankyrin 1).

Within the 3′ untranslated region of the human TANGO 181 cDNA described above is a 260 base pair sequence (Genbank Accession Number Z36802) previously identified as part of a gene that appears to be preferentially expressed in pancreatic cancer and chronic pancreatitis (Gress et al. (1996) Oncogene 13:1819-30). Thus, TANGO 181 nucleic acids and polypeptides may be useful for the diagnosis and/or treatment of chronic pancreatitis and pancreatic cancer (as well as other cancers). In addition, modulators of TANGO 181 expression or activity may be useful in the treatment of such disorders.

TANGO 181 and TANGO 182 are highly homologous to teh C. elegans protein C42C1.9

TANGO 182

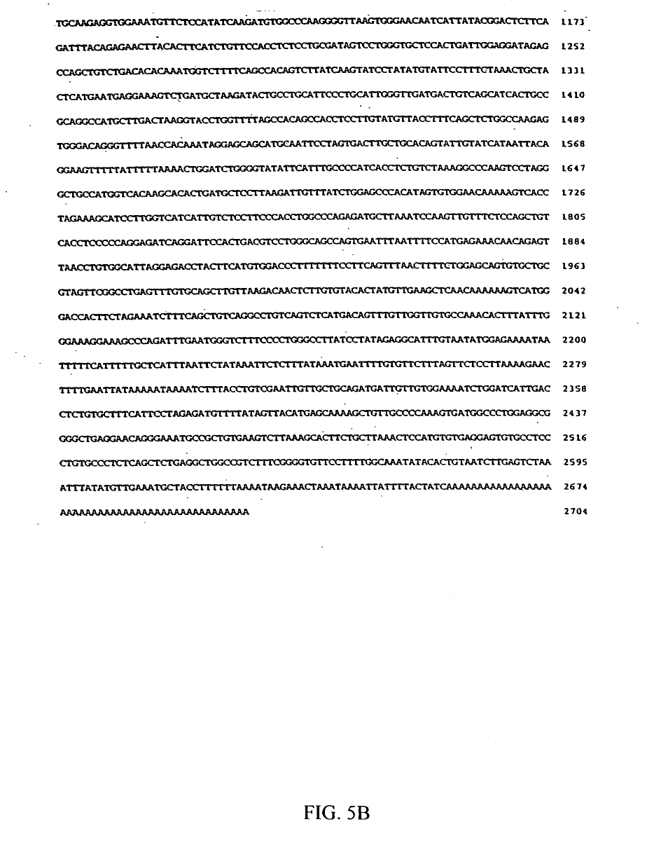

The human TANGO 182 cDNA of SEQ ID NO:3 has a 1044 nucleotide open reading frame (SEQ ID NO:14) encoding a 348 amino acid protein (SEQ ID NO:25). The cDNA and protein sequences of human TANGO 182 are shown in FIG. 5.

Human TANGO 182 is predicted to be a secreted protein having a 23 amino acid signal sequence (amino acids 1-23 of SEQ ID NO:25; SEQ ID NO:66) followed by a 325 amino acid mature protein (amino acids 24-348 of SEQ ID NO:25; SEQ ID NO:78). TANGO 182 is predicted to have a molecular weight of 39.2 kDa prior to cleavage of its signal peptide and a molecular weight of 36.1 kDa subsequent to cleavage of its signal peptide.

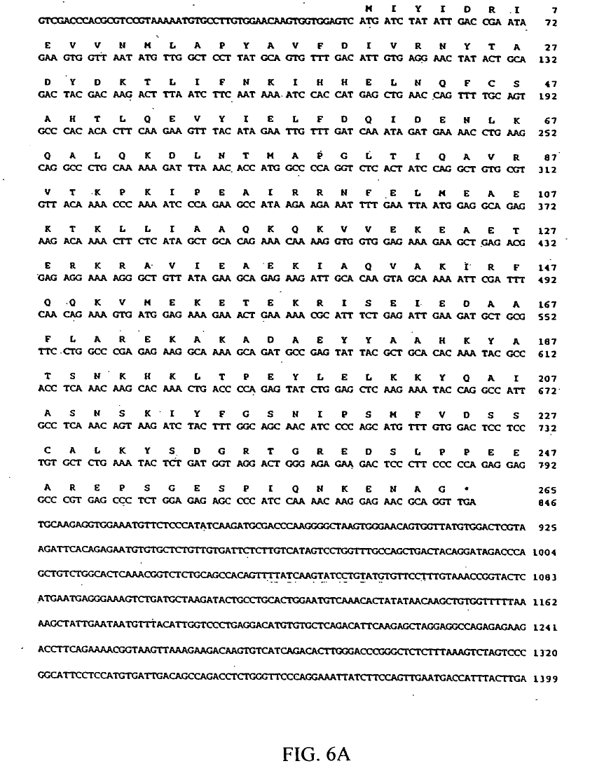

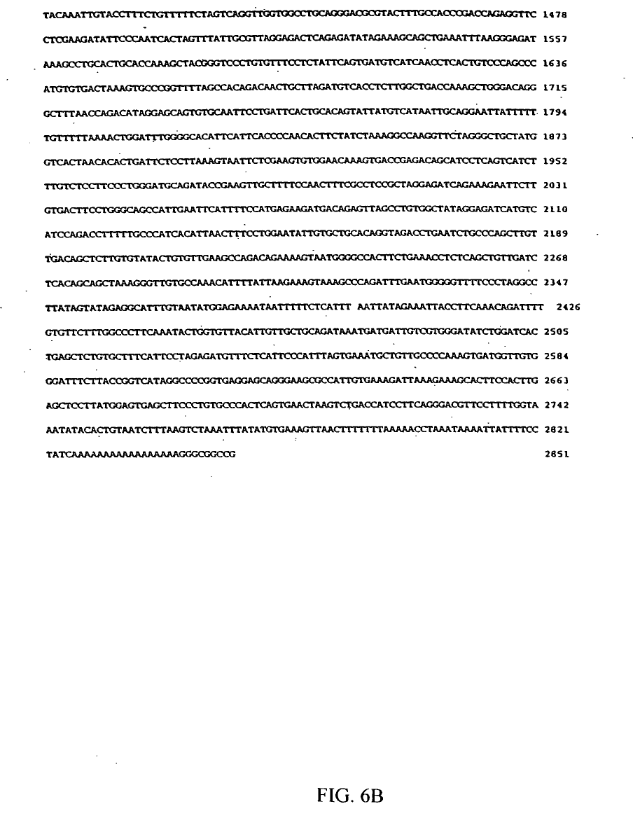

The murine TANGO 182 partial cDNA of SEQ ID NO:36 has an 825 nucleotide open reading frame (SEQ ID NO:46) encoding a 275 amino acid protein (SEQ ID NO:56). The partial cDNA and protein sequences of murine TANGO 182 are shown in FIG. 6.

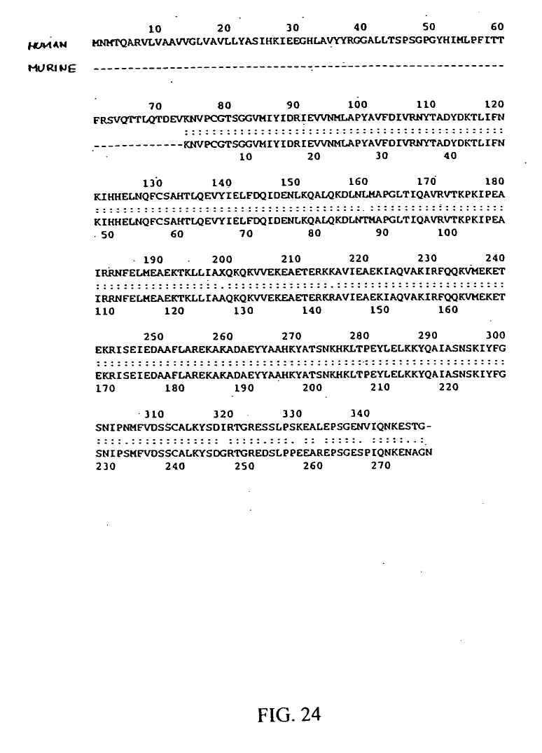

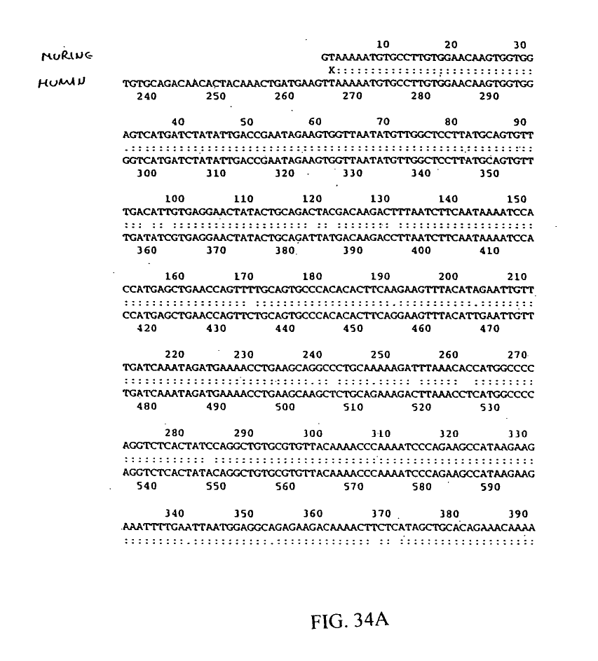

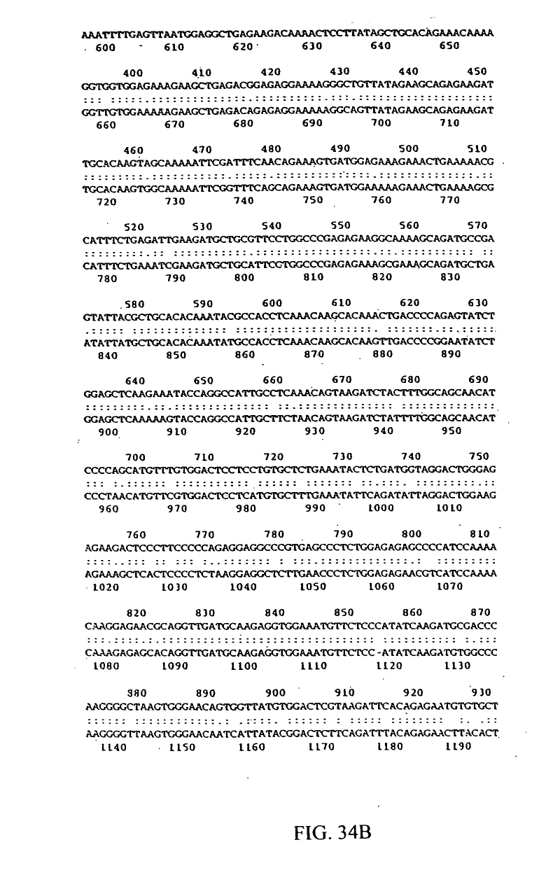

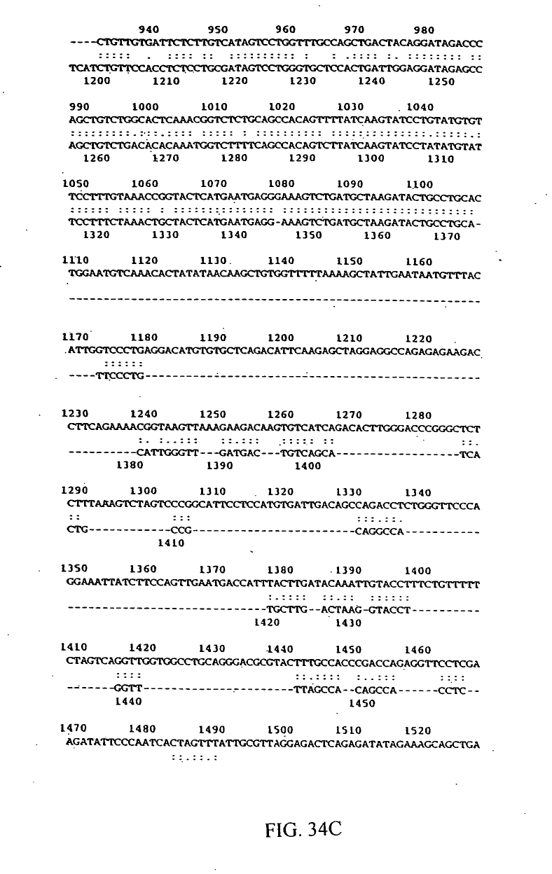

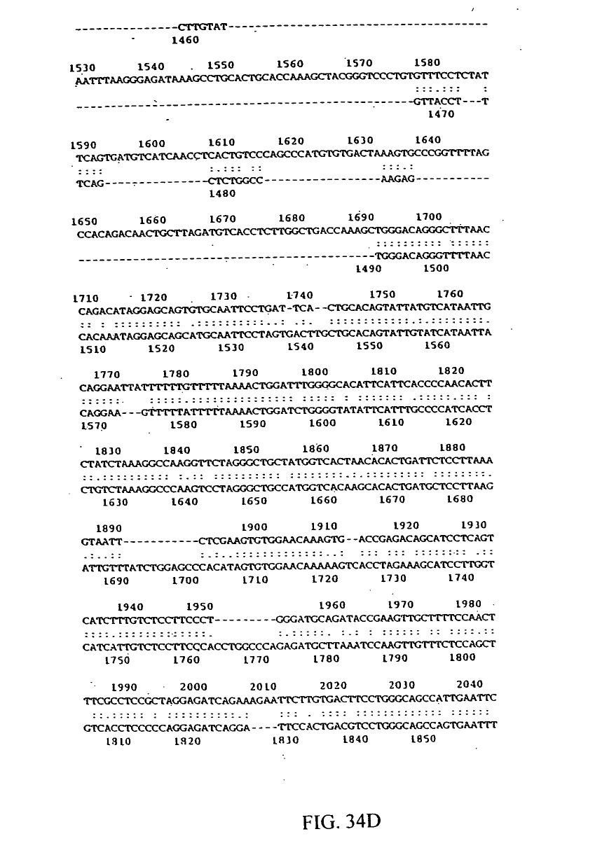

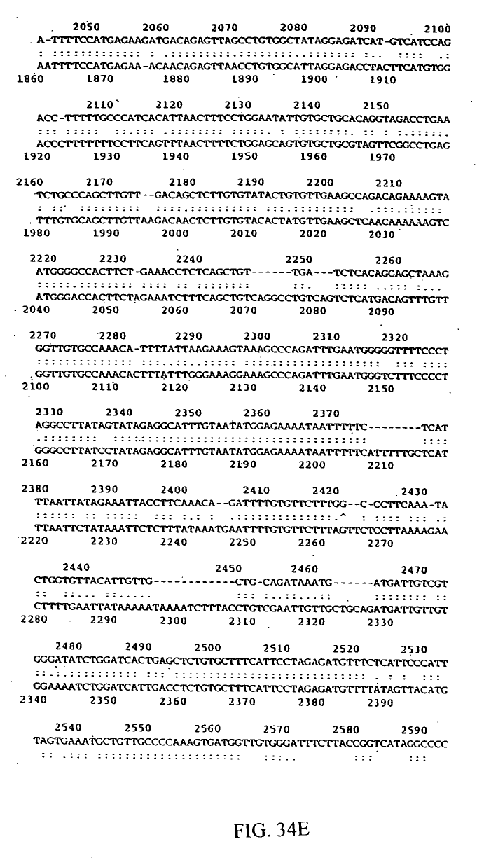

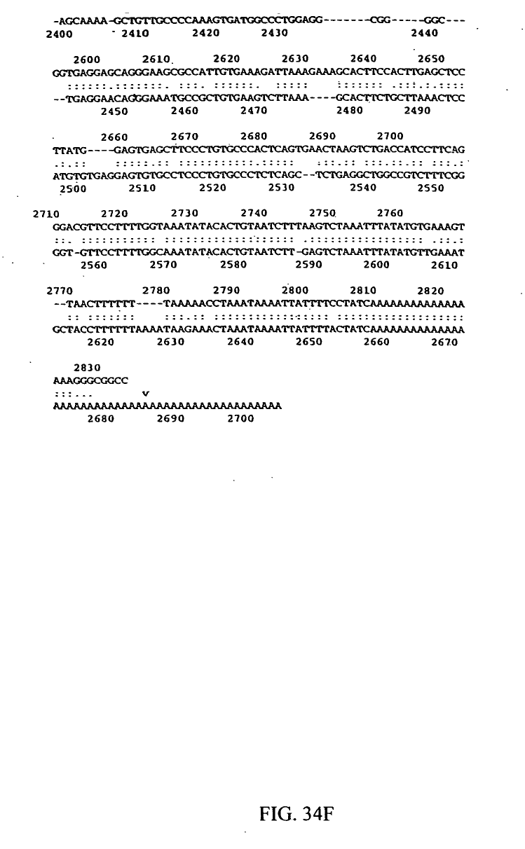

FIG. 24 depicts an alignment of the predicted amino acids sequences of human (SEQ ID NO:25) and murine (SEQ ID NO:56; partial) TANGO 182 (75.1% identity). FIG. 34 depicts an alignment of the cDNA sequences of human (SEQ ID NO:3) and murine (SEQ ID NO:36; partial) TANGO 182 (67.6% identity). The pair of cysteines at amino acids 78 and 130 might be important for disulfide bond formation. The single cysteine at amino acid 312 might enable TANGO 182 to form homodimers (or heterodimers with TANGO 181).

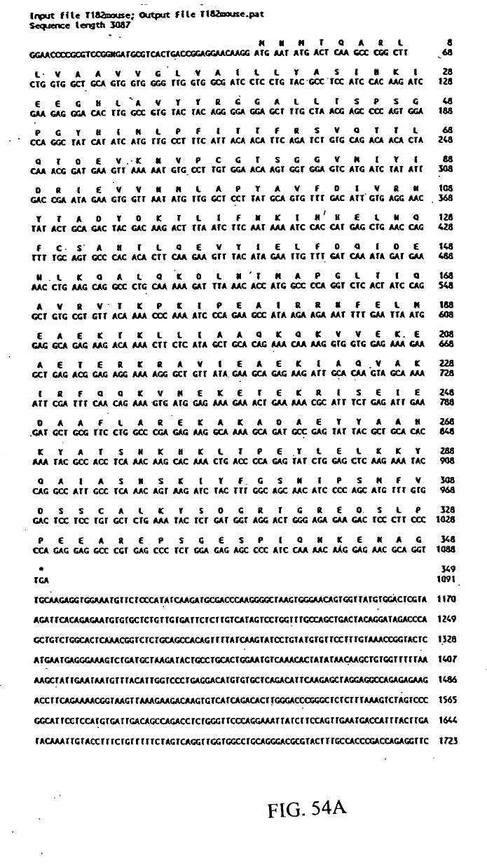

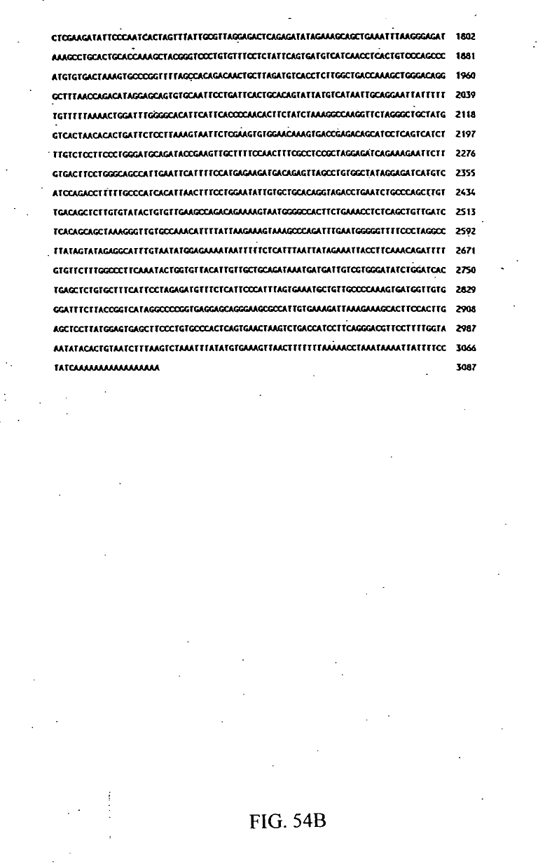

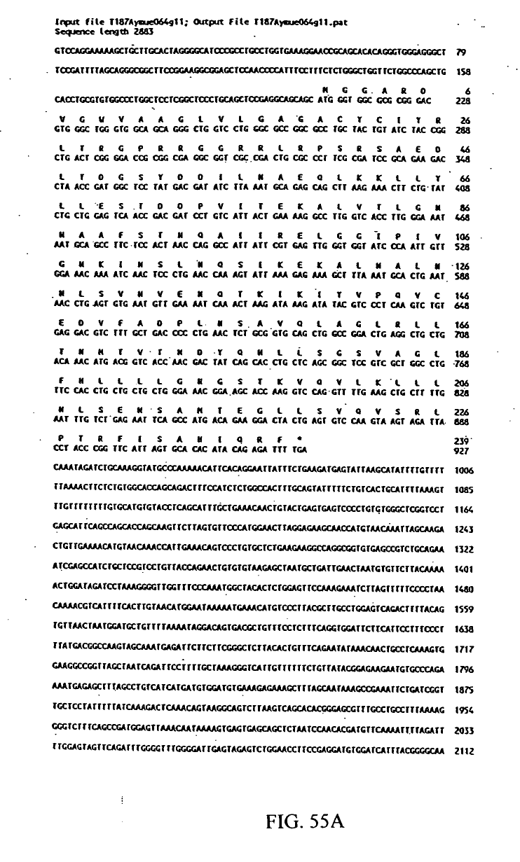

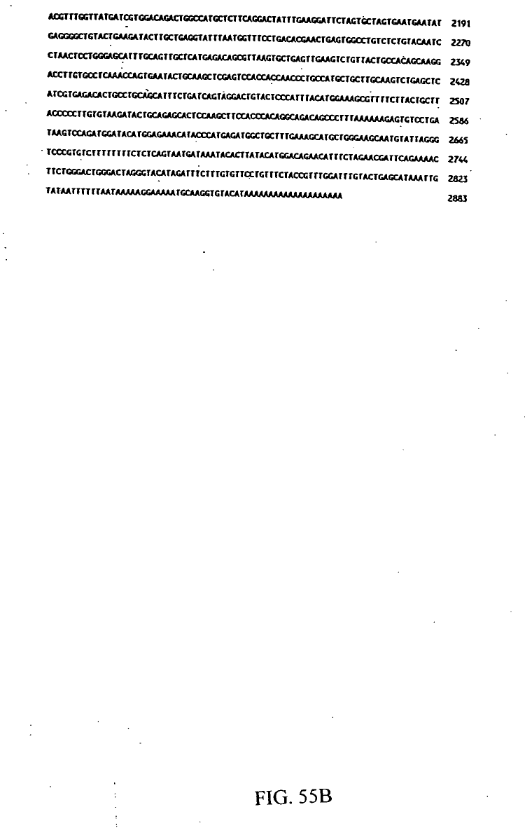

The cDNA sequence (SEQ ID NO:______) and predicted amino acid sequence (SEQ ID NO:______) of a full-length murine TANGO 182 clone are shown in FIG. 54.

TANGO 182 maps to human chromosomal location 10q24 between markers D10S566 and D10S540. In mice, TANGO 182 maps to chromosome 10 bwtween D10S198 and D10S192 (129.8 to 131.2 cM).

Northern analysis of human TANGO 182 mRNA expression revealed the presence of a 2.8 kb transcript that is expressed at a high level placenta and a somewhat lower level in liver, kidney, and pancreas. This transcript is expressed at a low level in heart, brain, lung, and skeletal muscle.

Murine in situ expression analysis revealed that TANGO 182 is expressed at a high level in testis in adult mice. Little or no expression was detected in adult brain, liver, kidney, ovary, heart, lung, spleen, fat, muscle, skin, stomach, duodenum, colon, pancreas, thymus, pituitary, or eye by in situ analysis. In situ expression analysis of embryos revealed ubiquitous, low level expression at stages E12.5, E13.5, and E14.5.

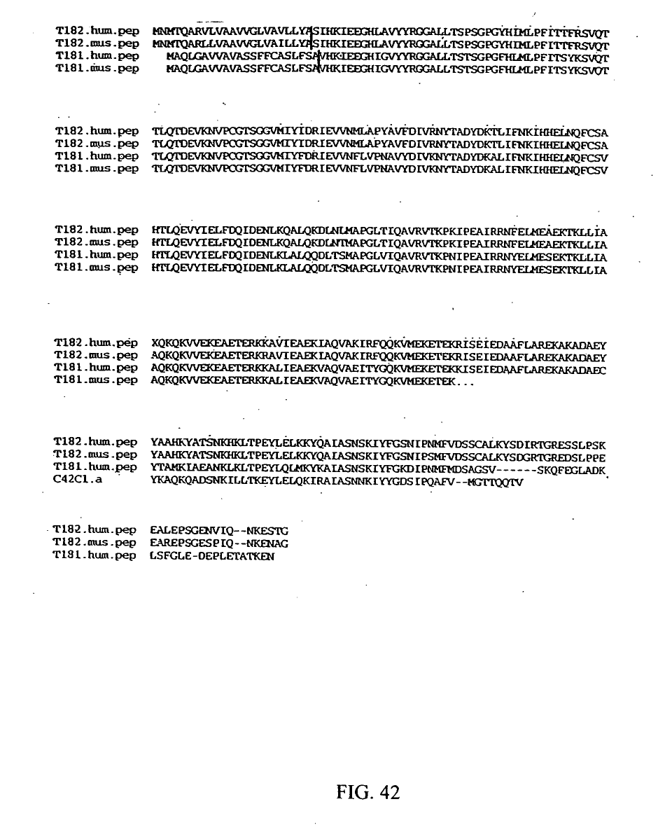

Both human and mouse TANGO 182 are quite similar to human and murine TANGO 181 at the amino acid level (FIG. 42). Thus, TANGO 182, like TANGO 181, may be useful for the diagnosis and/or treatment of pancreatic cancer and chronic pancreatitis as well as other cancers. In addition, TANGO 182 bears some similarity to a C. elegans protein C42C1.9 (Genbank Accession Number AF043695) that is encoded by a gene that is present in the same operon as a gene encoding a mitochondrial carrier protein. Since genes within the same operon are often co-regulated and encode proteins involved in the same physiological state, TANGO 182 may play a role in metabolism. Thus, TANGO 182 nucleic acids and polypeptides as well as antibodies directed against TANGO 182 may be useful in the diagnosis and treatment of metabolic disorders. In addition, modulators of TANGO 182 expression or activity may be useful in the treatment of such disorders.

TANGO 183

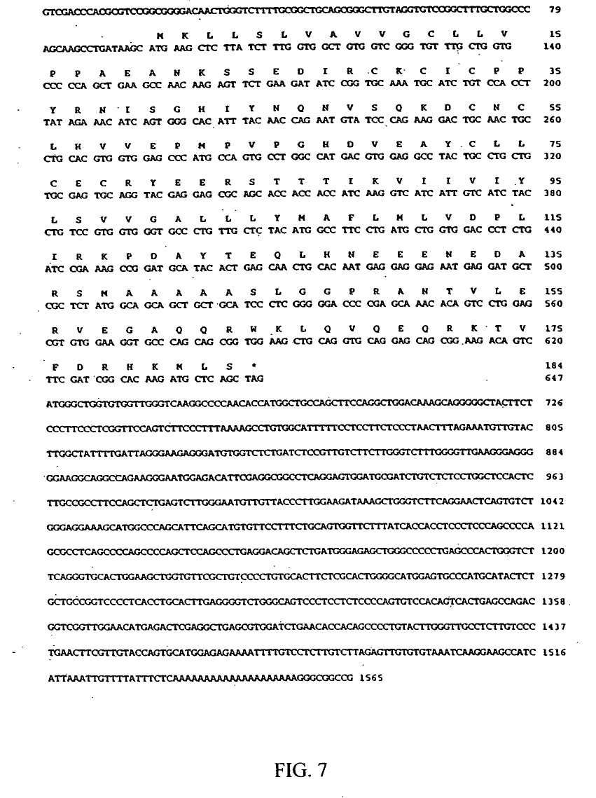

The human TANGO 183 cDNA of SEQ ID NO:4 has a 549 nucleotide open reading frame (SEQ ID NO:15) encoding a 183 amino acid protein (SEQ ID NO:26). The cDNA and protein sequences of human TANGO 183 are shown in FIG. 7.

Human TANGO 183 is predicted to be a transmembrane protein having a 20 amino acid signal sequence (amino acids 1-20 of SEQ ID NO:26; SEQ ID NO:67) followed by a 163 amino acid mature protein (amino acids 21-183 of SEQ ID NO:26; SEQ ID NO:79) having a 69 amino acid extracellular domain (amino acids 21-89 of SEQ ID NO:26; SEQ ID NO:88), a 23 amino acid transmembrane domain (amino acids 90-112 of SEQ ID NO:26; SEQ ID NO:94), and a 71 amino acid cytoplasmic domain (amino acids 113-183 of SEQ ID NO 26; SEQ ID NO: 102). There are 8 conserved cysteines in the extracellular domain. TANGO 183 has a high porportion of charged amino acids in the predicted extracellular (18%, not including histidines) and cytoplasmic (32%) domains. Human TANGO 183 is predicted to have a molecular weight of 20.6 kDa prior to cleavage of its signal peptide and a molecular weight of 18.1 kDa subsequent to cleavage of its signal peptide.

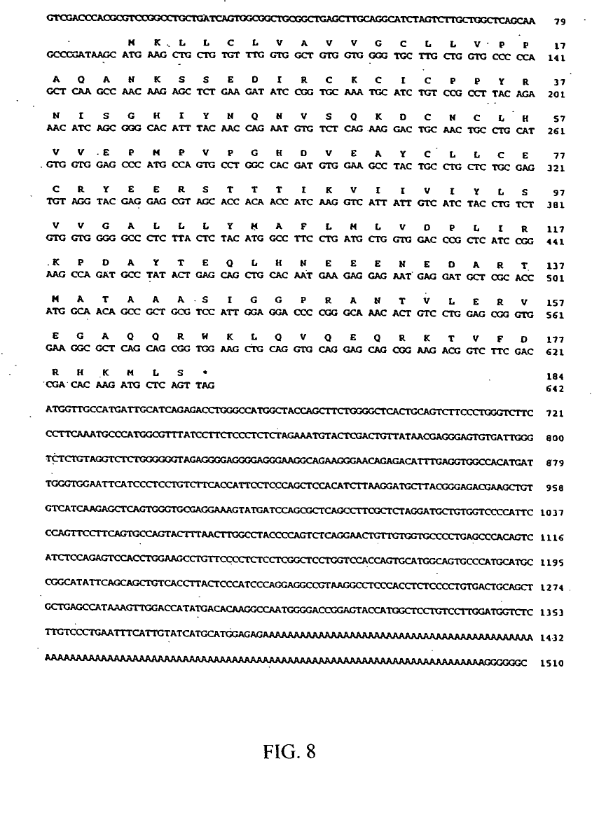

The murine TANGO 183 cDNA of SEQ ID NO:37 has a 549 nucleotide open reading frame (SEQ ID NO:47) encoding a 183 amino acid protein (SEQ ID NO:57). The cDNA and protein sequences of murine TANGO 183 are shown in FIG. 8.

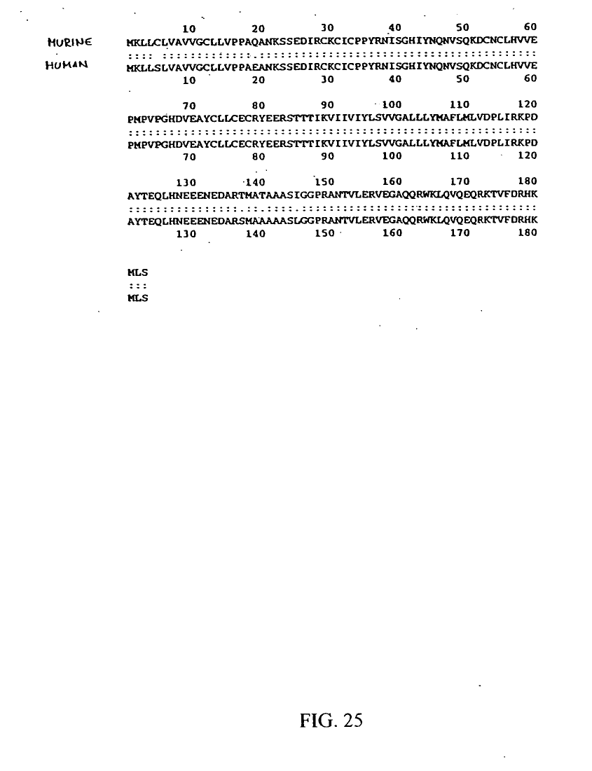

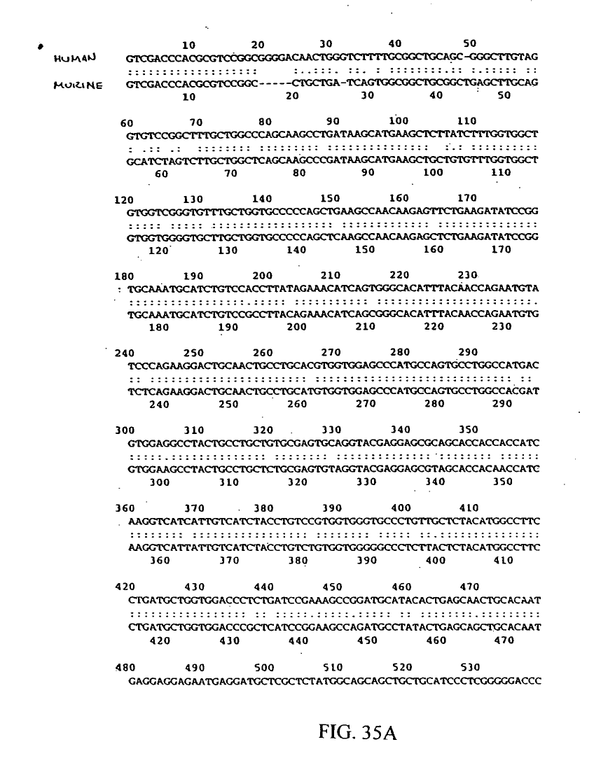

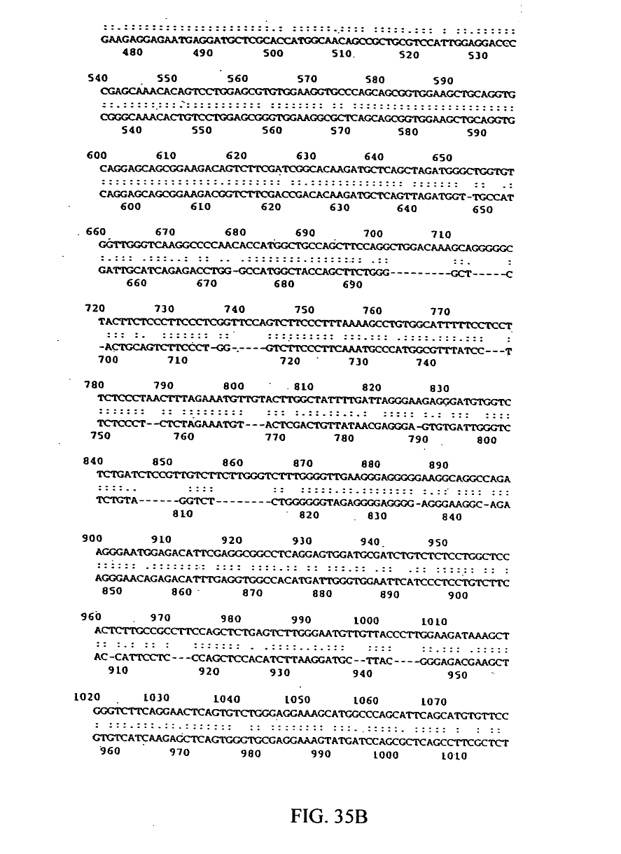

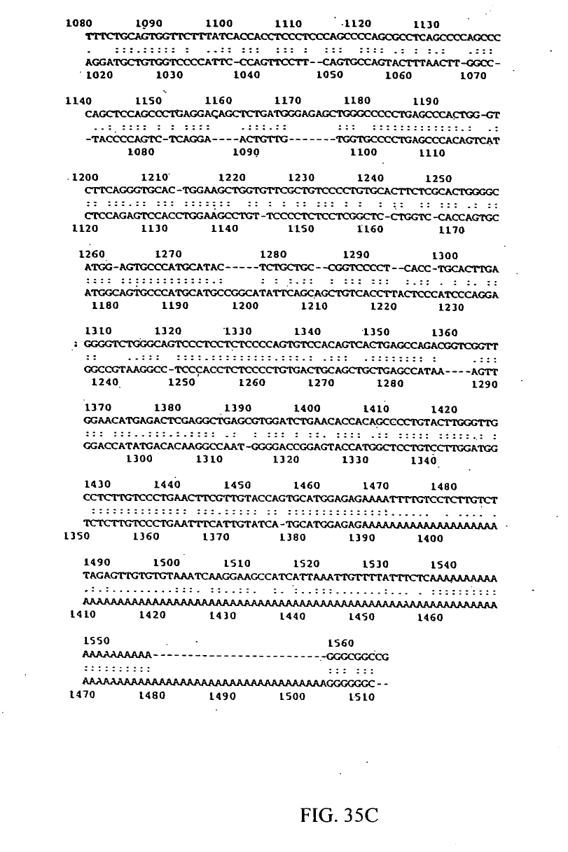

FIG. 25 depicts an alignment of the predicted amino acids sequences of human (SEQ ID NO:26) and murine (SEQ ID NO:57) TANGO 183 (97.3% identity). FIG. 35 depicts an alignment of the cDNA sequences of human (SEQ ID NO:4) and murine (SEQ ID NO:37) TANGO 183 (71.7% identity). The conserved cysteine residues are particularly important and are preferably retained in functional variants.

Northern analysis of human TANGO 183 mRNA expression revealed the presence of a 1.6 kb transcript that is expressed at a high level in brain, kidney, pancreas, and heart; at a moderate level in liver and skeletal muscle, and at a low level in placenta and lung.

The nucleic acid sequence of TANGO 183 is related to a sequence tagged site at chromosomal location 11p15.4, and TANGO may map to this site.

The predicted cytoplasmic domain of TANGO 183 has a relatively high number of charged residues (32%). This suggests that TANGO 183 may non-covalently, e.g., electrostatically, associate with an intracellular molecule such as a cytoskeletal component. Accordingly, TANGO 183 may itself be involved in maintaining the structural integrity of cells in which it is expressed. If so, aberrant TANGO 183 protein or aberrantly regulated TANGO 183 could be involved in alterations in cellular morphology, e.g., alterations associated with metastasis. Accordingly, TANGO 183 nucleic acid molecules and polypeptides as well as anti-TANGO 183 antibodies and modulators of TANGO 183 expression or activity may be useful in the treatment of disorders associated with aberrant cell development or cell differentiation, e.g., cancer, or cell migration, e.g., tumor metastasis.

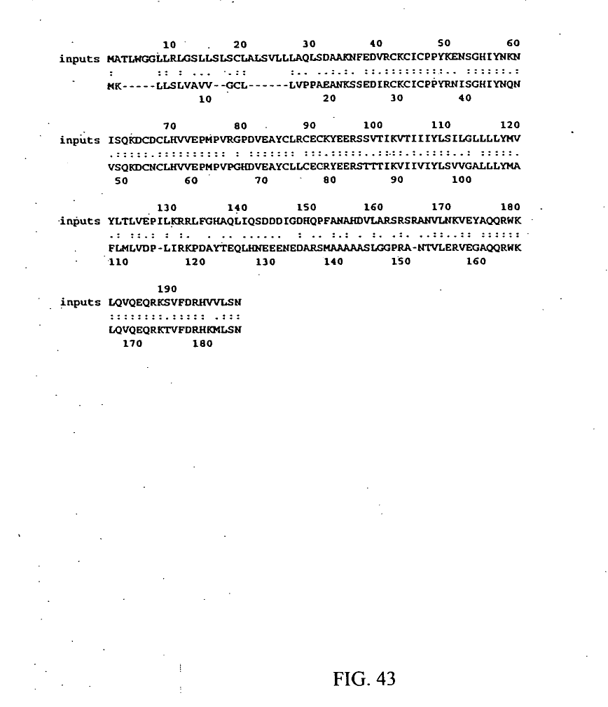

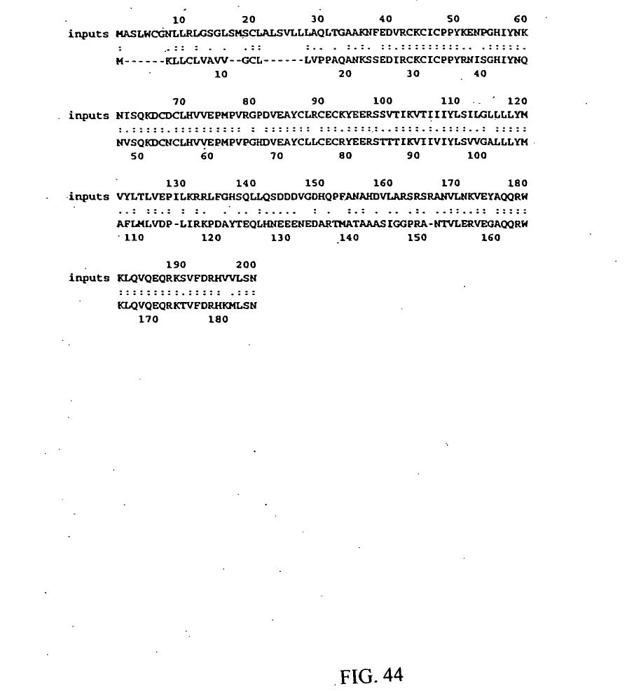

TANGO 183 and TANGO 184 are related and may play similar functional roles. FIG. 43 depicts an alignment of the amino acid sequences of human TANGO 184 (SEQ ID NO:27) and human TANGO 183 (SEQ ID NO:26). FIG. 44 depicts an alignment of the amino acid sequences of murine TANGO 184 (SEQ ID NO:58) and murine TANGO 183 (SEQ ID NO:57).

TANGO 183 is related to C. elegans R12C12.6 (GenBank Accession NO. U23510).

TANGO 184

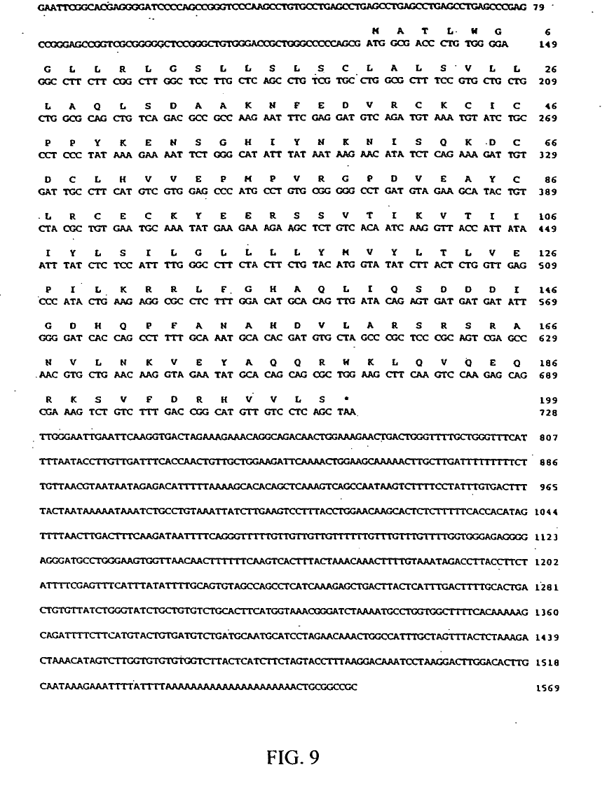

The human TANGO 184 cDNA of SEQ ID NO:5 has a 594 nucleotide open reading frame (SEQ ID NO:16) encoding a 198 amino acid protein (SEQ ID NO:27). The cDNA and protein sequences of human TANGO 184 are shown in FIG. 9.

Human TANGO 184 is predicted to be a transmembrane protein having a 28 amino acid signal sequence (amino acids 1-28 of SEQ ID NO:27; SEQ ID NO:68) followed by a 170 amino acid mature protein (amino acids 29-198 of SEQ ID NO:27; SEQ ID NO:80) having a 74 amino acid extracellular domain (amino acids 29-102 of SEQ ID NO: 27; SEQ ID NO:89), a 23 amino acid transmembrane domain (amino acids 103-125 of SEQ ID NO:27; SEQ ID NO:95), and a 73 amino acid cytoplasmic domain (amino acids 126-198 of SEQ ID NO 27; SEQ ID NO:103). TANGO 184 has a high porportion of charged amino acids in the predicted extracellular (31%) and cytoplasmic (29%) domains. Notably, the transmembrane regions include charged residues. Human TANGO 184 is predicted to have a molecular weight of 22.5 kDa prior to cleavage of its signal peptide and a molecular weight of 18.9 kDa subsequent to cleavage of its signal peptide.

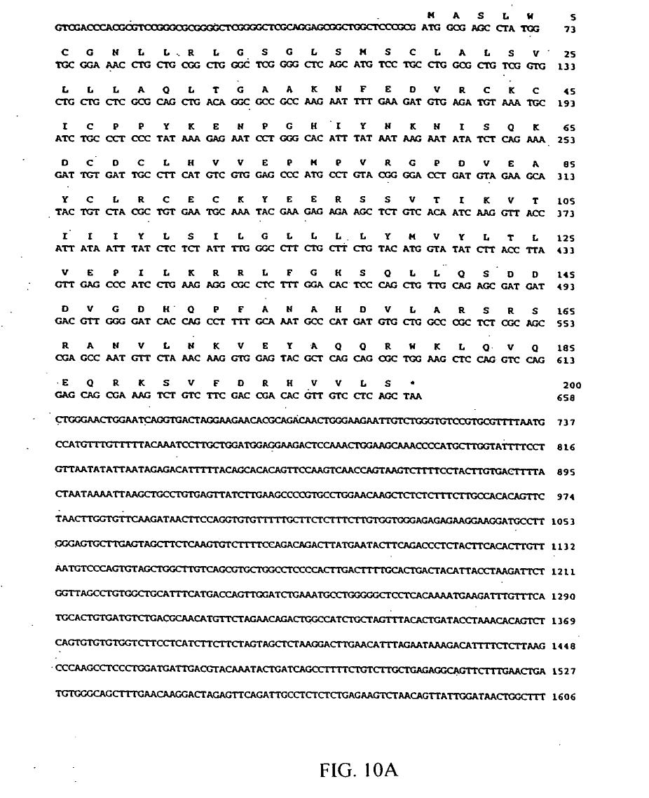

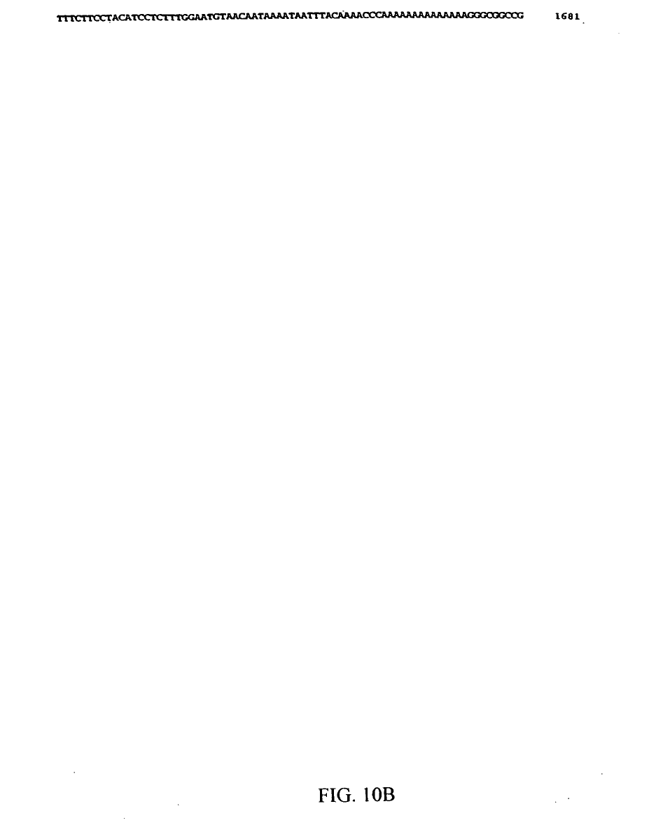

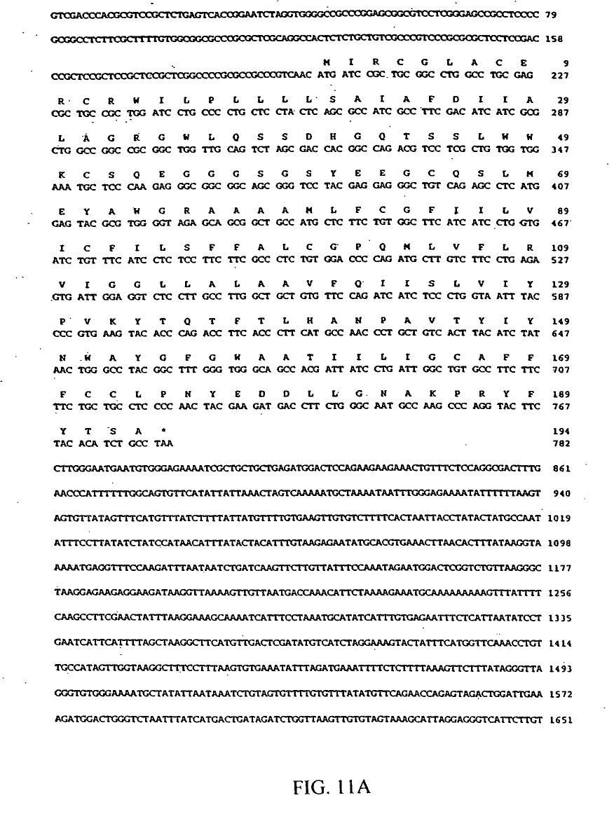

The murine TANGO 184 cDNA of SEQ ID NO:38 has a 357 nucleotide open reading frame (SEQ ID NO:48) encoding a 199 amino acid protein (SEQ ID NO:58). The cDNA and protein sequences of murine TANGO 184 are shown in FIG. 10.

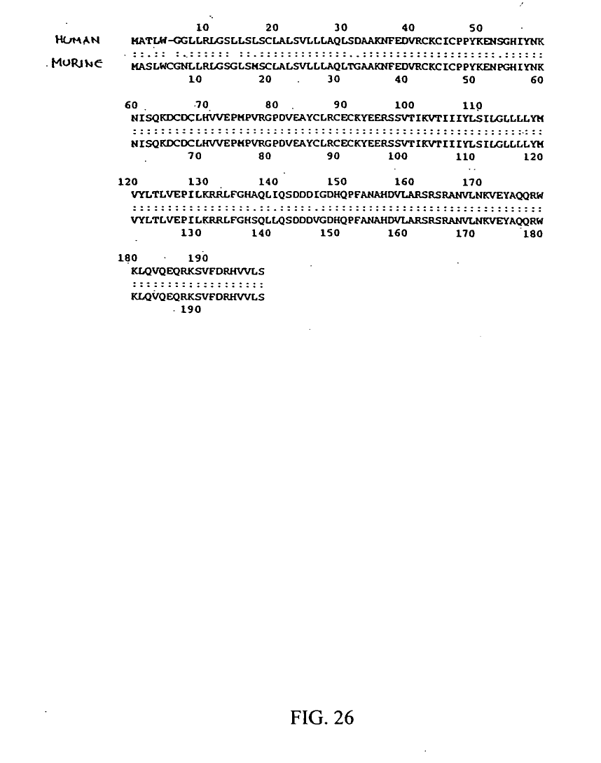

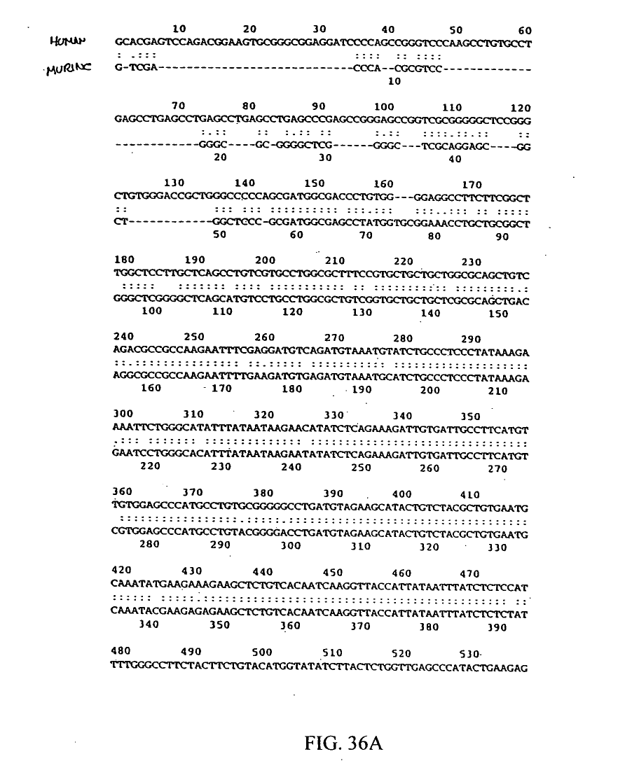

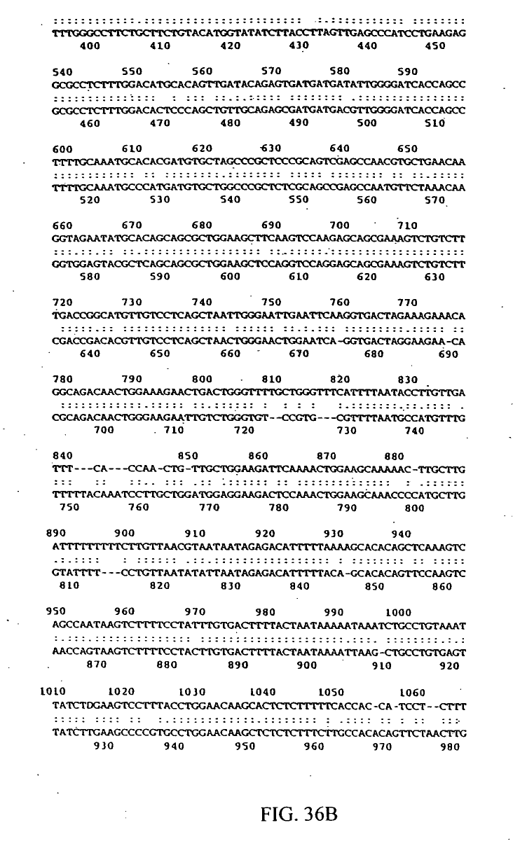

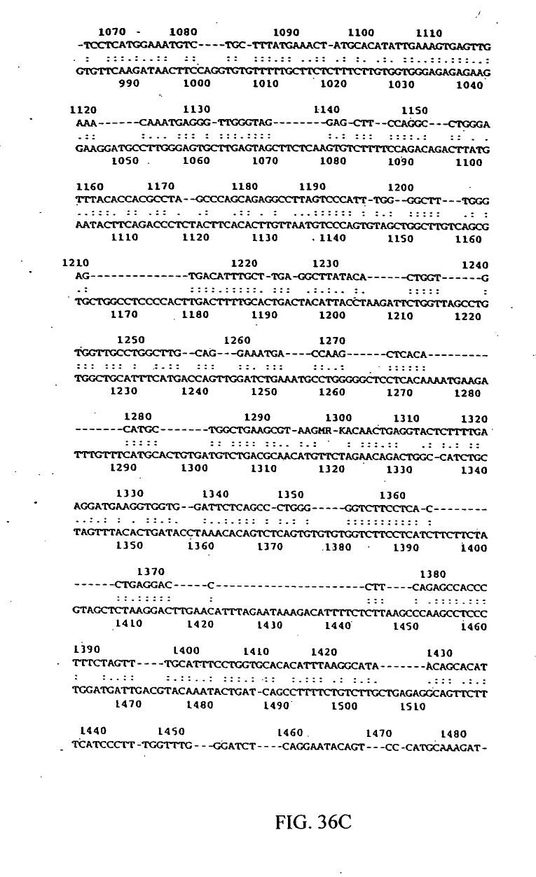

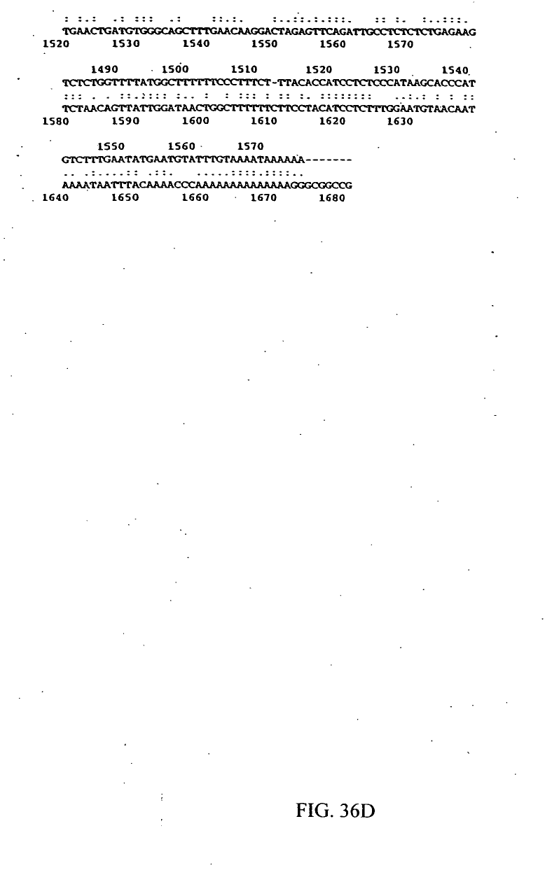

FIG. 26 depicts an alignment of the predicted amino acids sequences of human (SEQ ID NO:27) and murine (SEQ ID NO:58) TANGO 184 (94.5% identity). FIG. 36 depicts an alignment of the cDNA sequences of human (SEQ ID NO:5) and murine (SEQ ID NO:38) TANGO 184 (63.8% identity).

Northern analysis of human TANGO 184 mRNA expression revealed the presence of a 2 kb transcript that is expressed at a high level in heart brain, placenta, skeletal muscle, kidney, and pancreas; and at a low level in lung and liver. There are two alternative polyA sites: nucleotide 1000 and nucleotide 2000.

In situ analysis of TANGO 184 expression in adult mice revel expression in the brain (moderate, ubiquitous expression), spinal cord (weak expression in the region of the grey matter) submandibular gland (strong, ubiquitous expression), stomach (weak expression in the muscle region), Kidney (weak, ubiquitous expression in the cortex and medulla, stronger expression in papilla), adrenal gland (weak ubiquitous expression), thymus (weak expression in cortex), lymph node (moderate ubiquitous expression) spleen (weak expression in follicles), skeletal muscle/smooth muscle (diaphragm), testis (strong expression in the area surrounding the seminiferous tubules), ovaries (weak expression) placenta (moderate, ubiquitous expression). This analysis did not reveal significant expression in white fat, brown fat, heart, lung, liver, pancreas, colon, small intestine, and bladder. In embryonic tissue, this analysis revealed expression at E13.5 (weak to moderate ubiquitous expression with higher expression in the brain and liver), E14.5 (weak to moderate ubiquitous expression with higher expression in the brain and liver), E15.5 (moderate ubiquitous expression with higer expression in the brain), E16.5 (weak to moderate ubiquitous expression with higher expression in the brain, spinal cord, brown fat, submandibular gland, lung, stomach, and intestines), E18.5 (weak to moderate ubiquitous expression with higher expression in the brain, spinal cord, brown fat, submandibular gland, lung, stomach, and intestines), and P1.5 (weak ubiquitous expression with higer expression in brain, submandibular gland, olfactory epithelium, and stomach).

The predicted cytoplasmic domain of TANGO 184 has a relatively high number of charged residues (29%). This suggests that TANGO 184 may non-covalently, e.g., electrostatically, associate with an intracellular molecule such as a cytoskeletal component. Accordingly, TANGO 184 may itself be involved in maintaining the structural integrity of cells in which it is expressed. If so, aberrant TANGO 184 protein or aberrantly regulated TANGO 184 could be involved in alterations in cellular morphology, e.g., alterations associated with metastasis. Accordingly, TANGO 184 nucleic acid molecules and polypeptides as well as anti-TANGO 184 antibodies and modulators of TANGO 184 expression or activity may be useful in the treatment of disorders associated with aberrant cell development or cell differentiation, e.g., cancer, or cell migration, e.g., tumor metastasis.

TANGO 185

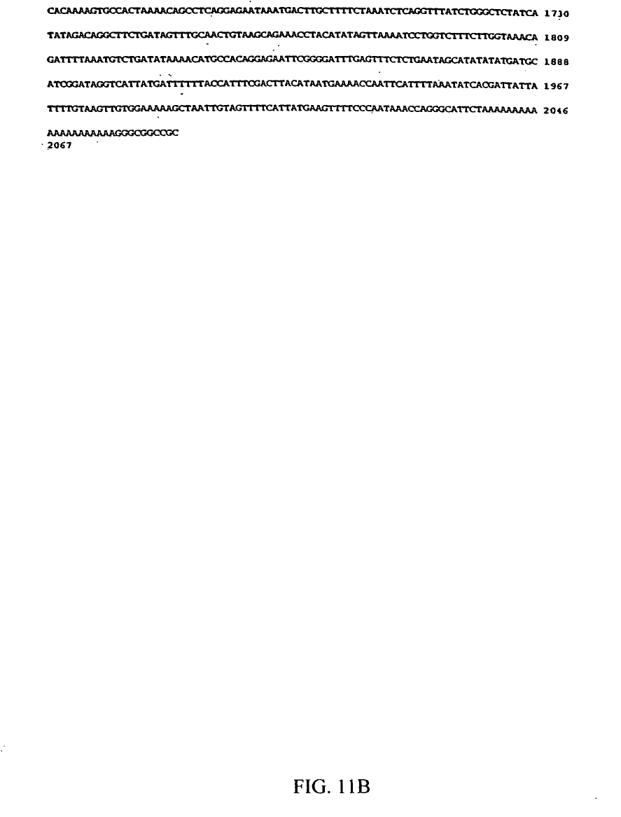

The human TANGO 185 cDNA of SEQ ID NO:6 has a 579 nucleotide open reading frame (SEQ ID NO:17) encoding a 193 amino acid protein (SEQ ID NO:28). The cDNA and protein sequences of human TANGO 185 are shown in FIG. 11.

Human TANGO 185 is predicted to be a transmembrane protein having a 24 amino acid signal sequence (amino acids 1-24 of SEQ ID NO:28; SEQ ID NO:69) followed by a 169 amino acid mature protein (amino acids 25-193 of SEQ ID NO:28; SEQ ID NO:81) having two extracellular domains, one having 51 amino acids (amino acids 25-75 of SEQ ID NO:28; SEQ ID NO:90), and a second having 19 amino acids (amino acids 132-150 of SEQ ID NO:28; SEQ ID NO:91); three transmembrane domains, one having 27 amino acids (amino acids 76-102 of SEQ ID NO:28; SEQ ID NO:96), a second having 22 amino acids (amino acids 110-131 of SEQ ID NO:28; SEQ ID NO:97), the third having 24 amino acids (amino acids 151-174 of SEQ ID NO:28; SEQ ID NO:98); and two cytoplasmic domains, one having 7 amino acids (amino acids 103-109 of SEQ ID NO:28; SEQ ID NO:104), and a second having 19 amino acids (amino acids 175-193 of SEQ ID NO:28; SEQ ID NO:105). The predicted 22 amino acid transmembrane domain and the predicted 24 amino acid domain, along with the predicted 7 amino acid cytoplasmic domain may form one hydrophobic domain that passes through the membrane twice. TANGO 185 is predicted to have a molecular weight of 21.4 kDa prior to cleavage of its signal peptide and a molecular weight of 18.8 kDa subsequent to cleavage of its signal peptide. Notably, the transmembrane regions have charged residues.

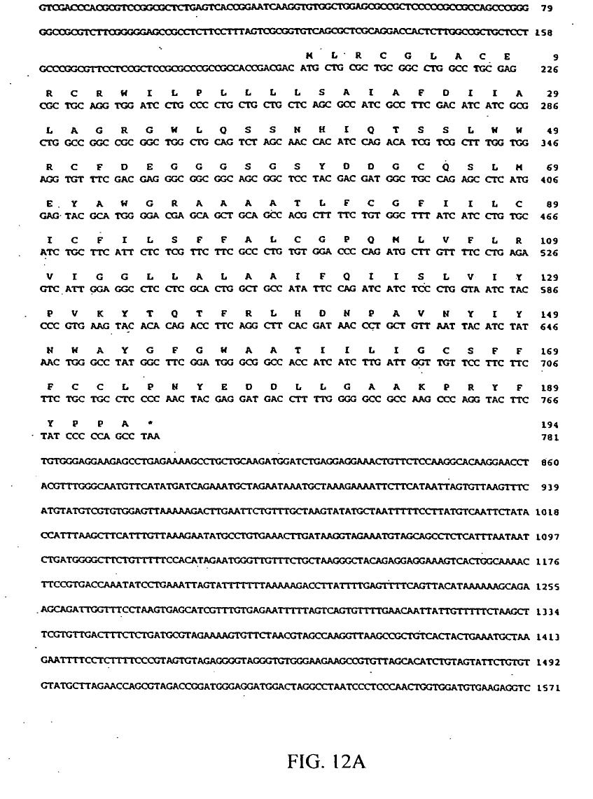

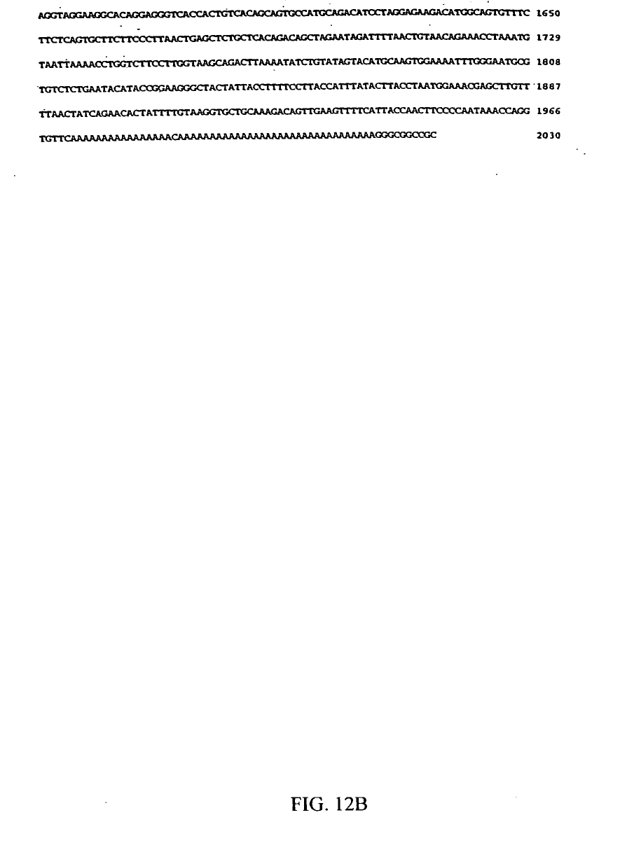

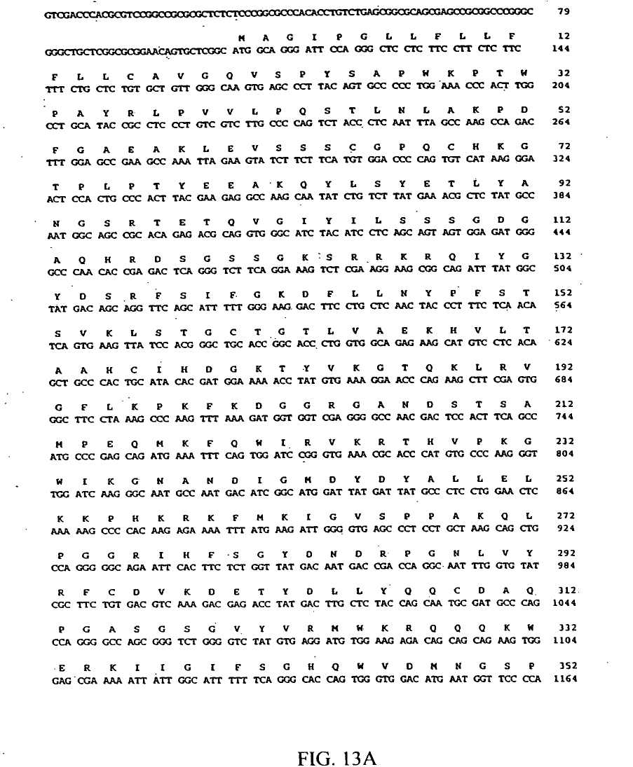

The murine TANGO 185 cDNA of SEQ ID NO:39 has a 579 nucleotide open reading frame (SEQ ID NO:49) encoding a 193 amino acid protein (SEQ ID NO:59). The cDNA and protein sequences of murine TANGO 185 are shown in FIG. 12.

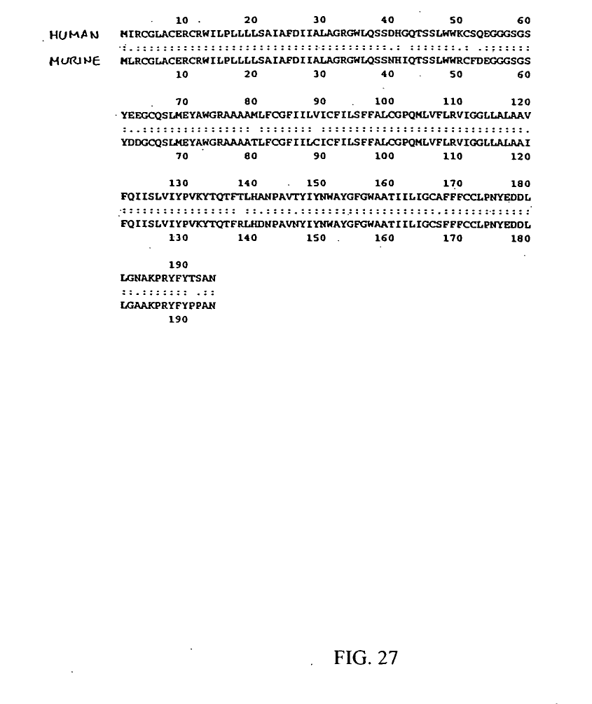

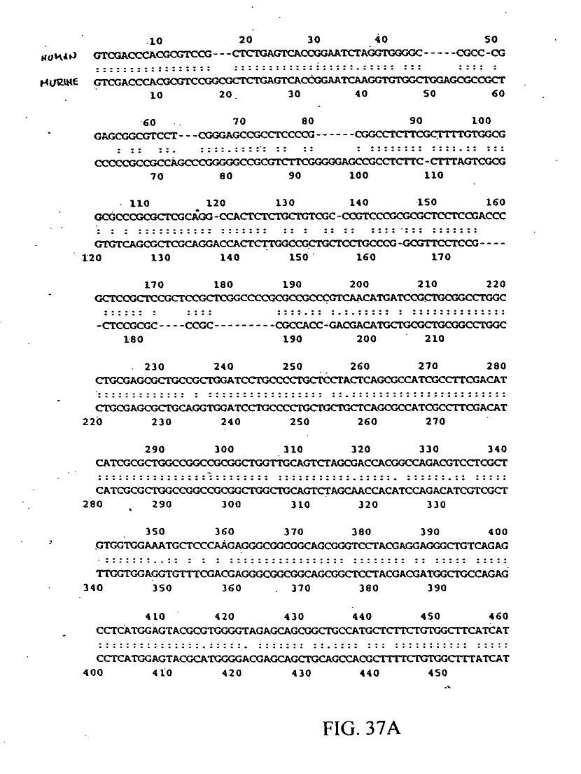

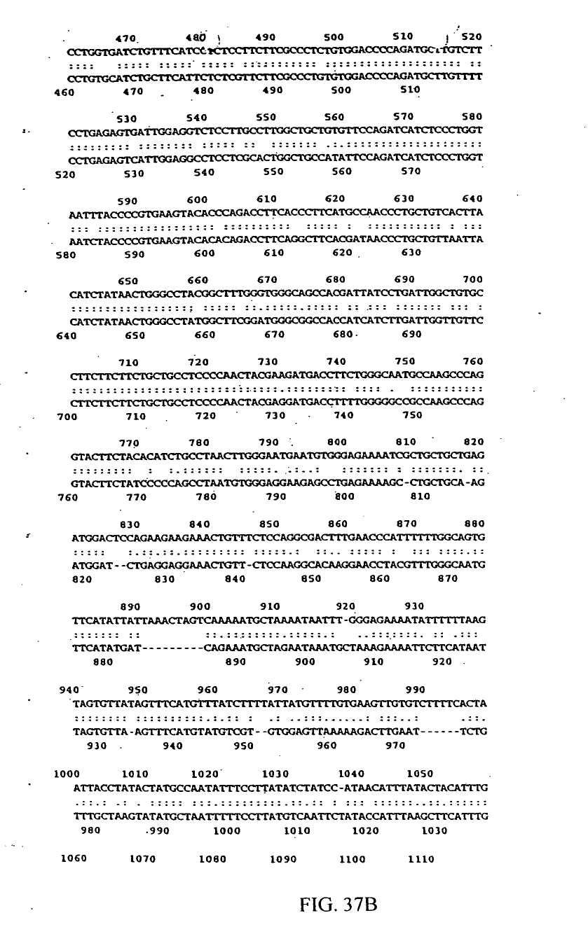

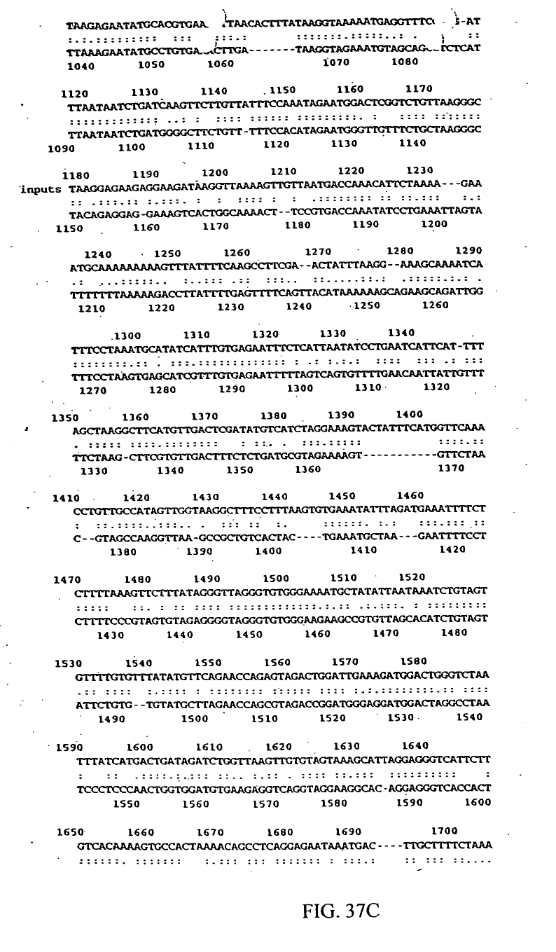

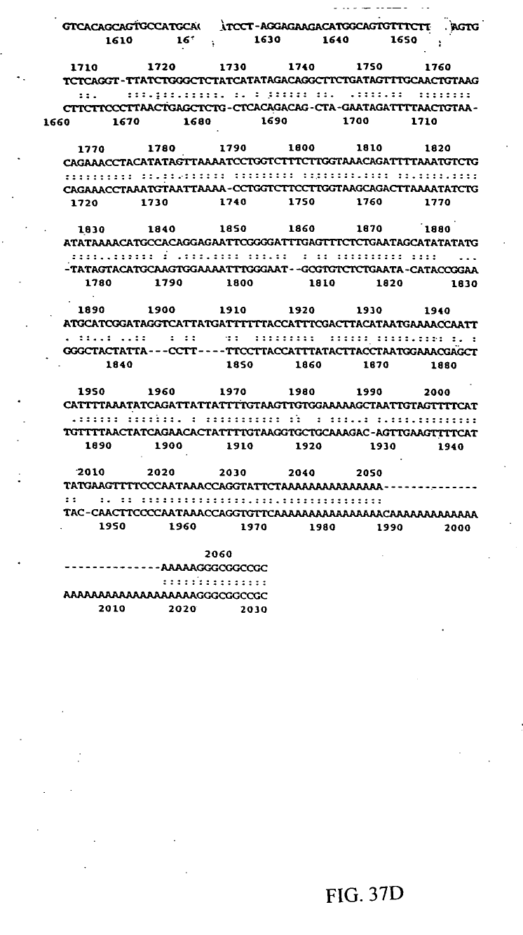

FIG. 27 depicts an alignment of the predicted amino acids sequences of human (SEQ ID NO:28) and murine (SEQ ID NO:59) TANGO 185 (90.7% identity). FIG. 37 depicts an alignment of the cDNA sequences of human (SEQ ID NO:6) and murine (SEQ ID NO:39) TANGO 185 (71.1% identity)

Human TANGO 185 maps to chromosome 6.

Northern analysis of human TANGO 185 mRNA expression revealed the presence of 2.2 kb major transcript and a 4.2 kb minor transcript. This analysis also revealed that the 2.3 kb transcript is expressed at a high level in heart, placenta, and pancreas; at a moderate level in lung, liver, and kidney; and at a very low level, if at all, in brain and skeletal muscle. The 4.2 kb transcript is expressed at a low level in placenta.

In situ analysis of TANGO 185 expression in adult mice revealed expression in the brain (choroid plexus), submamandibular gland (ubiquitous expression), white fat (weak expression, possible mammary gland expression), stomach (mucosal epithelium), kidney (medulla-cortex transition and medullary rays), colon (weak expression in the epithelium), small intestine (villi), thymus (low level expression), bladder (mucosal epithelium), and placenta (ubiquitous expresion in decidua region). This analysis did not reveal significant expression in adult eye and harderian gland, brown fat, heart, lung, liver, spleen, pancreas, skeletal muscle, testes, and ovaries.

In situ analysis of TANGO 185 embryonic expression in mice revealed expression at E13.5 (high level expression the skin and submaxillary gland and low level ubiquitous expression in the liver); E14.5 (high level expression in the choroid plexus of the lateral and fourth ventricles, skin, epithelium of the oral cavity, follicles of vibrissa, submaxillary gland, stomach, and heart; expression in lung (especially the developing large airways) and liver (ubiquitous expression)). At E15.5 the observed expression pattern is nearly identical to that at E14.5 except that there is expression in the region outlining the intestinal tract and lung expression is ubiguitous with higher expression in the region outlining the large airways.

At E16.5 high level expression is observed in skin choroid plexus, the lining of the oral and nasal cavity, esophagus, bladder, stomach, intestine, large vessels of the heart, large airways of the lung, and the region outlining the vertebrae. Lower ubiquitous expression is present in the heart, lung and thymus. A somewhat higher, multifocal expression is present in the thymus.

At E18.5 the expression pattern is identical to that observed at E16.5 except that expression is also observed in developing hair follicles.

At P1.5 the expression pattern is identical to that observed at E16.5 except that there is no long significant expression in the region outlining the vertebrae.

The expression pattern of TANGO 185 during eubryonic development suggests that TANGO 185 expression is strongly associated with squamous and mucosal epithelial cells.

The expression pattern of TANGO 185 suggests that it is involved in cell development and/or cell differentiation. Accordingly, TANGO 185 nucleic acid molecules and polypeptides as well as anti-TANGO 185 antibodies and modulators of TANGO 185 expression or activity may be useful in the treatment of disorders associated with aberrant cell development or cell differentiation, e.g., cancer. There is evidence that TANGO 185 is expressed in prostate cells. Thus, TANGO 185 nucleic acid molecules and polypeptides as well as anti-TANGO 185 antibodies and modulators of TANGO 185 expression or activity may be useful in the treatment of prostate cancer.

TANGO 186

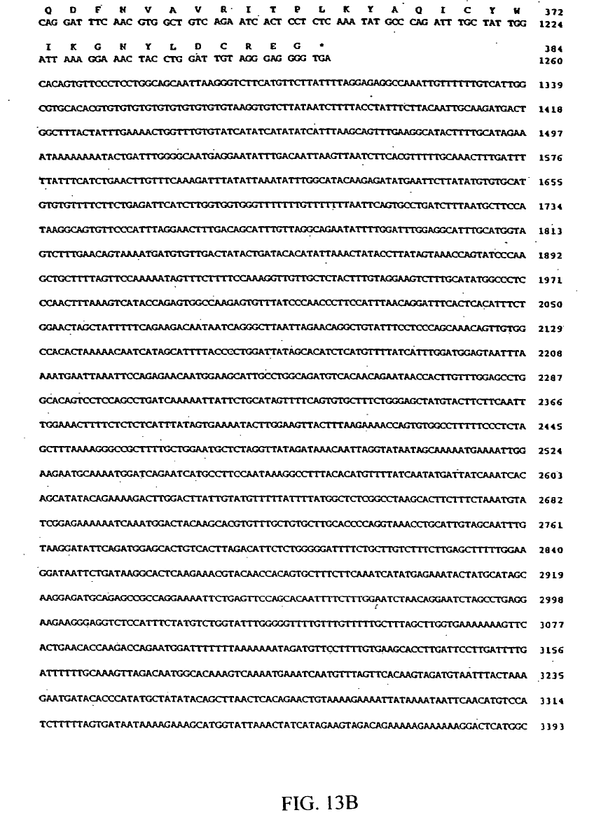

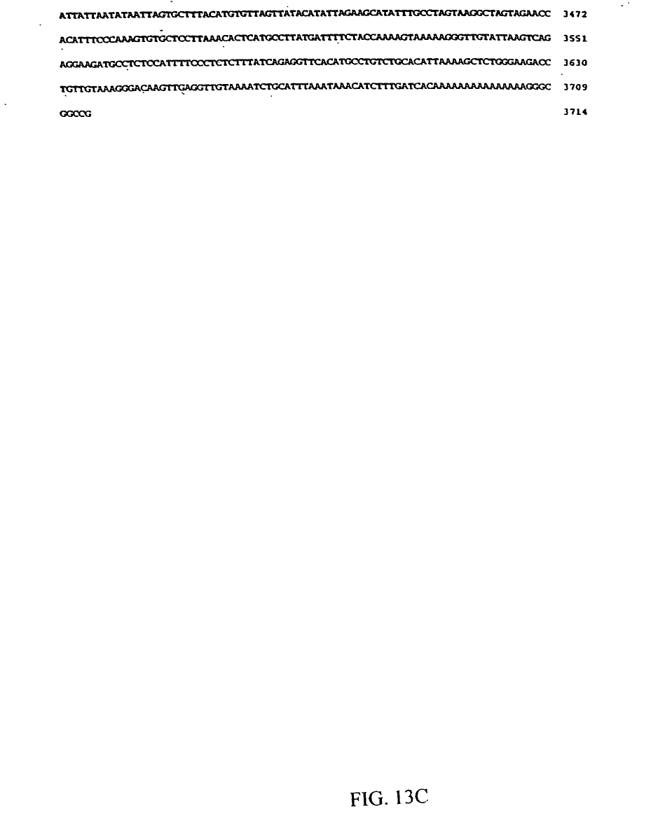

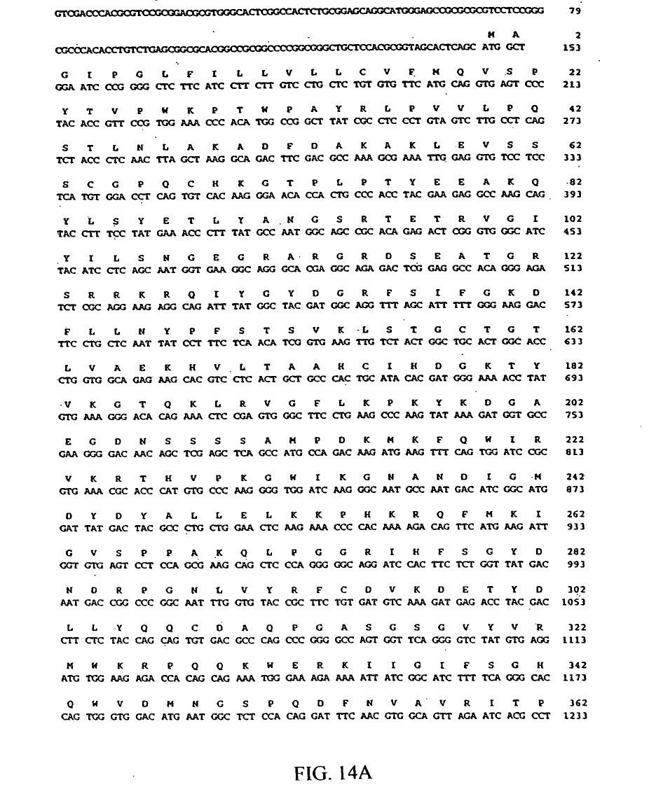

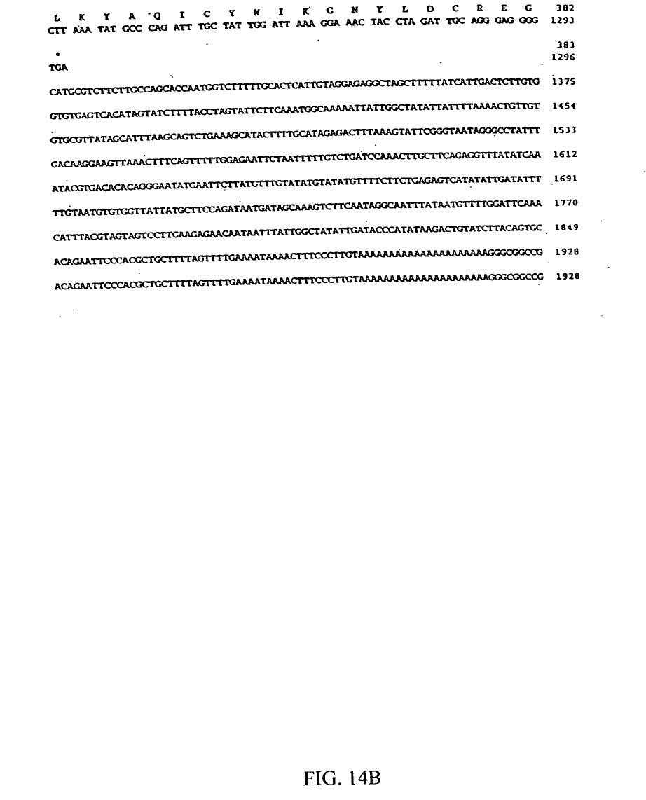

The human TANGO 186 cDNA of SEQ ID NO:7 has a 1149 nucleotide open reading frame (SEQ ID NO:18) encoding a 383 amino acid protein (SEQ ID NO:29). The cDNA and protein sequences of human TANGO 186 are shown in FIG. 13.

Human TANGO 186 is predicted to be a secreted protein having a 20 amino acid signal sequence (amino acids 1-20 of SEQ ID NO:29; SEQ ID NO:70) followed by a 363 amino acid mature protein (amino acids 21-383 of SEQ ID NO:29; SEQ ID NO:82). There are eight cysteines in mature TANGO 186. Some or all of these might be involved in disulfide bond formation. Human TANGO 186 is predicted to have a molecular weight of 43.0 kDa prior to cleavage of its signal peptide and a molecular weight of 40.3 kDa subsequent to cleavage of its signal peptide.

The murine TANGO 186 cDNA of SEQ ID NO:40 has a 1146 nucleotide open reading frame (SEQ ID NO:50) encoding a 382 amino acid protein (SEQ ID NO:60). The cDNA and protein sequences of murine TANGO 186 are shown in FIG. 14. Conserved cysteine residues are particularly important and are preferably retained in functional variants

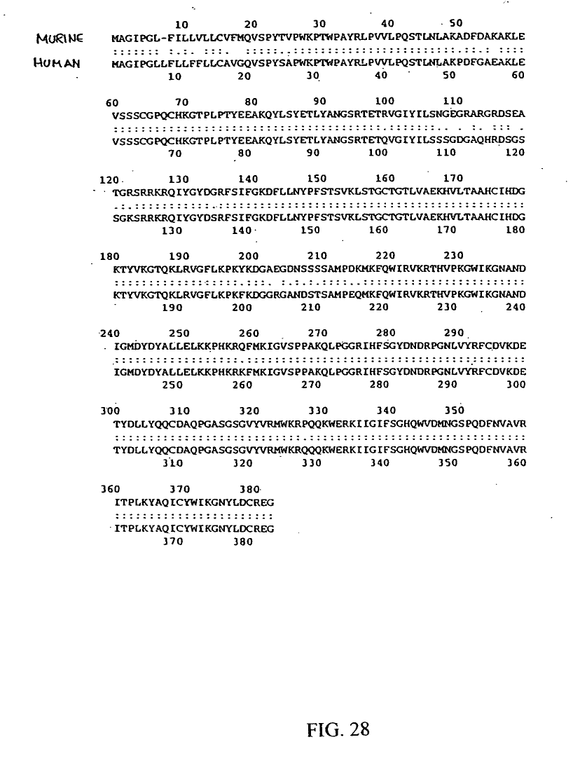

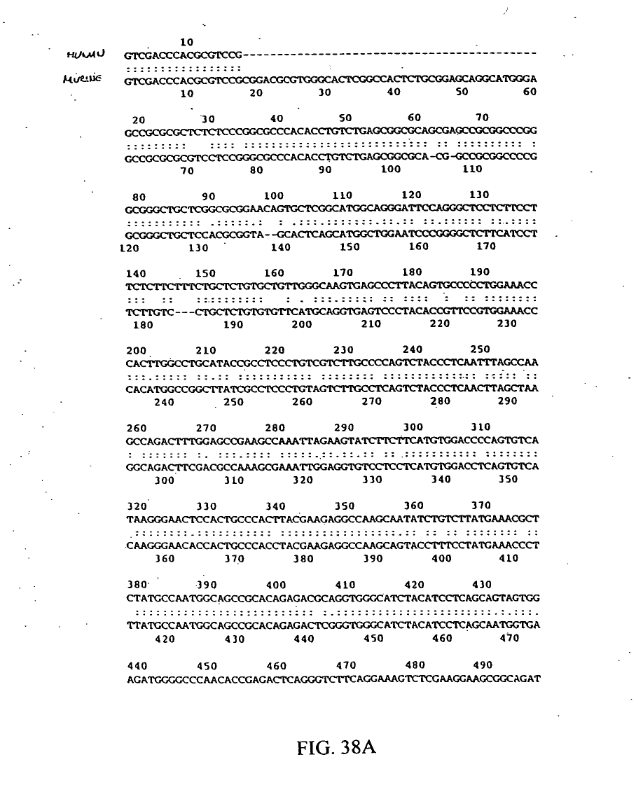

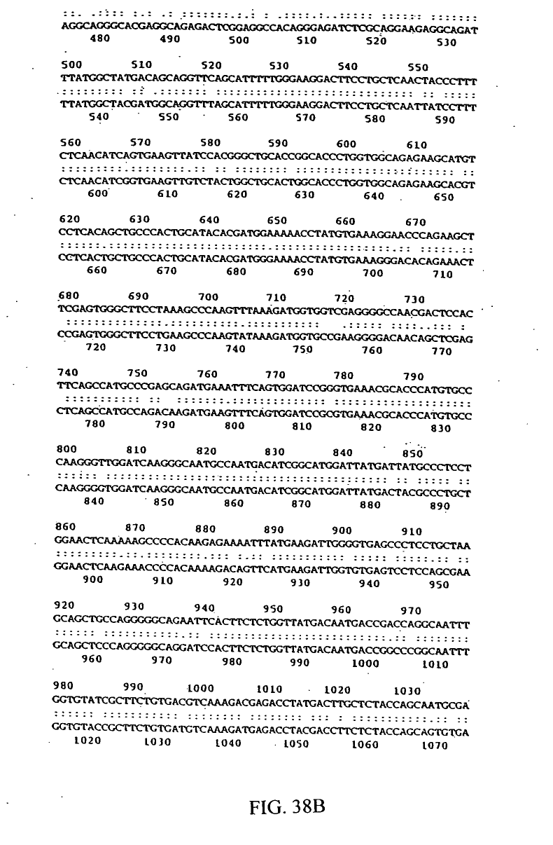

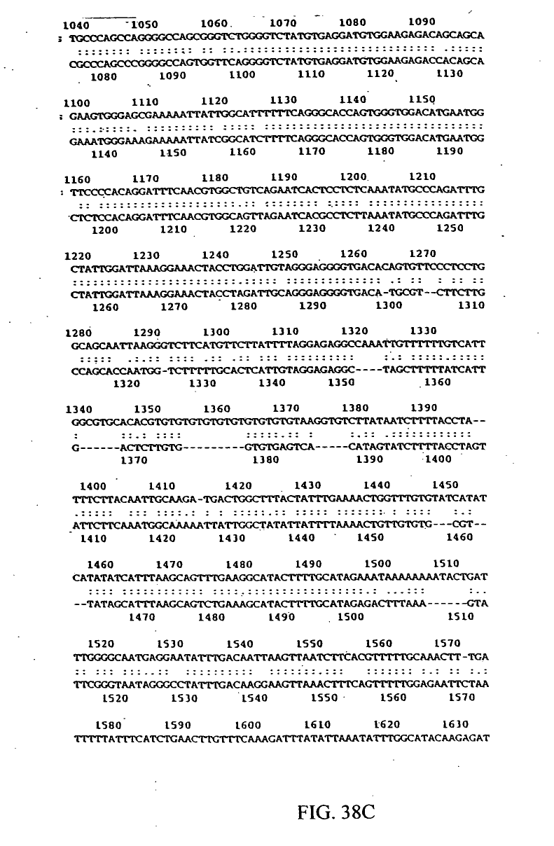

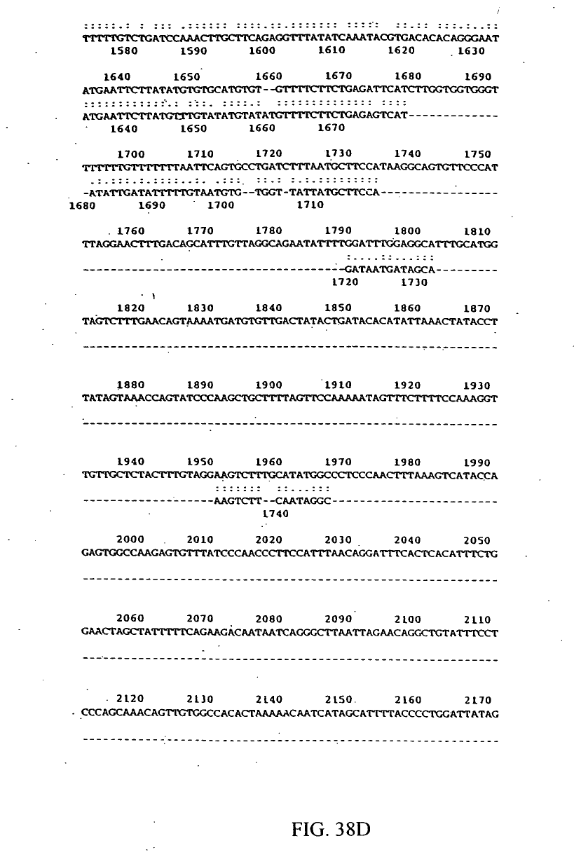

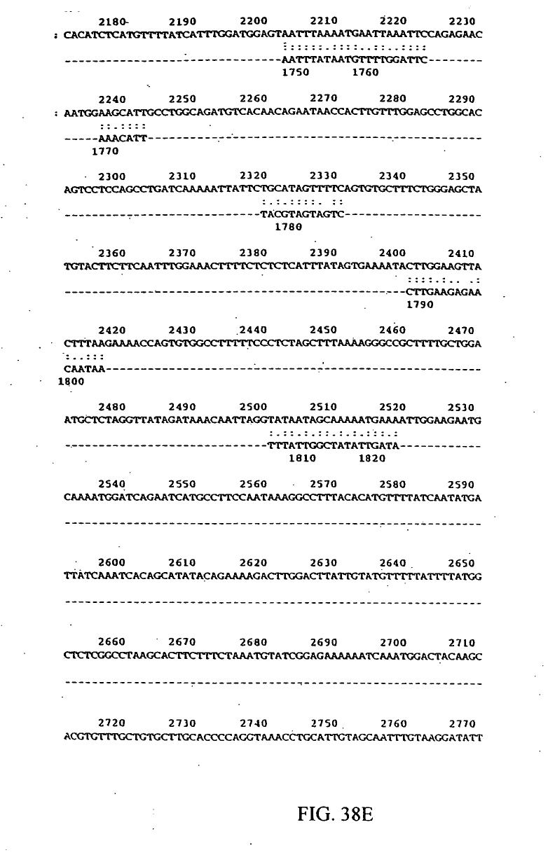

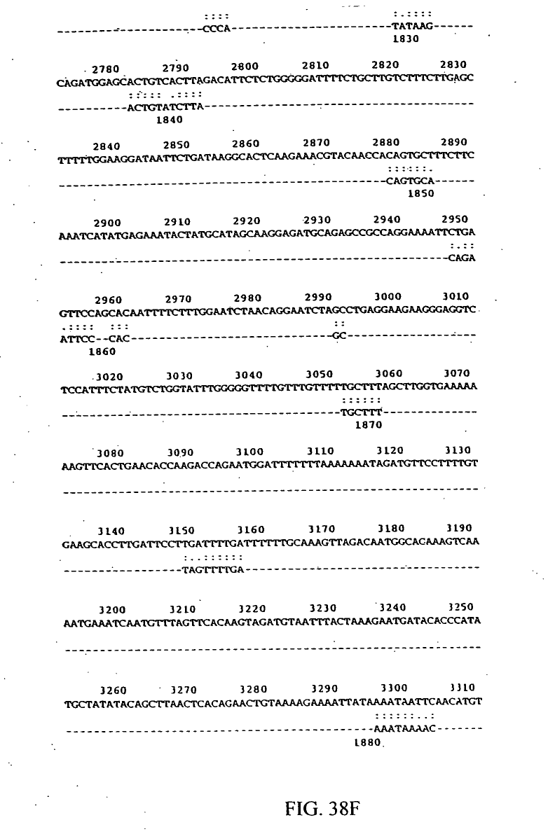

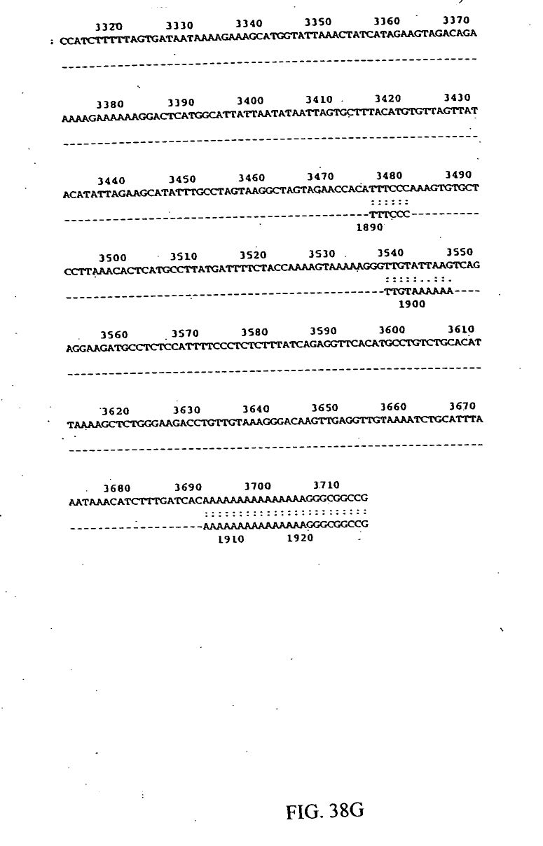

FIG. 28 depicts an alignment of the predicted amino acids sequences of human (SEQ ID NO:29) and murine (SEQ ID NO:60) TANGO 186 (90.9% identity). FIG. 38 depicts an alignment of the cDNA sequences of human (SEQ ID NO:7) and murine (SEQ ID NO:40) TANGO 186 (41.6% identity). The human and murine TANGO 186 proteins are highly similar except within three portions: the signal sequence, a hinge region at amino acids 108-123, and a hinge region at amino acids 198-216. Within these three portions the proteins are only about 50% identical. Outside of these three portions the proteins are about 97.3% identical.

TANGO 186 maps to human chromosome 11q14.

Northern analysis of human TANGO 186 mRNA expression revealed the presence of a 1.8 kb transcript and a 4 kb transcript. Both transcripts are expressed at a low level in heart, lung, liver, skeletal muscle, kidney, and pancreas and at a very low level in brain.

In situ analysis of TANGO 186 in adult mice revealed that TANGO 186 is expressed in brain (olfactory bulb), spleen (low level ubiquitous signal), small intestine (very strong signal in villi and submucosa), colon (ubiquitous signal), kidney (cortical and medullary region), lung (bronchial epithelium), eye (iris and cornea), placenta (strong signal in the outer membrane). This analysis did not detect expression in adult pancreas, heart, skeletal muscle, diaphragm, esophagus, liver, and thymus.

In situ expression analysis of murine embryonic sagittal sections revealed expression at stage E13.5 in epithelium of the lower and upper lip, cartilage primordium of basisphenoid bone, cartilage condensation of sacral vertebral body (centrum), small intestine, and heart. At stage E14.5, in addition to the expression observed at stage E13.5, expression was also observed in: eye (or cartilage around eye), Meckel's cartilage, and cartilage of the limb digits. At stage E15.5 expression was observed in vibrissae of the snout, kidney (embryonic glomeruli), cartilage of the limb digits, cartilage of the vertebral column, heart, eye, and small intestine. At stage E16.5 the observed expression pattern was similar to that observed at E15.5, but there was a notable reduction in signal from cartilage, epithelium of upper and lower lip, and heart. Also at stage E16.5 low level signal was observed in the lung, and a strong signal was still observed in the small intestine. At stage E17.5 expression of TANGO 186 was observed to be more ubiquitous. However, expression in cartilage was observed to decrease with the exception of ossification within cartilage primordium of body of mandible. At stage E17.5 strong expression continued to be observed in the small intestine. The expression pattern at stage P1.5 was observed to be very similar to that observed at stage E17.5 with expression being nearly ubiquitous with the notable exceptions of the brain and spinal cord in which little or no expression was observed. At stage P1.5 the highest expression observed was in the in the small intestine, lung, and kidney.

Overall, the in situ expression analysis of adult and embryonic tissue revealed that expression is first observed in the developing cartilage, small intestine, and heart with the cartilage expression being most striking in the developing vertebral column and jaw area. Strong expression in the cartilage of the vertebral column and developing digits was observed through stage E16.5. Subsequently, cartilage expression was observed to decrease with some exceptions in the jaw area. Other embryonic tissue in which the observed expression was notable include the kidney, specifically the embryonic glomeruli, and the lung. These tissues continue to have strong expression in the adult with expression in the kidney also being observed in the medullary region and lung expression becoming restricted to the bronchial epithelium. Expression of TANGO 186 becomes more ubiquitous through P1.5 with the most noticeable exception being the brain and spinal cord. In the adult, however, signal is observed in the olfactory bulb.

In a murine LPS disease model, increasaed TANGO 186 expression was observed in the brain 2 and 8 hours after LPS treatment. Decrease TANGO 186 expression was observed at these same time points in the kidney. TANGO 186 expression was also observed in the gastric mucosa.

As discussed above, murine in situ expression analysis demonstrates that TANGO 186 is expressed in cartilage throughout the embryo, suggesting that TANGO 186 is a regulatory molecule that plays a role in a bone formation (e.g., condensation of cartilage). Accordingly, TANGO 186 nucleic acid molecules and polypeptides as well as anti-TANGO 186 antibodies and modulators of TANGO 186 expression or activity may be useful in the diagnosis and treatment of bone and cartilage disorders (e.g., osteogenesis imperfecta and broken bones, cartilage degradation, and bone degradation). Moreover, many bone morphogenic proteins and TGF-β family members are regulated by extracellular proteins, e.g., noggin and chordin. Thus, TANGO 186, which is expressed in the heart, may play a role in heart development, and TANGO 186 nucleic acid molecules and polypeptides as well as anti-TANGO 186 antibodies and modulators of TANGO 186 expression or activity may be useful in the diagnosis and treatment of developmental disorders of the heart, e.g., valve malformation.

There is some seqeunce similarity between TANGO 186 and a Bacillus serine protease. Thus, TANGO 186 may have serine protease activity.

TANGO 188

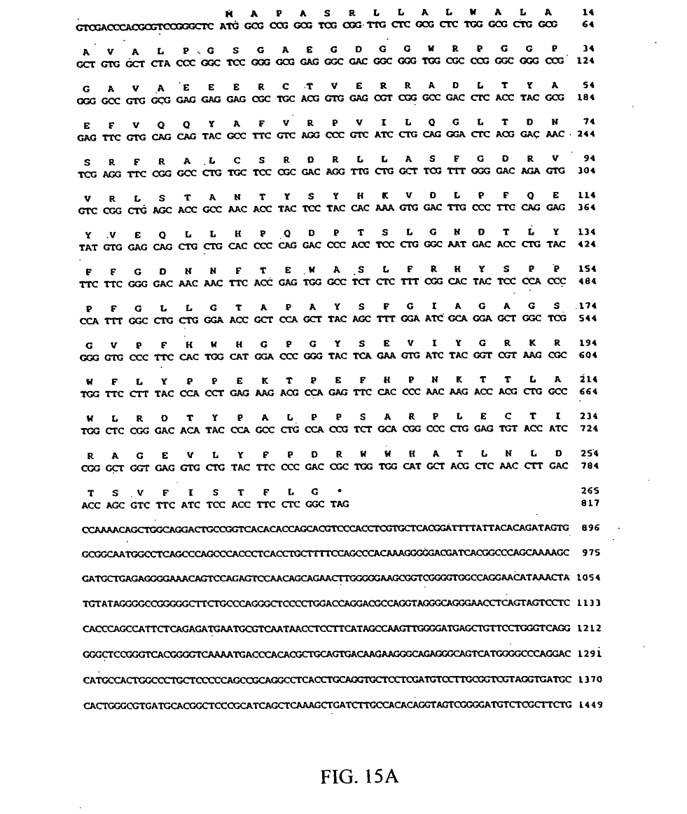

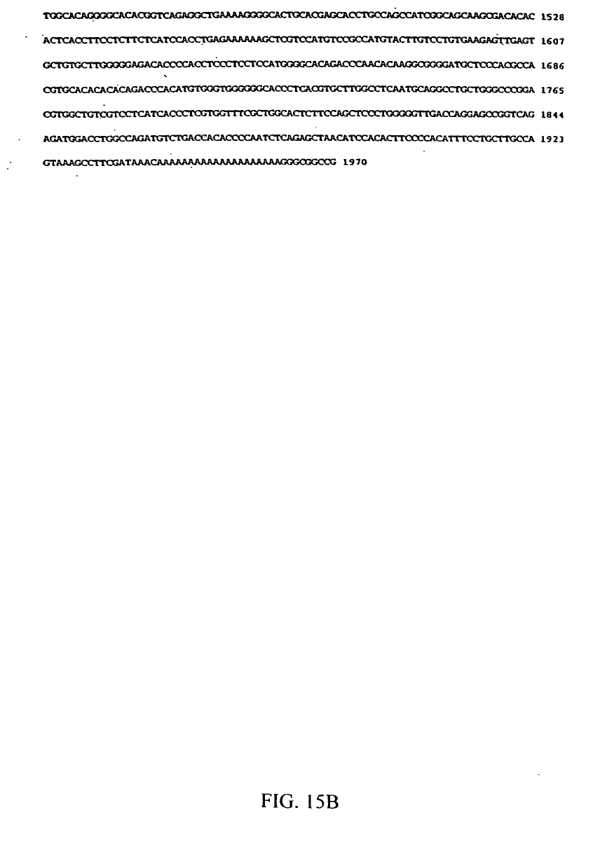

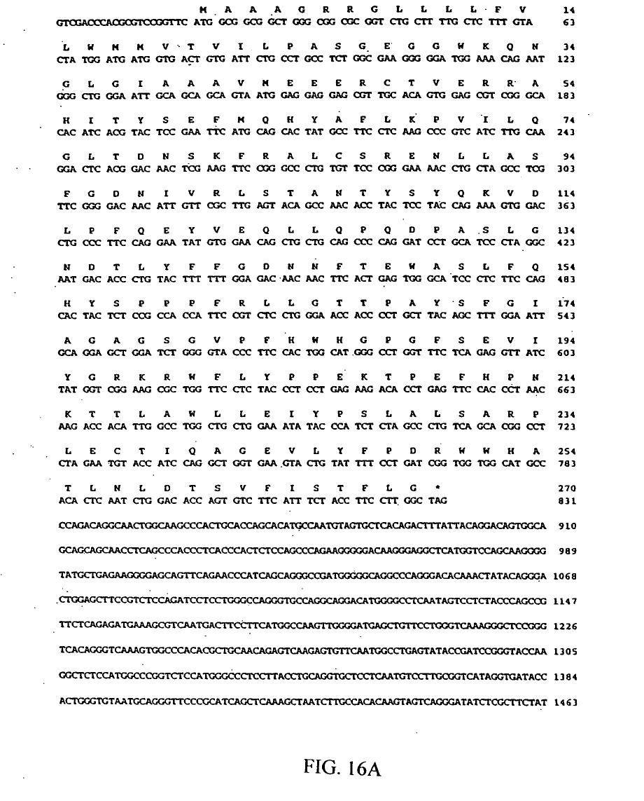

The human TANGO 188 cDNA of SEQ ID NO:8 has a 792 nucleotide open reading frame (SEQ ID NO:19) encoding a 264 amino acid protein (SEQ ID NO:30). The cDNA and protein sequences of human TANGO 188 are shown in FIG. 15.

Human TANGO 188 is predicted to be a secreted protein having a 23 amino acid signal sequence (amino acids 1-23 of SEQ ID NO:30; SEQ ID NO:71) followed by a 241 amino acid mature protein (amino acids 24-264 of SEQ ID NO:30; SEQ ID NO:83). Human TANGO 188 is predicted to have a molecular weight of 29.5 kDa, prior to cleavage of its signal peptide.

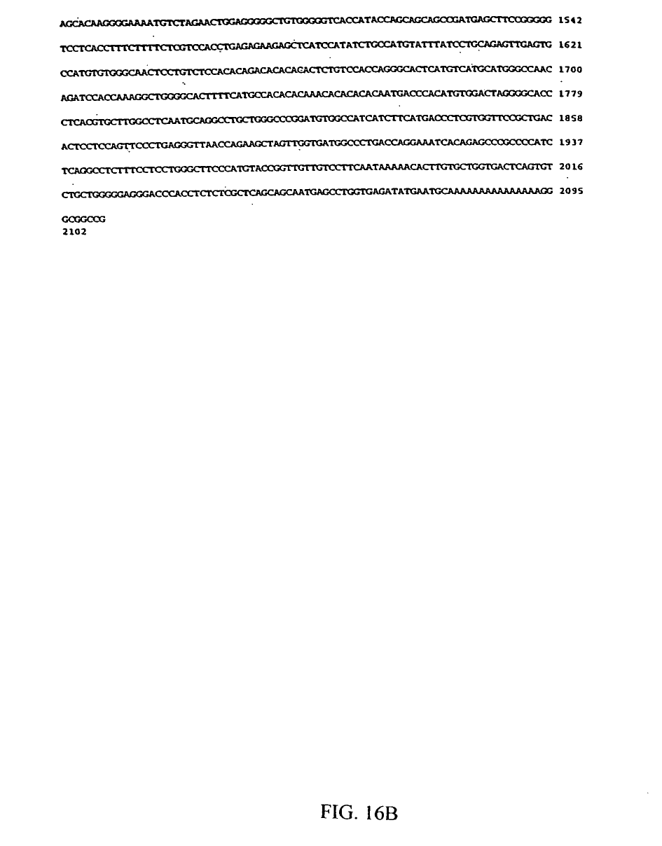

The murine TANGO 188 cDNA of SEQ ID NO:41 has an 807 nucleotide open reading frame (SEQ ID NO:51) encoding a 269 amino acid protein (SEQ ID NO:61). The cDNA and protein sequences of murine TANGO 188 are shown in FIG. 16.

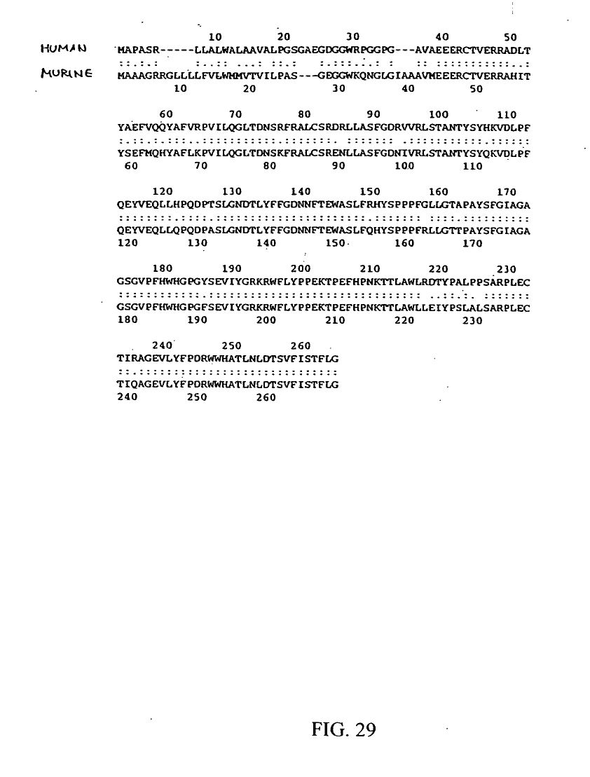

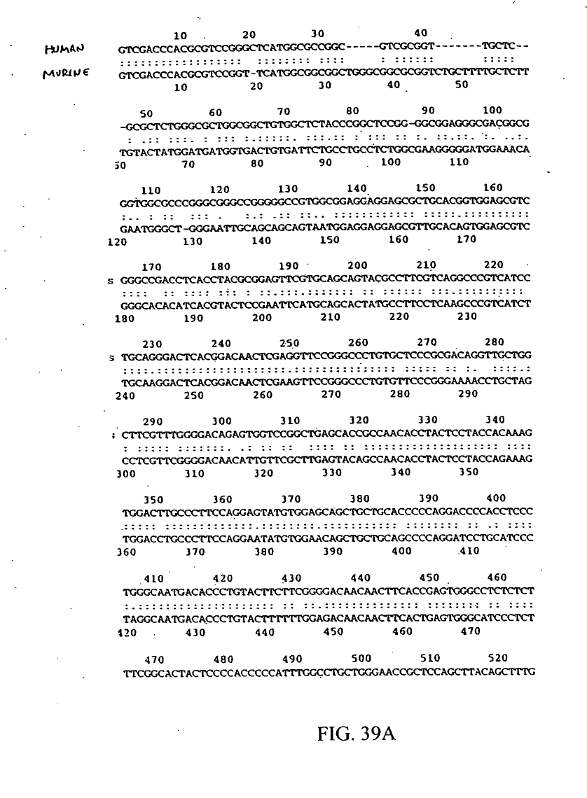

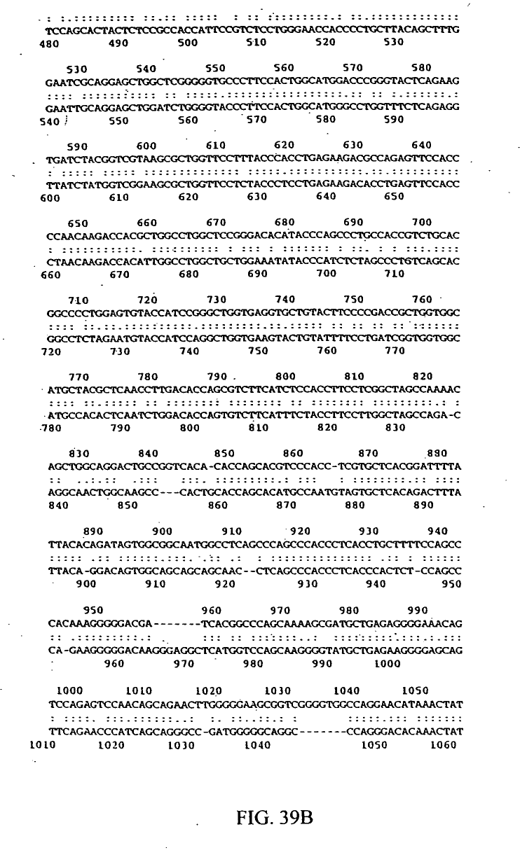

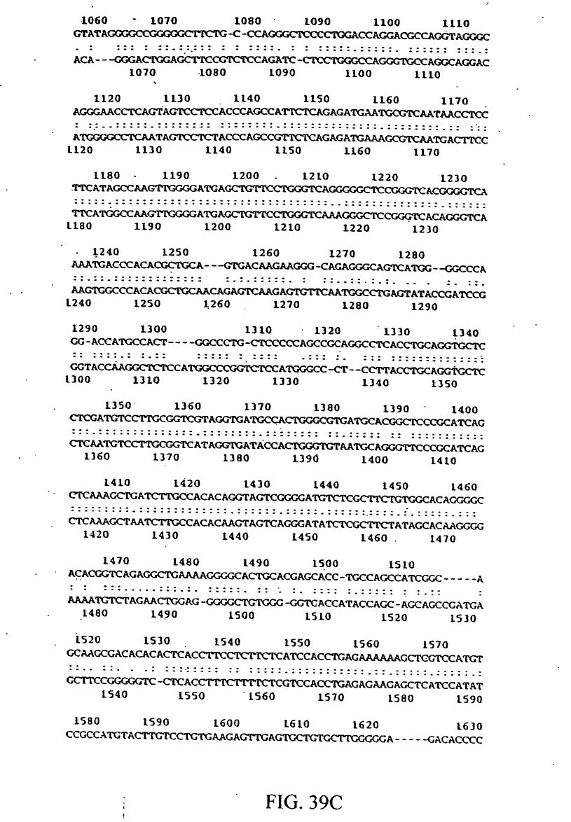

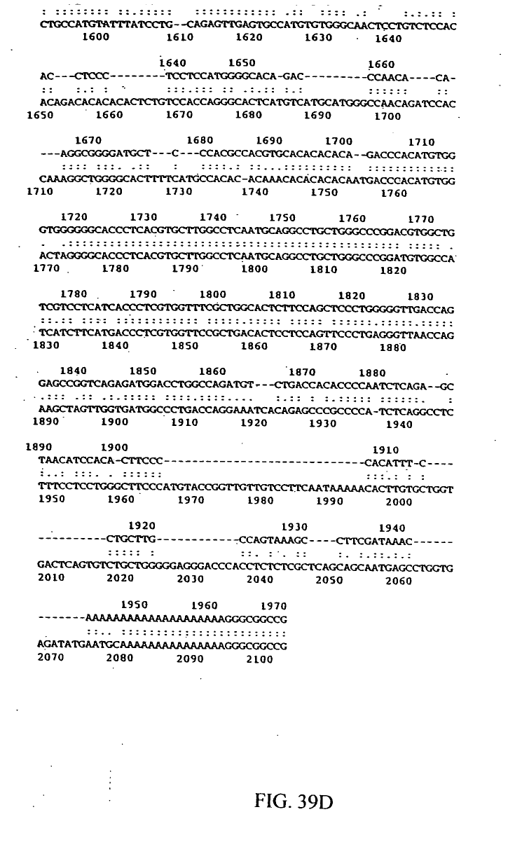

FIG. 29 depicts an alignment of the predicted amino acids sequences of human (SEQ ID NO:30) and murine (SEQ ID NO:61) TANGO 188 (80.5% identity). FIG. 39 depicts an alignment of the cDNA sequences of human (SEQ ID NO:8) and murine (SEQ ID NO:41) TANGO 188 (71.8% identity).

TANGO 188 maps to human chromosome 16p13.3.

Northern analysis of human TANGO 188 mRNA expression revealed the presence of 2.0 kB transcript that is expressed at a low level in heart and pancreas and at a very low level, if at all, in brain, placenta, lung, liver, skeletal muscle, and kidney.

In situ analysis of TANGO 188 expression in adult mice did not detect significant expression in in the bladder, placenta, pancreas, eye, heart, liver, thymus, spleen, kidney, lung, brain, skeletal muscle/diaphragm, colon, or small intestine. In situ analysis of TANGO 188 expression in embryos revealed no significant expression at 13.5, E14.5, E15.5, E16.5, E17.5, or P1.5. However, in the case of both adult mice and embryos, expression of TANGO 188 may have been obscured by a high background signal.

TANGO 188 is transcribed in an anti-sense relationship to NY-CO-7 (Scanlon et al. (1998) Int. J. Cancer 76:652-58). Accordingly, TANGO 188 may have utility as a marker for colon cancer, and TANGO 188 nucleic acid molecules and polypeptides as well as anti-TANGO 188 antibodies and modulators of TANGO 188 expression or activity may be useful in the diagnosis and treatment of colon cancer or other types of cancer.

The gene encoding the C. elegans homologue of NY-CO-7 is present in the same operon as a gene encoding a mitochondrial import protein. Since genes within the same operon are often co-regulated and encode proteins involved in the same physiological state, TANGO 188 may be a mitochondrial import protein or may be involved in some other mitochondrial function. Thus, TANGO 188 nucleic acids and polypeptides as well as antibodies directed against TANGO 188 and modulators of TANGO 188 expression or activity may be useful in the diagnosis and treatment of disorders associated with defects in mitochondrial function.

TANGO 188 appears to be the homologue of a C. elegans protein that is present in the same operon as a gene encoding a protein that bears some similarity to SnF8p, a yeast zinc finger protein that is likely a transcription factor involved in expression of genes encoding certain proteins involved in respiration and metabolism. Since genes within the same operon are often co-regulated and encode proteins involved in the same physiological state, TANGO 188 may play a role in respiration or metabolism. Thus, TANGO 188 nucleic acids and polypeptides as well as antibodies directed against TANGO 188 and modulators of TANGO 188 expression or activity may be useful in the diagnosis and treatment of disorders associated with defects in cell respiration or metabolism.

TANGO 189

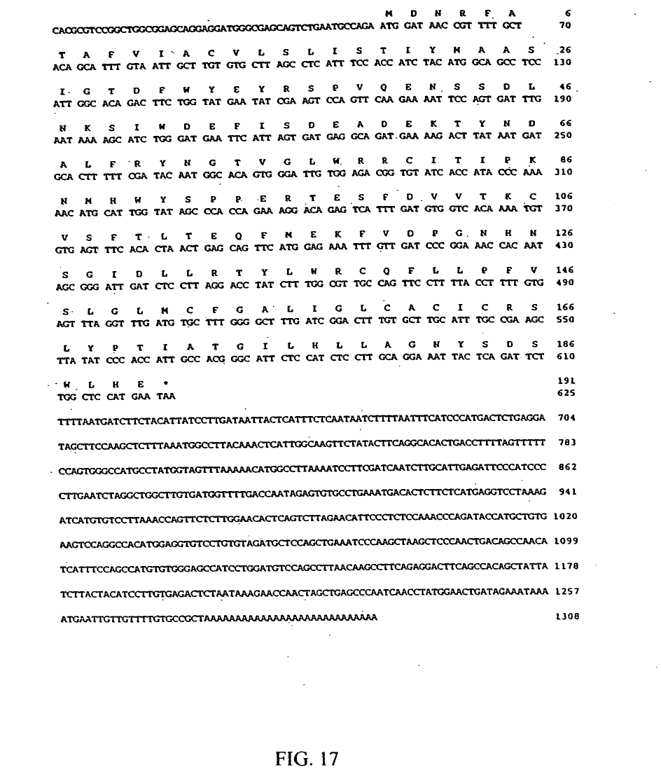

The human TANGO 189 cDNA of SEQ ID NO:9 has a 759 nucleotide open reading frame (SEQ ID NO:20) encoding a 253 amino acid protein (SEQ ID NO:31). The cDNA and protein sequences of human TANGO 189 are shown in FIG. 17.

The human TANGO 189 cDNA described above (SEQ ID NO:9; FIG. 17) represents one splice variant of TANGO 189 (splice variant 1A). There exists a second splice variant of human TANGO 189 (splice variant 1B). The cDNA sequence of this splice variant is the same the cDNA sequence of human TANGO 189 described above, except that nucleotides 674-1087 are missing. This splice variant cDNA encodes a 184 amino acid protein having a predicted molecular weight of 21.1 kDa prior to cleavage of the predicted signal sequence. Both splice variant 1A and splice variant 1B appear to arise from a 2.1 kB transcript which is 2055 nucleotides long, not including the polyA sequence. This transcript encodes a 253 amino acid protein having a predicted molecular weight of 28.6 kDa, not including the predicted signal sequence.

The 2.1 kb TANGO 189 transcript encodes a human TANGO 189 protein that is predicted to be a transmembrane protein having a 24 or 25 amino acid signal sequence (amino acids 1-24 or 1-25 of SEQ ID NO:31; SEQ ID NO:72 and SEQ ID NO:73) followed by a 227 or 226 amino acid mature protein (amino acids 25-251 or 26-251 of SEQ ID NO:31; SEQ ID NO:84 and SEQ ID NO:85) having a first extracellular domain of 114 or 115 amino acids (amino acids 25-138 or 26-138 of SEQ ID NO:31; SEQ ID NO:92 and SEQ ID NO:93), followed by a first transmembrane domain (amino acids 139-164 of SEQ ID NO:31; SEQ ID NO:99), a first cytoplasmic domain (amino acids 165-177 of SEQ ID NO:31; SEQ ID NO:106), a second transmembrane domain (amino acids 178-195 of SEQ ID NO:31; SEQ ID NO:100), a second extracellular domain (amino acids 196-211 of SEQ ID NO:31; SEQ ID NO:108), a third transmembrane domain (amino acids 212-237 of SEQ ID NO:31; SEQ ID NO:101), and a second cytoplasmic domain (amino acids 238-253 of SEQ ID NO:31; SEQ ID NO:107). The protein encoded by this 2.1 kb TANGO 189 transcript is predicted to have a molecular weight of 21.8 kDa prior to cleavage of its signal peptide and a molecular weight of 25.2 kDa subsequent to cleavage of its signal peptide. The predicted domain structure of the protein encoded splice variant 1A is identical to that of the protein encoded by the 2.1 kb transcript up to amino acid 181. The predicted domain structure of the protein encoded splice variant 1B is identical to that of the protein encoded by the 2.1 kb transcript up to amino acid 180.

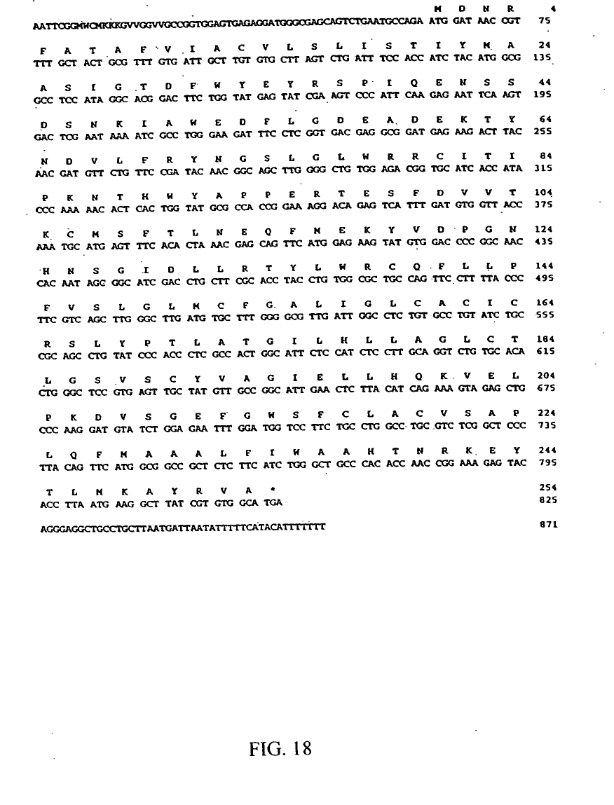

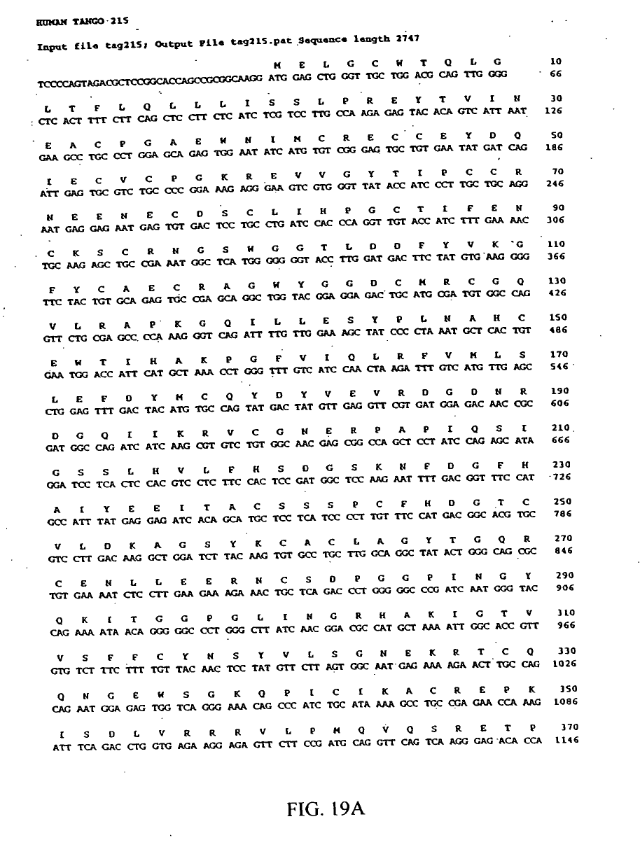

The murine TANGO 189 cDNA of SEQ ID NO:42 has a 759 nucleotide open reading frame (SEQ ID NO:52) encoding a 253 amino acid protein (SEQ ID NO:62). The cDNA and protein sequences of murine TANGO 189 are shown in FIG. 18.

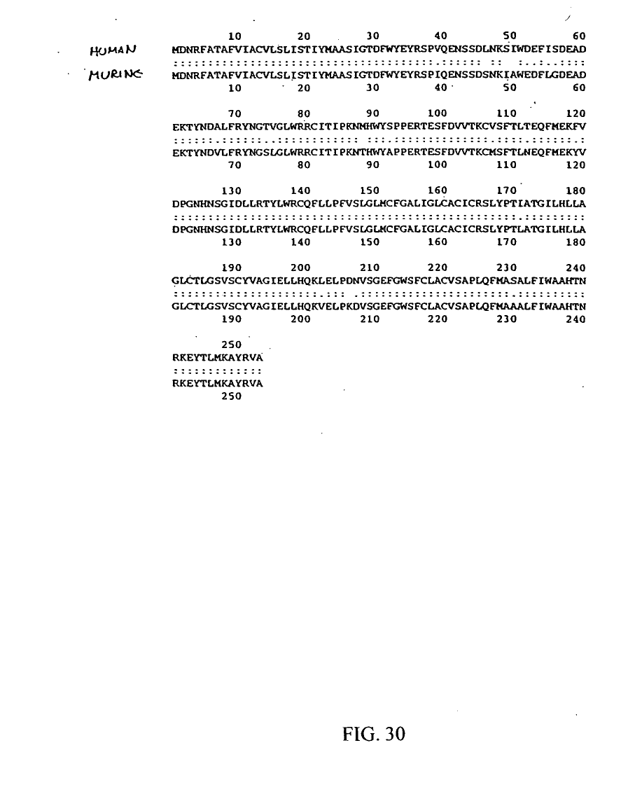

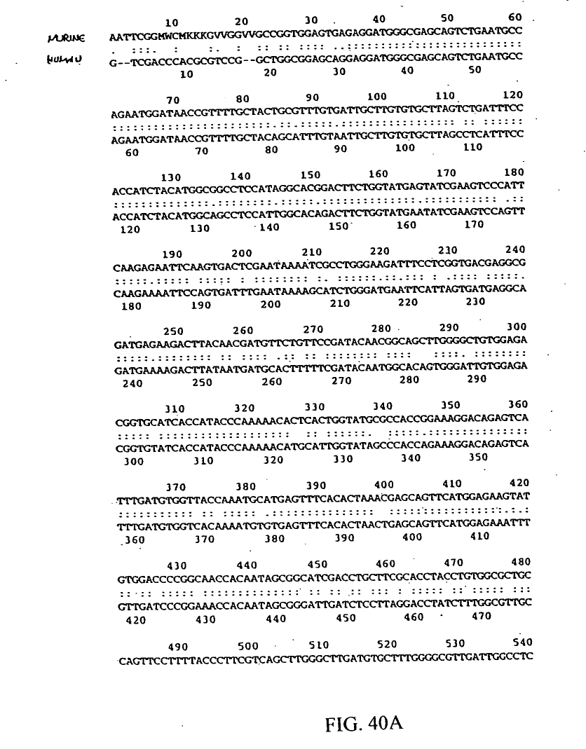

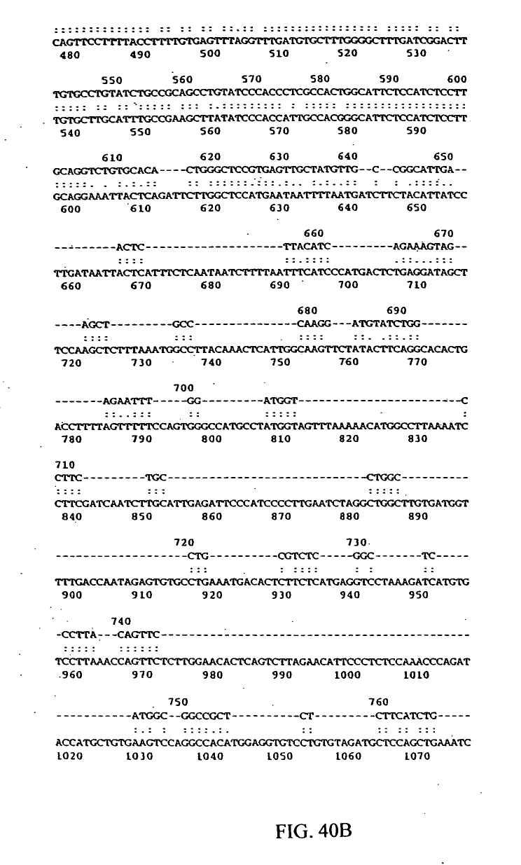

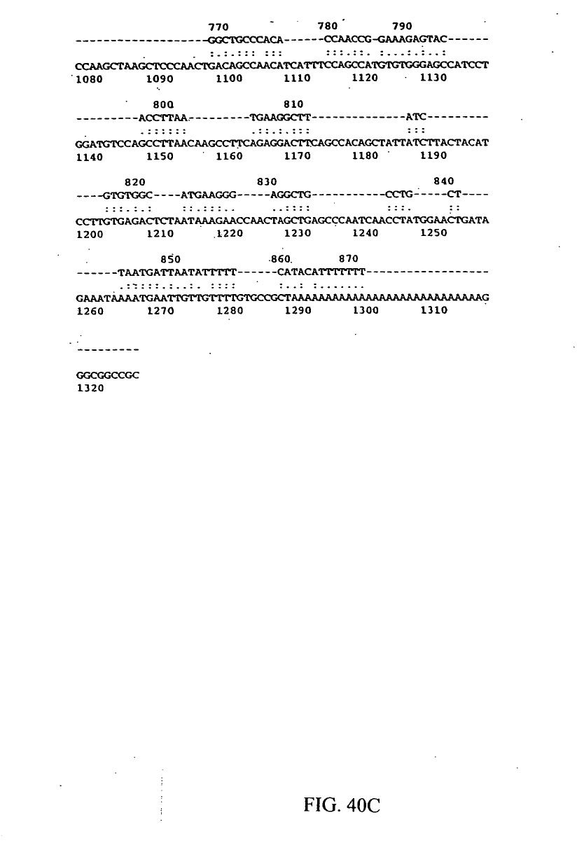

FIG. 30 depicts an alignment of the predicted amino acids sequences of human (SEQ ID NO:31; splice variant 1A) and murine (SEQ ID NO:62) TANGO 189 (91.7% idenity). FIG. 40 depicts an alignment of the cDNA sequences of human (SEQ ID NO:9; splice variant 1A) and murine (SEQ ID NO:42) TANGO 189 (51.8% identity).

Northern analysis of human TANGO 189 mRNA expression revealed the presence of one major transcript (2.1 kb) and four minor transcripts (3.4. kb, 4.2 kb, 6 kb, and 7 kb). The 2.1 kB transcript is expressed at a high level in brain, spinal cord, and testis; expressed at a low level in heart, placenta, skeletal muscle, kidney, pancreas, lung, thyroid, lymph node, trachea, adrenal, bone marrow, spleen, ovary, and prostate; and expressed at a very low level in liver, stomach, thymus, small intestine, colon, peripheral blood lymphocytes. The 3.4. kb, 4.2 kb, 6 kb, and 7 kb transcripts are expressed at a moderate level in brain and spinal cord; and are not expressed in testis. The 4.6 and 7 kb transcripts are expressed at a moderate level in peripheral blood lymphocytes.

Murine in situ expression analysis revealed that TANGO 189 is expressed strongly and almost ubiquitously expressed in the mouse embryo. Tissues with the highest expreession during embryogenesis are the brain, spinal chord, and small intestine. Expression decreases in most if not all tissues by postnatal day 1.5 but tissues of highest expression remain the brain, spinal chord, and small intestine. This pattern continues into the adult mouse with expression in most tissues decreasing even more, some to background levels. Of the adult tissue tested, the brain, spleen, small intestine, and retina, have the highest signal. High level expression is observed in the folowing adult tissues: placenta (ubiquitous), small intestine (except villi), eye (retina), brain (ubiquitous). Lower expression is observed in: bladder (stronger signal in the transitional epithelium), kidney, thymus, liver, placenta, spleen, and colon. Expression was not observed in: heart, skeletal muscle, diaphragm, lung, and pancreas. Embryonic expresion was observed at stages E13.5 through E17.5 (high ubiquitous signal, brain, spinal chord, small intestine have the strongest signal) and P1.5 (ubiquitous signal decreased in intensity, brain, spinal chord, small intestine, and kidney have the strongest signal).

TANGO 189 is useful as a tissue-specific marker. The expression of TANGO 189 may be altered in a variety of disease states (e.g., cancer). Thus, TANGO 189 nucleic acid molecules and polypeptides as well as anti-TANGO 189 antibodies and modulators of TANGO 189 disorders cell proliferation and differentiation.

TANGO 215

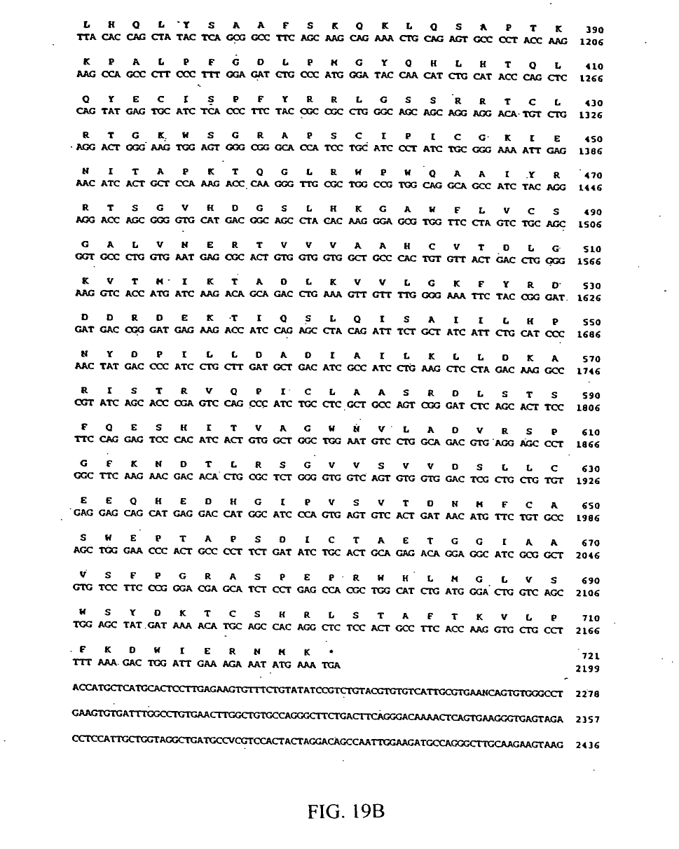

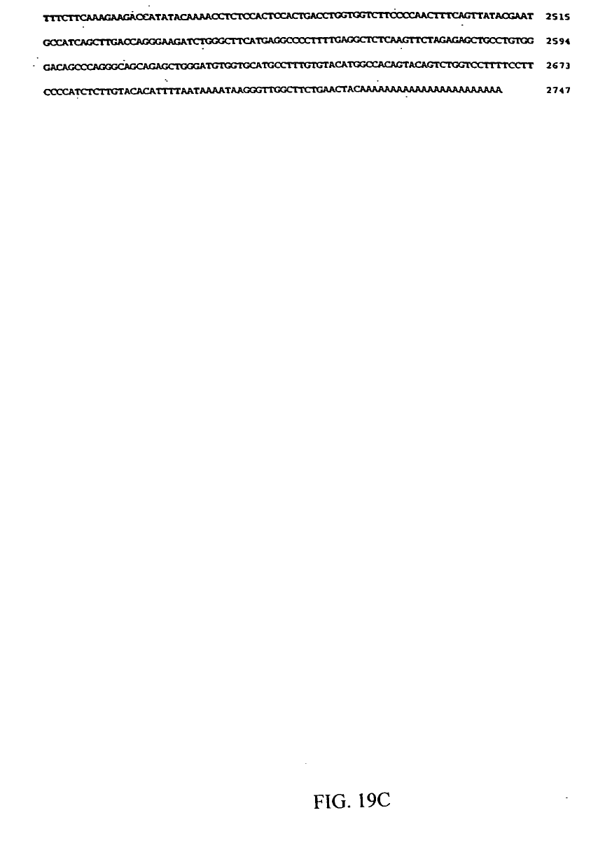

The human TANGO 215 cDNA of SEQ ID NO:10 has a 2160 nucleotide open reading frame (SEQ ID NO:21) encoding a 720 amino acid protein (SEQ ID NO:32). The cDNA and protein sequences of human TANGO 215 are shown in FIG. 19.

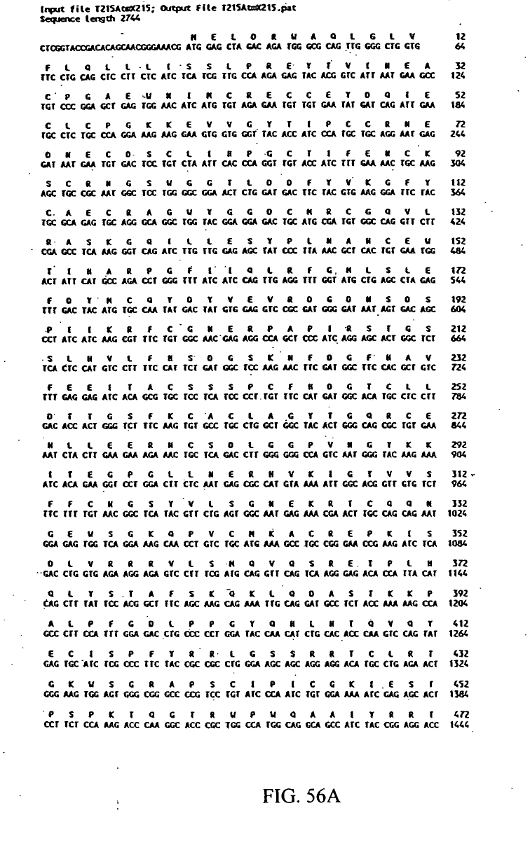

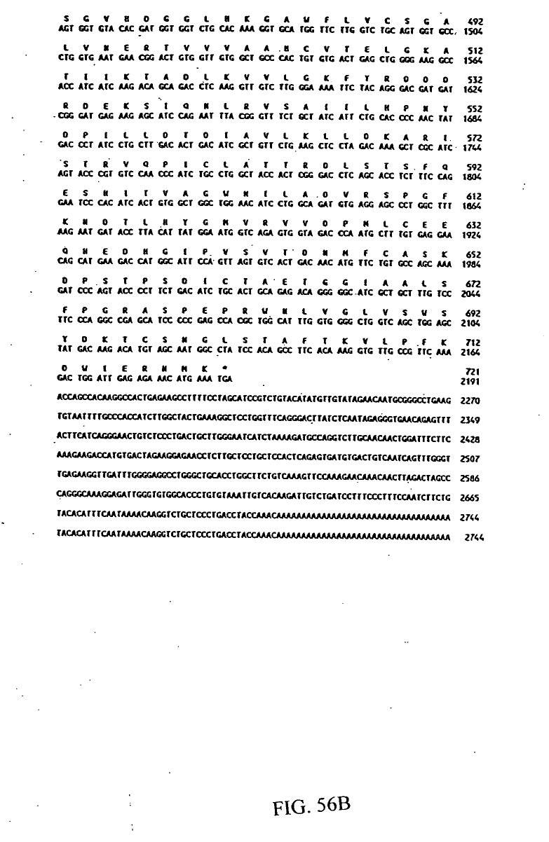

The cDNA sequence (SEQ ID NO:______) and predicted amino acid sequence (SEQ ID NO:______) of a full-length murine TANGO 181 clone are shown in FIG. 56.

Human TANGO 215 is predicted to be a wholly secreted protein having a 21 amino acid signal sequence (amino acids 1-21 of SEQ ID NO:32; SEQ ID NO:74) followed by a 699 amino acid mature protein (amino acids 22-720 of SEQ ID NO:32; SEQ ID NO:86). TANGO 215 is predicted to have a molecular weight of 80.3 kDa prior to cleavage of its signal peptide and a molecular weight of 77.6 kDa subsequent to cleavage of its signal peptide.

TANGO 215 is related to Clr/Cls (C1q) and MASP1/MASP2 (mannose-binding lectin-associated serine protease) proteases, all of which are involved in the alternative pathway pathway of immune response.

TANGO 215 may be a theronine protease. There is a threonine in the sequence TGG at amino acid 664-666 of human and murine TANGO 215. This sequence is within a region having similarity to the active site of certain proteases.

Human TANGO 215 is predicted to have CUB domain (amino acids 128-236 of SEQ ID NO:32), an EGF domain (amino acids 239-271 of SEQ ID NO:32), a small consensus repeat (SCR) domain (amino acids 280-342 of SEQ ID NO:32), a partial SCR domain (amino acids 408-442 of SEQ ID NO:32), and a serine protease domain (amino acids 461-720 of SEQ ID NO:32).

Northern analysis of human TANGO 215 mRNA expression revealed the presence of a 2.7 kb transcript in heart, brain, and placenta.

In situ analysis of TANGO 215 expression in adult mice revealed expression in the brain (cortex and caudate putamen), kidney (cortex, most likely within the glomeruli), bladder (ubiquitous expression), liver (possibly within vessels), and placenta (outer membrane region). This analysis did not detect expression in the lung, small intestine, pancreas, thymus, eye, heart, or muscle/diaphragm.

In situ analysis of TANGO 215 in embryos revealed expression at E13.5 in developing limbs and vertebrae. At E14.5 the observed expression pattern was similar to that at E13.5 except that expression was observed in the muscle surrounding abdomen, the skin, and the jaw. At E15.5 expression was observed in the developing kidney and bladder and outer layer of the tongue. At later ages, E16.5 through P1.5, expression is observed in the smooth muscle layer of the small intestine, the portal regions of the liver, and the large airways of the lungs. Expression in the brain is absent until E18.5 when expression is apparent in the caudate putamen. Expression remains strong at P1.5 in the vertebrae, tail, and sternum and possibly the muscle between developing bones.

The region of human TANGO 215 from amino acid 280 to the end is predicted to be the human homologue of Limilus Factor C (27% identity). Thus, this region of TANGO 215 is predicted to include an effector domain (serine protease domain) and, perhaps, an LPS sensing domain. Thus, TANGO 215 may sense and respond to LPS with the response to the presence of LPS being activation of serine protease activity. Accordingly, TANGO 215 nucleic acids and polypeptides as well as antibodies directed against TANGO 215 and modulators of TANGO 215 expression or activity may be useful in the diagnosis and treatment sepsis.

CUB domains are extracellular domains of about 110 amino acids. CUB domains are found in functionally diverse, mostly developmentally regulated proteins. Most contain four cysteines that are involved in two disulfide bonds (C1-C2 and C3-C4). SCR domains are also known as complement control protein (CCP) modules. EGF domains are commonly involved in receptor-ligand interactions. CUB, EGF, and SCR domains are commonly involved in protein-protein interaction. Because these domains are present in TANGO 215, it is predicted to interact with one or more other proteins. The presence of these domains in TANGO 215 suggests that TANGO 215 is involved in development, perhaps bone and cartilage morphogenesis. TANGO 215 nucleic acid molecules and polypeptides as well as anti-TANGO 215 antibodies and modulators of TANGO 215 expression or activity may be useful in the treatment of developmental disorders.

TANGO 187

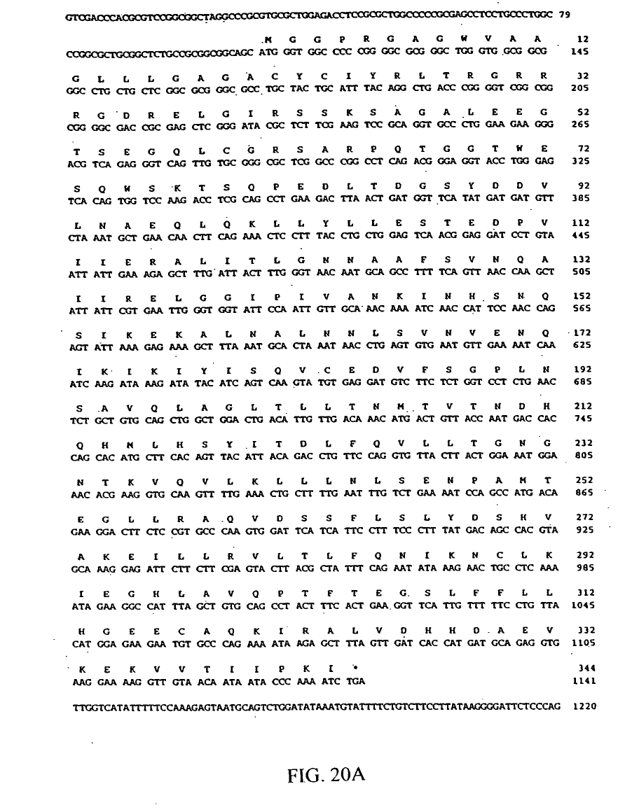

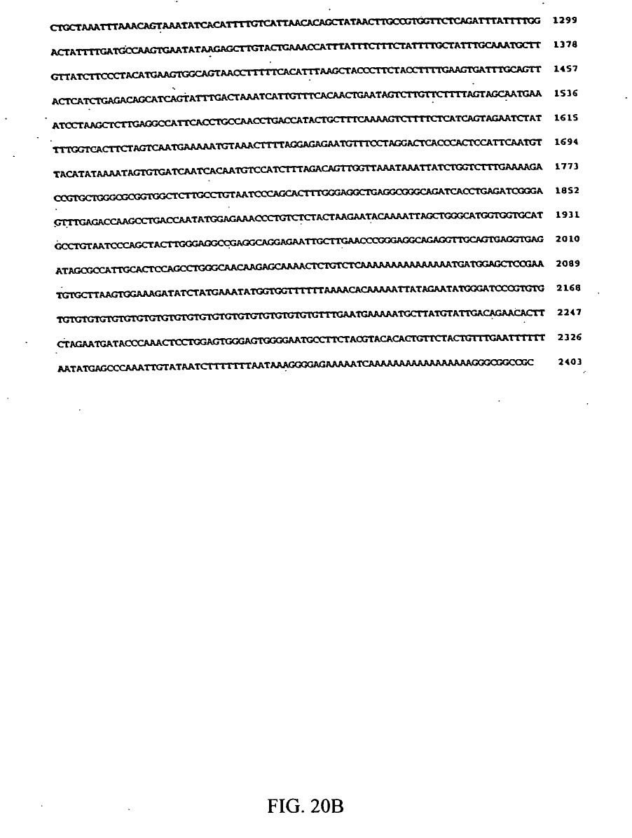

The human TANGO 187-1/3 cDNA of SEQ ID NO:11 has a 1032 nucleotide open reading frame (SEQ ID NO:22) encoding a 343 amino acid protein (SEQ ID NO:33). The cDNA and protein sequences of human TANGO 187-1/3 are shown in FIG. 20.

Human TANGO 187-1/3 is predicted to be a wholly secreted protein having a 20 amino acid signal sequence (amino acids 1-20 of SEQ ID NO:33; SEQ ID NO:75) followed by a 323 amino acid mature protein (amino acids 21-343 of SEQ ID NO:33; SEQ ID NO:87). Human TANGO 187-1/3 is predicted to have a molecular weight of 37.5 kDa prior to cleavage of its signal peptide and a molecular weight of 35.9 kDa subsequent to cleavage of its signal peptide.

The TANGO 187-1/3 cDNA described upon actually represents one of 8 different TANGO 187 splice variants. Each variant contains none, one, two or three of three variant regions. These regions are referred to as region 1, region 2, and region 3, and each of the various forms of TANGO 187 is referred to by including a reference to the variant regions present. Thus, the form of TANGO 187 described above is TANGO 187-1/3 because it includes regions 1 and 3.

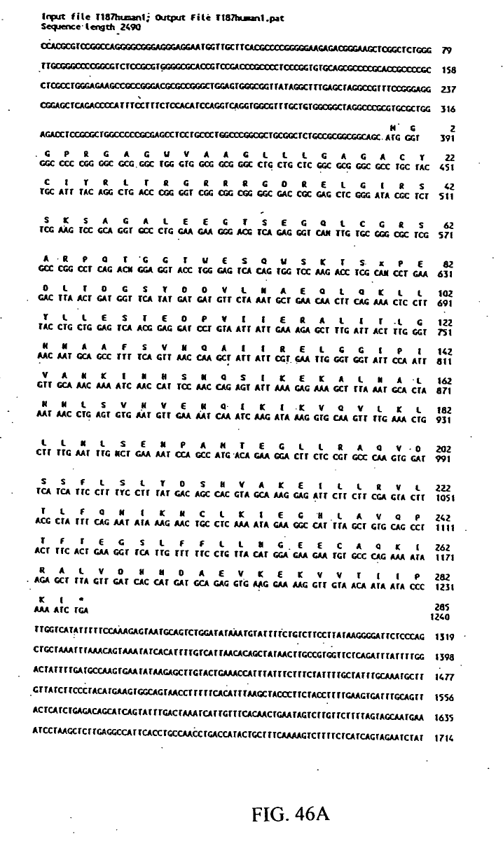

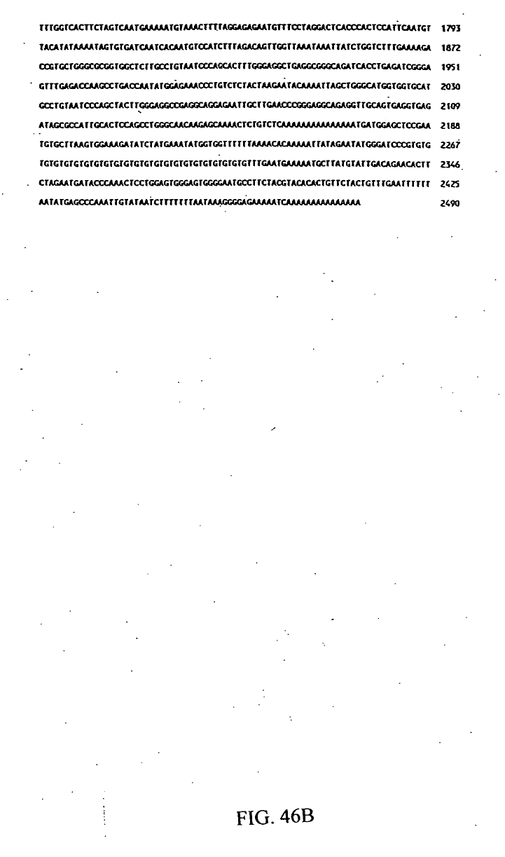

FIG. 46 depicts the cDNA sequence (SEQ ID NO:______) and predicted amino acid sequence (SEQ ID NO:______) of TANGO 187-1.

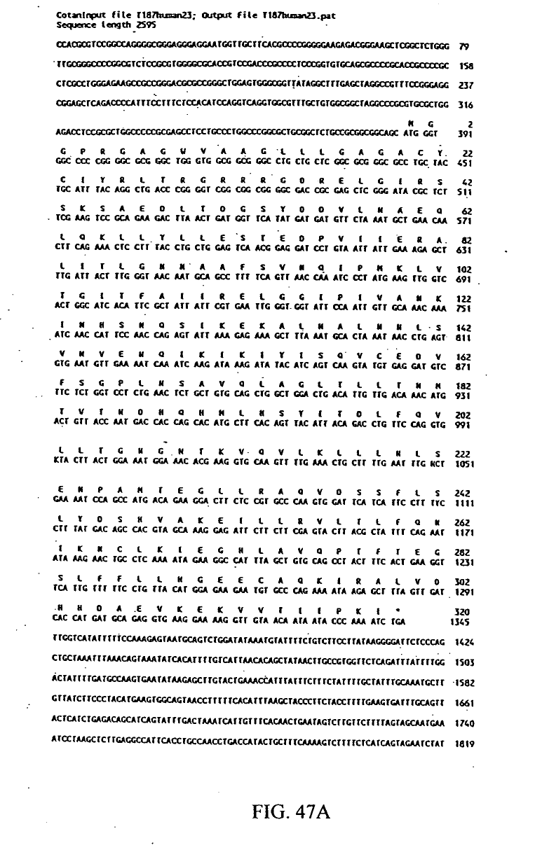

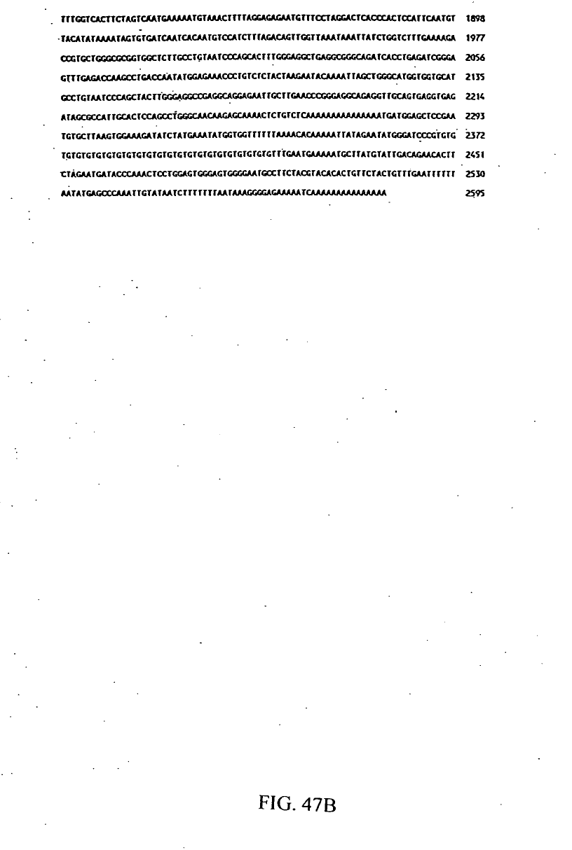

FIG. 47 depicts the cDNA sequence (SEQ ID NO:______) and predicted amino acid sequence (SEQ ID NO:______) of TANGO 187-2/3.

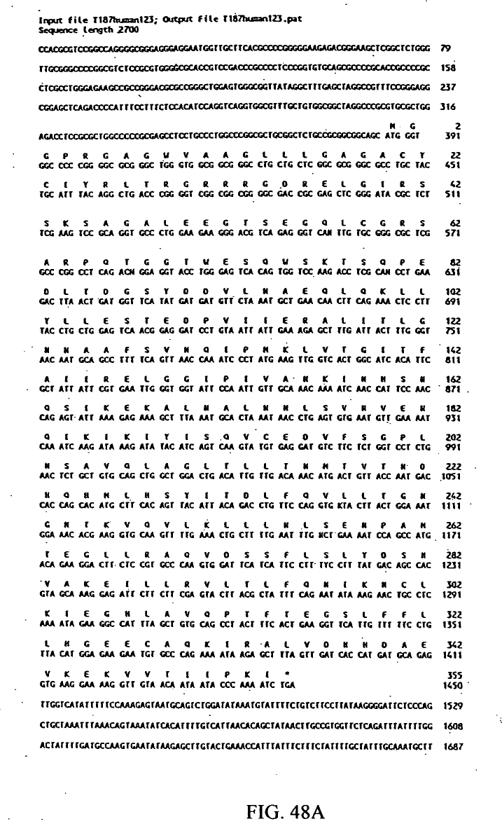

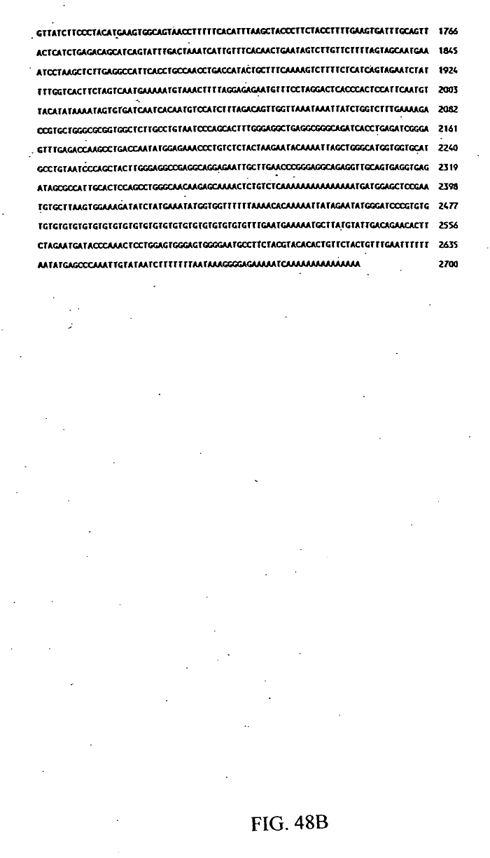

FIG. 48 depicts the cDNA sequence (SEQ ID NO:______) and predicted amino acid sequence (SEQ ID NO:______) of TANGO 187-1/2/3.

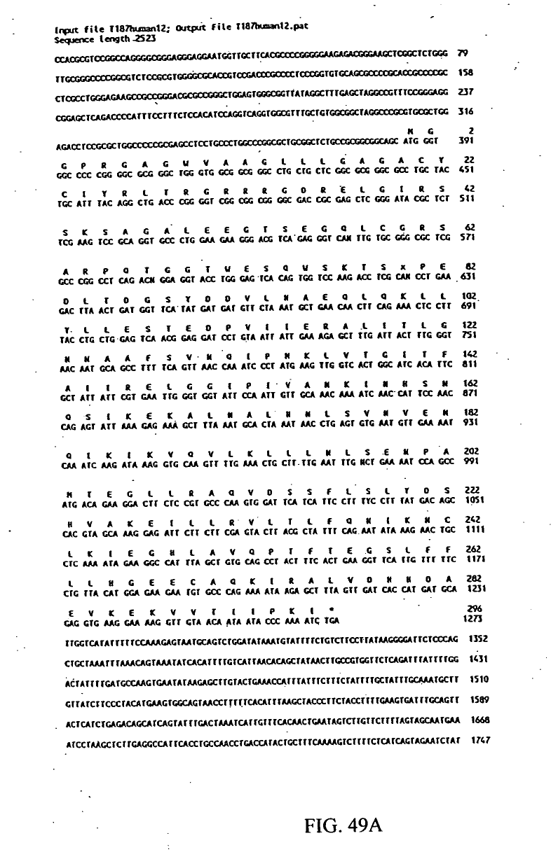

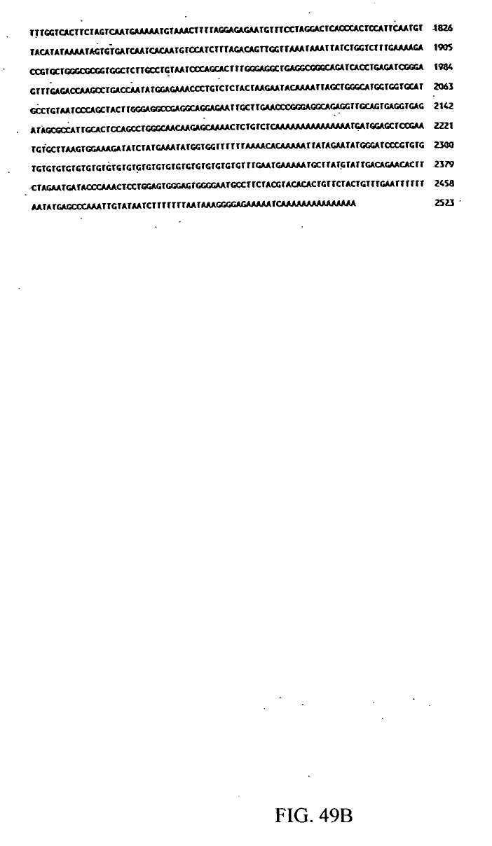

FIG. 49 depicts the cDNA sequence (SEQ ID NO:______) and predicted amino acid sequence (SEQ ID NO:______) of TANGO 187-1/2.

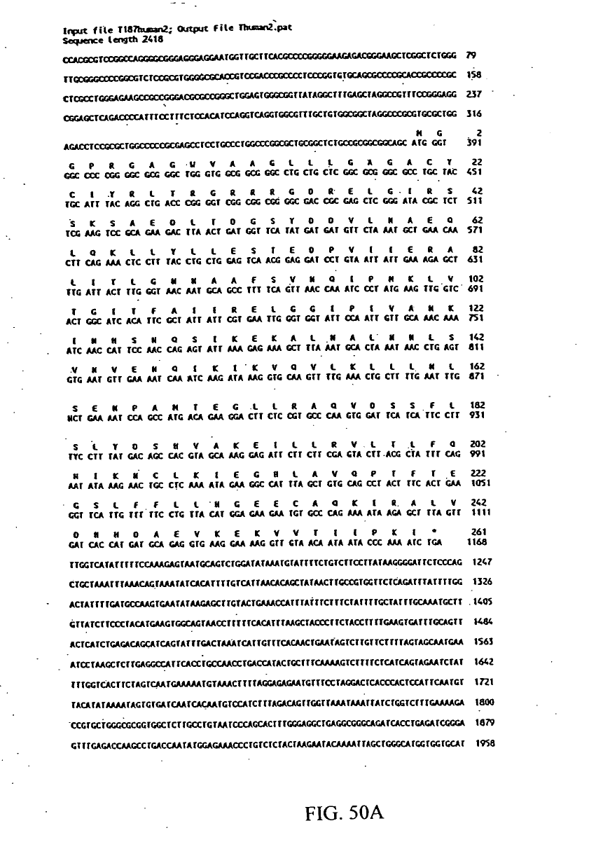

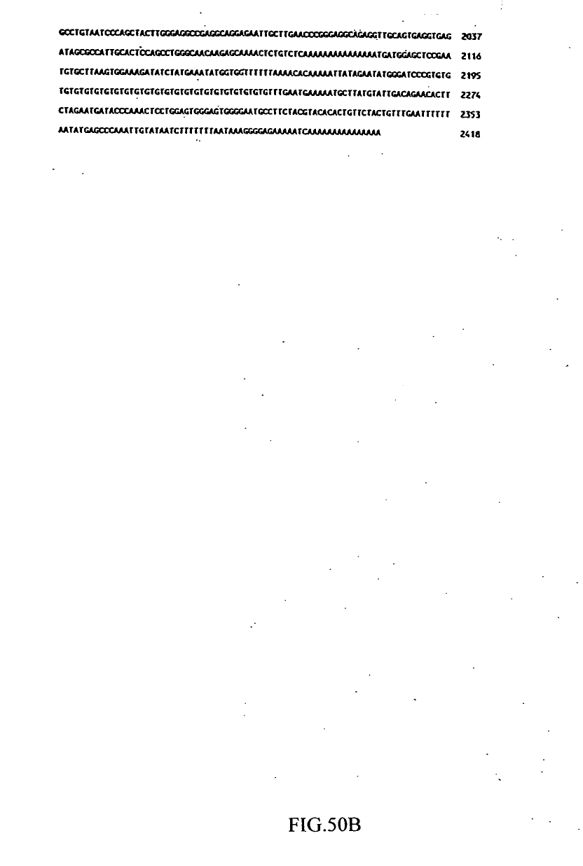

FIG. 50 depicts the cDNA sequence (SEQ ID NO:______) and predicted amino acid sequence (SEQ ID NO:______) of TANGO 187-2.

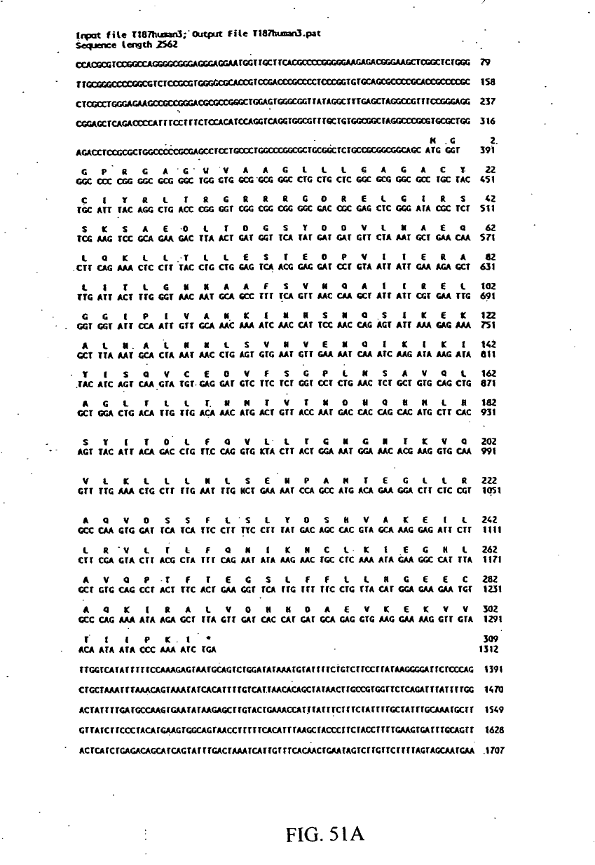

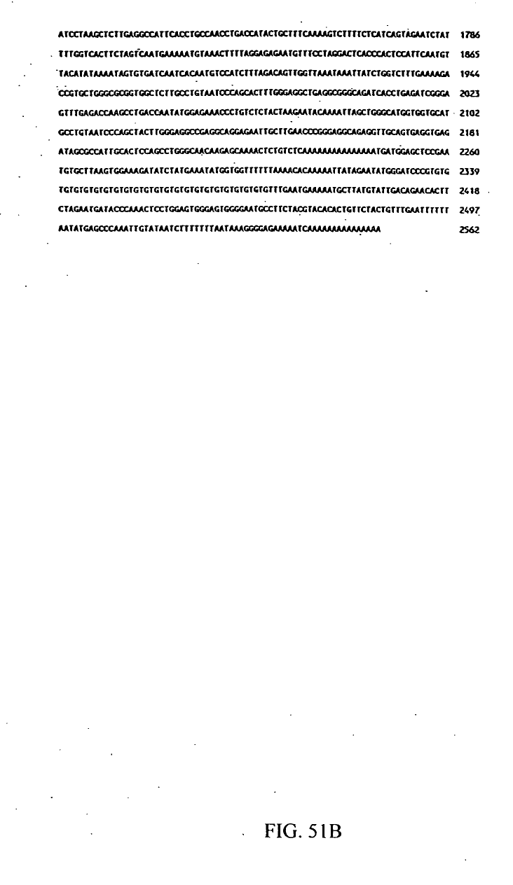

FIG. 51 depicts the cDNA sequence (SEQ ID NO:______) and predicted amino acid sequence (SEQ ID NO:______) of TANGO 187-3.

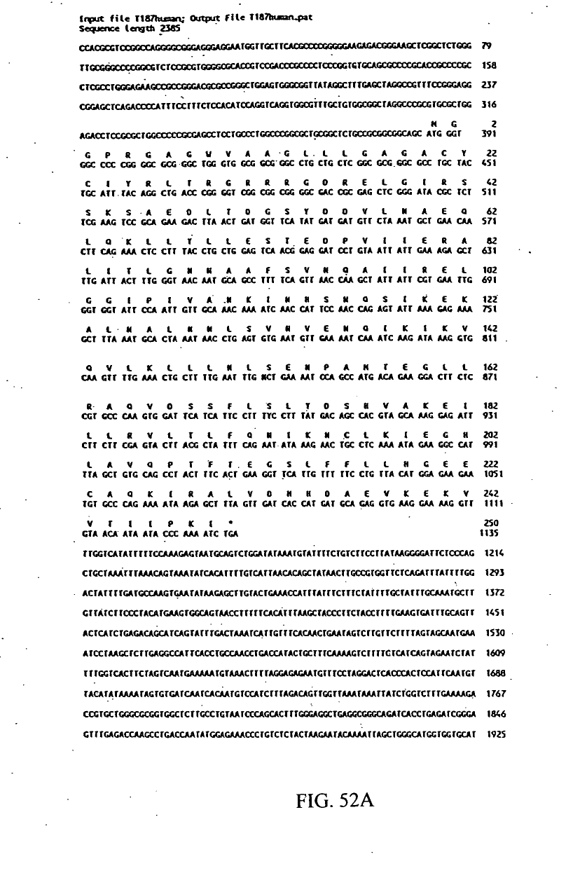

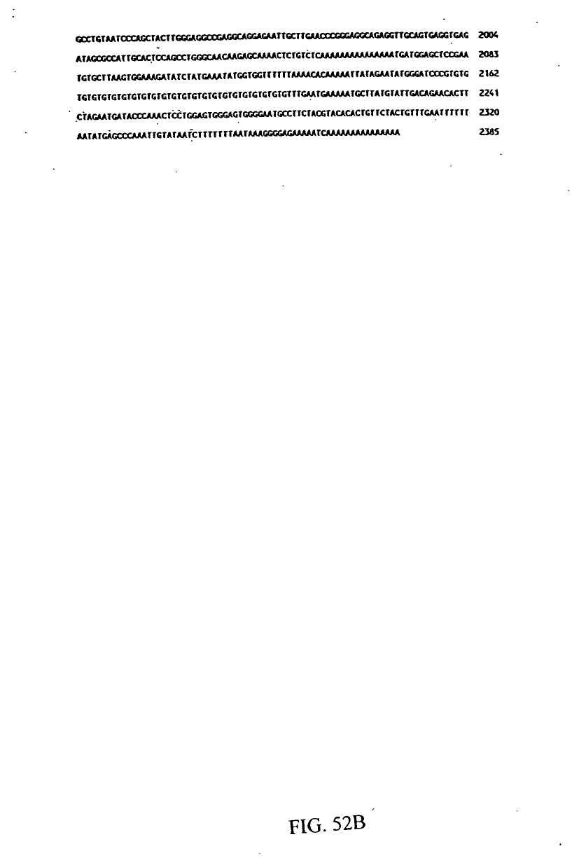

FIG. 52 depicts the cDNA sequence (SEQ ID NO:______) and predicted amino acid sequence (SEQ ID NO:______) of TANGO 187. This form does not include any of the three variant regions.

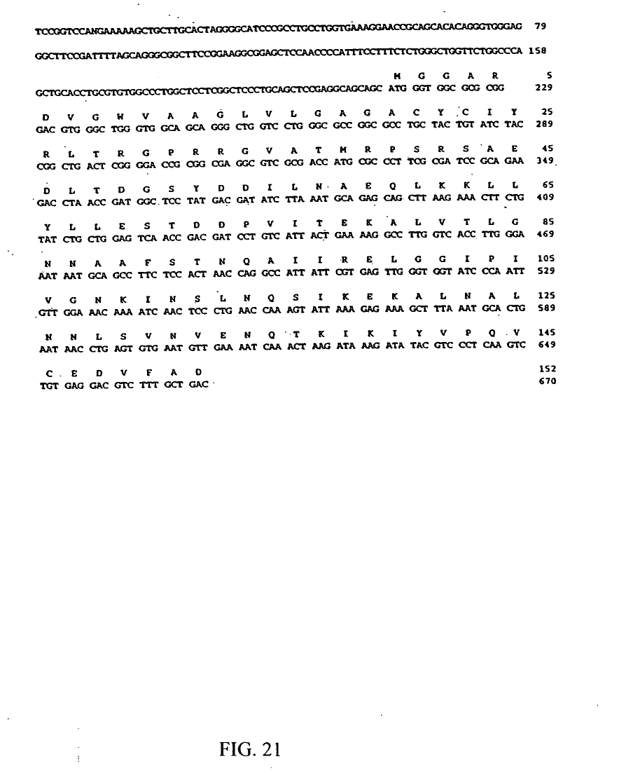

The murine TANGO 187 cDNA of SEQ ID NO:43 is only a partial sequence. This cDNA has an open reading frame extending from nucleotide 73 to the end of the available sequence (SEQ ID NO:53) encoding a 152 amino acid protein (SEQ ID NO:63). The partial cDNA and protein sequences of murine TANGO 187 are shown in FIG. 21.

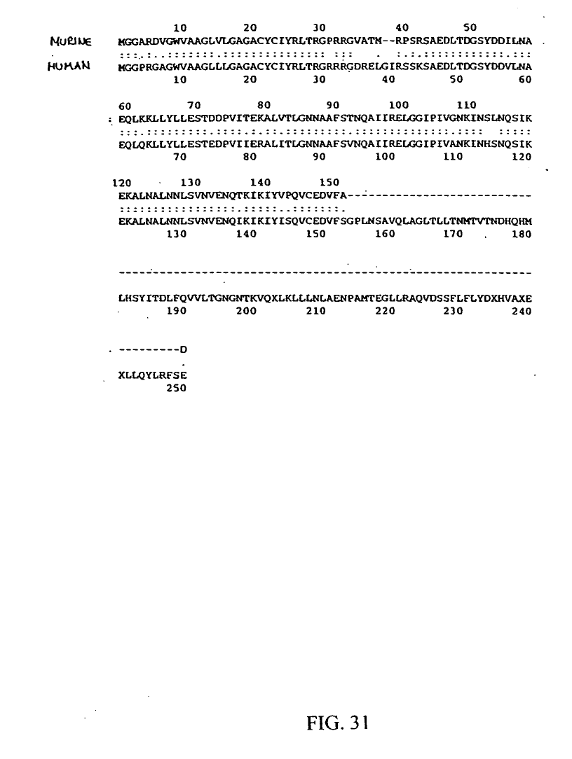

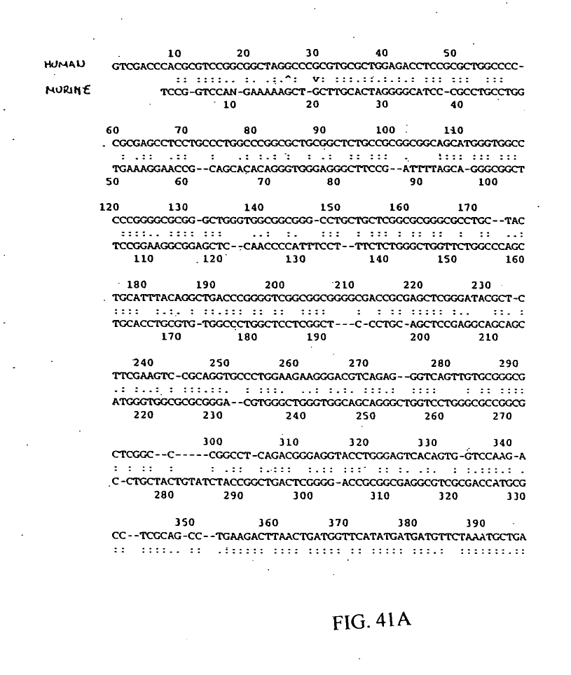

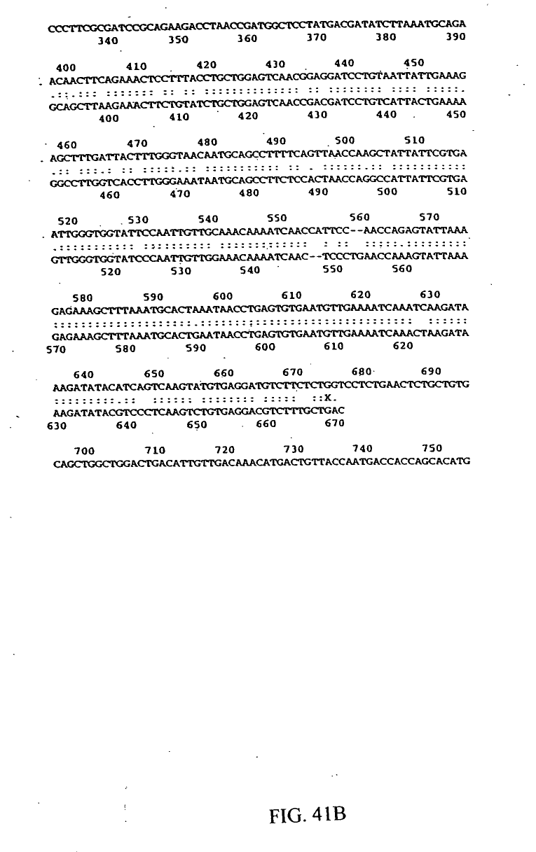

FIG. 31 depicts an alignment of the predicted amino acid sequences of human (SEQ ID NO:33) and murine (SEQ ID NO:63; partial) TANGO 187 (50.4% identity). FIG. 41 depicts an alignment of the cDNA sequences of human (SEQ ID NO:11) and murine (SEQ ID NO:43; partial) TANGO 187 (66.0% identity).

Northern analysis of human TANGO 187 mRNA expression revealed the presence of 1.3 and 2.4 kb transcripts that are approximately equally expressed at a low level in heart, brain, lung, liver, and smooth muscle and at a moderate level in kidney and placenta.

In situ analysis of TANGO 187 expression in adult mice revealed that TANGO 187 is expressed in brain (weak, ubiquitous signal), eye and harderian gland (weak signal in the retina), submandibular gland (weak, ubiquitous signal), stomach (weak, ubiquitous signal), kidney (weak, ubiquitous signal), adrenal gland (low level, ubiquitous expression), colon (low level, ubiquitous expression), small intestine (low level, ubiquitous expression), thymus (moderate level, ubiquitous expression in the cortical region with lower expression in the medulla), lymph node (ubiquitous expression), spleen (low level ubiquitous expression with lower expression in the follicles, bladder (moderate expression in the mucosal epithelium), testes (moderate, ubiquitous expression signal that defines the seminiferous vesicles). In this analysis, TANGO 187 expression was not detectable in the spinal cord, brown fat, heart, lung, liver, pancreas, skeletal muscle, and ovaries.

In situ analysis of TANGO 187 expression in embryos at E13.5 revealed ubiquitous expression with the strongest expression in the brain and spinal cord. A punctate expression pattern was observed in the lungs suggestive of higher expression in the developing large airways. At E14.5 the expression pattern was similar to that observed at E13.5 except that expression was observed in the developing olfactory system and the eye at a level similar to that observed in the brain and spinal cord. Expression is also present at E14.5 in the epithelium of the tongue, the dermis of the snout, the kidneys and the stomach. At E15.5 low level ubiquitous expression was observed with the highest expression in the brain, spinal cord, eye, and olfactory system. Slightly lower expression was observed in the lung (ubiquitous expression) and kidney (cortical region) than in the aforementioned neuronal tissues. At E16.5 the observed expression pattern is identical to that seen at E15.5 except TANGO 187 expression is observed in the thymus and the mucosal portion of the stomach. At E18.5 TANGO 187 continues to be highest in neuronal tissue with lower expression in the hind brain and spinal cord than in the forebrain with the neopallial cortex having the highest signal. At E16.5 expression is observed in the thymus and small intestine. At P1.5 the observed expression pattern is nearly identical to that at E18.5 except that expression in the the lung and stomach has decreased. At P1.5 expression is highest in the brain, eye, olfactory epithelium and kidney.

Tango 187 contain a region moderately similar to an armadillo/beta-catenin repeat. Such repeats are thought to be involved in protein-protein interactions.

TABLE 1
Summary of Human TANGO 180, TANGO 181, TANGO
182, TANGO 183, TANGO 184, TANGO 185, TANGO
186, TANGO 187, TANGO 188, TANGO 189, and TANGO
215 Sequence Information.

| | | | | | Accession |
|---|---|---|---|---|---|
| Gene | cDNA | ORF | Protein | FIG. | No. |
| TANGO | SEQ ID | SEQ ID | SEQ ID | | ATCC |
| 180 | NO: 1 | NO: 12 | NO: 23 | | 98900 |
| TANGO | SEQ ID | SEQ ID | SEQ ID | | ATCC |
| 181 | NO: 2 | NO: 13 | NO: 24 | | 98900 |
| TANGO | SEQ ID | SEQ ID | SEQ ID | | ATCC |
| 182 | NO: 3 | NO: 14 | NO: 25 | | 98900 |
| TANGO | SEQ ID | SEQ ID | SEQ ID | | ATCC |
| 183 | NO: 4 | NO: 15 | NO: 26 | | 98900 |
| TANGO | SEQ ID | SEQ ID | SEQ ID | | ATCC |
| 184 | NO: 5 | NO: 16 | NO: 27 | | 98900 |
| TANGO | SEQ ID | SEQ ID | SEQ ID | FIG. | ATCC |
| 185 | NO: 6 | NO: 17 | NO: 28 | 11 | 98901 |
| TANGO | SEQ ID | SEQ ID | SEQ ID | FIG. | ATCC |
| 186 | NO: 7 | NO: 18 | NO: 29 | 13 | 98901 |
| TANGO | SEQ ID | SEQ ID | SEQ ID | FIG. | ATCC |
| 188 | NO: 8 | NO: 19 | NO: 30 | 15 | 98901 |
| TANGO | SEQ ID | SEQ ID | SEQ ID | FIG. | ATCC |
| 189 | NO: 9 | NO: 20 | NO: 31 | 17 | 98901 |
| TANGO | SEQ ID | SEQ ID | SEQ ID | FIG. | ATCC |
| 215 | NO: 10 | NO: 21 | NO: 32 | 19 | 98899 |
| TANGO | SEQ ID | SEQ ID | SEQ ID | FIG. | ATCC |
| 187-1/3 | NO: 11 | NO: 22 | NO: 33 | 20 | 98901 |
| TANGO | SEQ ID | SEQ ID | SEQ ID | FIG. | ATCC |
| 187-1 | NO: | NO: | NO: | 46 | |
| TANGO | SEQ ID | SEQ ID | SEQ ID | FIG. | ATCC |
| 187-2/3 | NO: | NO: | NO: | 47 | |
| TANGO | SEQ ID | SEQ ID | SEQ ID | FIG. | ATCC |
| 187- | NO: | NO: | NO: | 48 | |
| 1/2/3 | | | | | |
| TANGO | SEQ ID | SEQ ID | SEQ ID | FIG. | ATCC |
| 187-1/2 | NO: | NO: | NO: | 49 | |
| TANGO | SEQ ID | SEQ ID | SEQ ID | FIG. | ATCC |
| 187-2 | NO: | NO: | NO: | 50 | |
| TANGO | SEQ ID | SEQ ID | SEQ ID | FIG. | ATCC |
| 187-3 | NO: | NO: | NO: | 51 | |

TABLE 2
Summary of Domains of Human TANGO 180, TANGO
181, TANGO 182, TANGO 183, TANGO 184, TANGO
185, TANGO 186, TANGO 187, TANGO 188, TANGO
189, and TANGO 215.

| | Signal | | Extra- | Trans- | |
|---|---|---|---|---|---|
| | Se- | Mature | cellular | membrane | Cytoplasmic |
| Protein | quence | Protein | Domain | Domain | Domain |
| TANGO | aa 1-22 | aa 23-189 | — | — | — |
| 180 | SEQ ID | SEQ ID | | | |
| | NO: 64 | NO: 76 | | | |
| TANGO | aa 1-22 | aa 23-339 | — | — | — |
| 181 | SEQ ID | SEQ ID | | | |
| | NO: 65 | NO: 77 | | | |
| TANGO | aa 1-23 | aa 24-348 | — | — | — |
| 182 | SEQ ID | SEQ ID | | | |
| | NO: 66 | NO: 78 | | | |
| TANGO | aa 1-20 | aa 21-183 | aa 21-89 | aa 90-112 | aa 113-183 |
| 183 | SEQ ID | SEQ ID | SEQ ID | SEQ ID | SEQ ID |
| | NO: 67 | NO: 79 | NO: 88 | NO: 94 | NO: 102 |
| TANGO | aa 1-28 | aa 29-198 | aa 29-102 | aa 103-125 | aa 126-198 |
| 184 | SEQ ID | SEQ ID | SEQ ID | SEQ ID | SEQ ID |
| | NO: 68 | NO: 80 | NO: 89 | NO: 95 | NO: 103 |
| TANGO | aa 1-24 | aa 25-193 | aa 25-75 | aa 76-102 | aa 103-109 |
| 185 | SEQ ID | SEQ ID | SEQ ID | SEQ ID | SEQ ID |
| | NO: 69 | NO: 81 | NO: 90 | NO: 96 | NO: 104 |
| | | | and aa | and | and |
| | | | 131-150 | aa 110-131 | aa 175-193 |
| | | | SEQ ID | SEQ ID | SEQ ID |
| | | | NO: 91 | NO: 97 | NO: 105 |
| | | | | and | |
| | | | | aa 151-174 | |
| | | | | SEQ ID | |
| | | | | NO: 98 | |
| TANGO | aa 1-20 | aa 21-383 | — | — | — |
| 186 | SEQ ID | SEQ ID | | | |
| | NO: 70 | NO: 82 | | | |
| TANGO | aa 1-23 | aa 24-264 | — | — | — |
| 188 | SEQ ID | SEQ ID | | | |
| | NO: 71 | NO: 83 | | | |
| TANGO | aa 1-24 | aa 25-251 | aa 25-138 | aa 139-164 | aa 165-177 |
| 189 | SEQ ID | SEQ ID | SEQ ID | SEQ ID | SEQ ID |
| | NO: 72 | NO: 84 | NO: 92 | NO: 99 | NO: 106 |
| | or | or | or | and | and |
| | aa 1-25 | aa 26-251 | aa 26-138 | aa 178-195 | aa 238-253 |
| | SEQ ID | SEQ ID | SEQ ID | SEQ ID | SEQ ID |
| | NO: 73 | NO: 85 | NO: 93 | NO: 100 | NO: 107 |
| | | | and aa | and | |
| | | | 196-211 | aa 212-237 | |
| | | | SEQ ID | SEQ ID | |
| | | | NO: 108 | NO: 101 | |
| TANGO | aa 1-21 | aa 22-720 | — | — | — |
| 215 | SEQ ID | SEQ ID | | | |
| | NO: 74 | NO: 86 | | | |
| TANGO | aa 1-20 | aa 21-343 | — | — | — |
| 187-1/3 | SEQ ID | SEQ ID | | | |
| | NO: 75 | NO: 87 | | | |

TABLE 3
Summary of Murine TANGO 180, TANGO 180, TANGO
181, TANGO 182, TANGO 183, TANGO 184, TANGO
185, TANGO 186, TANGO 188, TANGO 189, and TANGO
187 Sequence Information

| | | | | | AA | NA |
|---|---|---|---|---|---|---|
| | | | | | align. | align. |
| | | | | | with | with |
| Gene | cDNA | ORF | Protein | FIG. | human | human |
| TANGO | SEQ ID | SEQ ID | SEQ ID | | | |
| 180 | NO: 34 | NO: 44 | NO: 54 | | | |
| TANGO | SEQ ID | SEQ ID | SEQ ID | | | |
| 181 | NO: 35 | NO: 45 | NO: 55 | | | |
| (partial) | | | | | | |
| TANGO | SEQ ID | SEQ ID | SEQ ID | | | |
| 182 | NO: 36 | NO: 46 | NO: 56 | | | |
| (partial) | | | | | | |
| TANGO | SEQ ID | SEQ ID | SEQ ID | | | |
| 183 | NO: 37 | NO: 47 | NO: 57 | | | |
| TANGO | SEQ ID | SEQ ID | SEQ ID | FIG. | | |
| 184 | NO: 38 | NO: 48 | NO: 58 | 10 | | |
| TANGO | SEQ ID | SEQ ID | SEQ ID | FIG. | | |
| 185 | NO: 39 | NO: 49 | NO: 59 | 12 | | |
| TANGO | SEQ ID | SEQ ID | SEQ ID | FIG. | | |
| 186 | NO: 40 | NO: 50 | NO: 60 | 14 | | |
| TANGO | SEQ ID | SEQ ID | SEQ ID | FIG. | | |
| 188 | NO: 41 | NO: 51 | NO: 61 | 16 | | |
| TANGO | SEQ ID | SEQ ID | SEQ ID | FIG. | | |
| 189 | NO: 42 | NO: 52 | NO: 62 | 18 | | |
| TANGO | SEQ ID | SEQ ID | SEQ ID | FIG. | | |
| 187 | NO: 43 | NO: 53 | NO: 63 | 21 | | |
| (partial) | | | | | | |
| TANGO | SEQ ID | SEQ ID | SEQ ID | FIG. | | |
| 181 | NO: | NO: | NO: | 53 | | |
| TANGO | SEQ ID | SEQ ID | SEQ ID | FIG. | | |
| 182 | NO: | NO: | NO: | 54 | | |
| TANGO | SEQ ID | SEQ ID | SEQ ID | FIG. | | |
| 187 | NO: | NO: | NO: | 55 | | |
| TANGO | SEQ ID | SEQ ID | SEQ ID | FIG. | | |
| 215 | NO: | NO: | NO: | 56 | | |

Various aspects of the invention are described in further detail in the following subsections

I. Isolated Nucleic Acid Molecules

One aspect of the invention pertains to isolated nucleic acid molecules that encode a polypeptide of the invention or a biologically active portion thereof, as well as nucleic acid molecules sufficient for use as hybridization probes to identify nucleic acid molecules encoding a polypeptide of the invention and fragments of such nucleic acid molecules suitable for use as PCR primers for the amplification or mutation of nucleic acid molecules. As used herein, the term “nucleic acid molecule” is intended to include DNA molecules (e.g., cDNA or genomic DNA) and RNA molecules (e.g., mRNA) and analogs of the DNA or RNA generated using nucleotide analogs. The nucleic acid molecule can be single-stranded or double-stranded, but preferably is double-stranded DNA.

An “isolated” nucleic acid molecule is one which is separated from other nucleic acid molecules which are present in the natural source of the nucleic acid molecule. Preferably, an “isolated” nucleic acid molecule is free of sequences (preferably protein encoding sequences) which naturally flank the nucleic acid (i.e., sequences located at the 5′ and 3′ ends of the nucleic acid) in the genomic DNA of the organism from which the nucleic acid is derived. For example, in various embodiments, the isolated nucleic acid molecule can contain less than about 5 kB, 4 kB, 3 kB, 2 kB, 1 kB, 0.5 kB or 0.1 kB of nucleotide sequences which naturally flank the nucleic acid molecule in genomic DNA of the cell from which the nucleic acid is derived. Moreover, an “isolated” nucleic acid molecule, such as a cDNA molecule, can be substantially free of other cellular material, or culture medium when produced by recombinant techniques, or substantially free of chemical precursors or other chemicals when chemically synthesized.

A nucleic acid molecule of the present invention, e.g., a nucleic acid molecule having the nucleotide sequence of any of SEQ ID Nos:1-22, 34-43, and ______-______ or the cDNA of a clone deposited as any of ATCC 98899, 98900, and 989001, or a complement thereof, can be isolated using standard molecular biology techniques and the sequence information provided herein. Using all or a portion of the nucleic acid sequences of any of SEQ ID NOs:1-22, 34-43, and ______-______ or the cDNA of a clone deposited as any of ATCC 98899, 98900, and 989001 as a hybridization probe, nucleic acid molecules of the invention can be isolated using standard hybridization and cloning techniques (e.g., as described in Sambrook et al., eds., Molecular Cloning: A Laboratory Manual, 2nd ed., Cold Spring Harbor Laboratory, Cold Spring Harbor Laboratory Press, Cold Spring Harbor, N.Y., 1989).

A nucleic acid molecule of the invention can be amplified using cDNA, mRNA or genomic DNA as a template and appropriate oligonucleotide primers according to standard PCR amplification techniques. The nucleic acid so amplified can be cloned into an appropriate vector and characterized by DNA sequence analysis. Furthermore, oligonucleotides corresponding to all or a portion of a nucleic acid molecule of the invention can be prepared by standard synthetic techniques, e.g., using an automated DNA synthesizer.

In another preferred embodiment, an isolated nucleic acid molecule of the invention comprises a nucleic acid molecule which is a complement of the nucleotide sequence shown in SEQ ID NOs:1-22, 34-43, and ______-______ or the cDNA of a clone deposited as ATCC 98899, 98900, and 989001, or a portion thereof. A nucleic acid molecule which is complementary to a given nucleotide sequence is one which is sufficiently complementary to the given nucleotide sequence that it can hybridize to the given nucleotide sequence thereby forming a stable duplex.

Moreover, a nucleic acid molecule of the invention can comprise only a portion of a nucleic acid sequence encoding a full length polypeptide of the invention for example, a fragment which can be used as a probe or primer or a fragment encoding a biologically active portion of a polypeptide of the invention. The nucleotide sequence determined from the cloning one gene allows for the generation of probes and primers designed for use in identifying and/or cloning homologues in other cell types, e.g., from other tissues, as well as homologues from other mammals. The probe/primer typically comprises substantially purified oligonucleotide. The oligonucleotide typically comprises a region of nucleotide sequence that hybridizes under stringent conditions to at least about 12, preferably about 25, more preferably about 50, 75, 100, 125, 150, 175, 200, 250, 300, 350 or 400 consecutive nucleotides of the sense or anti-sense sequence of any of SEQ ID NOs:1-22, 34-43, and ______-______ or the cDNA of a clone deposited as ATCC 98899, 98900, and 989001 or of a naturally occurring mutant of any of SEQ NOs:1-22, 34-43, and ______-______ or the cDNA of a clone deposited as ATCC 98899, 98900, and 989001.

Probes based on the sequence of a nucleic acid molecule of the invention can be used to detect transcripts or genomic sequences encoding the same protein molecule encoded by a selected nucleic acid molecule. The probe comprises a label group attached thereto, e.g., a radioisotope, a fluorescent compound, an enzyme, or an enzyme co-factor. Such probes can be used as part of a diagnostic test kit for identifying cells or tissues which mis-express the protein, such as by measuring levels of a nucleic acid molecule encoding the protein in a sample of cells from a subject, e.g., detecting mRNA levels or determining whether a gene encoding the protein has been mutated or deleted.

A nucleic acid fragment encoding a “biologically active portion” of a polypeptide of the invention can be prepared by isolating a portion of any of SEQ ID NOs:1-22, 34-43, and ______-______ or the nucleotide sequence of the cDNA of a clone deposited as ATCC 98899, 98900, and 989001 which encodes a polypeptide having a biological activity, expressing the encoded portion of the polypeptide protein (e.g., by recombinant expression in vitro) and assessing the activity of the encoded portion of the polypeptide.

The invention further encompasses nucleic acid molecules that differ from the nucleotide sequence of SEQ ID NOs:1-22, 34-43, and ______-______ or the cDNA of a clone of ATCC 98899, 98900, and 989001 due to degeneracy of the genetic code and thus encode the same protein as that encoded by the nucleotide sequence shown in any of SEQ ID NOs:1-22, 34-43, and ______-______ or the cDNA of a clone deposited as ATCC 98899, 98900, and 989001.

In addition to the nucleotide sequences shown in SEQ ID NOs:1-22, 34-43, and ______-______ and present in cDNA's of the clones deposited of ATCC 98899, 98900, and 989001, it will be appreciated by those skilled in the art that DNA sequence polymorphisms that lead to changes in the amino acid sequence may exist within a population (e.g., the human population). Such genetic polymorphisms may exist among individuals within a population due to natural allelic variation. An allele is one of a group of genes which occur alternatively at a given genetic locus. As used herein, the phrase “allelic variant” refers to a nucleotide sequence which occurs at a given locus or to a polypeptide encoded by the nucleotide sequence. As used herein, the terms “gene” and “recombinant gene” refer to nucleic acid molecules comprising an open reading frame encoding a polypeptide of the invention. Such natural allelic variations can typically result in 1-5% variance in the nucleotide sequence of a given gene. Alternative alleles can be identified by sequencing the gene of interest in a number of different individuals. This can be readily carried out by using hybridization probes to identify the same genetic locus in a variety of individuals. Any and all such nucleotide variations and resulting amino acid polymorphisms or variations that are the result of natural allelic variation and that do not alter the functional activity are intended to be within the scope of the invention.

Moreover, nucleic acid molecules encoding proteins of the invention from other species (homologues), which have a nucleotide sequence which differs from that of the human protein described herein are intended to be within the scope of the invention. Nucleic acid molecules corresponding to natural allelic variants and homologues of a cDNA of the invention can be isolated based on their identity to the human nucleic acid molecule disclosed herein using the human cDNAs, or a portion thereof, as a hybridization probe according to standard hybridization techniques under stringent hybridization conditions. For example, a cDNA encoding a soluble form of a membrane-bound protein of the invention isolated based on its hybridization to a nucleic acid molecule encoding all or part of the membrane-bound form. Likewise, a cDNA encoding a membrane-bound form can be isolated based on its hybridization to a nucleic acid molecule encoding all or part of the soluble form.

Accordingly, in another embodiment, an isolated nucleic acid molecule of the invention is at least 300 (325, 350, 375, 400, 425, 450, 500, 550, 600, 650, 700, 800, 900, 1000, or 1290) nucleotides in length and hybridizes under stringent conditions to the nucleic acid molecule comprising the nucleotide sequence, preferably the coding sequence, of any of SEQ ID NOs:1-22, 34-43, and ______-______ the cDNA of a clone deposited as ATCC 98899, 98900, and 989001, or a complement thereof.

As used herein, the term “hybridizes under stringent conditions” is intended to describe conditions for hybridization and washing under which nucleotide sequences at least 60% (65%, 70%, preferably 75%) identical to each other typically remain hybridized to each other. Such stringent conditions are known to those skilled in the art and can be found in Current Protocols in Molecular Biology, John Wiley & Sons, N.Y. (1989), 6.3.1-6.3.6. A preferred, non-limiting example of stringent hybridization conditions are hybridization in 6× sodium chloride/sodium citrate (SSC) at about 45° C., followed by one or more washes in 0.2×SSC, 0.1% SDS at 50-65° C. Preferably, an isolated nucleic acid molecule of the invention that hybridizes under stringent conditions to the sequence of any of SEQ ID NOs:1-22, 34-43, and ______-______, the cDNA of ATCC 98899, 98900, and 989001, or the complement thereof, corresponds to a naturally-occurring nucleic acid molecule. As used herein, a “naturally-occurring” nucleic acid molecule refers to an RNA or DNA molecule having a nucleotide sequence that occurs in nature (e.g., encodes a natural protein).

In addition to naturally-occurring allelic variants of a nucleic acid molecule of the invention sequence that may exist in the population, the skilled artisan will further appreciate that changes can be introduced by mutation thereby leading to changes in the amino acid sequence of the encoded protein, without altering the biological activity of the protein. For example, one can make nucleotide substitutions leading to amino acid substitutions at “non-essential” amino acid residues. A “non-essential” amino acid residue is a residue that can be altered from the wild-type sequence without altering the biological activity, whereas an “essential” amino acid residue is required for biological activity. For example, amino acid residues that are not conserved or only semi-conserved among homologues of various species may be non-essential for activity and thus would be likely targets for alteration. Alternatively, amino acid residues that are conserved among the homologues of various species (e.g., murine and human) may be essential for activity and thus would not be likely targets for alteration. Conserved cysteine residues are particularly important and are preferably retained in functional variants

Accordingly, another aspect of the invention pertains to nucleic acid molecules encoding a polypeptide of the invention that contain changes in amino acid residues that are not essential for activity. Such polypeptides differ in amino acid sequence from SEQ ID NOs:23-33, 54-63, and ______-______ yet retain biological activity. In one embodiment, the isolated nucleic acid molecule includes a nucleotide sequence encoding a protein that includes an amino acid sequence that is at least about 45% identical, 65%, 75%, 85%, 95%, or 98% identical to the amino acid sequence of any of SEQ ID Nos:23-3, 54-63, and ______-______.

An isolated nucleic acid molecule encoding a variant protein can be created by introducing one or more nucleotide substitutions, additions or deletions into the nucleotide sequence of SEQ ID NOs:1-22, 34-43, and ______-______ the cDNA of a clone deposited of ATCC 98899, 98900, and 989001 such that one or more amino acid substitutions, additions or deletions are introduced into the encoded protein. Mutations can be introduced by standard techniques, such as site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Alternatively, mutations can be introduced randomly along all or part of the coding sequence, such as by saturation mutagenesis, and the resultant mutants can be screened for biological activity to identify mutants that retain activity. Following mutagenesis, the encoded protein can be expressed recombinantly and the activity of the protein can be determined.

In a preferred embodiment, a mutant polypeptide that is a variant of a polypeptide of the invention can be assayed for: (1) the ability to form protein:protein interactions with proteins in a signalling pathway of the polypeptide of the invention; (2) the ability to bind a ligand of the polypeptide of the invention; or (3) the ability to bind to an intracellular target protein of the polypeptide of the invention. In yet another preferred embodiment, the mutant polypeptide can be assayed for the ability to modulate cellular proliferation or cellular differentiation.

The present invention encompasses antisense nucleic acid molecules, i.e., molecules which are complementary to a sense nucleic acid encoding a polypeptide of the invention, e.g., complementary to the coding strand of a double-stranded cDNA molecule or complementary to an mRNA sequence. Accordingly, an antisense nucleic acid can hydrogen bond to a sense nucleic acid. The antisense nucleic acid can be complementary to an entire coding strand, or to only a portion thereof, e.g., all or part of the protein coding region (or open reading frame). An antisense nucleic acid molecule can be antisense to all or part of a noncoding region of the coding strand of a nucleotide sequence encoding a polypeptide of the invention. The noncoding regions (“5′ and 3′ untranslated regions”) are the 5′ and 3′ sequences which flank the coding region and are not translated into amino acids.

An antisense oligonucleotide can be, for example, about 5, 10, 15, 20, 25, 30, 35, 40, 45 or 50 nucleotides in length. An antisense nucleic acid of the invention can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. Examples of modified nucleotides which can be used to generate the antisense nucleic acid include 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N-6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding a selected polypeptide of the invention to thereby inhibit expression, e.g., by inhibiting transcription and/or translation. The hybridization can be by conventional nucleotide complementarity to form a stable duplex, or, for example, in the case of an antisense nucleic acid molecule which binds to DNA duplexes, through specific interactions in the major groove of the double helix. An example of a route of administration of antisense nucleic acid molecules of the invention includes direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface, e.g., by linking the antisense nucleic acid molecules to peptides or antibodies which bind to cell surface receptors or antigens. The antisense nucleic acid molecules can also be delivered to cells using the vectors described herein. To achieve sufficient intracellular concentrations of the antisense molecules, vector constructs in which the antisense nucleic acid molecule is placed under the control of a strong pol II or pol III promoter are preferred.

An antisense nucleic acid molecule of the invention can be an α-anomeric nucleic acid molecule. An α-anomeric nucleic acid molecule forms specific double-stranded hybrids with complementary RNA in which, contrary to the usual β-units, the strands run parallel to each other (Gaultier et al. (1987) Nucleic Acids Res. 15:6625-6641). The antisense nucleic acid molecule can also comprise a 2′-o-methylribonucleotide (Inoue et al. (1987) Nucleic Acids Res. 15:6131-6148) or a chimeric RNA-DNA analogue (Inoue et al. (1987) FEBS Lett. 215:327-330).

The invention also encompasses ribozymes. Ribozymes are catalytic RNA molecules with ribonuclease activity which are capable of cleaving a single-stranded nucleic acid, such as an mRNA, to which they have a complementary region. Thus, ribozymes (e.g., hammerhead ribozymes (described in Haselhoff and Gerlach (1988) Nature 334:585-591)) can be used to catalytically cleave mRNA transcripts to thereby inhibit translation of the protein encoded by the mRNA. A ribozyme having specificity for a nucleic acid molecule encoding a polypeptide of the invention can be designed based upon the nucleotide sequence of a cDNA disclosed herein. For example, a derivative of a Tetrahymena L-19 IVS RNA can be constructed in which the nucleotide sequence of the active site is complementary to the nucleotide sequence to be cleaved in a Cech et al. U.S. Pat. No. 4,987,071; and Cech et al. U.S. Pat. No. 5,116,742. Alternatively, an mRNA encoding a polypeptide of the invention can be used to select a catalytic RNA having a specific ribonuclease activity from a pool of RNA molecules. See, e.g., Bartel and Szostak (1993) Science 261:1411-1418.

The invention also encompasses nucleic acid molecules which form triple helical structures. For example, expression of a polypeptide of the invention can be inhibited by targeting nucleotide sequences complementary to the regulatory region of the gene encoding the polypeptide (e.g., the promoter and/or enhancer) to form triple helical structures that prevent transcription of the gene in target cells. See generally Helene (1991) Anticancer Drug Des. 6(6):569-84; Helene (1992) Ann. N.Y. Acad. Sci. 660:27-36; and Maher (1992) Bioassays 14(12):807-15.

In preferred embodiments, the nucleic acid molecules of the invention can be modified at the base moiety, sugar moiety or phosphate backbone to improve, e.g., the stability, hybridization, or solubility of the molecule. For example, the deoxyribose phosphate backbone of the nucleic acids can be modified to generate peptide nucleic acids (see Hyrup et al. (1996) Bioorganic & Medicinal Chemistry 4(1): 5-23). As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup et al. (1996), supra; Perry-O'Keefe et al. (1996) Proc. Natl. Acad. Sci. USA 93: 14670-675.

PNAs can be used in therapeutic and diagnostic applications. For example, PNAs can be used as antisense or antigene agents for sequence-specific modulation of gene expression by, e.g., inducing transcription or translation arrest or inhibiting replication. PNAs can also be used, e.g., in the analysis of single base pair mutations in a gene by, e.g., PNA directed PCR clamping; as artificial restriction enzymes when used in combination with other enzymes, e.g., S1 nucleases (Hyrup (1996), supra; or as probes or primers for DNA sequence and hybridization (Hyrup (1996), supra; Perry-O'Keefe et al. (1996) Proc. Natl. Acad. Sci. USA 93: 14670-675).

In another embodiment, PNAs can be modified, e.g., to enhance their stability or cellular uptake, by attaching lipophilic or other helper groups to PNA, by the formation of PNA-DNA chimeras, or by the use of liposomes or other techniques of drug delivery known in the art. For example, PNA-DNA chimeras can be generated which may combine the advantageous properties of PNA and DNA. Such chimeras allow DNA recognition enzymes, e.g., RNAse H and DNA polymerases, to interact with the DNA portion while the PNA portion would provide high binding affinity and specificity. PNA-DNA chimeras can be linked using linkers of appropriate lengths selected in terms of base stacking, number of bonds between the nucleobases, and orientation (Hyrup (1996), supra). The synthesis of PNA-DNA chimeras can be performed as described in Hyrup (1996), supra, and Finn et al. (1996). Nucleic Acids Res. 24(17):3357-63. For example, a DNA chain can be synthesized on a solid support using standard phosphoramidite coupling chemistry and modified nucleoside analogs. Compounds such as 5′-(4-methoxytrityl)amino-5′-deoxy-thymidine phosphoramidite can be used as a link between the PNA and the 5′ end of DNA (Mag et al. (1989) Nucleic Acids Res. 17:5973-88). PNA monomers are then coupled in a stepwise manner to produce a chimeric molecule with a 5′ PNA segment and a 3′ DNA segment (Finn et al. (1996) Nucleic Acids Res. 24(17):3357-63). Alternatively, chimeric molecules can be synthesized with a 5′ DNA segment and a 3′ PNA segment (Peterser et al. (1975) Bioorganic Med. Chem. Lett. 5:1119-11124).

In other embodiments, the oligonucleotide may include other appended groups such as peptides (e.g., for targeting host cell receptors in vivo), or agents facilitating transport across the cell membrane (see, e.g., Letsinger et al. (1989) Proc. Natl. Acad. Sci. USA 86:6553-6556; Lemaitre et al. (1987) Proc. Natl. Acad. Sci. USA 84:648-652; PCT Publication No. WO 88/09810) or the blood-brain barrier (see, e.g., PCT Publication No. WO 89/10134). In addition, oligonucleotides can be modified with hybridization-triggered cleavage agents (see, e.g., Krol et al. (1988) Bio/Techniques 6:958-976) or intercalating agents (see, e.g., Zon (1988) Pharm. Res. 5:539-549). To this end, the oligonucleotide may be conjugated to another molecule, e.g., a peptide, hybridization triggered cross-linking agent, transport agent, hybridization-triggered cleavage agent, etc.

II. Isolated Proteins and Antibodies

One aspect of the invention pertains to isolated proteins, and biologically active portions thereof, as well as polypeptide fragments suitable for use as immunogens to raise antibodies directed against a polypeptide of the invention. In one embodiment, the native polypeptide can be isolated from cells or tissue sources by an appropriate purification scheme using standard protein purification techniques. In another embodiment, polypeptides of the invention are produced by recombinant DNA techniques. Alternative to recombinant expression, a polypeptide of the invention can be synthesized chemically using standard peptide synthesis techniques.

An “isolated” or “purified” protein or biologically active portion thereof is substantially free of cellular material or other contaminating proteins from the cell or tissue source from which the protein is derived, or substantially free of chemical precursors or other chemicals when chemically synthesized. The language “substantially free of cellular material” includes preparations of protein in which the protein is separated from cellular components of the cells from which it is isolated or recombinantly produced. Thus, protein that is substantially free of cellular material includes preparations of protein having less than about 30%, 20%, 10%, or 5% (by dry weight) of heterologous protein (also referred to herein as a “contaminating protein”). When the protein or biologically active portion thereof is recombinantly produced, it is also preferably substantially free of culture medium, i.e., culture medium represents less than about 20%, 10%, or 5% of the volume of the protein preparation. When the protein is produced by chemical synthesis, it is preferably substantially free of chemical precursors or other chemicals, i.e., it is separated from chemical precursors or other chemicals which are involved in the synthesis of the protein. Accordingly such preparations of the protein have less than about 30%, 20%, 10%, 5% (by dry weight) of chemical precursors or compounds other than the polypeptide of interest.

Biologically active portions of a polypeptide of the invention include polypeptides comprising amino acid sequences sufficiently identical to or derived from the amino acid sequence of the protein (e.g., the amino acid sequence shown in any of SEQ ID Nos:23-33, 54-63, and ______-______ which include fewer amino acids than the full length protein, and exhibit at least one activity of the corresponding full-length protein. Typically, biologically active portions comprise a domain or motif with at least one activity of the corresponding protein. A biologically active portion of a protein of the invention can be a polypeptide which is, for example, 10, 25, 50, 100 or more amino acids in length. Moreover, other biologically active portions, in which other regions of the protein are deleted, can be prepared by recombinant techniques and evaluated for one or more of the functional activities of the native form of a polypeptide of the invention.

Preferred polypeptides have the amino acid sequence of any of SEQ ID Nos:23-33, 54-63, and ______-______. Other useful proteins are substantially identical (e.g., at least about 45%, preferably 55%, 65%, 75%, 85%, 95%, or 99%) to any of SEQ ID Nos:22-33, 54-63, and ______-______ and retain the functional activity of the protein of the corresponding naturally-occurring protein yet differ in amino acid sequence due to natural allelic variation or mutagenesis.

To determine the percent identity of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=# of identical positions/total # of positions (e.g., overlapping positions)×100). Preferably, the two sequences are the same length.

The determination of percent homology between two sequences can be accomplished using a mathematical algorithm. A preferred, non-limiting example of a mathematical algorithm utilized for the comparison of two sequences is the algorithm of Karlin and Altschul (1990) Proc. Natl. Acad. Sci. USA 87:2264-2268, modified as in Karlin and Altschul (1993) Proc. Natl. Acad. Sci. USA 90:5873-5877. Such an algorithm is incorporated into the NBLAST and XBLAST programs of Altschul, et al. (1990) J. Mol. Biol. 215:403-410. BLAST nucleotide searches can be performed with the NBLAST program, score=100, wordlength=12 to obtain nucleotide sequences homologous to a nucleic acid molecules of the invention. BLAST protein searches can be performed with the XBLAST program, score=50, wordlength=3 to obtain amino acid sequences homologous to a protein molecules of the invention. To obtain gapped alignments for comparison purposes, Gapped BLAST can be utilized as described in Altschul et al. (1997) Nucleic Acids Res. 25:3389-3402. Alternatively, PSI-Blast can be used to perform an iterated search which detects distant relationships between molecules. Id. When utilizing BLAST, Gapped BLAST, and PSI-Blast programs, the default parameters of the respective programs (e.g., XBLAST and NBLAST) can be used. See http://www.ncbi.nlm.nih.gov. Another preferred, non-limiting example of a mathematical algorithm utilized for the comparison of sequences is the algorithm of Myers and Miller, (1988) CABIOS 4:11-17. Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

The percent identity between two sequences can be determined using techniques similar to those described above, with or without allowing gaps. In calculating percent identity, only exact matches are counted.

The invention also provides chimeric or fusion proteins. As used herein, a “chimeric protein” or “fusion protein” comprises all or part (preferably biologically active) of a polypeptide of the invention operably linked to a heterologous polypeptide (i.e., a polypeptide other than the same polypeptide of the invention). Within the fusion protein, the term “operably linked” is intended to indicate that the polypeptide of the invention and the heterologous polypeptide are fused in-frame to each other. The heterologous polypeptide can be fused to the N-terminus or C-terminus of the polypeptide of the invention.

One useful fusion protein is a GST fusion protein in which the polypeptide of the invention is fused to the C-terminus of GST sequences. Such fusion proteins can facilitate the purification of a recombinant polypeptide of the invention.

In another embodiment, the fusion protein contains a heterologous signal sequence at its N-terminus. For example, the native signal sequence of a polypeptide of the invention can be removed and replaced with a signal sequence from another protein. For example, the gp67 secretory sequence of the baculovirus envelope protein can be used as a heterologous signal sequence (Current Protocols in Molecular Biology, Ausubel et al., eds., John Wiley & Sons, 1992). Other examples of eukaryotic heterologous signal sequences include the secretory sequences of melittin and human placental alkaline phosphatase (Stratagene; La Jolla, Calif.). In yet another example, useful prokaryotic heterologous signal sequences include the phoA secretory signal (Sambrook et al., supra) and the protein A secretory signal (Pharmacia Biotech; Piscataway, N.J.).

In yet another embodiment, the fusion protein is an immunoglobulin fusion protein in which all or part of a polypeptide of the invention is fused to sequences derived from a member of the immunoglobulin protein family. The immunoglobulin fusion proteins of the invention can be incorporated into pharmaceutical compositions and administered to a subject to inhibit an interaction between a ligand (soluble or membrane-bound) and a protein on the surface of a cell (receptor), to thereby suppress signal transduction in vivo. The immunoglobulin fusion protein can be used to affect the bioavailability of a cognate ligand of a polypeptide of the invention. Inhibition of ligand/receptor interaction may be useful therapeutically, both for treating proliferative and differentiative disorders and for modulating (e.g. promoting or inhibiting) cell survival. Moreover, the immunoglobulin fusion proteins of the invention can be used as immunogens to produce antibodies directed against a polypeptide of the invention in a subject, to purify ligands and in screening assays to identify molecules which inhibit the interaction of receptors with ligands.

Chimeric and fusion protein of the invention can be produced by standard recombinant DNA techniques. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and reamplified to generate a chimeric gene sequence (see, e.g., Ausubel et al., supra). Moreover, many expression vectors are commercially available that already encode a fusion moiety (e.g., a GST polypeptide). A nucleic acid encoding a polypeptide of the invention can be cloned into such an expression vector such that the fusion moiety is linked in-frame to the polypeptide of the invention.

A signal sequence of a polypeptide of the invention (SEQ ID NOs:64-75) can be used to facilitate secretion and isolation of the secreted protein or other proteins of interest. Signal sequences are typically characterized by a core of hydrophobic amino acids which are generally cleaved from the mature protein during secretion in one or more cleavage events. Such signal peptides contain processing sites that allow cleavage of the signal sequence from the mature proteins as they pass through the secretory pathway. Thus, the invention pertains to the described polypeptides having a signal sequence, as well as to the signal sequence itself and to the polypeptide in the absence of the signal sequence (i.e., the cleavage products). In one embodiment, a nucleic acid sequence encoding a signal sequence of the invention can be operably linked in an expression vector to a protein of interest, such as a protein which is ordinarily not secreted or is otherwise difficult to isolate. The signal sequence directs secretion of the protein, such as from a eukaryotic host into which the expression vector is transformed, and the signal sequence is subsequently or concurrently cleaved. The protein can then be readily purified from the extracellular medium by art recognized methods. Alternatively, the signal sequence can be linked to the protein of interest using a sequence which facilitates purification, such as with a GST domain.

In another embodiment, the signal sequences of the present invention can be used to identify regulatory sequences, e.g., promoters, enhancers, repressors. Since signal sequences are the most amino-terminal sequences of a peptide, it is expected that the nucleic acids which flank the signal sequence on its amino-terminal side will be regulatory sequences which affect transcription. Thus, a nucleotide sequence which encodes all or a portion of a signal sequence can be used as a probe to identify and isolate signal sequences and their flanking regions, and these flanking regions can be studied to identify regulatory elements therein.

The present invention also pertains to variants of the polypeptides of the invention. Such variants have an altered amino acid sequence which can function as either agonists (mimetics) or as antagonists. Variants can be generated by mutagenesis, e.g., discrete point mutation or truncation. An agonist can retain substantially the same, or a subset, of the biological activities of the naturally occurring form of the protein. An antagonist of a protein can inhibit one or more of the activities of the naturally occurring form of the protein by, for example, competitively binding to a downstream or upstream member of a cellular signaling cascade which includes the protein of interest. Thus, specific biological effects can be elicited by treatment with a variant of limited function. Treatment of a subject with a variant having a subset of the biological activities of the naturally occurring form of the protein can have fewer side effects in a subject relative to treatment with the naturally occurring form of the protein.

Variants of a protein of the invention which function as either agonists (mimetics) or as antagonists can be identified by screening combinatorial libraries of mutants, e.g., truncation mutants, of the protein of the invention for agonist or antagonist activity. In one embodiment, a variegated library of variants is generated by combinatorial mutagenesis at the nucleic acid level and is encoded by a variegated gene library. A variegated library of variants can be produced by, for example, enzymatically ligating a mixture of synthetic oligonucleotides into gene sequences such that a degenerate set of potential protein sequences is expressible as individual polypeptides, or alternatively, as a set of larger fusion proteins (e.g., for phage display). There are a variety of methods which can be used to produce libraries of potential variants of the polypeptides of the invention from a degenerate oligonucleotide sequence. Methods for synthesizing degenerate oligonucleotides are known in the art (see, e.g., Narang (1983) Tetrahedron 39:3; Itakura et al. (1984) Annu. Rev. Biochem. 53:323; Itakura et al. (1984) Science 198:1056; Ike et al. (1983) Nucleic Acid Res. 11:477).

In addition, libraries of fragments of the coding sequence of a polypeptide of the invention can be used to generate a variegated population of polypeptides for screening and subsequent selection of variants. For example, a library of coding sequence fragments can be generated by treating a double stranded PCR fragment of the coding sequence of interest with a nuclease under conditions wherein nicking occurs only about once per molecule, denaturing the double stranded DNA, renaturing the DNA to form double stranded DNA which can include sense/antisense pairs from different nicked products, removing single stranded portions from reformed duplexes by treatment with S1 nucleate, and ligating the resulting fragment library into an expression vector. By this method, an expression library can be derived which encodes N-terminal and internal fragments of various sizes of the protein of interest.

Several techniques are known in the art for screening gene products of combinatorial libraries made by point mutations or truncation, and for screening cDNA libraries for gene products having a selected property. The most widely used techniques, which are amenable to high through-put analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a technique which enhances the frequency of functional mutants in the libraries, can be used in combination with the screening assays to identify variants of a protein of the invention (Arkin and Yourvan (1992) Proc. Natl. Acad. Sci. USA 89:7811-7815; Delgrave et al. (1993) Protein Engineering 6(3):327-331).

An isolated polypeptide of the invention, or a fragment thereof, can be used as an immunogen to generate antibodies using standard techniques for polyclonal and monoclonal antibody preparation. The full-length polypeptide or protein can be used or, alternatively, the invention provides antigenic peptide fragments for use as immunogens. The antigenic peptide of a protein of the invention comprises at least 8 (preferably 10, 15, 20, or 30) amino acid residues of the amino acid sequence shown in any of SEQ ID Nos:23-33, 54-64, and ______-______ and encompasses an epitope of the protein such that an antibody raised against the peptide forms a specific immune complex with the protein.

Preferred epitopes encompassed by the antigenic peptide are regions that are located on the surface of the protein, e.g., hydrophilic regions, rather than hydrophobic regions, e.g., transmembrane domains. The hydrophilicity of a protein sequence can be easily determined using readily available programs.

An immunogen typically is used to prepare antibodies by immunizing a suitable subject, (e.g., rabbit, goat, mouse or other mammal). An appropriate immunogenic preparation can contain, for example, recombinantly expressed chemically synthesized polypeptide. The preparation can further include an adjuvant, such as Freund's complete or incomplete adjuvant, or similar immunostimulatory agent.

Accordingly, another aspect of the invention pertains to antibodies directed against a polypeptide of the invention. The term “antibody” as used herein refers to immunoglobulin molecules and immunologically active portions of immunoglobulin molecules, i.e., molecules that contain an antigen binding site which specifically binds an antigen, such as a polypeptide of the invention. A molecule which specifically binds to a given polypeptide of the invention is a molecule which binds the polypeptide, but does not substantially bind other molecules in a sample, e.g., a biological sample, which naturally contains the polypeptide. Examples of immunologically active portions of immunoglobulin molecules include F(ab) and F(ab′)₂ fragments which can be generated by treating the antibody with an enzyme such as pepsin. The invention provides polyclonal and monoclonal antibodies. The term “monoclonal antibody” or “monoclonal antibody composition”, as used herein, refers to a population of antibody molecules that contain only one species of an antigen binding site capable of immunoreacting with a particular epitope.

Polyclonal antibodies can be prepared as described above by immunizing a suitable subject with a polypeptide of the invention as an immunogen. The antibody titer in the immunized subject can be monitored over time by standard techniques, such as with an enzyme linked immunosorbent assay (ELISA) using immobilized polypeptide. If desired, the antibody molecules can be isolated from the mammal (e.g., from the blood) and further purified by well-known techniques, such as protein A chromatography to obtain the IgG fraction. At an appropriate time after immunization, e.g., when the specific antibody titers are highest, antibody-producing cells can be obtained from the subject and used to prepare monoclonal antibodies by standard techniques, such as the hybridoma technique originally described by Kohler and Milstein (1975) Nature 256:495-497, the human B cell hybridoma technique (Kozbor et al. (1983) Immunol. Today 4:72), the EBV-hybridoma technique (Cole et al. (1985), Monoclonal Antibodies and Cancer Therapy, Alan R. Liss, Inc., pp. 77-96) or trioma techniques. The technology for producing hybridomas is well known (see generally Current Protocols in Immunology (1994) Coligan et al. (eds.) John Wiley & Sons, Inc., New York, N.Y.). Hybridoma cells producing a monoclonal antibody of the invention are detected by screening the hybridoma culture supernatants for antibodies that bind the polypeptide of interest, e.g., using a standard ELISA assay.

Alternative to preparing monoclonal antibody-secreting hybridomas, a monoclonal antibody directed against a polypeptide of the invention can be identified and isolated by screening a recombinant combinatorial immunoglobulin library (e.g., an antibody phage display library) with the polypeptide of interest. Kits for generating and screening phage display libraries are commercially available (e.g., the Pharmacia Recombinant Phage Antibody System, Catalog No. 27-9400-01; and the Stratagene SurfZAP™ Phage Display Kit, Catalog No. 240612). Additionally, examples of methods and reagents particularly amenable for use in generating and screening antibody display library can be found in, for example, U.S. Pat. No. 5,223,409; PCT Publication No. WO 92/18619; PCT Publication No. WO 91/17271; PCT Publication No. WO 92/20791; PCT Publication No. WO 92/15679; PCT Publication No. WO 93/01288; PCT Publication No. WO 92/01047; PCT Publication No. WO 92/09690; PCT Publication No. WO 90/02809; Fuchs et al. (1991) Bio/Technology 9:1370-1372; Hay et al. (1992) Hum. Antibod. Hybridomas 3:81-85; Huse et al. (1989) Science 246:1275-1281; Griffiths et al. (1993) EMBO J. 12:725-734.

Additionally, recombinant antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art, for example using methods described in PCT Publication No. WO 87/02671; European Patent Application 184,187; European Patent Application 171,496; European Patent Application 173,494; PCT Publication No. WO 86/01533; U.S. Pat. No. 4,816,567; European Patent Application 125,023; Better et al. (1988) Science 240:1041-1043; Liu et al. (1987) Proc. Natl. Acad. Sci. USA 84:3439-3443; Liu et al. (1987) J. Immunol. 139:3521-3526; Sun et al. (1987) Proc. Natl. Acad. Sci. USA 84:214-218; Nishimura et al. (1987) Canc. Res. 47:999-1005; Wood et al. (1985) Nature 314:446-449; and Shaw et al. (1988) J. Natl. Cancer Inst. 80:1553-1559); Morrison (1985) Science 229:1202-1207; Oi et al. (1986) Bio/Techniques 4:214; U.S. Pat. No. 5,225,539; Jones et al. (1986) Nature 321:552-525; Verhoeyan et al. (1988) Science 239:1534; and Beidler et al. (1988) J. Immunol. 141:4053-4060.

Completely human antibodies are particularly desirable for therapeutic treatment of human patients. Such antibodies can be produced using transgenic mice which are incapable of expressing endogenous immunoglobulin heavy and light chains genes, but which can express human heavy and light chain genes. The transgenic mice are immunized in the normal fashion with a selected antigen, e.g., all or a portion of a polypeptide of the invention. Monoclonal antibodies directed against the antigen can be obtained using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA and IgE antibodies. For an overview of this technology for producing human antibodies, see Lonberg and Huszar (1995, Int. Rev. Immunol. 13:65-93). For a detailed discussion of this technology for producing human antibodies and human monoclonal antibodies and protocols for producing such antibodies, see, e.g., U.S. Pat. No. 5,625,126; U.S. Pat. No. 5,633,425; U.S. Pat. No. 5,569,825; U.S. Pat. No. 5,661,016; and U.S. Pat. No. 5,545,806. In addition, companies such as Abgenix, Inc. (Freemont, Calif.), can be engaged to provide human antibodies directed against a selected antigen using technology similar to that described above.

Completely human antibodies which recognize a selected epitope can be generated using a technique referred to as “guided selection.” In this approach a selected non-human monoclonal antibody, e.g., a murine antibody, is used to guide the selection of a completely human antibody recognizing the same epitope.

An antibody directed against a polypeptide of the invention (e.g., monoclonal antibody) can be used to isolate the polypeptide by standard techniques, such as affinity chromatography or immunoprecipitation. Moreover, such an antibody can be used to detect the protein (e.g., in a cellular lysate or cell supernatant) in order to evaluate the abundance and pattern of expression of the polypeptide. The antibodies can also be used diagnostically to monitor protein levels in tissue as part of a clinical testing procedure, e.g., to, for example, determine the efficacy of a given treatment regimen. Detection can be facilitated by coupling the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

III. Recombinant Expression Vectors and Host Cells

Another aspect of the invention pertains to vectors, preferably expression vectors, containing a nucleic acid encoding a polypeptide of the invention (or a portion thereof). As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors, expression vectors, are capable of directing the expression of genes to which they are operably linked. In general, expression vectors of utility in recombinant DNA techniques are often in the form of plasmids (vectors). However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell. This means that the recombinant expression vectors include one or more regulatory sequences, selected on the basis of the host cells to be used for expression, which is operably linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to include promoters, enhancers and other expression control elements (e.g., polyadenylation signals). Such regulatory sequences are described, for example, in Goeddel, Gene Expression Technology: Methods in Enzymology 185, Academic Press, San Diego, Calif. (1990). Regulatory sequences include those which direct constitutive expression of a nucleotide sequence in many types of host cell and those which direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein.

The recombinant expression vectors of the invention can be designed for expression of a polypeptide of the invention in prokaryotic or eukaryotic cells, e.g., bacterial cells such as E. coli, insect cells (using baculovirus expression vectors), yeast cells or mammalian cells. Suitable host cells are discussed further in Goeddel, supra. Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith and Johnson (1988) Gene 67:31-40), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein.

Examples of suitable inducible non-fusion E. coli expression vectors include pTrc (Amann et al., (1988) Gene 69:301-315) and pET 11d (Studier et al., Gene Expression Technology: Methods in Enzymology 185, Academic Press, San Diego, Calif. (1990) 60-89). Target gene expression from the pTrc vector relies on host RNA polymerase transcription from a hybrid trp-lac fusion promoter. Target gene expression from the pET 11d vector relies on transcription from a T7 gn10-lac fusion promoter mediated by a coexpressed viral RNA polymerase (T7 gn1). This viral polymerase is supplied by host strains BL21(DE3) or HMS174(DE3) from a resident λ prophage harboring a T7 gn1 gene under the transcriptional control of the lacUV 5 promoter.

One strategy to maximize recombinant protein expression in E. coli is to express the protein in a host bacteria with an impaired capacity to proteolytically cleave the recombinant protein (Gottesman, Gene Expression Technology: Methods in Enzymology 185, Academic Press, San Diego, Calif. (1990) 119-128). Another strategy is to alter the nucleic acid sequence of the nucleic acid to be inserted into an expression vector so that the individual codons for each amino acid are those preferentially utilized in E. coli (Wada et al. (1992) Nucleic Acids Res. 20:2111-2118). Such alteration of nucleic acid sequences of the invention can be carried out by standard DNA synthesis techniques.

In another embodiment, the expression vector is a yeast expression vector. Examples of vectors for expression in yeast S. cerivisae include pYepSec1 (Baldari et al. (1987) EMBO J. 6:229-234), pMFa (Kurjan and Herskowitz, (1982) Cell 30:933-943), pJRY88 (Schultz et al. (1987) Gene 54:113-123), pYES2 (Invitrogen Corporation, San Diego, Calif.), and pPicZ (Invitrogen Corp, San Diego, Calif.).

Alternatively, the expression vector is a baculovirus expression vector. Baculovirus vectors available for expression of proteins in cultured insect cells (e.g., Sf 9 cells) include the pAc series (Smith et al. (1983) Mol. Cell Biol. 3:2156-2165) and the pVL series (Lucklow and Summers (1989) Virology 170:31-39).

In yet another embodiment, a nucleic acid of the invention is expressed in mammalian cells using a mammalian expression vector. Examples of mammalian expression vectors include pCDM8 (Seed (1987) Nature 329:840) and pMT2PC (Kaufman et al. (1987) EMBO J. 6:187-195). When used in mammalian cells, the expression vector's control functions are often provided by viral regulatory elements. For example, commonly used promoters are derived from polyoma, Adenovirus 2, cytomegalovirus and Simian Virus 40. For other suitable expression systems for both prokaryotic and eukaryotic cells see chapters 16 and 17 of Sambrook et al., supra.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert et al. (1987) Genes Dev. 1:268-277), lymphoid-specific promoters (Calame and Eaton (1988) Adv. Immunol. 43:235-275), in particular promoters of T cell receptors (Winoto and Baltimore (1989) EMBO J. 8:729-733) and immunoglobulins (Banerji et al. (1983) Cell 33:729-740; Queen and Baltimore (1983) Cell 33:741-74.8), neuron-specific promoters (e.g., the neurofilament promoter; Byrne and Ruddle (1989) Proc. Natl. Acad. Sci. USA 86:5473-5477), pancreas-specific promoters (Edlund et al. (1985) Science 230:912-916), and mammary gland-specific promoters (e.g., milk whey promoter; U.S. Pat. No. 4,873,316 and European Application Publication No. 264,166). Developmentally-regulated promoters are also encompassed, for example the murine hox promoters (Kessel and Gruss (1990) Science 249:374-379) and the α-fetoprotein promoter (Campes and Tilghman (1989) Genes Dev. 3:537-546).

The invention further provides a recombinant expression vector comprising a DNA molecule of the invention cloned into the expression vector in an antisense orientation. That is, the DNA molecule is operably linked to a regulatory sequence in a manner which allows for expression (by transcription of the DNA molecule) of an RNA molecule which is antisense to the mRNA encoding a polypeptide of the invention. Regulatory sequences operably linked to a nucleic acid cloned in the antisense orientation can be chosen which direct the continuous expression of the antisense RNA molecule in a variety of cell types, for instance viral promoters and/or enhancers, or regulatory sequences can be chosen which direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see Weintraub et al. (Reviews—Trends in Genetics, Vol. 1(1) 1986).

Another aspect of the invention pertains to host cells into which a recombinant expression vector of the invention has been introduced. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic (e.g., E. coli) or eukaryotic (e.g., an insect cell, a yeast cell or a mammalian cell) cell.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid into a host cell, including calcium phosphate or calcium chloride co-precipitation, DEAE-dextran-mediated transfection, lipofection, or electroporation. Suitable methods for transforming or transfecting host cells can be found in Sambrook, et al. (supra), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., for resistance to antibiotics) is generally introduced into the host cells along with the gene of interest. Preferred selectable markers include those which confer resistance to drugs, such as G418, hygromycin and methotrexate. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce a polypeptide of the invention. Accordingly, the invention further provides methods for producing a polypeptide of the invention using the host cells of the invention. In one embodiment, the method comprises culturing the host cell of invention (into which a recombinant expression vector encoding a polypeptide of the invention has been introduced) in a suitable medium such that the polypeptide is produced. In another embodiment, the method further comprises isolating the polypeptide from the medium or the host cell.

The host cells of the invention can also be used to produce nonhuman transgenic animals. For example, in one embodiment, a host cell of the invention is a fertilized oocyte or an embryonic stem cell into which a sequences encoding a polypeptide of the invention have been introduced. Such host cells can then be used to create non-human transgenic animals in which exogenous sequences encoding a polypeptide of the invention have been introduced into their genome or homologous recombinant animals in which endogenous encoding a polypeptide of the invention sequences have been altered. Such animals are useful for studying the function and/or activity of the polypeptide and for identifying and/or evaluating modulators of polypeptide activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, etc. A transgene is exogenous DNA which is integrated into the genome of a cell from which a transgenic animal develops and which remains in the genome of the mature animal, thereby directing the expression of an encoded gene product in one or more cell types or tissues of the transgenic animal. As used herein, an “homologous recombinant animal” is a non-human animal, preferably a mammal, more preferably a mouse, in which an endogenous gene has been altered by homologous recombination between the endogenous gene and an exogenous DNA molecule introduced into a cell of the animal, e.g., an embryonic cell of the animal, prior to development of the animal.

A transgenic animal of the invention can be created by introducing nucleic acid encoding a polypeptide of the invention (or a homologue thereof) into the male pronuclei of a fertilized oocyte, e.g., by microinjection, retroviral infection, and allowing the oocyte to develop in a pseudopregnant female foster animal. Intronic sequences and polyadenylation signals can also be included in the transgene to increase the efficiency of expression of the transgene. A tissue-specific regulatory sequence(s) can be operably linked to the transgene to direct expression of the polypeptide of the invention to particular cells. Methods for generating transgenic animals via embryo manipulation and microinjection, particularly animals such as mice, have become conventional in the art and are described, for example, in U.S. Pat. Nos. 4,736,866 and 4,870,009, U.S. Pat. No. 4,873,191 and in Hogan, Manipulating the Mouse Embryo, (Cold Spring Harbor Laboratory Press, Cold Spring Harbor, N.Y., 1986). Similar methods are used for production of other transgenic animals. A transgenic founder animal can be identified based upon the presence of the transgene in its genome and/or expression of mRNA encoding the transgene in tissues or cells of the animals. A transgenic founder animal can then be used to breed additional animals carrying the transgene. Moreover, transgenic animals carrying the transgene can further be bred to other transgenic animals carrying other transgenes.

To create an homologous recombinant animal, a vector is prepared which contains at least a portion of a gene encoding a polypeptide of the invention into which a deletion, addition or substitution has been introduced to thereby alter, e.g., functionally disrupt, the gene. In a preferred embodiment, the vector is designed such that, upon homologous recombination, the endogenous gene is functionally disrupted (i.e., no longer encodes a functional protein; also referred to as a “knock out” vector). Alternatively, the vector can be designed such that, upon homologous recombination, the endogenous gene is mutated or otherwise altered but still encodes functional protein (e.g., the upstream regulatory region can be altered to thereby alter the expression of the endogenous protein). In the homologous recombination vector, the altered portion of the gene is flanked at its 5′ and 3′ ends by additional nucleic acid of the gene to allow for homologous recombination to occur between the exogenous gene carried by the vector and an endogenous gene in an embryonic stem cell. The additional flanking nucleic acid sequences are of sufficient length for successful homologous recombination with the endogenous gene. Typically, several kilobases of flanking DNA (both at the 5′ and 3′ ends) are included in the vector (see, e.g., Thomas and Capecchi (1987) Cell 51:503 for a description of homologous recombination vectors). The vector is introduced into an embryonic stem cell line (e.g., by electroporation) and cells in which the introduced gene has homologously recombined with the endogenous gene are selected (see, e.g., Li et al. (1992) Cell 69:915). The selected cells are then injected into a blastocyst of an animal (e.g., a mouse) to form aggregation chimeras (see, e.g., Bradley in Teratocarcinomas and Embryonic Stem Cells: A Practical Approach, Robertson, ed. (IRL, Oxford, 1987) pp. 113-152). A chimeric embryo can then be implanted into a suitable pseudopregnant female foster animal and the embryo brought to term. Progeny harboring the homologously recombined DNA in their germ cells can be used to breed animals in which all cells of the animal contain the homologously recombined DNA by germline transmission of the transgene. Methods for constructing homologous recombination vectors and homologous recombinant animals are described further in Bradley (1991) Current Opinion in Bio/Technology 2:823-829 and in PCT Publication NOS. WO 90/11354, WO 91/01140, WO 92/0968, and WO 93/04169.

In another embodiment, transgenic non-human animals can be produced which contain selected systems which allow for regulated expression of the transgene. One example of such a system is the cre/loxP recombinase system of bacteriophage P1. For a description of the cre/loxP recombinase system, see, e.g., Lakso et al. (1992) Proc. Natl. Acad. Sci. USA 89:6232-6236. Another example of a recombinase system is the FLP recombinase system of Saccharomyces cerevisiae (O'Gorman et al. (1991) Science 251:1351-1355. If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut et al. (1997) Nature 385:810-813 and PCT Publication NOS. WO 97/07668 and WO 97/07669.

IV. Pharmaceutical Compositions

The nucleic acid molecules, polypeptides, and antibodies (also referred to herein as “active compounds”) of the invention can be incorporated into pharmaceutical compositions suitable for administration. Such compositions typically comprise the nucleic acid molecule, protein, or antibody and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

The invention includes methods for preparing pharmaceutical compositions for modulating the expression or activity of a polypeptide or nucleic acid of the invention. Such methods comprise formulating a pharmaceutically acceptable carrier with an agent which modulates expression or activity of a polypeptide or nucleic acid of the invention. Such compositions can further include additionl active agents. Thus, the invention further includes methods for preparing a pharmaceutical composition by formulating a pharmaceutically acceptable carrier with an agent which modulates expression or activity of a polypeptide or nucleic acid of the invention and one or more addtional active compounds.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersions. For intravenous administration, suitable carriers include physiological saline, bacteriostatic water, Cremophor EL™ (BASF; Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., a polypeptide or antibody) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from a pressurized container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

For antibodies, the preferred dosage is 0.1 mg/kg to 100 mg/kg of body weight (generally 10 mg/kg to 20 mg/kg). If the antibody is to act in the brain, a dosage of 50 mg/kg to 100 mg/kg is usually appropriate. Generally, partially human antibodies and fully human antibodies have a longer half-life within the human body than other antibodies. Accordingly, lower dosages and less frequent administration is often possible. Modifications such as lipidation can be used to stabilize antibodies and to enhance uptake and tissue penetration (e.g., into the brain). A method for lipidation of antibodies is described by Cruikshank et al. ((1997) J. Acquired Immune Deficiency Syndromes and Human Retrovirology 14:193).

The nucleic acid molecules of the invention can be inserted into vectors and used as gene therapy vectors. Gene therapy vectors can be delivered to a subject by, for example, intravenous injection, local administration (U.S. Pat. No. 5,328,470) or by stereotactic injection (see, e.g., Chen et al. (1994) Proc. Natl. Acad. Sci. USA 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g. retroviral vectors, the pharmaceutical preparation can include one or more cells which produce the gene delivery system.

The pharmaceutical compositions can be included in a container, pack, or dispenser together with instructions for administration.

V. Uses and Methods of the Invention

The nucleic acid molecules, proteins, protein homologues, and antibodies described herein can be used in one or more of the following methods: a) screening assays; b) detection assays (e.g., chromosomal mapping, tissue typing, forensic biology); c) predictive medicine (e.g., diagnostic assays, prognostic assays, monitoring clinical trials, and pharmacogenomics); and d) methods of treatment (e.g., therapeutic and prophylactic). For example, polypeptides of the invention can to used to (i) modulate cellular proliferation; (ii) modulate cellular differentiation; and (iii) modulate cell survival. The isolated nucleic acid molecules of the invention can be used to express proteins (e.g., via a recombinant expression vector in a host cell in gene therapy applications), to detect mRNA (e.g., in a biological sample) or a genetic lesion, and to modulate activity of a polypeptide of the invention. In addition, the polypeptides of the invention can be used to screen drugs or compounds which modulate activity or expression of a polypeptide of the invention as well as to treat disorders characterized by insufficient or excessive production of a protein of the invention or production of a form of a protein of the invention which has decreased or aberrant activity compared to the wild type protein. In addition, the antibodies of the invention can be used to detect and isolate a protein of the invention and modulate activity of a protein of the invention.

This invention further pertains to novel agents identified by the above-described screening assays and uses thereof for treatments as described herein.

A. Screening Assays

The invention provides a method (also referred to herein as a “screening assay”) for identifying modulators, i.e., candidate or test compounds or agents (e.g., peptides, peptidomimetics, small molecules or other drugs) which bind to polypeptide of the invention or have a stimulatory or inhibitory effect on, for example, expression or activity of a polypeptide of the invention.

In one embodiment, the invention provides assays for screening candidate or test compounds which bind to or modulate the activity of the membrane-bound form of a polypeptide of the invention or biologically active portion thereof. The test compounds of the present invention can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds (Lam (1997) Anticancer Drug Des. 12:145).

Examples of methods for the synthesis of molecular libraries can be found in the art, for example in: DeWitt et al. (1993) Proc. Natl. Acad. Sci. USA 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and Gallop et al. (1994) J. Med. Chem. 37:1233.

Libraries of compounds may be presented in solution (e.g., Houghten (1992) Bio/Techniques 13:412-421), or on beads (Lam (1991) Nature 354:82-84), chips (Fodor (1993) Nature 364:555-556), bacteria (U.S. Pat. No. 5,223,409), spores (U.S. Pat. Nos. 5,571,698; 5,403,484; and 5,223,409), plasmids (Cull et al. (1992) Proc. Natl. Acad. Sci. USA 89:1865-1869) or phage (Scott and Smith (1990) Science 249:386-390; Devlin (1990) Science 249:404-406; Cwirla et al. (1990) Proc. Natl. Acad. Sci. USA 87:6378-6382; and Felici (1991) J. Mol. Biol. 222:301-310).

In one embodiment, an assay is a cell-based assay in which a cell which expresses a membrane-bound form of a polypeptide of the invention, or a biologically active portion thereof, on the cell surface is contacted with a test compound and the ability of the test compound to bind to the polypeptide determined. The cell, for example, can be a yeast cell or a cell of mammalian origin. Determining the ability of the test compound to bind to the polypeptide can be accomplished, for example, by coupling the test compound with a radioisotope or enzymatic label such that binding of the test compound to the polypeptide or biologically active portion thereof can be determined by detecting the labeled compound in a complex. For example, test compounds can be labeled with ¹²⁵I, ³¹S, ¹⁴C, or ³H, either directly or indirectly, and the radioisotope detected by direct counting of radioemmission or by scintillation counting. Alternatively, test compounds can be enzymatically labeled with, for example, horseradish peroxidase, alkaline phosphatase, or luciferase, and the enzymatic label detected by determination of conversion of an appropriate substrate to product. In a preferred embodiment, the assay comprises contacting a cell which expresses a membrane-bound form of a polypeptide of the invention, or a biologically active portion thereof, on the cell surface with a known compound which binds the polypeptide to form an assay mixture, contacting the assay mixture with a test compound, and determining the ability of the test compound to interact with the polypeptide, wherein determining the ability of the test compound to interact with the polypeptide comprises determining the ability of the test compound to preferentially bind to the polypeptide or a biologically active portion thereof as compared to the known compound.

In another embodiment, an assay is a cell-based assay comprising contacting a cell expressing a membrane-bound form of a polypeptide of the invention, or a biologically active portion thereof, on the cell surface with a test compound and determining the ability of the test compound to modulate (e.g., stimulate or inhibit) the activity of the polypeptide or biologically active portion thereof. Determining the ability of the test compound to modulate the activity of the polypeptide or a biologically active portion thereof can be accomplished, for example, by determining the ability of the polypeptide protein to bind to or interact with a target molecule.

Determining the ability of a polypeptide of the invention to bind to or interact with a target molecule can be accomplished by one of the methods described above for determining direct binding. As used herein, a “target molecule” is a molecule with which a selected polypeptide (e.g., a polypeptide of the invention binds or interacts with in nature, for example, a molecule on the surface of a cell which expresses the selected protein, a molecule on the surface of a second cell, a molecule in the extracellular milieu, a molecule associated with the internal surface of a cell membrane or a cytoplasmic molecule. A target molecule can be a polypeptide of the invention or some other polypeptide or protein. For example, a target molecule can be a component of a signal transduction pathway which facilitates transduction of an extracellular signal (e.g., a signal generated by binding of a compound to a polypeptide of the invention) through the cell membrane and into the cell or a second intercellular protein which has catalytic activity or a protein which facilitates the association of downstream signaling molecules with a polypeptide of the invention. Determining the ability of a polypeptide of the invention to bind to or interact with a target molecule can be accomplished by determining the activity of the target molecule. For example, the activity of the target molecule can be determined by detecting induction of a cellular second messenger of the target (e.g., intracellular Ca²⁺, diacylglycerol, IP3, etc.), detecting catalytic/enzymatic activity of the target on an appropriate substrate, detecting the induction of a reporter gene (e.g., a regulatory element that is responsive to a polypeptide of the invention operably linked to a nucleic acid encoding a detectable marker, e.g. luciferase), or detecting a cellular response, for example, cellular differentiation, or cell proliferation.

In yet another embodiment, an assay of the present invention is a cell-free assay comprising contacting a polypeptide of the invention or biologically active portion thereof with a test compound and determining the ability of the test compound to bind to the polypeptide or biologically active portion thereof. Binding of the test compound to the polypeptide can be determined either directly or indirectly as described above. In a preferred embodiment, the assay includes contacting the polypeptide of the invention or biologically active portion thereof with a known compound which binds the polypeptide to form an assay mixture, contacting the assay mixture with a test compound, and determining the ability of the test compound to interact with the polypeptide, wherein determining the ability of the test compound to interact with the polypeptide comprises determining the ability of the test compound to preferentially bind to the polypeptide or biologically active portion thereof as compared to the known compound.

In another embodiment, an assay is a cell-free assay comprising contacting a polypeptide of the invention or biologically active portion thereof with a test compound and determining the ability of the test compound to modulate (e.g., stimulate or inhibit) the activity of the polypeptide or biologically active portion thereof. Determining the ability of the test compound to modulate the activity of the polypeptide can be accomplished, for example, by determining the ability of the polypeptide to bind to a target molecule by one of the methods described above for determining direct binding. In an alternative embodiment, determining the ability of the test compound to modulate the activity of the polypeptide can be accomplished by determining the ability of the polypeptide of the invention to further modulate the target molecule. For example, the catalytic/enzymatic activity of the target molecule on an appropriate substrate can be determined as previously described.

In yet another embodiment, the cell-free assay comprises contacting a polypeptide of the invention or biologically active portion thereof with a known compound which binds the polypeptide to form an assay mixture, contacting the assay mixture with a test compound, and determining the ability of the test compound to interact with the polypeptide, wherein determining the ability of the test compound to interact with the polypeptide comprises determining the ability of the polypeptide to preferentially bind to or modulate the activity of a target molecule.

The cell-free assays of the present invention are amenable to use of both a soluble form or the membrane-bound form of a polypeptide of the invention. In the case of cell-free assays comprising the membrane-bound form of the polypeptide, it may be desirable to utilize a solubilizing agent such that the membrane-bound form of the polypeptide is maintained in solution. Examples of such solubilizing agents include non-ionic detergents such as n-octylglucoside, n-dodecylglucoside, n-dodecylmaltoside, octanoyl-N-methylglucamide, decanoyl-N-methylglucamide, Triton X-100, Triton X-114, Thesit, Isotridecypoly(ethylene glycol ether)n, 3-[(3-cholamidopropyl)dimethylamminio]-1-propane sulfonate (CHAPS), 3-[(3-cholamidopropyl)dimethylamminio]-2-hydroxy-1-propane sulfonate (CHAPSO), or N-dodecyl=N,N-dimethyl-3-ammonio-1-propane sulfonate.

In more than one embodiment of the above assay methods of the present invention, it may be desirable to immobilize either the polypeptide of the invention or its target molecule to facilitate separation of complexed from uncomplexed forms of one or both of the proteins, as well as to accommodate automation of the assay. Binding of a test compound to the polypeptide, or interaction of the polypeptide with a target molecule in the presence and absence of a candidate compound, can be accomplished in any vessel suitable for containing the reactants. Examples of such vessels include microtitre plates, test tubes, and micro-centrifuge tubes. In one embodiment, a fusion protein can be provided which adds a domain that allows one or both of the proteins to be bound to a matrix. For example, glutathione-S-transferase fusion proteins or glutathione-S-transferase fusion proteins can be adsorbed onto glutathione sepharose beads (Sigma Chemical; St. Louis, Mo.) or glutathione derivatized microtitre plates, which are then combined with the test compound or the test compound and either the non-adsorbed target protein or A polypeptide of the invention, and the mixture incubated under conditions conducive to complex formation (e.g., at physiological conditions for salt and pH). Following incubation, the beads or microtitre plate wells are washed to remove any unbound components and complex formation is measured either directly or indirectly, for example, as described above. Alternatively, the complexes can be dissociated from the matrix, and the level of binding or activity of the polypeptide of the invention can be determined using standard techniques.

Other techniques for immobilizing proteins on matrices can also be used in the screening assays of the invention. For example, either the polypeptide of the invention or its target molecule can be immobilized utilizing conjugation of biotin and streptavidin. Biotinylated polypeptide of the invention or target molecules can be prepared from biotin-NHS(N-hydroxy-succinimide) using techniques well known in the art (e.g., biotinylation kit, Pierce Chemicals; Rockford, Ill.), and immobilized in the wells of streptavidin-coated 96 well plates (Pierce Chemical). Alternatively, antibodies reactive with the polypeptide of the invention or target molecules but which do not interfere with binding of the polypeptide of the invention to its target molecule can be derivatized to the wells of the plate, and unbound target or polypeptidede of the invention trapped in the wells by antibody conjugation. Methods for detecting such complexes, in addition to those described above for the GST-immobilized complexes, include immunodetection of complexes using antibodies reactive with the polypeptide of the invention or target molecule, as well as enzyme-linked assays which rely on detecting an enzymatic activity associated with the polypeptide of the invention or target molecule.

In another embodiment, modulators of expression of a polypeptide of the invention are identified in a method in which a cell is contacted with a candidate compound and the expression of the selected mRNA or protein (i.e., the mRNA or protein corresponding to a polypeptide or nucleic acid of the invention) in the cell is determined. The level of expression of the selected mRNA or protein in the presence of the candidate compound is compared to the level of expression of the selected mRNA or protein in the absence of the candidate compound. The candidate compound can then be identified as a modulator of expression of the polypeptide of the invention based on this comparison. For example, when expression of the selected mRNA or protein is greater (statistically significantly greater) in the presence of the candidate compound than in its absence, the candidate compound is identified as a stimulator of the selected mRNA or protein expression. Alternatively, when expression of the selected mRNA or protein is less (statistically significantly less) in the presence of the candidate compound than in its absence, the candidate compound is identified as an inhibitor of the selected mRNA or protein expression. The level of the selected mRNA or protein expression in the cells can be determined by methods described herein.

In yet another aspect of the invention, a polypeptide of the inventions can be used as “bait proteins” in a two-hybrid assay or three hybrid assay (see, e.g., U.S. Pat. No. 5,283,317; Zervos et al. (1993) Cell 72:223-232; Madura et al. (1993) J. Biol. Chem. 268:12046-12054; Bartel et al. (1993) Bio/Techniques 14:920-924; Iwabuchi et al. (1993) Oncogene 8:1693-1696; and PCT Publication No. WO 94/10300), to identify other proteins, which bind to or interact with the polypeptide of the invention and modulate activity of the polypeptide of the invention. Such binding proteins are also likely to be involved in the propagation of signals by the polypeptide of the inventions as, for example, upstream or downstream elements of a signaling pathway involving the polypeptide of the invention.

This invention further pertains to novel agents identified by the above-described screening assays and uses thereof for treatments as described herein.

B. Detection Assays

Portions or fragments of the cDNA sequences identified herein (and the corresponding complete gene sequences) can be used in numerous ways as polynucleotide reagents. For example, these sequences can be used to: (i) map their respective genes on a chromosome and, thus, locate gene regions associated with genetic disease; (ii) identify an individual from a minute biological sample (tissue typing); and (iii) aid in forensic identification of a biological sample. These applications are described in the subsections below.

1. Chromosome Mapping

Once the sequence (or a portion of the sequence) of a gene has been isolated, this sequence can be used to map the location of the gene on a chromosome. Accordingly, nucleic acid molecules described herein or fragments thereof, can be used to map the location of the corresponding genes on a chromosome. The mapping of the sequences to chromosomes is an important first step in correlating these sequences with genes associated with disease.

Briefly, genes can be mapped to chromosomes by preparing PCR primers (preferably 15-25 bp in length) from the sequence of a gene of the invention. Computer analysis of the sequence of a gene of the invention can be used to rapidly select primers that do not span more than one exon in the genomic DNA, thus complicating the amplification process. These primers can then be used for PCR screening of somatic cell hybrids containing individual human chromosomes. Only those hybrids containing the human gene corresponding to the gene sequences will yield an amplified fragment. For a review of this technique, see D'Eustachio et al. ((1983) Science 220:919-924).

PCR mapping of somatic cell hybrids is a rapid procedure for assigning a particular sequence to a particular chromosome. Three or more sequences can be assigned per day using a single thermal cycler. Using the nucleic acid sequences of the invention to design oligonucleotide primers, sublocalization can be achieved with panels of fragments from specific chromosomes. Other mapping strategies which can similarly be used to map a gene to its chromosome include in situ hybridization (described in Fan et al. (1990) Proc. Natl. Acad. Sci. USA 87:6223-27), pre-screening with labeled flow-sorted chromosomes, and pre-selection by hybridization to chromosome specific cDNA libraries. Fluorescence in situ hybridization (FISH) of a DNA sequence to a metaphase chromosomal spread can further be used to provide a precise chromosomal location in one step. For a review of this technique, see Verma et al., (Human Chromosomes: A Manual of Basic Techniques (Pergamon Press, New York, 1988)).

Reagents for chromosome mapping can be used individually to mark a single chromosome or a single site on that chromosome, or panels of reagents can be used for marking multiple sites and/or multiple chromosomes. Reagents corresponding to noncoding regions of the genes actually are preferred for mapping purposes. Coding sequences are more likely to be conserved within gene families, thus increasing the chance of cross hybridizations during chromosomal mapping.

Once a sequence has been mapped to a precise chromosomal location, the physical position of the sequence on the chromosome can be correlated with genetic map data. (Such data are found, for example, in V. McKusick, Mendelian Inheritance in Man, available on-line through Johns Hopkins University Welch Medical Library). The relationship between genes and disease, mapped to the same chromosomal region, can then be identified through linkage analysis (co-inheritance of physically adjacent genes), described in, e.g., Egeland et al. (1987) Nature 325:783-787.

Moreover, differences in the DNA sequences between individuals affected and unaffected with a disease associated with a gene of the invention can be determined. If a mutation is observed in some or all of the affected individuals but not in any unaffected individuals, then the mutation is likely to be the causative agent of the particular disease. Comparison of affected and unaffected individuals generally involves first looking for structural alterations in the chromosomes such as deletions or translocations that are visible from chromosome spreads or detectable using PCR based on that DNA sequence. Ultimately, complete sequencing of genes from several individuals can be performed to confirm the presence of a mutation and to distinguish mutations from polymorphisms.

2. Tissue Typing

The nucleic acid sequences of the present invention can also be used to identify individuals from minute biological samples. The United States military, for example, is considering the use of restriction fragment length polymorphism (RFLP) for identification of its personnel. In this technique, an individual's genomic DNA is digested with one or more restriction enzymes, and probed on a Southern blot to yield unique bands for identification. This method does not suffer from the current limitations of “Dog Tags” which can be lost, switched, or stolen, making positive identification difficult. The sequences of the present invention are useful as additional DNA markers for RFLP (described in U.S. Pat. No. 5,272,057).

Furthermore, the sequences of the present invention can be used to provide an alternative technique which determines the actual base-by-base DNA sequence of selected portions of an individual's genome. Thus, the nucleic acid sequences described herein can be used to prepare two PCR primers from the 5′ and 3′ ends of the sequences. These primers can then be used to amplify an individual's DNA and subsequently sequence it.

Panels of corresponding DNA sequences from individuals, prepared in this manner, can provide unique individual identifications, as each individual will have a unique set of such DNA sequences due to allelic differences. The sequences of the present invention can be used to obtain such identification sequences from individuals and from tissue. The nucleic acid sequences of the invention uniquely represent portions of the human genome. Allelic variation occurs to some degree in the coding regions of these sequences, and to a greater degree in the noncoding regions. It is estimated that allelic variation between individual humans occurs with a frequency of about once per each 500 bases. Each of the sequences described herein can, to some degree, be used as a standard against which DNA from an individual can be compared for identification purposes. Because greater numbers of polymorphisms occur in the noncoding regions, fewer sequences are necessary to differentiate individuals. For example, the noncoding sequences of SEQ ID NO:1 can comfortably provide positive individual identification with a panel of perhaps 10 to 1,000 primers which each yield a noncoding amplified sequence of 100 bases. If predicted coding sequences, such as those in SEQ ID NO:3 are used, a more appropriate number of primers for positive individual identification would be 500-2,000.

If a panel of reagents from the nucleic acid sequences described herein is used to generate a unique identification database for an individual, those same reagents can later be used to identify tissue from that individual. Using the unique identification database, positive identification of the individual, living or dead, can be made from extremely small tissue samples.

3. Use of Partial Gene Sequences in Forensic Biology

DNA-based identification techniques can also be used in forensic biology. Forensic biology is a scientific field employing genetic typing of biological evidence found at a crime scene as a means for positively identifying, for example, a perpetrator of a crime. To make such an identification, PCR technology can be used to amplify DNA sequences taken from very small biological samples such as tissues, e.g., hair or skin, or body fluids, e.g., blood, saliva, or semen found at a crime scene. The amplified sequence can then be compared to a standard, thereby allowing identification of the origin of the biological sample.

The sequences of the present invention can be used to provide polynucleotide reagents, e.g., PCR primers, targeted to specific loci in the human genome, which can enhance the reliability of DNA-based forensic identifications by, for example, providing another “identification marker” (i.e. another DNA sequence that is unique to a particular individual). As mentioned above, actual base sequence information can be used for identification as an accurate alternative to patterns formed by restriction enzyme generated fragments. Sequences targeted to noncoding regions are particularly appropriate for this use as greater numbers of polymorphisms occur in the noncoding regions, making it easier to differentiate individuals using this technique. Examples of polynucleotide reagents include the nucleic acid sequences of the invention or portions thereof, e.g., fragments derived from noncoding regions having a length of at least 20 or 30 bases.

The nucleic acid sequences described herein can further be used to provide polynucleotide reagents, e.g., labeled or labelable probes which can be used in, for example, an in situ hybridization technique, to identify a specific tissue, e.g., brain tissue. This can be very useful in cases where a forensic pathologist is presented with a tissue of unknown origin. Panels of such probes can be used to identify tissue by species and/or by organ type.

C. Predictive Medicine

The present invention also pertains to the field of predictive medicine in which diagnostic assays, prognostic assays, pharmacogenomics, and monitoring clinical trails are used for prognostic (predictive) purposes to thereby treat an individual prophylactically. Accordingly, one aspect of the present invention relates to diagnostic assays for determining expression of a polypeptide or nucleic acid of the invention and/or activity of a polypeptide of the invention, in the context of a biological sample (e.g., blood, serum, cells, tissue) to thereby determine whether an individual is afflicted with a disease or disorder, or is at risk of developing a disorder, associated with aberrant expression or activity of a polypeptide of the invention. The invention also provides for prognostic (or predictive) assays for determining whether an individual is at risk of developing a disorder associated with aberrant expression or activity of a polypeptide of the invention. For example, mutations in a gene of the invention can be assayed in a biological sample. Such assays can be used for prognostic or predictive purpose to thereby prophylactically treat an individual prior to the onset of a disorder characterized by or associated with aberrant expression or activity of a polypeptide of the invention.

Another aspect of the invention provides methods for expression of a nucleic acid or polypeptide of the invention or activity of a polypeptide of the invention in an individual to thereby select appropriate therapeutic or prophylactic agents for that individual (referred to herein as “pharmacogenomics”). Pharmacogenomics allows for the selection of agents (e.g., drugs) for therapeutic or prophylactic treatment of an individual based on the genotype of the individual (e.g., the genotype of the individual examined to determine the ability of the individual to respond to a particular agent).

Yet another aspect of the invention pertains to monitoring the influence of agents (e.g., drugs or other compounds) on the expression or activity of a polypeptide of the invention in clinical trials.

These and other agents are described in further detail in the following sections.

1. Diagnostic Assays

An exemplary method for detecting the presence or absence of a polypeptide or nucleic acid of the invention in a biological sample involves obtaining a biological sample from a test subject and contacting the biological sample with a compound or an agent capable of detecting a polypeptide or nucleic acid (e.g., mRNA, genomic DNA) of the invention such that the presence of a polypeptide or nucleic acid of the invention is detected in the biological sample. A preferred agent for detecting mRNA or genomic DNA encoding a polypeptide of the invention is a labeled nucleic acid probe capable of hybridizing to mRNA or genomic DNA encoding a polypeptide of the invention. The nucleic acid probe can be, for example, a full-length cDNA, such as the nucleic acid of SEQ ID NOs:1-22, 34-43, and ______-______ or a portion thereof, such as an oligonucleotide of at least 15, 30, 50, 100, 250 or 500 nucleotides in length and sufficient to specifically hybridize under stringent conditions to a mRNA or genomic DNA encoding a polypeptide of the invention. Other suitable probes for use in the diagnostic assays of the invention are described herein.

A preferred agent for detecting A polypeptide of the invention is an antibody capable of binding to A polypeptide of the invention, preferably an antibody with a detectable label. Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin. The term “biological sample” is intended to include tissues, cells and biological fluids isolated from a subject, as well as tissues, cells and fluids present within a subject. That is, the detection method of the invention can be used to detect mRNA, protein, or genomic DNA in a biological sample in vitro as well as in vivo. For example, in vitro techniques for detection of mRNA include Northern hybridizations and in situ hybridizations. In vitro techniques for detection of A polypeptide of the invention include enzyme linked immunosorbent assays (ELISAs), Western blots, immunoprecipitations and immunofluorescence. In vitro techniques for detection of genomic DNA include Southern hybridizations. Furthermore, in vivo techniques for detection of a polypeptide of the invention include introducing into a subject a labeled antibody directed against the polypeptide. For example, the antibody can be labeled with a radioactive marker whose presence and location in a subject can be detected by standard imaging techniques.

In one embodiment, the biological sample contains protein molecules from the test subject. Alternatively, the biological sample can contain mRNA molecules from the test subject or genomic DNA molecules from the test subject. A preferred biological sample is a peripheral blood leukocyte sample isolated by conventional means from a subject.

In another embodiment, the methods further involve obtaining a control biological sample from a control subject, contacting the control sample with a compound or agent capable of detecting a polypeptide of the invention or mRNA or genomic DNA encoding a polypeptide of the invention, such that the presence of the polypeptide or mRNA or genomic DNA encoding the polypeptide is detected in the biological sample, and comparing the presence of the polypeptide or mRNA or genomic DNA encoding the polypeptide in the control sample with the presence of the polypeptide or mRNA or genomic DNA encoding the polypeptide in the test sample.

The invention also encompasses kits for detecting the presence of a polypeptide or nucleic acid of the invention in a biological sample (a test sample). Such kits can be used to determine if a subject is suffering from or is at increased risk of developing a disorder associated with aberrant expression of a polypeptide of the invention (e.g., an immunological disorder). For example, the kit can comprise a labeled compound or agent capable of detecting the polypeptide or mRNA encoding the polypeptide in a biological sample and means for determining the amount of the polypeptide or mRNA in the sample (e.g., an antibody which binds the polypeptide or an oligonucleotide probe which binds to DNA or mRNA encoding the polypeptide). Kits can also include instruction for observing that the tested subject is suffering from or is at risk of developing a disorder associated with aberrant expression of the polypeptide if the amount of the polypeptide or mRNA encoding the polypeptide is above or below a normal level.

For antibody-based kits, the kit can comprise, for example: (1) a -first antibody (e.g., attached to a solid support) which binds to a polypeptide of the invention; and, optionally, (2) a second, different antibody which binds to either the polypeptide or the first antibody and is conjugated to a detectable agent.

For oligonucleotide-based kits, the kit can comprise, for example: (1) an oligonucleotide, e.g., a detectably labeled oligonucleotide, which hybridizes to a nucleic acid sequence encoding a polypeptide of the invention or (2) a pair of primers useful for amplifying a nucleic acid molecule encoding a polypeptide of the invention.

The kit can also comprise, e.g., a buffering agent, a preservative, or a protein stabilizing agent. The kit can also comprise components necessary for detecting the detectable agent (e.g., an enzyme or a substrate). The kit can also contain a control sample or a series of control samples which can be assayed and compared to the test sample contained. Each component of the kit is usually enclosed within an individual container and all of the various containers are within a single package along with instructions for observing whether the tested subject is suffering from or is at risk of developing a disorder associated with aberrant expression of the polypeptide.

2. Prognostic Assays

The methods described herein can furthermore be utilized as diagnostic or prognostic assays to identify subjects having or at risk of developing a disease or disorder associated with aberrant expression or activity of a polypeptide of the invention. For example, the assays described herein, such as the preceding diagnostic assays or the following assays, can be utilized to identify a subject having or at risk of developing a disorder associated with aberrant expression or activity of a polypeptide of the invention. Alternatively, the prognostic assays can be utilized to identify a subject having or at risk for developing such a disease or disorder. Thus, the present invention provides a method in which a test sample is obtained from a subject and a polypeptide or nucleic acid (e.g., mRNA, genomic DNA) of the invention is detected, wherein the presence of the polypeptide or nucleic acid is diagnostic for a subject having or at risk of developing a disease or disorder associated with aberrant expression or activity of the polypeptide. As used herein, a “test sample” refers to a biological sample obtained from a subject of interest. For example, a test sample can be a biological fluid (e.g., serum), cell sample, or tissue.

Furthermore, the prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with aberrant expression or activity of a polypeptide of the invention. For example, such methods can be used to determine whether a subject can be effectively treated with a specific agent or class of agents (e.g., agents of a type which decrease activity of the polypeptide). Thus, the present invention provides methods for determining whether a subject can be effectively treated with an agent for a disorder associated with aberrant expression or activity of a polypeptide of the invention in which a test sample is obtained and the polypeptide or nucleic acid encoding the polypeptide is detected (e.g., wherein the presence of the polypeptide or nucleic acid is diagnostic for a subject that can be administered the agent to treat a disorder associated with aberrant-expression or activity of the polypeptide).

The methods of the invention can also be used to detect genetic lesions or mutations in a gene of the invention, thereby determining if a subject with the lesioned gene is at risk for a disorder characterized aberrant expression or activity of a polypeptide of the invention. In preferred embodiments, the methods include detecting, in a sample of cells from the subject, the presence or absence of a genetic lesion or mutation characterized by at least one of an alteration affecting the integrity of a gene encoding the polypeptide of the invention, or the mis-expression of the gene encoding the polypeptide of the invention. For example, such genetic lesions or mutations can be detected by ascertaining the existence of at least one of: 1) a deletion of one or more nucleotides from the gene; 2) an addition of one or more nucleotides to the gene; 3) a substitution of one or more nucleotides of the gene; 4) a chromosomal rearrangement of the gene; 5) an alteration in the level of a messenger RNA transcript of the gene; 6) an aberrant modification of the gene, such as of the methylation pattern of the genomic DNA; 7) the presence of a non-wild type splicing pattern of a messenger RNA transcript of the gene; 8) a non-wild type level of a the protein encoded by the gene; 9) an allelic loss of the gene; and 10) an inappropriate post-translational modification of the protein encoded by the gene. As described herein, there are a large number of assay techniques known in the art which can be used for detecting lesions in a gene.

In certain embodiments, detection of the lesion involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran et al. (1988) Science 241:1077-1080; and Nakazawa et al. (1994) Proc. Natl. Acad. Sci. USA 91:360-364), the latter of which can be particularly useful for detecting point mutations in a gene (see, e.g., Abravaya et al. (1995) Nucleic Acids Res. 23:675-682). This method can include the steps of collecting a sample of cells from a patient, isolating nucleic acid (e.g., genomic, mRNA or both) from the cells of the sample, contacting the nucleic acid sample with one or more primers which specifically hybridize to the selected gene under conditions such that hybridization and amplification of the gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication (Guatelli et al. (1990) Proc. Natl. Acad. Sci. USA 87:1874-1878), transcriptional amplification system (Kwoh, et al. (1989) Proc. Natl. Acad. Sci. USA 86:1173-1177), Q-Beta Replicase (Lizardi et al. (1988) Bio/Technology 6:1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in a selected gene from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, e.g., U.S. Pat. No. 5,498,531) can be used to score for the presence of specific mutations by development or loss of a ribozyme cleavage site.

In other embodiments, genetic mutations can be identified by hybridizing a sample and control nucleic acids, e.g., DNA or RNA, to high density arrays containing hundreds or thousands of oligonucleotides probes (Cronin et al. (1996) Human Mutation 7:244-255; Kozal et al. (1996) Nature Medicine 2:753-759). For example, genetic mutations can be identified in two-dimensional arrays containing light-generated DNA probes as described in Cronin et al., supra. Briefly, a first hybridization array of probes can be used to scan through long stretches of DNA in a sample and control to identify base changes between the sequences by making linear arrays of sequential overlapping probes. This step allows the identification of point mutations. This step is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the selected gene and detect mutations by comparing the sequence of the sample nucleic acids with the corresponding wild-type (control) sequence. Examples of sequencing reactions include those based on techniques developed by Maxim and Gilbert ((1977) Proc. Natl. Acad. Sci. USA 74:560) or Sanger ((1977) Proc. Natl. Acad. Sci. USA 74:5463). It is also contemplated that any of a variety of automated sequencing procedures can be utilized when performing the diagnostic assays ((1995) Bio/Techniques 19:448), including sequencing by mass spectrometry (see, e.g., PCT Publication No. WO 94/16101; Cohen et al. (1996) Adv. Chromatogr. 36:127-162; and Griffin et al. (1993) Appl. Biochem. Biotechnol. 38:147-159).

Other methods for detecting mutations in a selected gene include methods in which protection from cleavage agents is used to detect mismatched bases in RNA/RNA or RNA/DNA heteroduplexes (Myers et al. (1985) Science 230:1242). In general, the technique of “mismatch cleavage” entails providing heteroduplexes formed by hybridizing (labeled) RNA or DNA containing the wild-type sequence with potentially mutant RNA or DNA obtained from a tissue sample. The double-stranded duplexes are treated with an agent which cleaves single-stranded regions of the duplex such as which will exist due to basepair mismatches between the control and sample strands. RNA/DNA duplexes can be treated with RNase to digest mismatched regions, and DNA/DNA hybrids can be treated with S1 nuclease to digest mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, e.g., Cotton et al. (1988) Proc. Natl. Acad. Sci. USA 85:4397; Saleeba et al. (1992) Methods Enzymol. 217:286-295. In a preferred embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in cDNAs obtained from samples of cells. For example, the mutY enzyme of E. coli cleaves A at G/A mismatches and the thymidine DNA glycosylase from HeLa cells cleaves T at G/T mismatches (Hsu et al. (1994) Carcinogenesis 15:1657-1662). According to an exemplary embodiment, a probe based on a selected sequence, e.g., a wild-type sequence, is hybridized to a cDNA or other DNA product from a test cell(s). The duplex is treated with a DNA mismatch repair enzyme, and the cleavage products, if any, can be detected from electrophoresis protocols or the like. See, e.g., U.S. Pat. No. 5,459,039.

In other embodiments, alterations in electrophoretic mobility will be used to identify mutations in genes. For example, single strand conformation polymorphism (SSCP) may be used to detect differences in electrophoretic mobility between mutant and wild type nucleic acids (Orita et al. (1989) Proc. Natl. Acad. Sci. USA 86:2766; see also Cotton (1993) Mutat. Res. 285:125-144; Hayashi (1992) Genet. Anal. Tech. Appl. 9:73-79). Single-stranded DNA fragments of sample and control nucleic acids will be denatured and allowed to renature. The secondary structure of single-stranded nucleic acids varies according to sequence, and the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In a preferred embodiment, the subject method utilizes heteroduplex analysis to separate double stranded heteroduplex molecules on the basis of changes in electrophoretic mobility (Keen et al. (1991) Trends Genet. 7:5).

In yet another embodiment, the movement of mutant or wild-type fragments in polyacrylamide gels containing a gradient of denaturant is assayed using denaturing gradient gel electrophoresis (DGGE) (Myers et al. (1985) Nature 313:495). When DGGE is used as the method of analysis, DNA will be modified to insure that it does not completely denature, for example by adding a ‘GC clamp of approximately 40 bp of high-melting GC-rich DNA by PCR. In a further embodiment, a temperature gradient is used in place of a denaturing gradient to identify differences in the mobility of control and sample DNA (Rosenbaum and Reissner (1987) Biophys. Chem. 265:12753).

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions which permit hybridization only if a perfect match is found (Saiki et al. (1986) Nature 324:163); Saiki et al. (1989) Proc. Natl. Acad. Sci. USA 86:6230). Such allele specific oligonucleotides are hybridized to PCR amplified target DNA or a number of different mutations when the oligonucleotides are attached to the hybridizing membrane and hybridized with labeled target DNA.

Alternatively, allele specific amplification technology which depends on selective PCR amplification may be used in conjunction with the instant invention. Oligonucleotides used as primers for specific amplification may carry the mutation of interest in the center of the molecule (so that amplification depends on differential hybridization) (Gibbs et al. (1989) Nucleic Acids Res. 17:2437-2448) or at the extreme 3′ end of one primer where, under appropriate conditions, mismatch can prevent or reduce polymerase extension (Prossner (1993) Tibtech 11:238). In addition, it may be desirable to introduce a novel restriction site in the region of the mutation to create cleavage-based detection (Gasparini et al. (1992) Mol. Cell Probes 6:1). It is anticipated that in certain embodiments amplification may also be performed using Taq ligase for amplification (Barany (1991) Proc. Natl. Acad. Sci. USA 88:189). In such cases, ligation will occur only if there is a perfect match at the 3′ end of the 5′ sequence making it possible to detect the presence of a known mutation at a specific site by looking for the presence or absence of amplification.

The methods described herein may be performed, for example, by utilizing pre-packaged diagnostic kits comprising at least one probe nucleic acid or antibody reagent described herein, which may be conveniently used, e.g., in clinical settings to diagnose patients exhibiting symptoms or family history of a disease or illness involving a gene encoding a polypeptide of the invention.

Furthermore, any cell type or tissue, preferably peripheral blood leukocytes, in which the polypeptide of the invention is expressed may be utilized in the prognostic assays described herein.

3. Pharmacogenomics

Agents, or modulators which have a stimulatory or inhibitory effect on activity or expression of a polypeptide of the invention as identified by a screening assay described herein can be administered to individuals to treat (prophylactically or therapeutically) disorders associated with aberrant activity of the polypeptide. In conjunction with such treatment, the pharmacogenomics (i.e., the study of the relationship between an individual's genotype and that individual's response to a foreign compound or drug) of the individual may be considered. Differences in metabolism of therapeutics can lead to severe toxicity or therapeutic failure by altering the relation between dose and blood concentration of the pharmacologically active drug. Thus, the pharmacogenomics of the individual permits the selection of effective agents (e.g., drugs) for prophylactic or therapeutic treatments based on a consideration of the individual's genotype. Such pharmacogenomics can further be used to determine appropriate dosages and therapeutic regimens. Accordingly, the activity of a polypeptide of the invention, expression of a nucleic acid of the invention, or mutation content of a gene of the invention in an individual can be determined to thereby select appropriate agent(s) for therapeutic or prophylactic treatment of the individual.

Pharmacogenomics deals with clinically significant hereditary variations in the response to drugs due to altered drug disposition and abnormal action in affected persons. See, e.g., Linder (1997) Clin. Chem. 43(2):254-266. In general, two types of pharmacogenetic conditions can be differentiated. Genetic conditions transmitted as a single factor altering the way drugs act on the body are referred to as “altered drug action.” Genetic conditions transmitted as single factors altering the way the body acts on drugs are referred to as “altered drug metabolism”. These pharmacogenetic conditions can occur either as rare defects or as polymorphisms. For example, glucose-6-phosphate dehydrogenase deficiency (G6PD) is a common inherited enzymopathy in which the main clinical complication is haemolysis after ingestion of oxidant drugs (anti-malarials, sulfonamides, analgesics, nitrofurans) and consumption of fava beans.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome P450 enzymes CYP2D6 and CYP2C19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C19 quite frequently experience exaggerated drug response and side effects when they receive standard doses. If a metabolite is the active therapeutic moiety, a PM will show no therapeutic response, as demonstrated for the analgesic effect of codeine mediated by its CYP2D6-formed metabolite morphine. The other extreme are the so called ultra-rapid metabolizers who do not respond to standard doses. Recently, the molecular basis of ultra-rapid metabolism has been identified to be due to CYP2D6 gene amplification.

Thus, the activity of a polypeptide of the invention, expression of a nucleic acid encoding the polypeptide, or mutation content of a gene encoding the polypeptide in an individual can be determined to thereby select appropriate agent(s) for therapeutic or prophylactic treatment of the individual. In addition, pharmacogenetic studies can be used to apply genotyping of polymorphic alleles encoding drug-metabolizing enzymes to the identification of an individual's drug responsiveness phenotype. This knowledge, when applied to dosing or drug selection, can avoid adverse reactions or therapeutic failure and thus enhance therapeutic or prophylactic efficiency when treating a subject with a modulator of activity or expression of the polypeptide, such as a modulator identified by one of the exemplary screening assays described herein.

4. Monitoring of Effects During Clinical Trials

Monitoring the influence of agents (e.g., drugs, compounds) on the expression or activity of a polypeptide of the invention (e.g., the ability to modulate aberrant cell proliferation and/or differentiation) can be applied not only in basic drug screening, but also in clinical trials. For example, the effectiveness of an agent, as determined by a screening assay as described herein, to increase gene expression, protein levels or protein activity, can be monitored in clinical trials of subjects exhibiting decreased gene expression, protein levels, or protein activity. Alternatively, the effectiveness of an agent, as determined by a screening assay, to decrease gene expression, protein levels or protein activity, can be monitored in clinical trials of subjects exhibiting increased gene expression, protein levels, or protein activity. In such clinical trials, expression or activity of a polypeptide of the invention and preferably, that of other polypeptide that have been implicated in for example, a cellular proliferation disorder, can be used as a marker of the immune responsiveness of a particular cell.

For example, and not by way of limitation, genes, including those of the invention, that are modulated in cells by treatment with an agent (e.g., compound, drug or small molecule) which modulates activity or expression of a polypeptide of the invention (e.g., as identified in a screening assay described herein) can be identified. Thus, to study the effect of agents on cellular proliferation disorders, for example, in a clinical trial, cells can be isolated and RNA prepared and analyzed for the levels of expression of a gene of the invention and other genes implicated in the disorder. The levels of gene expression (i.e., a gene expression pattern) can be quantified by Northern blot analysis or RT-PCR, as described herein, or alternatively by measuring the amount of protein produced, by one of the methods as described herein, or by measuring the levels of activity of a gene of the invention or other genes. In this way, the gene expression pattern can serve as a marker, indicative of the physiological response of the cells to the agent. Accordingly, this response state may be determined before, and at various points during, treatment of the individual with the agent.

In a preferred embodiment, the present invention provides a method for monitoring the effectiveness of treatment of a subject with an agent (e.g., an agonist, antagonist, peptidomimetic, protein, peptide, nucleic acid, small molecule, or other drug candidate identified by the screening assays described herein) comprising the steps of (i) obtaining a pre-administration sample from a subject prior to administration of the agent; (ii) detecting the level of the polypeptide or nucleic acid of the invention in the preadministration sample; (iii) obtaining one or more post-administration samples from the subject; (iv) detecting the level the of the polypeptide or nucleic acid of the invention in the post-administration samples; (v) comparing the level of the polypeptide or nucleic acid of the invention in the pre-administration sample with the level of the polypeptide or nucleic acid of the invention in the post-administration sample or samples; and (vi) altering the administration of the agent to the subject accordingly. For example, increased administration of the agent may be desirable to increase the expression or activity of the polypeptide to higher levels than detected, i.e., to increase the effectiveness of the agent. Alternatively, decreased administration of the agent may be desirable to decrease expression or activity of the polypeptide to lower levels than detected, i.e., to decrease the effectiveness of the agent.

C. Methods of Treatment

The present invention provides for both prophylactic and therapeutic methods of treating a subject at risk of (or susceptible to) a disorder or having a disorder associated with aberrant expression or activity of a polypeptide of the invention.

1. Prophylactic Methods

In one aspect, the invention provides a method for preventing in a subject, a disease or condition associated with an aberrant expression or activity of a polypeptide of the invention, by administering to the subject an agent which modulates expression or at least one activity of the polypeptide. Subjects at risk for a disease which is caused or contributed to by aberrant expression or activity of a polypeptide of the invention can be identified by, for example, any or a combination of diagnostic or prognostic assays as described herein. Administration of a prophylactic agent can occur prior to the manifestation of symptoms characteristic of the aberrancy, such that a disease or disorder is prevented or, alternatively, delayed in its progression. Depending on the type of aberrancy, for example, an agonist or antagonist agent can be used for treating the subject. The appropriate agent can be determined based on screening assays described herein.

2. Therapeutic Methods

Another aspect of the invention pertains to methods of modulating expression or activity of a polypeptide of the invention for therapeutic purposes. The modulatory method of the invention involves contacting a cell with an agent that modulates one or more of the activities of the polypeptide. An agent that modulates activity can be an agent as described herein, such as a nucleic acid or a protein, a naturally-occurring cognate ligand of the polypeptide, a peptide, a peptidomimetic, or other small molecule. In one embodiment, the agent stimulates one or more of the biological activities of the polypeptide. Examples of such stimulatory agents include the active polypeptide of the invention and a nucleic acid molecule encoding the polypeptide of the invention that has been introduced into the cell. In another embodiment, the agent inhibits one or more of the biological activities of the polypeptide of the invention. Examples of such inhibitory agents include antisense nucleic acid molecules and antibodies. These modulatory methods can be performed in vitro (e.g., by culturing the cell with the agent) or, alternatively, in vivo (e.g, by administering the agent to a subject). As such, the present invention provides methods of treating an individual afflicted with a disease or disorder characterized by aberrant expression or activity a polypeptide of the invention. In one embodiment, the method involves administering an agent (e.g., an agent identified by a screening assay described herein), or combination of agents that modulates (e.g., upregulates or down-regulates) expression or activity. In another embodiment, the method involves administering a polypeptide of the invention or a nucleic acid molecule of the invention as therapy to compensate for reduced or aberrant expression or activity of the polypeptide.

Stimulation of activity is desirable in situations in which activity or expression is abnormally low down-regulated and/or in which increased activity is likely to have a beneficial effect. Conversely, inhibition of activity is desirable in situations in which activity or expression is abnormally high or upregulated and/or in which decreased activity is likely to have a beneficial effect.

This invention is further illustrated by the following examples which should not be construed as limiting. The contents of all references, patents and published patent applications cited throughout this application are hereby incorporated by reference.

### EXAMPLES

TANGO 180, TANGO 181, TANGO 182, TANGO 183, TANGO 184, TANGO 185, TANGO 186, TANGO 188, TANGO 189 and TANGO 187, were identified in a human prostate epithelial cell library. TANGO 215 was identified in a human prostate stromal cell library.

TANGO 180, TANGO 181, TANGO 182, TANGO 183, TANGO 184, TANGO 185, TANGO 186, TANGO 188, TANGO 189, TANGO 215, and TANGO 187 were identified by first analyzing clones present in the two libraries to identify EST sequences which potentially encode a signal peptide having at least 15 amino acids. Selected clones which include an EST sequence that appeared to encode a signal peptide having at least 15 amino acids were used to assemble additional EST sequences to form potential full-length gene sequences. The assembled full-length gene sequences were then used to identify actual full-length clones in the two libraries.

Deposit of Clones

Clones containing cDNA molecules encoding TANGO 180, TANGO 181, TANGO 182, TANGO 183, TANGO 184, TANGO 185, TANGO 186, TANGO 188, TANGO 189, TANGO 215 and TANGO 187 were deposited with the American Type Culture Collection (Manassas, Va.) as composite deposits.

Clones encoding TANGO 180, TANGO 181, TANGO 182 and TANGO 183, and TANGO 184 were deposited on Sep. 25, 1998 with the American Type Culture Collection under accession number ATCC 98901, from which each clone comprising a particular cDNA clone is obtainable. This deposit is a mixture of five strains, each carrying one recombinant plasmid harboring a particular cDNA clone. To distinguish the strains and isolate a strain harboring a particular cDNA clone, one can first streak out an aliquot of the mixture to single colonies on nutrient medium (e.g., LB plates) supplemented with 100 μg/ml ampicillin, grow single colonies, and then extract the plasmid DNA using a standard minipreparation procedure. Next, one can digest a sample of the DNA minipreparation with a combination of the restriction enzymes Sal I and Not I and resolve the resultant products on a 0.8% agarose gel using standard DNA electrophoresis conditions. The digest will liberate fragments as follows:

| | |
|---|---|
| TANGO 180 (EpT180) | 1.2 kb and 2.7 kb |
| TANGO 181 (EpT181) | 4.5 kb and 2.7 kb |
| TANGO 182 (EpT182) | two 2.7 kb fragments |
| TANGO 183 (EpT183) | 1.6 kb and 2.7 kb |
| TANGO 184 (EpT184) | 4.5 kb |

The identity of the strains can be inferred from the fragments liberated.

Clones encoding TANGO 185, TANGO 186, TANGO 187, TANGO 188 and TANGO 189 (splice variant 1) were deposited on Sep. 25, 1998 with the American Type Culture Collection under accession number ATCC 98900, from which each stain comprising a particular cDNA clone is obtainable. The deposit is a mixture of five strains, each carrying one recombinant plasmid harboring a particular cDNA clone. To distinguish the strains and isolate a strain harboring a particular cDNA clone, one can first streak out an aliquot of the mixture to single colonies on nutrient medium (e.g., LB plates) supplemented with 100 μg/ml ampicillin, grow single colonies, and then extract the plasmid DNA using a standard minipreparation procedure. Next, one can digest a sample of the DNA minipreparation with a combination of the restriction enzymes Sal I and Not I and resolve the resultant products on a 0.8% agarose gel using standard DNA electrophoresis conditions. The digest will liberate one vector fragment of 2.7 kb common to all strains, and one insert-specific fragment as follows:

| | |
|---|---|
| TANGO 185 (EpT185) | 2.1 kb |
| TANGO 186 (EpT186) | 3.7 kb |
| TANGO 187 (EpT187) | 2.6 kb |
| TANGO 188 (EpT188) | 2.0 kb |
| TANGO 189 (EpT189sv1) | 1.3 kb |

The identity of the strains can be inferred from the fragments liberated.

A clone encoding TANGO 215 and four other clones were deposited on Sep. 25, 1998 with the American Type Culture Collection under accession number ATCC 98899, from which the srrain comprising the TANGO 215 cDNA clone is obtainable. To distinguish the strains and isolate a strain harboring the TANGO 215 cDNA clone, one can first streak out an aliquot of the mixture to single colonies on nutrient medium (e.g., LB plates) supplemented with 100 μg/ml ampicillin, grow single colonies, and then extract the plasmid DNA using a standard minipreparation procedure. Next, one can digest a sample of the DNA minipreparation with a combination of the restriction enzymes Sal I and Not I and resolve the resultant products on a 0.8% agarose gel using standard DNA electrophoresis conditions.

The digest will liberate one vector fragment of 2.7 kb common to all strains, and one insert-specific fragment as follows:

TANGO 215 (EpT215) 2.8 kb

The identity of the strain harboring the TANGO 215 cDNA clone can be inferred from the fragments liberated.

Equivalents

The contents of all references, patents and published patent applications cited throughout this application are hereby incorporated by reference. Those skilled in the art will recognize, or be able to ascertain using no more than routine experimentation, many equivalents to the specific embodiments of the invention described herein. Such equivalents are intended to be encompassed by the following claims.

What is claimed is:

1. An isolated nucleic acid molecule selected from the group consisting of:
a) a nucleic acid molecule comprising a nucleotide sequence which is at least 55% identical to the nucleotide sequence of any of SEQ ID NOs:1-22, 34-43, and ______-______, the cDNA insert of a plasmid deposited with the ATCC as any of Accession Numbers 98899, 98900, and 98901, or a complement thereof;
b) a nucleic acid molecule comprising a fragment of at least 300 nucleotides of the nucleotide sequence of any of SEQ ID NOs:1-22, 34-43, and ______-______, the cDNA insert of a plasmid deposited with the ATCC as any of Accession Numbers 98899, 98900, and 98901, or a complement thereof;
c) a nucleic acid molecule which encodes a polypeptide comprising the amino acid sequence of any of SEQ ID Nos:23-33, 54-63, and ______-______ or an amino acid sequence encoded by the cDNA insert of a plasmid deposited with the ATCC as any of Accession Numbers 98899, 98900, and 98901;
d) a nucleic acid molecule which encodes a fragment of a polypeptide comprising the amino acid sequence of any of SEQ ID NOs:23-33, 54-63, and ______-______ wherein the fragment comprises at least 15 contiguous amino acids of any of SEQ ID NOs:23-33, 54-63, and ______-______ or the polypeptide encoded by the cDNA insert of a plasmid deposited with the ATCC as any of Accession Numbers 98899, 98900, and 98901; and
e) a nucleic acid molecule which encodes a naturally occurring allelic variant of a polypeptide comprising the amino acid sequence of any of SEQ ID NOs:23-33, 54-63, and ______-______ or an amino acid sequence encoded by the cDNA insert of a plasmid deposited with ATCC as any of Accession Numbers 98899, 98900, and 98901, wherein the nucleic acid molecule hybridizes to a nucleic acid molecule comprising any of SEQ ID Nos:1-22, 34-43, and ______-______ or a complement thereof under stringent conditions.

2. The isolated nucleic acid molecule of claim 1, which is selected from the group consisting of:
a) a nucleic acid molecule comprising the nucleotide sequence of any of SEQ ID NO:1-22 and 34-43, the cDNA insert of a plasmid deposited with the ATCC as any of Accession Numbers 98899, 98900, and 98901, or a complement thereof; and
b) a nucleic acid molecule which encodes a polypeptide comprising the amino acid sequence of any of SEQ ID Nos:23-33, 54-63, and ______-______ or an amino acid sequence encoded by the cDNA insert of a plasmid deposited with the ATCC as any of Accession Numbers 98899, 98900, and 98901.

3. The nucleic acid molecule of claim 1 further comprising vector nucleic acid sequences.

4. The nucleic acid molecule of claim 1 further comprising nucleic acid sequences encoding a heterologous polypeptide.

5. A host cell which contains the nucleic acid molecule of claim 1.

6. The host cell of claim 5 which is a mammalian host cell.

7. A non-human mammalian host cell containing the nucleic acid molecule of claim 1.

8. An isolated polypeptide selected from the group consisting of:
a) a fragment of a polypeptide comprising the amino acid sequence of any of SEQ ID Nos:23-33, 54-63, and ______-______, wherein the fragment comprises at least 15 contiguous amino acids of any of SEQ ID Nos:23-33 and 54-63, and ______-______;
b) a naturally occurring allelic variant of a polypeptide comprising the amino acid sequence of any of SEQ ID Nos:23-33, 54-63, and ______-______ or an amino acid sequence encoded by the cDNA insert of a plasmid deposited with the ATCC as any of Accession Numbers 98899, 98900, and 98901, wherein the polypeptide is encoded by a nucleic acid molecule which hybridizes to a nucleic acid molecule comprising any of SEQ ID Nos:1-22, 34-43, and ______-______ or a complement thereof under stringent conditions; and
c) a polypeptide which is encoded by a nucleic acid molecule comprising a nucleotide sequence which is at least 55% identical to a nucleic acid comprising the nucleotide sequence of any of SEQ ID Nos:1-22, 34-43, and ______-______ or a complement thereof.

9. The isolated polypeptide of claim 8 comprising the amino acid sequence of any of SEQ ID Nos:23-33, 54-63, and ______-______ or an amino acid sequence encoded by the cDNA insert of a plasmid deposited with the ATCC as any of Accession Numbers 98899, 98900, and 98901.

10. The polypeptide of claim 8 further comprising heterologous amino acid sequences.

11. An antibody which selectively binds to a polypeptide of claim 8.

12. A method for producing a polypeptide selected from the group consisting of:
a) a polypeptide comprising the amino acid sequence of any of SEQ ID Nos:23-33, 54-63, and ______-______ or an amino acid sequence encoded by the cDNA insert of a plasmid deposited with the ATCC as any of Accession Numbers 98899, 98900, and 98901;
b) a polypeptide comprising a fragment of the amino acid sequence of any of SEQ ID Nos:23-33, 54-63, and ______-______ or an amino acid sequence encoded by the cDNA insert of a plasmid deposited with the ATCC as any of Accession Numbers 98899, 98900, and 98901, wherein the fragment comprises at least 15 contiguous amino acids of any of SEQ ID Nos:23-33, 54-63, and ______-______ or an amino acid sequence encoded by the cDNA insert of a plasmid deposited with the ATCC as any of Accession Numbers 98899, 98900, and 98901; and
c) a naturally occurring allelic variant of a polypeptide comprising the amino acid sequence of any of SEQ ID Nos:23-33, 54-63, and ______-______ or an amino acid sequence encoded by the cDNA insert of a plasmid deposited with the ATCC as any of Accession Numbers 98899, 98900, and 98901, wherein the polypeptide is encoded by a nucleic acid molecule which hybridizes to a nucleic acid molecule comprising the nucleotide sequence of any of SEQ ID Nos:1-22, 54-63, and ______-______ or a complement thereof under stringent conditions;
comprising culturing the host cell of claim 5 under conditions in which the nucleic acid molecule is expressed.

13. A method for detecting the presence of a polypeptide of claim 8 in a sample, comprising:
a) contacting the sample with a compound which selectively binds to a polypeptide of claim 8; and
b) determining whether the compound binds to the polypeptide in the sample.

14. The method of claim 13, wherein the compound which binds to the polypeptide is an antibody.

15. A kit comprising a compound which selectively binds to a polypeptide of claim 8 and instructions for use.

16. A method for detecting the presence of a nucleic acid molecule of claim 1 in a sample, comprising the steps of:
a) contacting the sample with a nucleic acid probe or primer which selectively hybridizes to the nucleic acid molecule; and
b) determining whether the nucleic acid probe or primer binds to a nucleic acid molecule in the sample.

17. The method of claim 16, wherein the sample comprises mRNA molecules and is contacted with a nucleic acid probe.

18. A kit comprising a compound which selectively hybridizes to a nucleic acid molecule of claim 1 and instructions for use.

19. A method for identifying a compound which binds to a polypeptide of claim 8 comprising the steps of:
a) contacting a polypeptide, or a cell expressing a polypeptide of claim 8 with a test compound; and
b) determining whether the polypeptide binds to the test compound.

20. The method of claim 19, wherein the binding of the test compound to the polypeptide is detected by a method selected from the group consisting of:
a) detection of binding by direct detecting of the binding of the test compound to the polypeptide binding; and
b) detection of binding using a competition binding assay.

21. A method for modulating the activity of a polypeptide of claim 8 comprising contacting a polypeptide or a cell expressing a polypeptide of claim 8 with a compound which binds to the polypeptide in a sufficient concentration to modulate the activity of the polypeptide.

22. A method for identifying a compound which modulates the activity of a polypeptide of claim 8, comprising:
a) contacting a polypeptide of claim 8 with a test compound; and
b) determining the effect of the test compound on the activity of the polypeptide to thereby identify a compound which modulates the activity of the polypeptide.


SEQUENCE LISTING (138 sequences)

SEQ ID NO: 1 - DNA, 1565 bp, Homo sapiens
  CDS (96)...(644)
gtcgacccacgcgtccggcggggacaactgggtcttttgcggctgcagcgggcttgtaggtgtccggctttgctggcccagcaagcctgataagcatgaagctcttatctttggtggctgtggtcgggtgtttgctggtgcccccagctgaagccaacaagagttctgaagatatccggtgcaaatgcatctgtccaccttatagaaacatcagtgggcacatttacaaccagaatgtatcccagaaggactgcaactgcctgcacgtggtggagcccatgccagtgcctggccatgacgtggaggcctactgcctgctgtgcgagtgcaggtacgaggagcgcagcaccaccaccatcaaggtcatcattgtcatctacctgtccgtggtgggtgccctgttgctctacatggccttcctgatgctggtggaccctctgatccgaaagccggatgcatacactgagcaactgcacaatgaggaggagaatgaggatgctcgctctatggcagcagctgctgcatccctcgggggaccccgagcaaacacagtcctggagcgtgtggaaggtgcccagcagcggtggaagctgcaggtgcaggagcagcggaagacagtcttcgatcggcacaagatgctcagctagatgggctggtgtggttgggtcaaggccccaacaccatggctgccagcttccaggctggacaaagcagggggctacttctcccttccctcggttccagtcttccctttaaaagcctgtggcatttttcctccttctccctaactttagaaatgttgtacttggctattttgattagggaagagggatgtggtctctgatctccgttgtcttcttgggtctttggggttgaagggagggggaaggcaggccagaagggaatggagacattcgaggcggcctcaggagtggatgcgatctgtctctcctggctccactcttgccgccttccagctctgagtcttgggaatgttgttacccttggaagataaagctgggtcttcaggaactcagtgtctgggaggaaagcatggcccagcattcagcatgtgttcctttctgcagtggttctttatcaccacctccctcccagccccagcgcctcagccccagccccagctccagccctgaggacagctctgatgggagagctgggccccctgagcccactgggtcttcagggtgcactggaagctggtgttcgctgtcccctgtgcacttctcgcactggggcatggagtgcccatgcatactctgctgccggtcccctcacctgcacttgaggggtctgggcagtccctcctctccccagtgtccacagtcactgagccagacggtcggttggaacatgagactcgaggctgagcgtggatctgaacaccacagcccctgtacttgggttgcctcttgtccctgaacttcgttgtaccagtgcatggagagaaaattttgtcctcttgtcttagagttgtgtgtaaatcaaggaagccatcattaaattgttttatttctcaaaaaaaaaaaaaaaaaaaagggcggccg

SEQ ID NO: 2 - DNA, 4605 bp, Homo sapiens
  CDS (125)...(1141)
  misc_feature (1)...(4605): n = A,T,C or G
tanggatcgaccacgcgtycgcccacgcgtccggtcgcgtgctgaggggtgtgacggttttcttgctcgtgggctcggacgagtacggagcgcctgcagggacagcctggataaaggctcactgatggctcagttgggagcagttgtggctgtggcttccagtttcttttgtgcatctctcttctcagctgtgcacaagatagaagagggacatattggggtatattacagaggcggtgccctgctgacttcgaccagcggccctggtttccatctcatgctccctttcatcacatcatataagtctgtgcagaccacactccagacagatgaggtgaagaatgtaccttgtgggactagtggtggtgtgatgatctactttgacagaattgaagtggtgaacttcctggtcccgaacgcagtgtatgatatagtgaagaactatactgctgactatgacaaggccctcatcttcaacaagatccaccacgaactgaaccagttctgcagtgtgcacacgcttcaagaggtctacattgagctgtttgatcagattgatgaaaatctcaaactggctttgcaacaggacctgacctccatggcccctgggctggtcattcaagctgtgcgggtaacaaagcccaacataccagaggcaatccgcagaaactacgagttgatggaaagtgagaagacaaagcttctcattgccgcccagaaacagaaggtggtggaaaaggaagcagagacagagcggaagaaggcgctcattgaggcagaaaaagtggcccaggtggctgagatcacctacgggcagaaggtgatggagaaggagactgagaagaagatttcagaaattgaagatgctgcatttctggcccgggagaaggcaaaggcagatgctgagtgctacactgctatgaaaatagccgaagccaataagctgaagctaacccctgaatatctgcagctgatgaagtacaaggccattgcttccaacagcaagatttactttggcaaagacattcctaacatgttcatggactctgcgggcagtgtgagcaagcagtttgaggggctagctgacaagctaagctttggcttagaagatgaacccttggagacggccactaaggagaattgaaaaaaacttgatatgactgcaaatgatacttaagcagatctttattttttaagatgaatcagaatgttcctccctccccgactaccttctctgactgtcttccagttactgtggtgaaaaagaagaaatgaacttaaatccactccctttctagggaaaggagggtggggactgatgatggggggttttatttcaggtaagcagtttatatgacttccaataagatttgtaaatcatgggcttgacctttgacctctagacactaattttatcctttgaggctggcttaattagggatgctgtcattaaggagagggagaaatgtagagtgttacctccaactcatttgatttcccttacttgggaaaatgcagtccagtgttctcacctctgcctccaaggtaggagatgtctgtgggtgaggctywkcaactgagcaaatatgtgcctgtgagtttgccagtagagctgtgaagaaacagctgcagagaacatttgaccttcctggcattcttgtctgcatgtgtgtgagttattttagaggtgtgctttcttgagccctcataaggaagtactggtgctaggttttgcaagatttkgtatacactttgctccttgccctagggctcagagtggtggtttctgactacatttctagagtcagagcttgatcaccacaactcaattatttcggcatcttttcacctatgctgngatttgttttttttttttcttctcaaaaattctgttcattggttccactcagcatcaagaagacagggacaaacaactcaagtgtcttaacagctgctggagtgggatccttgttatctcttagccactgcaggacctgcctgacaggttatgtgtgcacctcgagatgaagtgtctttctattattgtagagattctgtagtgaagaggtctgacaccatgtgtggaggaggaggaacgatcagtcaagagatgtcctggtcttaatgcctgtggcttgtgctgggagtgggtctgacttaagtgataaaaggrctctattcactaagtagcctgtgtttttaaatccagggctgcaggcagcaacgcaagtcaggctgaacattcagtctccagagacagctgtgtggagcaaatcaragttcatgcccaagtccccargttggaatggctgtgccaaaatccattcaargggttttctttttcattactaggtcagaacattttgagtcaccttgggagattcaggatggggagagcaaatttgaacaaaaggtttttcttatatcctgagattgaggggtagggggtgtccaacctgtatagcccatgggttgtgtctagaattaagtggagggcagctatctggagttaacttgcaagcatattggtgccctccatgaccacctctggcttaggactnnnnnnnnnnnnnnnnnnnnnnnnnnnnnnnnnnnnnnnnccccccccccgccaactcctatacccatcttccctaggtgaatctgtgaatggtcctttctggcagcaatccctgccttctttttgggcccatgcccagacttctggtttaaggaatggtcccagagcttgggccagcttgctcagaagttttgggagcattgagcctgcctagaaagatacagtgttagctccccttacttcaaagttgcccttctctgttcwgactcctgggacttctggtcctgggcacactttttgcaggcaacaaaatgtgcctgggagtgatggattttaatgtgctccagagtcctttcagaaggtggtcatttcccttggccgggcgcggtggctcacacctgtaatcccagcactttgggaggccaaggcaggcggatcacctgaggttaggagttcgagaccaccctggccaacatgcgaaaccccatctctacgaaaaatagaaatattagccgggcatggtgtcaggcacctgtaatcccagctacttgggaggctgaggcaggagaattgcttgaactcgggaggcagaggttgcagtgagccaagatcatgccatcccactctagcttgggcaatagagcaaggctccgtctcaagaaaagaaggtcatttcccaagactagcatagggagtatccatttaaaatacattcatcttcctcccatttccgtgctattaatcacttgttagagcaacatgacaatgcccagcatcccacttcccgaaaatgtctactccttctactctgagctcttgttgcctagacctyagaaaacaccaattcaccacagtagaaccgggagcagggatagctcagcttctctgaatagcacactttgctcaggtcttaacttgagggcctctccggtactaacatcctgcgatagcttgtcccatgagcacagaagagcctcagtagagtcaagtcctgctgcagctgccccaccccaagtttctatcatttcctctttaaacaaaaaatatgttatcctacacattagtgtcaatccaatggttgtctcttatctgcwaaatagcaaaatcatgaaaatcagctgttttatttgcataggncaactaacctgtctgtgtaactttgtttttattttaactcytmctagaaaatctaatcttaaaacatttgaattctaaacatgtaaaatgtgacagcctgcaattttgtagacagtgaagtaatggctgctatttataaatggaacntcnnnnnnnnnnnnnnnnnnnntatcaaaataagtamctgtttataaaattcagtttttgtagggttttccaaggaaaaatcaccttggttgaatgtttctcactcattaaactttgcagaagtgattcatattcagtactgtttttaatcactttttaaaatataaggaccgaatgcaaggaaaccaaagtttattaataatttttatataactaaaataaaatagatgtggagggatctgtgatcatataaaaagggagggttactgaaagaattttagcaatatattgattcaggaaaaggagctgttttataaatgatcattcactgttcctatggttctatgtatctttcaaaccgatacctttactatttaaagagcgtaaatagtgaaagtaagatggtcatacttactgactttatctatttaagtttgatggagataaactatatcttggctagtggctactgtgtctgtgaatgtaaccagtacttctttaagctctattcagtagggttccagccactgcttttttgttgtttctagccactgttttttttttcttgtttccttataaaacaggtaataaccaanaaaaaaaaaaaaagrkykscattcctttttatttctgcctttgtgtagactgatgattaagtaggcaaactacttctactttcaaagagccccaagggcttaatgaccccatttgtaactgagaaatgattcttgttttagtaatcattctataaatgatactgc

SEQ ID NO: 3 - DNA, 2704 bp, Homo sapiens
  CDS (48)...(1091)
gtcgacccacgcgtccggcggctgggcttcttctcagaggaacgagaatgaatatgactcaagcccgggttctggtggctgcagtggtggggttggtggctgtcctgctctacgcctccatccacaagattgaggagggccatctggctgtgtactacaggggaggagctttactaactagccccagtggaccaggctatcatatcatgttgcctttcattactacgttcagatctgtgcagacaacactacaaactgatgaagttaaaaatgtgccttgtggaacaagtggtggggtcatgatctatattgaccgaatagaagtggttaatatgttggctccttatgcagtgtttgatatcgtgaggaactatactgcagattatgacaagaccttaatcttcaataaaatccaccatgagctgaaccagttctgcagtgcccacacacttcaggaagtttacattgaattgtttgatcaaatagatgaaaacctgaagcaagctctgcagaaagacttaaacctcatggccccaggtctcactatacaggctgtgcgtgttacaaaacccaaaatcccagaagccataagaagaaattttgagttaatggaggctgagaagacaaaactccttatagctgcacagaaacaaaaggttgtggaaaaagaagctgagacagagaggaaaaaggcagttatagaagcagagaagattgcacaagtggcaaaaattcggtttcagcagaaagtgatggaaaaagaaactgaaaagcgcatttctgaaatcgaagatgctgcattcctggcccgagagaaagcgaaagcagatgctgaatattatgctgcacacaaatatgccacctcaaacaagcacaagttgaccccggaatatctggagctcaaaaagtaccaggccattgcttctaacagtaagatctattttggcagcaacatccctaacatgttcgtggactcctcatgtgctttgaaatattcagatattaggactggaagagaaagctcactcccctctaaggaggctcttgaaccctctggagagaacgtcatccaaaacaaagagagcacaggttgatgcaagaggtggaaatgttctccatatcaagatgtggcccaaggggttaagtgggaacaatcattatacggactcttcagatttacagagaacttacacttcatctgttccacctctcctgcgatagtcctgggtgctccactgattggaggatagagccagctgtctgacacacaaatggtcttttcagccacagtcttatcaagtatcctatatgtattcctttctaaactgctactcatgaatgaggaaagtctgatgctaagatactgcctgcattccctgcattgggttgatgactgtcagcatcactgccgcaggccatgcttgactaaggtacctggttttagccacagccacctccttgtatgttacctttcagctctggccaagagtgggacagggttttaaccacaaataggagcagcatgcaattcctagtgacttgctgcacagtattgtatcataattacaggaagtttttatttttaaaactggatctggggtatattcatttgccccatcacctctgtctaaaggcccaagtcctagggctgccatggtcacaagcacactgatgctccttaagattgtttatctggagcccacatagtgtggaacaaaaagtcacctagaaagcatccttggtcatcattgtctccttcccacctggcccagagatgcttaaatccaagttgtttctccagctgtcacctcccccaggagatcaggattccactgacgtcctgggcagccagtgaatttaattttccatgagaaacaacagagttaacctgtggcattaggagacctacttcatgtggaccctttttttccttcagtttaacttttctggagcagtgtgctgcgtagttcggcctgagtttgtgcagcttgttaagacaactcttgtgtacactatgttgaagctcaacaaaaaagtcatgggaccacttctagaaatctttcagctgtcaggcctgtcagtctcatgacagtttgttggttgtgccaaacactttatttgggaaaggaaagcccagatttgaatgggtctttcccctgggccttatcctatagaggcatttgtaatatggagaaaataatttttcatttttgctcatttaattctataaattctctttataaatgaattttgtgttctttagttctccttaaaagaacttttgaattataaaaataaaatctttacctgtcgaattgttgctgcagatgattgttgtggaaaatctggatcattgacctctgtgctttcattcctagagatgttttatagttacatgagcaaaagctgttgccccaaagtgatggccctggaggcggggctgaggaacagggaaatgccgctgtgaagtcttaaagcacttctgcttaaactccatgtgtgaggagtgtgcctccctgtgccctctcagctctgaggctggccgtctttcggggtgttccttttggcaaatatacactgtaatcttgagtctaaatttatatgttgaaatgctaccttttttaaaataagaaactaaataaaattattttactatcaaaaaaaaaaaaaaaaaaaaaaaaaaaaaaaaaaaaaaaaaaaaaaa

SEQ ID NO: 4 - DNA, 1565 bp, Homo sapiens; identical to SEQ ID NO: 1
  CDS (96)...(644)

SEQ ID NO: 5 - DNA, 1576 bp, Homo sapiens
  CDS (146)...(739)
gcacgagtccagacggaagtgcgggcggaggatccccagccgggtcccaagcctgtgcctgagcctgagcctgagcctgagcctgagcccgagccgggagccggtcgcgggggctccgggctgtgggaccgctgggcccccagcgatggcgaccctgtggggaggccttcttcggcttggctccttgctcagcctgtcgtgcctggcgctttccgtgctgctgctggcgcagctgtcagacgccgccaagaatttcgaggatgtcagatgtaaatgtatctgccctccctataaagaaaattctgggcatatttataataagaacatatctcagaaagattgtgattgccttcatgttgtggagcccatgcctgtgcgggggcctgatgtagaagcatactgtctacgctgtgaatgcaaatatgaagaaagaagctctgtcacaatcaaggttaccattataatttatctctccattttgggccttctacttctgtacatggtatatcttactctggttgagcccatactgaagaggcgcctctttggacatgcacagttgatacagagtgatgatgatattggggatcaccagccttttgcaaatgcacacgatgtgctagcccgctcccgcagtcgagccaacgtgctgaacaaggtagaatatgcacagcagcgctggaagcttcaagtccaagagcagcgaaagtctgtctttgaccggcatgttgtcctcagctaattgggaattgaattcaaggtgactagaaagaaacaggcagacaactggaaagaactgactgggttttgctgggtttcattttaataccttgttgatttcaccaactgttgctggaagattcaaaactggaagcaaaaacttgcttgatttttttttcttgttaacgtaataatagagacatttttaaaagcacacagctcaaagtcagccaataagtcttttcctatttgtgacttttactaataaaaataaatctgcctgtaaattatctdgaagtcctttacctggaacaagcactctctttttcaccaccatcctcttttcctcatggaaatgtctgctttatgaaactatgcacatattgaaagtgagttgaaacaaatgagggttgggtaggagcttccaggcctgggatttacaccacgcctagcccagcagaggccttagtcccatttggggctttgggagtgacatttgcttgaggcttatacactggtgtggttgcctggcttgcaggaaatgaccaagctcacacatgctggctgaagcgtaagmrkacaactgaggtactcttttgaaggatgaaggtggtggattctcagccctgggggtcttcctcacctgaggacccttcagagccaccctttctagtttgcatttcctggtgcacacatttaaggcataacagcacattcatccctttggtttgggatctcaggaatacagtcccatgcaaagattctctggttttatggcttttttccctttctttacaccatcctctcccataagcacccatgtctttgaatatgaatgtatttgtaaaataaaaaa

SEQ ID NO: 6 - DNA, 2067 bp, Homo sapiens
  CDS (201)...(779)
gtcgacccacgcgtccgctctgagtcaccggaatctaggtggggccgcccggagcggcgtcctcgggagccgcctccccgcggcctcttcgcttttgtggcggcgcccgcgctcgcaggccactctctgctgtcgcccgtcccgcgcgctcctccgacccgctccgctccgctccgctcggccccgcgccgcccgtcaacatgatccgctgcggcctggcctgcgagcgctgccgctggatcctgcccctgctcctactcagcgccatcgccttcgacatcatcgcgctggccggccgcggctggttgcagtctagcgaccacggccagacgtcctcgctgtggtggaaatgctcccaagagggcggcggcagcgggtcctacgaggagggctgtcagagcctcatggagtacgcgtggggtagagcagcggctgccatgctcttctgtggcttcatcatcctggtgatctgtttcatcctctccttcttcgccctctgtggaccccagatgcttgtcttcctgagagtgattggaggtctccttgccttggctgctgtgttccagatcatctccctggtaatttaccccgtgaagtacacccagaccttcacccttcatgccaaccctgctgtcacttacatctataactgggcctacggctttgggtgggcagccacgattatcctgattggctgtgccttcttcttctgctgcctccccaactacgaagatgaccttctgggcaatgccaagcccaggtacttctacacatctgcctaacttgggaatgaatgtgggagaaaatcgctgctgctgagatggactccagaagaagaaactgtttctccaggcgactttgaacccattttttggcagtgttcatattattaaactagtcaaaaatgctaaaataatttgggagaaaatattttttaagtagtgttatagtttcatgtttatcttttattatgttttgtgaagttgtgtcttttcactaattacctatactatgccaatatttccttatatctatccataacatttatactacatttgtaagagaatatgcacgtgaaacttaacactttataaggtaaaaatgaggtttccaagatttaataatctgatcaagttcttgttatttccaaatagaatggactcggtctgttaagggctaaggagaagaggaagataaggttaaaagttgttaatgaccaaacattctaaaagaaatgcaaaaaaaaagtttattttcaagccttcgaactatttaaggaaagcaaaatcatttcctaaatgcatatcatttgtgagaatttctcattaatatcctgaatcattcattttagctaaggcttcatgttgactcgatatgtcatctaggaaagtactatttcatggttcaaacctgttgccatagttggtaaggctttcctttaagtgtgaaatatttagatgaaattttctcttttaaagttctttatagggttagggtgtgggaaaatgctatattaataaatctgtagtgttttgtgtttatatgttcagaaccagagtagactggattgaaagatggactgggtctaatttatcatgactgatagatctggttaagttgtgtagtaaagcattaggagggtcattcttgtcacaaaagtgccactaaaacagcctcaggagaataaatgacttgcttttctaaatctcaggtttatctgggctctatcatatagacaggcttctgatagtttgcaactgtaagcagaaacctacatatagttaaaatcctggtctttcttggtaaacagattttaaatgtctgatataaaacatgccacaggagaattcggggatttgagtttctctgaatagcatatatatgatgcatcggataggtcattatgattttttaccatttcgacttacataatgaaaaccaattcattttaaatatcacgattattattttgtaagttgtggaaaaagctaattgtagttttcattatgaagttttcccaataaaccagggcattctaaaaaaaaaaaaaaaaaaaagggcggccgc

SEQ ID NO: 7 - DNA, 3714 bp, Homo sapiens
  CDS (109)...(1257)
gtcgacccacgcgtccggccgcgcgctctctcccggcgcccacacctgtctgagcggcgcagcgagccgcggcccgggcgggctgctcggcgcggaacagtgctcggcatggcagggattccagggctcctcttccttctcttctttctgctctgtgctgttgggcaagtgagcccttacagtgccccctggaaacccacttggcctgcataccgcctccctgtcgtcttgccccagtctaccctcaatttagccaagccagactttggagccgaagccaaattagaagtatcttcttcatgtggaccccagtgtcataagggaactccactgcccacttacgaagaggccaagcaatatctgtcttatgaaacgctctatgccaatggcagccgcacagagacgcaggtgggcatctacatcctcagcagtagtggagatggggcccaacaccgagactcagggtcttcaggaaagtctcgaaggaagcggcagatttatggctatgacagcaggttcagcatttttgggaaggacttcctgctcaactaccctttctcaacatcagtgaagttatccacgggctgcaccggcaccctggtggcagagaagcatgtcctcacagctgcccactgcatacacgatggaaaaacctatgtgaaaggaacccagaagcttcgagtgggcttcctaaagcccaagtttaaagatggtggtcgaggggccaacgactccacttcagccatgcccgagcagatgaaatttcagtggatccgggtgaaacgcacccatgtgcccaagggttggatcaagggcaatgccaatgacatcggcatggattatgattatgccctcctggaactcaaaaagccccacaagagaaaatttatgaagattggggtgagccctcctgctaagcagctgccagggggcagaattcacttctctggttatgacaatgaccgaccaggcaatttggtgtatcgcttctgtgacgtcaaagacgagacctatgacttgctctaccagcaatgcgatgcccagccaggggccagcgggtctggggtctatgtgaggatgtggaagagacagcagcagaagtgggagcgaaaaattattggcattttttcagggcaccagtgggtggacatgaatggttccccacaggatttcaacgtggctgtcagaatcactcctctcaaatatgcccagatttgctattggattaaaggaaactacctggattgtagggaggggtgacacagtgttccctcctggcagcaattaagggtcttcatgttcttattttaggagaggccaaattgttttttgtcattggcgtgcacacgtgtgtgtgtgtgtgtgtgtgtaaggtgtcttataatcttttacctatttcttacaattgcaagatgactggctttactatttgaaaactggtttgtgtatcatatcatatatcatttaagcagtttgaaggcatacttttgcatagaaataaaaaaaatactgatttggggcaatgaggaatatttgacaattaagttaatcttcacgtttttgcaaactttgatttttatttcatctgaacttgtttcaaagatttatattaaatatttggcatacaagagatatgaattcttatatgtgtgcatgtgtgttttcttctgagattcatcttggtggtgggtttttttgtttttttaattcagtgcctgatctttaatgcttccataaggcagtgttcccatttaggaactttgacagcatttgttaggcagaatattttggatttggaggcatttgcatggtagtctttgaacagtaaaatgatgtgttgactatactgatacacatattaaactataccttatagtaaaccagtatcccaagctgcttttagttccaaaaatagtttcttttccaaaggttgttgctctactttgtaggaagtctttgcatatggccctcccaactttaaagtcataccagagtggccaagagtgtttatcccaacccttccatttaacaggatttcactcacatttctggaactagctatttttcagaagacaataatcagggcttaattagaacaggctgtatttcctcccagcaaacagttgtggccacactaaaaacaatcatagcattttacccctggattatagcacatctcatgttttatcatttggatggagtaatttaaaatgaattaaattccagagaacaatggaagcattgcctggcagatgtcacaacagaataaccacttgtttggagcctggcacagtcctccagcctgatcaaaaattattctgcatagttttcagtgtgctttctgggagctatgtacttcttcaatttggaaacttttctctctcatttatagtgaaaatacttggaagttactttaagaaaaccagtgtggcctttttccctctagctttaaaagggccgcttttgctggaatgctctaggttatagataaacaattaggtataatagcaaaaatgaaaattggaagaatgcaaaatggatcagaatcatgccttccaataaaggcctttacacatgttttatcaatatgattatcaaatcacagcatatacagaaaagacttggacttattgtatgtttttattttatggctctcggcctaagcacttctttctaaatgtatcggagaaaaaatcaaatggactacaagcacgtgtttgctgtgcttgcaccccaggtaaacctgcattgtagcaatttgtaaggatattcagatggagcactgtcacttagacattctctgggggattttctgcttgtctttcttgagctttttggaaggataattctgataaggcactcaagaaacgtacaaccacagtgctttcttcaaatcatatgagaaatactatgcatagcaaggagatgcagagccgccaggaaaattctgagttccagcacaattttctttggaatctaacaggaatctagcctgaggaagaagggaggtctccatttctatgtctggtatttgggggttttgtttgtttttgctttagcttggtgaaaaaaagttcactgaacaccaagaccagaatggatttttttaaaaaaatagatgttccttttgtgaagcaccttgattccttgattttgattttttgcaaagttagacaatggcacaaagtcaaaatgaaatcaatgtttagttcacaagtagatgtaatttactaaagaatgatacacccatatgctatatacagcttaactcacagaactgtaaaagaaaattataaaataattcaacatgtccatctttttagtgataataaaagaaagcatggtattaaactatcatagaagtagacagaaaaagaaaaaaggactcatggcattattaatataattagtgctttacatgtgttagttatacatattagaagcatatttgcctagtaaggctagtagaaccacatttcccaaagtgtgctccttaaacactcatgccttatgattttctaccaaaagtaaaaagggttgtattaagtcagaggaagatgcctctccattttccctctctttatcagaggttcacatgcctgtctgcacattaaaagctctgggaagacctgttgtaaagggacaagttgaggttgtaaaatctgcatttaaataaacatctttgatcacaaaaaaaaaaaaaaagggcggccg

SEQ ID NO: 8 - DNA, 1970 bp, Homo sapiens
  CDS (23)...(814)
gtcgacccacgcgtccgggctcatggcgccggcgtcgcggttgctcgcgctctgggcgctggcggctgtggctctacccggctccggggcggagggcgacggcgggtggcgcccgggcgggccgggggccgtggcggaggaggagcgctgcacggtggagcgtcgggccgacctcacctacgcggagttcgtgcagcagtacgccttcgtcaggcccgtcatcctgcagggactcacggacaactcgaggttccgggccctgtgctcccgcgacaggttgctggcttcgtttggggacagagtggtccggctgagcaccgccaacacctactcctaccacaaagtggacttgcccttccaggagtatgtggagcagctgctgcacccccaggaccccacctccctgggcaatgacaccctgtacttcttcggggacaacaacttcaccgagtgggcctctctctttcggcactactccccacccccatttggcctgctgggaaccgctccagcttacagctttggaatcgcaggagctggctcgggggtgcccttccactggcatggacccgggtactcagaagtgatctacggtcgtaagcgctggttcctttacccacctgagaagacgccagagttccaccccaacaagaccacgctggcctggctccgggacacatacccagccctgccaccgtctgcacggcccctggagtgtaccatccgggctggtgaggtgctgtacttccccgaccgctggtggcatgctacgctcaaccttgacaccagcgtcttcatctccaccttcctcggctagccaaaacagctggcaggactgccggtcacacaccagcacgtcccacctcgtgctcacggattttattacacagatagtggcggcaatggcctcagcccagcccaccctcacctgcttttccagcccacaaagggggacgatcacggcccagcaaaagcgatgctgagaggggaaacagtccagagtccaacagcagaacttgggggaagcggtcggggtggccaggaacataaactatgtataggggccgggggcttctgcccagggctcccctggaccaggacgccaggtagggcagggaacctcagtagtcctccacccagccattctcagagatgaatgcgtcaataacctccttcatagccaagttggggatgagctgttcctgggtcagggggctccgggtcacggggtcaaaatgacccacacgctgcagtgacaagaagggcagagggcagtcatggggcccaggaccatgccactggccctgctcccccagccgcaggcctcacctgcaggtgctcctcgatgtccttgcggtcgtaggtgatgccactgggcgtgatgcacggctcccgcatcagctcaaagctgatcttgccacacaggtagtcggggatgtctcgcttctgtggcacaggggcacacggtcagaggctgaaaaggggcactgcacgagcacctgccagccatcggcagcaagcgacacacactcaccttcctcttctcatccacctgagaaaaaagctcgtccatgtccgccatgtacttgtcctgtgaagagttgagtgctgtgcttgggggagacaccccacctccctcctccatggggcacagacccaacacaaggcggggatgctcccacgccacgtgcacacacacagacccacatgtgggtggggggcaccctcacgtgcttggcctcaatgcaggcctgctgggcccggacgtggctgtcgtcctcatcaccctcgtggtttcgctggcactcttccagctccctgggggttgaccaggagccggtcagagatggacctggccagatgtctgaccacaccccaatctcagagctaacatccacacttccccacatttcctgcttgccagtaaagccttcgataaacaaaaaaaaaaaaaaaaaaaagggcggccg

SEQ ID NO: 9 - DNA, 2062 bp, Homo sapiens
  CDS (110)...(868)
  misc_feature (1)...(2062): n = A,T,C or G
gtggcctgagctagargstgrmgswgsmgggggvaggaggdtggbvkggtctgagctagagggtgaagctggcggancaggaggatgggcgagcagtctgaatgccagaatggataaccgttttgctacagcatttgtaattgcttgtgtgcttagcctcatttccaccatctacatggcagcctccattggcacagacttctggtatgaatatcgaagtccagttcaagaaaattccagtgatttgaataaaagcatctgggatgaattcattagtgatgaggcagatgaaaagacttataatgatgcactttttcgatacaatggcacagtgggattgtggagacggtgtatcaccatacccaaaaacatgcattggtatagcccaccagaaaggacagagtcatttgatgtggtcacaaaatgtgtgagtttcacactaactgagcagttcatggagaaatttgttgatcccggaaaccacaatagcgggattgatctccttaggacctatctttggcgttgccagttccttttaccttttgtgagtttaggtttgatgtgctttggggctttgatcggactttgtgcttgcatttgccgaagcttatatcccaccattgccacgggcattctccatctccttgcaggtctgtgtacactgggctcagtaagttgttatgttgctggaattgaactactccaccagaaactagagctccctgacaatgtatccggtgaatttggatggtccttctgcctggcttgtgtctctgctcccttacagttcatggcttctgctctcttcatctgggctgctcacaccaaccggaaagagtacaccttaatgaaggcatatcgtgtggcatgagcaagaaactgcctgctttacaattgccatttttatttttttaaaataatactgatattttccccacctctcaattgtttttaatttttatttgtggatataccattttattatgaaaatcnatntnatttatacacattcaccactaaatacacacttaataccactaaaatttatgtggtttactttaagcgatgccatctttcaaataaactaatctaggtctagacagaaagaaatggatagagacttgacacaaatttatgaaagaaaattgggagtaggaatgtgaccgaaaacaagttgtgctaatgtctgttagacttttcagtaaaactaaagtaactgtatctgttcaactaaaaactctatattagtttctttgggaaacctctcatcgtcaaaactttatgttcactttgctgttgtagatagccagtcaaccagcagtattagtgctgttttcaaagatttaagctctataaaattgggaaattatctaagatcattttccctaagcattgacacatagcttcatctgaggtgagatatggcagctgtttgtatctgcactgtgtctgtctacaaaaagtgaaaaatacagtgtttacttgaaattttaactttgtaactgcaagaattccagttcagccgggcgaggattagtattatttttaactctccgtaagattttcagtaccaccaaattgttttggattttttttctttcctcttcacataccagggttattaaaagtgtgctttctttttacattatattacagttacaaggtaaaattcctcaactgctatttatttattccagcccagtactataaagaacgtttcaccataatgaccctccagagctgggaaacctaccacaagatctaaagttctggctgtccattaacctccaactatggtctttatttcttgtggtaatatgatgtgcctttccttgcctaaatcccttcctggtgtgtatcaacattatttaatgtcttctaattcagtcatttttttataagtatgtctataaacattgaactttaaaaaacttatttatttattccactactgtagcaattgacagattaaaaaaatgtaacttcataatttcttaccataacctcaatgtcttttttaaaaaataaaattaaaaatgaaaagagaaanaaaaaaaaaaaa

SEQ ID NO: 10 - DNA, 2747 bp, Homo sapiens
  CDS (37)...(2196)
  misc_feature (1)...(2747): n = A,T,C or G
tccccagtagacgctccggcaccagccgcggcaaggatggagctgggttgctggacgcagttggggctcacttttcttcagctccttctcatctcgtccttgccaagagagtacacagtcattaatgaagcctgccctggagcagagtggaatatcatgtgtcgggagtgctgtgaatatgatcagattgagtgcgtctgccccggaaagagggaagtcgtgggttataccatcccttgctgcaggaatgaggagaatgagtgtgactcctgcctgatccacccaggttgtaccatctttgaaaactgcaagagctgccgaaatggctcatgggggggtaccttggatgacttctatgtgaaggggttctactgtgcagagtgccgagcaggctggtacggaggagactgcatgcgatgtggccaggttctgcgagccccaaagggtcagattttgttggaaagctatcccctaaatgctcactgtgaatggaccattcatgctaaacctgggtttgtcatccaactaagatttgtcatgttgagcctggagtttgactacatgtgccagtatgactatgttgaggttcgtgatggagacaaccgcgatggccagatcatcaagcgtgtctgtggcaacgagcggccagctcctatccagagcataggatcctcactccacgtcctcttccactccgatggctccaagaattttgacggtttccatgccatttatgaggagatcacagcatgctcctcatccccttgtttccatgacggcacgtgcgtccttgacaaggctggatcttacaagtgtgcctgcttggcaggctatactgggcagcgctgtgaaaatctccttgaagaaagaaactgctcagaccctgggggcccgatcaatgggtaccagaaaataacagggggccctgggcttatcaacggacgccatgctaaaattggcaccgttgtgtctttcttttgttacaactcctatgttcttagtggcaatgagaaaagaacttgccagcagaatggagagtggtcagggaaacagcccatctgcataaaagcctgccgagaaccaaagatttcagacctggtgagaaggagagttcttccgatgcaggttcagtcaagggagacaccattacaccagctatactcagcggccttcagcaagcagaaactgcagagtgcccctaccaagaagccagcccttccctttggagatctgcccatgggataccaacatctgcatacccagctccagtatgagtgcatctcacccttctaccgccgcctgggcagcagcaggaggacatgtctgaggactgggaagtggagtgggcgggcaccatcctgcatccctatctgcgggaaaattgagaacatcactgctccaaagacccaagggttgcgctggccgtggcaggcagccatctacaggaggaccagcggggtgcatgacggcagcctacacaagggagcgtggttcctagtctgcagcggtgccctggtgaatgagcgcactgtggtggtggctgcccactgtgttactgacctggggaaggtcaccatgatcaagacagcagacctgaaagttgttttggggaaattctaccgggatgatgaccgggatgagaagaccatccagagcctacagatttctgctatcattctgcatcccaactatgaccccatcctgcttgatgctgacatcgccatcctgaagctcctagacaaggcccgtatcagcacccgagtccagcccatctgcctcgctgccagtcgggatctcagcacttccttccaggagtcccacatcactgtggctggctggaatgtcctggcagacgtgaggagccctggcttcaagaacgacacactgcgctctggggtggtcagtgtggtggactcgctgctgtgtgaggagcagcatgaggaccatggcatcccagtgagtgtcactgataacatgttctgtgccagctgggaacccactgccccttctgatatctgcactgcagagacaggaggcatcgcggctgtgtccttcccgggacgagcatctcctgagccacgctggcatctgatgggactggtcagctggagctatgataaaacatgcagccacaggctctccactgccttcaccaaggtgctgccttttaaagactggattgaaagaaatatgaaatgaaccatgctcatgcactccttgagaagtgtttctgtatatccgtctgtacgtgtgtcattgcgtgaancagtgtgggcctgaagtgtgatttggcctgtgaacttggctgtgccagggcttctgacttcagggacaaaactcagtgaagggtgagtagacctccattgctggtaggctgatgccvcgtccactactaggacagccaattggaagatgccagggcttgcaagaagtaagtttcttcaaagaagaccatatacaaaacctctccactccactgacctggtggtcttccccaactttcagttatacgaatgccatcagcttgaccagggaagatctgggcttcatgaggccccttttgaggctctcaagttctagagagctgcctgtgggacagcccagggcagcagagctgggatgtggtgcatgcctttgtgtacatggccacagtacagtctggtccttttccttccccatctcttgtacacattttaataaaataagggttggcttctgaactacaaaaaaaaaaaaaaaaaaaaaaa

SEQ ID NO: 11 - DNA, 2403 bp, Homo sapiens
  CDS (110)...(1138)
gtcgacccacgcgtccggcggctaggcccgcgtgcgctggagacctccgcgctggcccccgcgagcctcctgccctggcccggcgctgcggctctgccgcggcggcagcatgggtggcccccggggcgcgggctgggtggcggcgggcctgctgctcggcgcgggcgcctgctactgcatttacaggctgacccggggtcggcggcggggcgaccgcgagctcgggatacgctcttcgaagtccgcaggtgccctggaagaagggacgtcagagggtcagttgtgcgggcgctcggcccggcctcagacgggaggtacctgggagtcacagtggtccaagacctcgcagcctgaagacttaactgatggttcatatgatgatgttctaaatgctgaacaacttcagaaactcctttacctgctggagtcaacggaggatcctgtaattattgaaagagctttgattactttgggtaacaatgcagccttttcagttaaccaagctattattcgtgaattgggtggtattccaattgttgcaaacaaaatcaaccattccaaccagagtattaaagagaaagctttaaatgcactaaataacctgagtgtgaatgttgaaaatcaaatcaagataaagatatacatcagtcaagtatgtgaggatgtcttctctggtcctctgaactctgctgtgcagctggctggactgacattgttgacaaacatgactgttaccaatgaccaccagcacatgcttcacagttacattacagacctgttccaggtgttacttactggaaatggaaacacgaaggtgcaagttttgaaactgcttttgaatttgtctgaaaatccagccatgacagaaggacttctccgtgcccaagtggattcatcattcctttccctttatgacagccacgtagcaaaggagattcttcttcgagtacttacgctatttcagaatataaagaactgcctcaaaatagaaggccatttagctgtgcagcctactttcactgaaggttcattgtttttcctgttacatggagaagaatgtgcccagaaaataagagctttagttgatcaccatgatgcagaggtgaaggaaaaggttgtaacaataatacccaaaatctgattggtcatatttttccaaagagtaatgcagtctggatataaatgtattttctgtcttccttataaggggattctcccagctgctaaatttaaacagtaaatatcacattttgtcattaacacagctataacttgccgtggttctcagatttattttggactattttgatgccaagtgaatataagagcttgtactgaaaccatttatttctttctattttgctatttgcaaatgcttgttatcttccctacatgaagtggcagtaacctttttcacatttaagctacccttctaccttttgaagtgatttgcagttactcatctgagacagcatcagtatttgactaaatcattgtttcacaactgaatagtcttgttcttttagtagcaatgaaatcctaagctcttgaggccattcacctgccaacctgaccatactgctttcaaaagtcttttctcatcagtagaatctattttggtcacttctagtcaatgaaaaatgtaaacttttaggagagaatgtttcctaggactcacccactccattcaatgttacatataaaatagtgtgatcaatcacaatgtccatctttagacagttggttaaataaattatctggtctttgaaaagaccgtgctgggcgcggtggctcttgcctgtaatcccagcactttgggaggctgaggcgggcagatcacctgagatcgggagtttgagaccaagcctgaccaatatggagaaaccctgtctctactaagaatacaaaattagctgggcatggtggtgcatgcctgtaatcccagctacttgggaggccgaggcaggagaattgcttgaacccgggaggcagaggttgcagtgaggtgagatagcgccattgcactccagcctgggcaacaagagcaaaactctgtctcaaaaaaaaaaaaaaatgatggagctccgaatgtgcttaagtggaaagatatctatgaaatatggtggttttttaaaacacaaaaattatagaatatgggatcccgtgtgtgtgtgtgtgtgtgtgtgtgtgtgtgtgtgtgtgtgtgtgtgtttgaatgaaaaatgcttatgtattgacagaacacttctagaatgatacccaaactcctggagtgggagtggggaatgccttctacgtacacactgttctactgtttgaattttttaatatgagcccaaattgtataatctttttttaataaaggggagaaaaatcaaaaaaaaaaaaaaaaagggcggccgc

SEQ ID NO: 12 - DNA, 549 bp, Homo sapiens
atgaagctcttatctttggtggctgtggtcgggtgtttgctggtgcccccagctgaagccaacaagagttctgaagatatccggtgcaaatgcatctgtccaccttatagaaacatcagtgggcacatttacaaccagaatgtatcccagaaggactgcaactgcctgcacgtggtggagcccatgccagtgcctggccatgacgtggaggcctactgcctgctgtgcgagtgcaggtacgaggagcgcagcaccaccaccatcaaggtcatcattgtcatctacctgtccgtggtgggtgccctgttgctctacatggccttcctgatgctggtggaccctctgatccgaaagccggatgcatacactgagcaactgcacaatgaggaggagaatgaggatgctcgctctatggcagcagctgctgcatccctcgggggaccccgagcaaacacagtcctggagcgtgtggaaggtgcccagcagcggtggaagctgcaggtgcaggagcagcggaagacagtcttcgatcggcacaagatgctcagc

SEQ ID NO: 13 - DNA, 1017 bp, Homo sapiens
atggctcagttgggagcagttgtggctgtggcttccagtttcttttgtgcatctctcttctcagctgtgcacaagatagaagagggacatattggggtatattacagaggcggtgccctgctgacttcgaccagcggccctggtttccatctcatgctccctttcatcacatcatataagtctgtgcagaccacactccagacagatgaggtgaagaatgtaccttgtgggactagtggtggtgtgatgatctactttgacagaattgaagtggtgaacttcctggtcccgaacgcagtgtatgatatagtgaagaactatactgctgactatgacaaggccctcatcttcaacaagatccaccacgaactgaaccagttctgcagtgtgcacacgcttcaagaggtctacattgagctgtttgatcagattgatgaaaatctcaaactggctttgcaacaggacctgacctccatggcccctgggctggtcattcaagctgtgcgggtaacaaagcccaacataccagaggcaatccgcagaaactacgagttgatggaaagtgagaagacaaagcttctcattgccgcccagaaacagaaggtggtggaaaaggaagcagagacagagcggaagaaggcgctcattgaggcagaaaaagtggcccaggtggctgagatcacctacgggcagaaggtgatggagaaggagactgagaagaagatttcagaaattgaagatgctgcatttctggcccgggagaaggcaaaggcagatgctgagtgctacactgctatgaaaatagccgaagccaataagctgaagctaacccctgaatatctgcagctgatgaagtacaaggccattgcttccaacagcaagatttactttggcaaagacattcctaacatgttcatggactctgcgggcagtgtgagcaagcagtttgaggggctagctgacaagctaagctttggcttagaagatgaacccttggagacggccactaaggagaat

SEQ ID NO: 14 - DNA, 1044 bp, Homo sapiens
atgaatatgactcaagcccgggttctggtggctgcagtggtggggttggtggctgtcctgctctacgcctccatccacaagattgaggagggccatctggctgtgtactacaggggaggagctttactaactagccccagtggaccaggctatcatatcatgttgcctttcattactacgttcagatctgtgcagacaacactacaaactgatgaagttaaaaatgtgccttgtggaacaagtggtggggtcatgatctatattgaccgaatagaagtggttaatatgttggctccttatgcagtgtttgatatcgtgaggaactatactgcagattatgacaagaccttaatcttcaataaaatccaccatgagctgaaccagttctgcagtgcccacacacttcaggaagtttacattgaattgtttgatcaaatagatgaaaacctgaagcaagctctgcagaaagacttaaacctcatggccccaggtctcactatacaggctgtgcgtgttacaaaacccaaaatcccagaagccataagaagaaattttgagttaatggaggctgagaagacaaaactccttatagctgcacagaaacaaaaggttgtggaaaaagaagctgagacagagaggaaaaaggcagttatagaagcagagaagattgcacaagtggcaaaaattcggtttcagcagaaagtgatggaaaaagaaactgaaaagcgcatttctgaaatcgaagatgctgcattcctggcccgagagaaagcgaaagcagatgctgaatattatgctgcacacaaatatgccacctcaaacaagcacaagttgaccccggaatatctggagctcaaaaagtaccaggccattgcttctaacagtaagatctattttggcagcaacatccctaacatgttcgtggactcctcatgtgctttgaaatattcagatattaggactggaagagaaagctcactcccctctaaggaggctcttgaaccctctggagagaacgtcatccaaaacaaagagagcacaggt

SEQ ID NO: 15 - DNA, 549 bp, Homo sapiens; identical to SEQ ID NO: 12

SEQ ID NO: 16 - DNA, 594 bp, Homo sapiens
atggcgaccctgtggggaggccttcttcggcttggctccttgctcagcctgtcgtgcctggcgctttccgtgctgctgctggcgcagctgtcagacgccgccaagaatttcgaggatgtcagatgtaaatgtatctgccctccctataaagaaaattctgggcatatttataataagaacatatctcagaaagattgtgattgccttcatgttgtggagcccatgcctgtgcgggggcctgatgtagaagcatactgtctacgctgtgaatgcaaatatgaagaaagaagctctgtcacaatcaaggttaccattataatttatctctccattttgggccttctacttctgtacatggtatatcttactctggttgagcccatactgaagaggcgcctctttggacatgcacagttgatacagagtgatgatgatattggggatcaccagccttttgcaaatgcacacgatgtgctagcccgctcccgcagtcgagccaacgtgctgaacaaggtagaatatgcacagcagcgctggaagcttcaagtccaagagcagcgaaagtctgtctttgaccggcatgttgtcctcagc

SEQ ID NO: 17 - DNA, 579 bp, Homo sapiens
atgatccgctgcggcctggcctgcgagcgctgccgctggatcctgcccctgctcctactcagcgccatcgccttcgacatcatcgcgctggccggccgcggctggttgcagtctagcgaccacggccagacgtcctcgctgtggtggaaatgctcccaagagggcggcggcagcgggtcctacgaggagggctgtcagagcctcatggagtacgcgtggggtagagcagcggctgccatgctcttctgtggcttcatcatcctggtgatctgtttcatcctctccttcttcgccctctgtggaccccagatgcttgtcttcctgagagtgattggaggtctccttgccttggctgctgtgttccagatcatctccctggtaatttaccccgtgaagtacacccagaccttcacccttcatgccaaccctgctgtcacttacatctataactgggcctacggctttgggtgggcagccacgattatcctgattggctgtgccttcttcttctgctgcctccccaactacgaagatgaccttctgggcaatgccaagcccaggtacttctacacatctgcc

SEQ ID NO: 18 - DNA, 1149 bp, Homo sapiens
atggcagggattccagggctcctcttccttctcttctttctgctctgtgctgttgggcaagtgagcccttacagtgccccctggaaacccacttggcctgcataccgcctccctgtcgtcttgccccagtctaccctcaatttagccaagccagactttggagccgaagccaaattagaagtatcttcttcatgtggaccccagtgtcataagggaactccactgcccacttacgaagaggccaagcaatatctgtcttatgaaacgctctatgccaatggcagccgcacagagacgcaggtgggcatctacatcctcagcagtagtggagatggggcccaacaccgagactcagggtcttcaggaaagtctcgaaggaagcggcagatttatggctatgacagcaggttcagcatttttgggaaggacttcctgctcaactaccctttctcaacatcagtgaagttatccacgggctgcaccggcaccctggtggcagagaagcatgtcctcacagctgcccactgcatacacgatggaaaaacctatgtgaaaggaacccagaagcttcgagtgggcttcctaaagcccaagtttaaagatggtggtcgaggggccaacgactccacttcagccatgcccgagcagatgaaatttcagtggatccgggtgaaacgcacccatgtgcccaagggttggatcaagggcaatgccaatgacatcggcatggattatgattatgccctcctggaactcaaaaagccccacaagagaaaatttatgaagattggggtgagccctcctgctaagcagctgccagggggcagaattcacttctctggttatgacaatgaccgaccaggcaatttggtgtatcgcttctgtgacgtcaaagacgagacctatgacttgctctaccagcaatgcgatgcccagccaggggccagcgggtctggggtctatgtgaggatgtggaagagacagcagcagaagtgggagcgaaaaattattggcattttttcagggcaccagtgggtggacatgaatggttccccacaggatttcaacgtggctgtcagaatcactcctctcaaatatgcccagatttgctattggattaaaggaaactacctggattgtagggagggg

SEQ ID NO: 19 - DNA, 792 bp, Homo sapiens
atggcgccggcgtcgcggttgctcgcgctctgggcgctggcggctgtggctctacccggctccggggcggagggcgacggcgggtggcgcccgggcgggccgggggccgtggcggaggaggagcgctgcacggtggagcgtcgggccgacctcacctacgcggagttcgtgcagcagtacgccttcgtcaggcccgtcatcctgcagggactcacggacaactcgaggttccgggccctgtgctcccgcgacaggttgctggcttcgtttggggacagagtggtccggctgagcaccgccaacacctactcctaccacaaagtggacttgcccttccaggagtatgtggagcagctgctgcacccccaggaccccacctccctgggcaatgacaccctgtacttcttcggggacaacaacttcaccgagtgggcctctctctttcggcactactccccacccccatttggcctgctgggaaccgctccagcttacagctttggaatcgcaggagctggctcgggggtgcccttccactggcatggacccgggtactcagaagtgatctacggtcgtaagcgctggttcctttacccacctgagaagacgccagagttccaccccaacaagaccacgctggcctggctccgggacacatacccagccctgccaccgtctgcacggcccctggagtgtaccatccgggctggtgaggtgctgtacttccccgaccgctggtggcatgctacgctcaaccttgacaccagcgtcttcatctccaccttcctcggc

SEQ ID NO: 20 - DNA, 759 bp, Homo sapiens
atggataaccgttttgctacagcatttgtaattgcttgtgtgcttagcctcatttccaccatctacatggcagcctccattggcacagacttctggtatgaatatcgaagtccagttcaagaaaattccagtgatttgaataaaagcatctgggatgaattcattagtgatgaggcagatgaaaagacttataatgatgcactttttcgatacaatggcacagtgggattgtggagacggtgtatcaccatacccaaaaacatgcattggtatagcccaccagaaaggacagagtcatttgatgtggtcacaaaatgtgtgagtttcacactaactgagcagttcatggagaaatttgttgatcccggaaaccacaatagcgggattgatctccttaggacctatctttggcgttgccagttccttttaccttttgtgagtttaggtttgatgtgctttggggctttgatcggactttgtgcttgcatttgccgaagcttatatcccaccattgccacgggcattctccatctccttgcaggtctgtgtacactgggctcagtaagttgttatgttgctggaattgaactactccaccagaaactagagctccctgacaatgtatccggtgaatttggatggtccttctgcctggcttgtgtctctgctcccttacagttcatggcttctgctctcttcatctgggctgctcacaccaaccggaaagagtacaccttaatgaaggcatatcgtgtggca

SEQ ID NO: 21 - DNA, 2160 bp, Homo sapiens
atggagctgggttgctggacgcagttggggctcacttttcttcagctccttctcatctcgtccttgccaagagagtacacagtcattaatgaagcctgccctggagcagagtggaatatcatgtgtcgggagtgctgtgaatatgatcagattgagtgcgtctgccccggaaagagggaagtcgtgggttataccatcccttgctgcaggaatgaggagaatgagtgtgactcctgcctgatccacccaggttgtaccatctttgaaaactgcaagagctgccgaaatggctcatgggggggtaccttggatgacttctatgtgaaggggttctactgtgcagagtgccgagcaggctggtacggaggagactgcatgcgatgtggccaggttctgcgagccccaaagggtcagattttgttggaaagctatcccctaaatgctcactgtgaatggaccattcatgctaaacctgggtttgtcatccaactaagatttgtcatgttgagcctggagtttgactacatgtgccagtatgactatgttgaggttcgtgatggagacaaccgcgatggccagatcatcaagcgtgtctgtggcaacgagcggccagctcctatccagagcataggatcctcactccacgtcctcttccactccgatggctccaagaattttgacggtttccatgccatttatgaggagatcacagcatgctcctcatccccttgtttccatgacggcacgtgcgtccttgacaaggctggatcttacaagtgtgcctgcttggcaggctatactgggcagcgctgtgaaaatctccttgaagaaagaaactgctcagaccctgggggcccgatcaatgggtaccagaaaataacagggggccctgggcttatcaacggacgccatgctaaaattggcaccgttgtgtctttcttttgttacaactcctatgttcttagtggcaatgagaaaagaacttgccagcagaatggagagtggtcagggaaacagcccatctgcataaaagcctgccgagaaccaaagatttcagacctggtgagaaggagagttcttccgatgcaggttcagtcaagggagacaccattacaccagctatactcagcggccttcagcaagcagaaactgcagagtgcccctaccaagaagccagcccttccctttggagatctgcccatgggataccaacatctgcatacccagctccagtatgagtgcatctcacccttctaccgccgcctgggcagcagcaggaggacatgtctgaggactgggaagtggagtgggcgggcaccatcctgcatccctatctgcgggaaaattgagaacatcactgctccaaagacccaagggttgcgctggccgtggcaggcagccatctacaggaggaccagcggggtgcatgacggcagcctacacaagggagcgtggttcctagtctgcagcggtgccctggtgaatgagcgcactgtggtggtggctgcccactgtgttactgacctggggaaggtcaccatgatcaagacagcagacctgaaagttgttttggggaaattctaccgggatgatgaccgggatgagaagaccatccagagcctacagatttctgctatcattctgcatcccaactatgaccccatcctgcttgatgctgacatcgccatcctgaagctcctagacaaggcccgtatcagcacccgagtccagcccatctgcctcgctgccagtcgggatctcagcacttccttccaggagtcccacatcactgtggctggctggaatgtcctggcagacgtgaggagccctggcttcaagaacgacacactgcgctctggggtggtcagtgtggtggactcgctgctgtgtgaggagcagcatgaggaccatggcatcccagtgagtgtcactgataacatgttctgtgccagctgggaacccactgccccttctgatatctgcactgcagagacaggaggcatcgcggctgtgtccttcccgggacgagcatctcctgagccacgctggcatctgatgggactggtcagctggagctatgataaaacatgcagccacaggctctccactgccttcaccaaggtgctgccttttaaagactggattgaaagaaatatgaaa

SEQ ID NO: 22 - DNA, 1029 bp, Homo sapiens
atgggtggcccccggggcgcgggctgggtggcggcgggcctgctgctcggcgcgggcgcctgctactgcatttacaggctgacccggggtcggcggcggggcgaccgcgagctcgggatacgctcttcgaagtccgcaggtgccctggaagaagggacgtcagagggtcagttgtgcgggcgctcggcccggcctcagacgggaggtacctgggagtcacagtggtccaagacctcgcagcctgaagacttaactgatggttcatatgatgatgttctaaatgctgaacaacttcagaaactcctttacctgctggagtcaacggaggatcctgtaattattgaaagagctttgattactttgggtaacaatgcagccttttcagttaaccaagctattattcgtgaattgggtggtattccaattgttgcaaacaaaatcaaccattccaaccagagtattaaagagaaagctttaaatgcactaaataacctgagtgtgaatgttgaaaatcaaatcaagataaagatatacatcagtcaagtatgtgaggatgtcttctctggtcctctgaactctgctgtgcagctggctggactgacattgttgacaaacatgactgttaccaatgaccaccagcacatgcttcacagttacattacagacctgttccaggtgttacttactggaaatggaaacacgaaggtgcaagttttgaaactgcttttgaatttgtctgaaaatccagccatgacagaaggacttctccgtgcccaagtggattcatcattcctttccctttatgacagccacgtagcaaaggagattcttcttcgagtacttacgctatttcagaatataaagaactgcctcaaaatagaaggccatttagctgtgcagcctactttcactgaaggttcattgtttttcctgttacatggagaagaatgtgcccagaaaataagagctttagttgatcaccatgatgcagaggtgaaggaaaaggttgtaacaataatacccaaaatc

SEQ ID NO: 23 - protein, 183 aa, Homo sapiens
MKLLSLVAVVGCLLVPPAEANKSSEDIRCKCICPPYRNISGHIYNQNVSQKDCNCLHVVEPMPVPGHDVEAYCLLCECRYEERSTTTIKVIIVIYLSVVGALLLYMAFLMLVDPLIRKPDAYTEQLHNEEENEDARSMAAAAASLGGPRANTVLERVEGAQQRWKLQVQEQRKTVFDRHKMLS

SEQ ID NO: 24 - protein, 339 aa, Homo sapiens
MAQLGAVVAVASSFFCASLFSAVHKIEEGHIGVYYRGGALLTSTSGPGFHLMLPFITSYKSVQTTLQTDEVKNVPCGTSGGVMIYFDRIEVVNFLVPNAVYDIVKNYTADYDKALIFNKIHHELNQFCSVHTLQEVYIELFDQIDENLKLALQQDLTSMAPGLVIQAVRVTKPNIPEAIRRNYELMESEKTKLLIAAQKQKVVEKEAETERKKALIEAEKVAQVAEITYGQKVMEKETEKKISEIEDAAFLAREKAKADAECYTAMKIAEANKLKLTPEYLQLMKYKAIASNSKIYFGKDIPNMFMDSAGSVSKQFEGLADKLSFGLEDEPLETATKEN

SEQ ID NO: 25 - protein, 348 aa, Homo sapiens
MNMTQARVLVAAVVGLVAVLLYASIHKIEEGHLAVYYRGGALLTSPSGPGYHIMLPFITTFRSVQTTLQTDEVKNVPCGTSGGVMIYIDRIEVVNMLAPYAVFDIVRNYTADYDKTLIFNKIHHELNQFCSAHTLQEVYIELFDQIDENLKQALQKDLNLMAPGLTIQAVRVTKPKIPEAIRRNFELMEAEKTKLLIAAQKQKVVEKEAETERKKAVIEAEKIAQVAKIRFQQKVMEKETEKRISEIEDAAFLAREKAKADAEYYAAHKYATSNKHKLTPEYLELKKYQAIASNSKIYFGSNIPNMFVDSSCALKYSDIRTGRESSLPSKEALEPSGENVIQNKESTG

SEQ ID NO: 26 - protein, 183 aa, Homo sapiens; identical to SEQ ID NO: 23

SEQ ID NO: 27 - protein, 198 aa, Homo sapiens
MATLWGGLLRLGSLLSLSCLALSVLLLAQLSDAAKNFEDVRCKCICPPYKENSGHIYNKNISQKDCDCLHVVEPMPVRGPDVEAYCLRCECKYEERSSVTIKVTIIIYLSILGLLLLYMVYLTLVEPILKRRLFGHAQLIQSDDDIGDHQPFANAHDVLARSRSRANVLNKVEYAQQRWKLQVQEQRKSVFDRHVVLS

SEQ ID NO: 28 - protein, 193 aa, Homo sapiens
MIRCGLACERCRWILPLLLLSAIAFDIIALAGRGWLQSSDHGQTSSLWWKCSQEGGGSGSYEEGCQSLMEYAWGRAAAAMLFCGFIILVICFILSFFALCGPQMLVFLRVIGGLLALAAVFQIISLVIYPVKYTQTFTLHANPAVTYIYNWAYGFGWAATIILIGCAFFFCCLPNYEDDLLGNAKPRYFYTSA

SEQ ID NO: 29 - protein, 383 aa, Homo sapiens
MAGIPGLLFLLFFLLCAVGQVSPYSAPWKPTWPAYRLPVVLPQSTLNLAKPDFGAEAKLEVSSSCGPQCHKGTPLPTYEEAKQYLSYETLYANGSRTETQVGIYILSSSGDGAQHRDSGSSGKSRRKRQIYGYDSRFSIFGKDFLLNYPFSTSVKLSTGCTGTLVAEKHVLTAAHCIHDGKTYVKGTQKLRVGFLKPKFKDGGRGANDSTSAMPEQMKFQWIRVKRTHVPKGWIKGNANDIGMDYDYALLELKKPHKRKFMKIGVSPPAKQLPGGRIHFSGYDNDRPGNLVYRFCDVKDETYDLLYQQCDAQPGASGSGVYVRMWKRQQQKWERKIIGIFSGHQWVDMNGSPQDFNVAVRITPLKYAQICYWIKGNYLDCREG

SEQ ID NO: 30 - protein, 264 aa, Homo sapiens
MAPASRLLALWALAAVALPGSGAEGDGGWRPGGPGAVAEEERCTVERRADLTYAEFVQQYAFVRPVILQGLTDNSRFRALCSRDRLLASFGDRVVRLSTANTYSYHKVDLPFQEYVEQLLHPQDPTSLGNDTLYFFGDNNFTEWASLFRHYSPPPFGLLGTAPAYSFGIAGAGSGVPFHWHGPGYSEVIYGRKRWFLYPPEKTPEFHPNKTTLAWLRDTYPALPPSARPLECTIRAGEVLYFPDRWWHATLNLDTSVFISTFLG

SEQ ID NO: 31 - protein, 253 aa, Homo sapiens
MDNRFATAFVIACVLSLISTIYMAASIGTDFWYEYRSPVQENSSDLNKSIWDEFISDEADEKTYNDALFRYNGTVGLWRRCITIPKNMHWYSPPERTESFDVVTKCVSFTLTEQFMEKFVDPGNHNSGIDLLRTYLWRCQFLLPFVSLGLMCFGALIGLCACICRSLYPTIATGILHLLAGLCTLGSVSCYVAGIELLHQKLELPDNVSGEFGWSFCLACVSAPLQFMASALFIWAAHTNRKEYTLMKAYRVA

SEQ ID NO: 32 - protein, 720 aa, Homo sapiens
MELGCWTQLGLTFLQLLLISSLPREYTVINEACPGAEWNIMCRECCEYDQIECVCPGKREVVGYTIPCCRNEENECDSCLIHPGCTIFENCKSCRNGSWGGTLDDFYVKGFYCAECRAGWYGGDCMRCGQVLRAPKGQILLESYPLNAHCEWTIHAKPGFVIQLRFVMLSLEFDYMCQYDYVEVRDGDNRDGQIIKRVCGNERPAPIQSIGSSLHVLFHSDGSKNFDGFHAIYEEITACSSSPCFHDGTCVLDKAGSYKCACLAGYTGQRCENLLEERNCSDPGGPINGYQKITGGPGLINGRHAKIGTVVSFFCYNSYVLSGNEKRTCQQNGEWSGKQPICIKACREPKISDLVRRRVLPMQVQSRETPLHQLYSAAFSKQKLQSAPTKKPALPFGDLPMGYQHLHTQLQYECISPFYRRLGSSRRTCLRTGKWSGRAPSCIPICGKIENITAPKTQGLRWPWQAAIYRRTSGVHDGSLHKGAWFLVCSGALVNERTVVVAAHCVTDLGKVTMIKTADLKVVLGKFYRDDDRDEKTIQSLQISAIILHPNYDPILLDADIAILKLLDKARISTRVQPICLAASRDLSTSFQESHITVAGWNVLADVRSPGFKNDTLRSGVVSVVDSLLCEEQHEDHGIPVSVTDNMFCASWEPTAPSDICTAETGGIAAVSFPGRASPEPRWHLMGLVSWSYDKTCSHRLSTAFTKVLPFKDWIERNMK

SEQ ID NO: 33 - protein, 343 aa, Homo sapiens
MGGPRGAGWVAAGLLLGAGACYCIYRLTRGRRRGDRELGIRSSKSAGALEEGTSEGQLCGRSARPQTGGTWESQWSKTSQPEDLTDGSYDDVLNAEQLQKLLYLLESTEDPVIIERALITLGNNAAFSVNQAIIRELGGIPIVANKINHSNQSIKEKALNALNNLSVNVENQIKIKIYISQVCEDVFSGPLNSAVQLAGLTLLTNMTVTNDHQHMLHSYITDLFQVLLTGNGNTKVQVLKLLLNLSENPAMTEGLLRAQVDSSFLSLYDSHVAKEILLRVLTLFQNIKNCLKIEGHLAVQPTFTEGSLFFLLHGEECAQKIRALVDHHDAEVKEKVVTIIPKI

SEQ ID NO: 34 - DNA, 1570 bp, Mus musculus
  CDS (84)...(659)
gtcgacccacgcgtccgggccggggtcctgagccggagccggagcgcgcgccgctgcccagccccgccgcgccggccccgcagatggtgactccgcggcccgcgcccgcccggggccccgcgctcctcctcctcctgctgctggccactgcgcgcgggcaggaacaggaccagaccaccgactggagggccaccctcaagaccatccgcaacggcatccacaagatagacacgtacctcaacgccgcgctggacctgctgggcggggaggacgggctctgccagtacaagtgcagcgacggatcgaagcctgttccacgctatggatataaaccatctccaccaaatggctgtggctctccactgtttggcgttcatctgaacataggtatcccttccctgaccaagtgctgcaaccagcacgacagatgctatgagacctgcgggaaaagcaagaacgactgtgacgaggagttccagtactgcctctccaagatctgcagagacgtgcagaagacgctcggactatctcagaacgtccaggcatgtgagacaacggtggagctcctctttgacagcgtcatccatttaggctgcaagccatacctggacagccagcgggctgcatgctggtgtcgttatgaagaaaaaacagatctataaagaccctgactgctggagagcaggcgagaatggaggatcatccttgccaaagatcggatgctttaacagcctaatgttgccttagttttgtgtcgatgggtcattttgagacctttctatactgtgtctttttttagaacctcaaagtgaaaacggtggggggccaggcagaaacagagggagagcatgcttgggatggggagcgagcaggacatccaagagcatgccttcctgagactcgctgtcttggtggctcccccaaactgggaagaaaagcttaagctcgtgtgacttggtgttcatagttgtacttaacaataaaaatgaaagcaaatgtaaaattcattgtaaggacttttcagcattattttattttgaaatacaggccaatcttcccttagaactattatttattttgaaatttcagatgtacatttatacctggaaaaactattaattctccatttttattatacataatgtgttgtttctctgaagcccactaagataggtataaatatgttactcaaaactacacggtttccaaatgtgcatctcttgtacagttggaatcacggttggtacttctctggagagacgccccaggacatctgagtgttgggatgtgcacagaattcagaagcccagcttcctgtctcacaaaccgcttagagtgaatgtccttcctctcctgctgtgagctctaggaatgacgggtttaacgggccaagccgagctctgaatcagtgcgctatctgctgctgaggttgtggttactccctcatccccgttttccatcttctatcctggagtagtgttaaaagtctgacattttctaatggaggtcttaataaaagctatttacttcttggtaaaaaaaaaaaaaaaaaaaaaaaaaagggcggccg

SEQ ID NO: 35 - DNA, 886 bp, Mus musculus
  CDS (167)...(886)
gtcgacccacgcgtccgcggacgcgtgggcgcggactgatggcgtcatcgaagcgactggcccggaaggaagtagggtgctgaggggtgtggcggtttctacggttgcacgggggttcggctgtgtacggagcgcctggagggacagcctggatacaggttcactgatggctcagttgggagctgttgtggccgtggcttccagtttcttttgtgcatctctcttctcagctgtgcacaagatagaagagggacatattggagtatattacagaggtggtgccctgctgacctccaccagtggcccgggtttccatctcatgctcccgttcatcacatcctataagtctgtacagaccactctccaaactgatgaagtgaagaacgtaccatgtggaaccagtggtggtgtgatgatctactttgacagaattgaagtggtgaacttcctggtcccaaatgcagtgtatgatatagtgaagaactatactgcagactatgacaaggccctcatcttcaacaagatccatcatgagcttaaccagttctgcagcgttcatactcttcaggaagtctatatcgagctgtttgatcaaattgatgaaaacctcaagttggctttgcagcaggacctgacttccatggcccctgggctggttatccaagctgtgcgagtgacaaagcccaatatacctgaggcaatccgcaggaactatgagctgatggaaagcgagaagacgaagcttctcattgcagcccagaagcagaaggtggtggaaaaggaggcagaaacagagaggaagaaggccctcattgaggcagaaaaagtggcacaggttgcagaaatcacctatgggcaaaaggtgatggagaaggagacagagaag

SEQ ID NO: 36 - DNA, 2851 bp, Mus musculus
  CDS (52)...(843)
gtcgacccacgcgtccgtaaaaatgtgccttgtggaacaagtggtggagtcatgatctatattgaccgaatagaagtggttaatatgttggctccttatgcagtgtttgacattgtgaggaactatactgcagactacgacaagactttaatcttcaataaaatccaccatgagctgaaccagttttgcagtgcccacacacttcaagaagtttacatagaattgtttgatcaaatagatgaaaacctgaagcaggccctgcaaaaagatttaaacaccatggccccaggtctcactatccaggctgtgcgtgttacaaaacccaaaatcccagaagccataagaagaaattttgaattaatggaggcagagaagacaaaacttctcatagctgcacagaaacaaaaggtggtggagaaagaagctgagacggagaggaaaagggctgttatagaagcagagaagattgcacaagtagcaaaaattcgatttcaacagaaagtgatggagaaagaaactgaaaaacgcatttctgagattgaagatgctgcgttcctggcccgagagaaggcaaaagcagatgccgagtattacgctgcacacaaatacgccacctcaaacaagcacaaactgaccccagagtatctggagctcaagaaataccaggccattgcctcaaacagtaagatctactttggcagcaacatccccagcatgtttgtggactcctcctgtgctctgaaatactctgatggtaggactgggagagaagactcccttcccccagaggaggcccgtgagccctctggagagagccccatccaaaacaaggagaacgcaggttgatgcaagaggtggaaatgttctcccatatcaagatgcgacccaaggggctaagtgggaacagtggttatgtggactcgtaagattcacagagaatgtgtgctctgttgtgattctcttgtcatagtcctggtttgccagctgactacaggatagacccagctgtctggcactcaaacggtctctgcagccacagttttatcaagtatcctgtatgtgttcctttgtaaaccggtactcatgaatgagggaaagtctgatgctaagatactgcctgcactggaatgtcaaacactatataacaagctgtggtttttaaaagctattgaataatgtttacattggtccctgaggacatgtgtgctcagacattcaagagctaggaggccagagagaagaccttcagaaaacggtaagttaaagaagacaagtgtcatcagacacttgggacccgggctctctttaaagtctagtcccggcattcctccatgtgattgacagccagacctctgggttcccaggaaattatcttccagttgaatgaccatttacttgatacaaattgtacctttctgtttttctagtcaggttggtggcctgcagggacgcgtactttgccacccgaccagaggttcctcgaagatattcccaatcactagtttattgcgttaggagactcagagatatagaaagcagctgaaatttaagggagataaagcctgcactgcaccaaagctacgggtccctgtgtttcctctattcagtgatgtcatcaacctcactgtcccagcccatgtgtgactaaagtgcccggttttagccacagacaactgcttagatgtcacctcttggctgaccaaagctgggacagggctttaaccagacataggagcagtgtgcaattcctgattcactgcacagtattatgtcataattgcaggaattattttttgtttttaaaactggatttggggcacattcattcaccccaacacttctatctaaaggccaaggttctagggctgctatggtcactaacacactgattctccttaaagtaattctcgaagtgtggaacaaagtgaccgagacagcatcctcagtcatctttgtctccttccctgggatgcagataccgaagttgcttttccaactttcgcctccgctaggagatcagaaagaattcttgtgacttcctgggcagccattgaattcattttccatgagaagatgacagagttagcctgtggctataggagatcatgtcatccagacctttttgcccatcacattaactttcctggaatattgtgctgcacaggtagacctgaatctgcccagcttgttgacagctcttgtgtatactgtgttgaagccagacagaaaagtaatggggccacttctgaaacctctcagctgttgatctcacagcagctaaagggttgtgccaaacattttattaagaaagtaaagcccagatttgaatgggggttttccctaggccttatagtatagaggcatttgtaatatggagaaaataatttttctcatttaattatagaaattaccttcaaacagattttgtgttctttggcccttcaaatactggtgttacattgttgctgcagataaatgatgattgtcgtgggatatctggatcactgagctctgtgctttcattcctagagatgtttctcattcccatttagtgaaatgctgttgccccaaagtgatggttgtgggatttcttaccggtcataggccccggtgaggagcagggaagcgccattgtgaaagattaaagaaagcacttccacttgagctccttatggagtgagcttccctgtgcccactcagtgaactaagtctgaccatccttcagggacgttccttttggtaaatatacactgtaatctttaagtctaaatttatatgtgaaagttaactttttttaaaaacctaaataaaattattttcctatcaaaaaaaaaaaaaaaaagggcggccg

SEQ ID NO: 37 - DNA, 1509 bp, Mus musculus
  CDS (90)...(638)
gtcgacccacgcgtccggcctgctgatcagtggcggctgcggctgagcttgcaggcatctagtcttgctggtcagcaagcccgataagcatgaagctgctgtgtttggtggctgtggtggggtgcttgctggtgcccccagctcaagccaacaagagctctgaagatatccggtgcaaatgcatctgtccgccttacagaaacatcagcgggcacatttacaaccagaatgtgtctcagaaggactgcaactgcctgcatgtggtggagcccatgccagtgcctggccacgatgtggaagcctactgcctgctctgcgagtgtaggtacgaggagcgtagcaccacaaccatcaaggtcattattgtcatctacctgtctgtggtgggggccctcttactctacatggccttcctgatgctggtggacccgctcatccggaagccagatgcctatactgagcagctgcacaatgaagaggagaatgaggatgctcgcaccatggcaacagccgctgcgtccattggaggaccccgggcaaacactgtcctggagcgggtggaaggcgctcagcagcggtggaagctgcaggtgcaggagcagcggaagacggtcttcgaccgacacaagatgctcagttagatggttgccatgattgcatcagagacctgggccatggctaccagcttctggggctcactgcagtcttccctgggtcttcccttcaaatgcccatggcgtttatccttctccctctctagaaatgtactcgactgttataacgagggagtgtgattgggtctctgtaggtctctggggggtagaggggaggggagggaaggcagaagggaacagagacatttgaggtggccacatgattgggtggaattcatccctcctgtcttcaccattcctcccagctccacatcttaaggatgcttacgggagacgaagctgtgtcatcaagagctcagtgggtgcgaggaaagtatgatccagcgctcagccttcgctctaggatgctgtggtccccattcccagttccttcagtgccagtactttaacttggcctaccccagtctcaggaactgttgtggtgcccctgagcccacagtcatctccagagtccacctggaagcctgttcccctctcctcggctcctggtccaccagtgcatggcagtgcccatgcatgccggcatattcagcagctgtcaccttactcccatcccaggaggccgtaaggcctcccacctctcccctgtgactgcagctgctgagccataaagttggaccatatgacacaaggccaatggggaccggagtaccatggctcctgtccttggatggtctcttgtccctgaatttcattgtatcatgcatggagagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaggggggc

SEQ ID NO: 38 - DNA, 1681 bp, Mus musculus
  CDS (59)...(655)
gtcgacccacgcgtccgggcgcggggctcggggctcgcaggagcggctggctcccgcgatggcgagcctatggtgcggaaacctgctgcggctgggctcggggctcagcatgtcctgcctggcgctgtcggtgctgctgctcgcgcagctgacaggcgccgccaagaattttgaagatgtgagatgtaaatgcatctgccctccctataaagagaatcctgggcacatttataataagaatatatctcagaaagattgtgattgccttcatgtcgtggagcccatgcctgtacggggacctgatgtagaagcatactgtctacgctgtgaatgcaaatacgaagagagaagctctgtcacaatcaaggttaccattataatttatctctctattttgggccttctgcttctgtacatggtatatcttaccttagttgagcccatcctgaagaggcgcctctttggacactcccagctgttgcagagcgatgatgacgttggggatcaccagccttttgcaaatgcccatgatgtgctggcccgctctcgcagccgagccaatgttctaaacaaggtggagtacgctcagcagcgctggaagctccaggtccaggagcagcgaaagtctgtcttcgaccgacacgttgtcctcagctaactgggaactggaatcaggtgactaggaagaacacgcagacaactgggaagaattgtctgggtgtccgtgcgttttaatgccatgtttgtttttacaaatccttgctggatggaggaagactccaaactggaagcaaaccccatgcttggtattttcctgttaatatattaatagagacatttttacagcacacagttccaagtcaaccagtaagtcttttcctacttgtgacttttactaataaaattaagctgcctgtgagttatcttgaagccccgtgcctggaacaagctctctctttcttgccacacagttctaacttggtgttcaagataacttccaggtgtgtttttgcttctctttcttgtggtgggagagagaaggaaggatgccttgggagtgcttgagtagcttctcaagtgtcttttccagacagacttatgaatacttcagaccctctacttcacacttgttaatgtcccagtgtagctggcttgtcagcgtgctggcctccccacttgacttttgcactgactacattacctaagattctggttagcctgtggctgcatttcatgaccagttggatctgaaatgcctgggggctcctcacaaaatgaagatttgtttcatgcactgtgatgtctgacgcaacatgttctagaacagactggccatctgctagtttacactgatacctaaacacagtctcagtgtgtgtggtcttcctcatcttcttctagtagctctaaggacttgaacatttagaataaagacattttctcttaagcccaagcctccctggatgattgacgtacaaatactgatcagccttttctgtcttgctgagaggcagttctttgaactgatgtgggcagctttgaacaaggactagagttcagattgcctctctctgagaagtctaacagttattggataactggcttttttcttcctacatcctctttggaatgtaacaataaaataatttacaaaacccaaaaaaaaaaaaaagggcggccg

SEQ ID NO: 39 - DNA, 2030 bp, Mus musculus
  CDS (200)...(778)
gtcgacccacgcgtccggcgctctgagtcaccggaatcaaggtgtggctggagcgccgctcccccgccgccagcccgggggccgcgtcttcgggggagccgcctcttcctttagtcgcggtgtcagcgctcgcaggaccactcttggccgctgctcctgcccggcgttcctccgctccgcgcccgccgccaccgacgacatgctgcgctgcggcctggcctgcgagcgctgcaggtggatcctgcccctgctgctgctcagcgccatcgccttcgacatcatcgcgctggccggccgcggctggctgcagtctagcaaccacatccagacatcgtcgctttggtggaggtgtttcgacgagggcggcggcagcggctcctacgacgatggctgccagagcctcatggagtacgcatggggacgagcagctgcagccacgcttttctgtggctttatcatcctgtgcatctgcttcattctctcgttcttcgccctgtgtggaccccagatgcttgttttcctgagagtcattggaggcctcctcgcactggctgccatattccagatcatctccctggtaatctaccccgtgaagtacacacagaccttcaggcttcacgataaccctgctgttaattacatctataactgggcctatggcttcggatgggcggccaccatcatcttgattggttgttccttcttcttctgctgcctccccaactacgaggatgaccttttgggggccgccaagcccaggtacttctatcccccagcctaatgtgggaggaagagcctgagaaaagcctgctgcaagatggatctgaggaggaaactgttctccaaggcacaaggaacctacgtttgggcaatgttcatatgatcagaaatgctagaataaatgctaaagaaaattcttcataattagtgttaagtttcatgtatgtcgtgtggagttaaaaagacttgaattctgtttgctaagtatatgctaatttttccttatgtcaattctataccatttaagcttcatttgttaaagaatatgcctgtgaaacttgataaggtagaaatgtagcagcctctcatttaataatctgatggggcttctgtttttccacatagaatgggttgtttctgctaagggctacagaggaggaaagtcactggcaaaacttccgtgaccaaatatcctgaaattagtatttttttaaaaagaccttattttgagttttcagttacataaaaaagcagaagcagattggtttcctaagtgagcatcgtttgtgagaatttttagtcagtgttttgaacaattattgtttttctaagcttcgtgttgactttctctgatgcgtagaaaagtgttctaacgtagccaaggttaagccgctgtcactactgaaatgctaagaattttcctcttttcccgtagtgtagaggggtagggtgtgggaagaagccgtgttagcacatctgtagtattctgtgtgtatgcttagaaccagcgtagaccggatgggaggatggactaggcctaatccctcccaactggtggatgtgaagaggtcaggtaggaaggcacaggagggtcaccactgtcacagcagtgccatgcagacatcctaggagaagacatggcagtgtttcttctcagtgcttcttcccttaactgagctctgctcacagacagctagaatagattttaactgtaacagaaacctaaatgtaattaaaacctggtcttccttggtaagcagacttaaaatatctgtatagtacatgcaagtggaaaatttgggaatgcgtgtctctgaatacataccggaagggctactattaccttttccttaccatttatacttacctaatggaaacgagcttgttttaactatcagaacactattttgtaaggtgctgcaaagacagttgaagttttcattaccaacttccccaataaaccaggtgttcaaaaaaaaaaaaaaaacaaaaaaaaaaaaaaaaaaaaaaaaaaaaaaaagggcggccgc

SEQ ID NO: 40 - DNA, 2007 bp, Mus musculus
  CDS (148)...(1293)
gtcgacccacgcgtccgcggacgcgtgggcactcggccactctgcggagcaggcatgggagccgcgcgcgtcctccgggcgcccacacctgtctgagcggcgcacggccgcggccccggcgggctgctccacgcggtagcactcagcatggctggaatcccggggctcttcatccttcttgtcctgctctgtgtgttcatgcaggtgagtccctacaccgttccgtggaaacccacatggccggcttatcgcctccctgtagtcttgcctcagtctaccctcaacttagctaaggcagacttcgacgccaaagcgaaattggaggtgtcctcctcatgtggacctcagtgtcacaagggaacaccactgcccacctacgaagaggccaagcagtacctttcctatgaaaccctttatgccaatggcagccgcacagagactcgggtgggcatctacatcctcagcaatggtgaaggcagggcacgaggcagagactcggaggccacagggagatctcgcaggaagaggcagatttatggctacgatggcaggtttagcatttttgggaaggacttcctgctcaattatcctttctcaacatcggtgaagttgtctactggctgcactggcaccctggtggcagagaagcacgtcctcactgctgcccactgcatacacgatgggaaaacctatgtgaaagggacacagaaactccgagtgggcttcctgaagcccaagtataaagatggtgccgaaggggacaacagctcgagctcagccatgccagacaagatgaagtttcagtggatccgcgtgaaacgcacccatgtgcccaaggggtggatcaagggcaatgccaatgacatcggcatggattatgactacgccctgctggaactcaagaaaccccacaaaagacagttcatgaagattggtgtgagtcctccagcgaagcagctcccagggggcaggatccacttctctggttatgacaatgaccggcccggcaatttggtgtaccgcttctgtgatgtcaaagatgagacctacgaccttctctaccagcagtgtgacgcccagcccggggccagtggttcaggggtctatgtgaggatgtggaagagaccacagcagaaatgggaaagaaaaattatcggcatcttttcagggcaccagtgggtggacatgaatggctctccacaggatttcaacgtggcagttagaatcacgcctcttaaatatgcccagatttgctattggattaaaggaaactacctagattgcagggaggggtgacatgcgtcttcttgccagcaccaatggtctttttgcactcattgtaggagaggctagctttttatcattgactcttgtggtgtgagtcacatagtatcttttacctagtattcttcaaatggcaaaaattattggctatattattttaaaactgttgtgtgcgttatagcatttaagcagtctgaaagcatacttttgcatagagactttaaagtattcgggtaatagggcctatttgacaaggaagttaaactttcagtttttggagaattctaatttttgtctgatccaaacttgcttcagaggtttatatcaaatacgtgacacacagggaatatgaattcttatgtttgtatatgtatatgttttcttctgagagtcatatattgatatttttgtaatgtgtggttattatgcttccagataatgatagcaaagtcttcaataggcaatttataatgttttggattcaaacatttacgtagtagtccttgaagagaacaataatttattggctatattgatacccatataagactgtatcttacagtgcacagaattcccacgctgcttttagttttgaaaataaaactttcccttgtaaaaaaaaaaaaaaaaaaaaagggcggccgacagaattcccacgctgcttttagttttgaaaataaaactttcccttgtaaaaaaaaaaaaaaaaaaaaagggcggccg

SEQ ID NO: 41 - DNA, 2102 bp, Mus musculus
  CDS (22)...(828)
gtcgacccacgcgtccggttcatggcggcggctgggcggcgcggtctgcttttgctctttgtactatggatgatggtgactgtgattctgcctgcctctggcgaagggggatggaaacagaatgggctgggaattgcagcagcagtaatggaggaggagcgttgcacagtggagcgtcgggcacacatcacgtactccgaattcatgcagcactatgccttcctcaagcccgtcatcttgcaaggactcacggacaactcgaagttccgggccctgtgttcccgggaaaacctgctagcctcgttcggggacaacattgttcgcttgagtacagccaacacctactcctaccagaaagtggacctgcccttccaggaatatgtggaacagctgctgcagccccaggatcctgcatccctaggcaatgacaccctgtacttttttggagacaacaacttcactgagtgggcatccctcttccagcactactctccgccaccattccgtctcctgggaaccacccctgcttacagctttggaattgcaggagctggatctggggtacccttccactggcatgggcctggtttctcagaggttatctatggtcggaagcgctggttcctctaccctcctgagaagacacctgagttccaccctaacaagaccacattggcctggctgctggaaatatacccatctctagccctgtcagcacggcctctagaatgtaccatccaggctggtgaagtactgtattttcctgatcggtggtggcatgccacactcaatctggacaccagtgtcttcatttctaccttccttggctagccagacaggcaactggcaagcccactgcaccagcacatgccaatgtagtgctcacagactttattacaggacagtggcagcagcagcaacctcagcccaccctcacccactctccagcccagaagggggacaagggaggctcatggtccagcaaggggtatgctgagaaggggagcagttcagaacccatcagcagggccgatgggggcaggcccagggacacaaactatacagggactggagcttccgtctccagatcctcctgggccagggtgccaggcaggacatggggcctcaatagtcctctacccagccgttctcagagatgaaagcgtcaatgacttccttcatggccaagttggggatgagctgttcctgggtcaaagggctccgggtcacagggtcaaagtggcccacacgctgcaacagagtcaagagtgttcaatggcctgagtataccgatccgggtaccaaggctctccatggcccggtctccatgggccctccttacctgcaggtgctcctcaatgtccttgcggtcataggtgataccactgggtgtaatgcagggttcccgcatcagctcaaagctaatcttgccacacaagtagtcagggatatctcgcttctatagcacaaggggaaaatgtctagaactggagggggctgtgggggtcaccataccagcagcagccgatgagcttccgggggtcctcacctttcttttctcgtccacctgagagaagagctcatccatatctgccatgtatttatcctgcagagttgagtgccatgtgtgggcaactcctgtctccacacagacacacacactctgtccaccagggcactcatgtcatgcatgggccaacagatccaccaaaggctggggcacttttcatgccacacacaaacacacacacaatgacccacatgtggactaggggcaccctcacgtgcttggcctcaatgcaggcctgctgggcccggatgtggccatcatcttcatgaccctcgtggttccgctgacactcctccagttccctgagggttaaccagaagctagttggtgatggccctgaccaggaaatcacagagcccgccccatctcaggcctctttcctcctgggcttcccatgtaccggttgttgtccttcaataaaaacacttgtgctggtgactcagtgtctgctgggggagggacccacctctctcgctcagcagcaatgagcctggtgagatatgaatgcaaaaaaaaaaaaaaagggcggccg

SEQ ID NO: 42 - DNA, 871 bp, Mus musculus
  CDS (64)...(822)
aattcggmwcmkkkgvvggvvgccggtggagtgagaggatgggcgagcagtctgaatgccagaatggataaccgttttgctactgcgtttgtgattgcttgtgtgcttagtctgatttccaccatctacatggcggcctccataggcacggacttctggtatgagtatcgaagtcccattcaagagaattcaagtgactcgaataaaatcgcctgggaagatttcctcggtgacgaggcggatgagaagacttacaacgatgttctgttccgatacaacggcagcttggggctgtggagacggtgcatcaccatacccaaaaacactcactggtatgcgccaccggaaaggacagagtcatttgatgtggttaccaaatgcatgagtttcacactaaacgagcagttcatggagaagtatgtggaccccggcaaccacaatagcggcatcgacctgcttcgcacctacctgtggcgctgccagttccttttacccttcgtcagcttgggcttgatgtgctttggggcgttgattggcctctgtgcctgtatctgccgcagcctgtatcccaccctcgccactggcattctccatctccttgcaggtctgtgcacactgggctccgtgagttgctatgttgccggcattgaactcttacatcagaaagtagagctgcccaaggatgtatctggagaatttggatggtccttctgcctggcctgcgtctcggctcccttacagttcatggcggccgctctcttcatctgggctgcccacaccaaccggaaagagtacaccttaatgaaggcttatcgtgtggcatgaagggaggctgcctgcttaatgattaatatttttcatacattttttt

SEQ ID NO: 43 - DNA, 670 bp, Mus musculus
  CDS (215)...(670)
  misc_feature (1)...(670): n = A,T,C or G
tccggtccangaaaaagctgcttgcactaggggcatcccgcctgcctggtgaaaggaaccgcagcacacagggtgggagggcttccgattttagcagggcggcttccggaaggcggagctccaaccccatttcctttctctgggctggttctggcccagctgcacctgcgtgtggccctggctcctcggctccctgcagctccgaggcagcagcatgggtggcgcgcgggacgtgggctgggtggcagcagggctggtcctgggcgccggcgcctgctactgtatctaccggctgactcggggaccgcggcgaggcgtcgcgaccatgcgcccttcgcgatccgcagaagacctaaccgatggctcctatgacgatatcttaaatgcagagcagcttaagaaacttctgtatctgctggagtcaaccgacgatcctgtcattactgaaaaggccttggtcaccttgggaaataatgcagccttctccactaaccaggccattattcgtgagttgggtggtatcccaattgttggaaacaaaatcaactccctgaaccaaagtattaaagagaaagctttaaatgcactgaataacctgagtgtgaatgttgaaaatcaaactaagataaagatatacgtccctcaagtctgtgaggacgtctttgctgac

SEQ ID NO: 44 - DNA, 576 bp, Mus musculus
atggtgactccgcggcccgcgcccgcccggggccccgcgctcctcctcctcctgctgctggccactgcgcgcgggcaggaacaggaccagaccaccgactggagggccaccctcaagaccatccgcaacggcatccacaagatagacacgtacctcaacgccgcgctggacctgctgggcggggaggacgggctctgccagtacaagtgcagcgacggatcgaagcctgttccacgctatggatataaaccatctccaccaaatggctgtggctctccactgtttggcgttcatctgaacataggtatcccttccctgaccaagtgctgcaaccagcacgacagatgctatgagacctgcgggaaaagcaagaacgactgtgacgaggagttccagtactgcctctccaagatctgcagagacgtgcagaagacgctcggactatctcagaacgtccaggcatgtgagacaacggtggagctcctctttgacagcgtcatccatttaggctgcaagccatacctggacagccagcgggctgcatgctggtgtcgttatgaagaaaaaacagatcta

SEQ ID NO: 45 - protein, 240 aa, Mus musculus
MAQLGAVVAVASSFFCASLFSAVHKIEEGHIGVYYRGGALLTSTSGPGFHLMLPFITSYKSVQTTLQTDEVKNVPCGTSGGVMIYFDRIEVVNFLVPNAVYDIVKNYTADYDKALIFNKIHHELNQFCSVHTLQEVYIELFDQIDENLKLALQQDLTSMAPGLVIQAVRVTKPNIPEAIRRNYELMESEKTKLLIAAQKQKVVEKEAETERKKALIEAEKVAQVAEITYGQKVMEKETEK

SEQ ID NO: 46 - protein, 264 aa, Mus musculus
MIYIDRIEVVNMLAPYAVFDIVRNYTADYDKTLIFNKIHHELNQFCSAHTLQEVYIELFDQIDENLKQALQKDLNTMAPGLTIQAVRVTKPKIPEAIRRNFELMEAEKTKLLIAAQKQKVVEKEAETERKRAVIEAEKIAQVAKIRFQQKVMEKETEKRISEIEDAAFLAREKAKADAEYYAAHKYATSNKHKLTPEYLELKKYQAIASNSKIYFGSNIPSMFVDSSCALKYSDGRTGREDSLPPEEAREPSGESPIQNKENAG

SEQ ID NO: 47 - protein, 183 aa, Mus musculus
MKLLCLVAVVGCLLVPPAQANKSSEDIRCKCICPPYRNISGHIYNQNVSQKDCNCLHVVEPMPVPGHDVEAYCLLCECRYEERSTTTIKVIIVIYLSVVGALLLYMAFLMLVDPLIRKPDAYTEQLHNEEENEDARTMATAAASIGGPRANTVLERVEGAQQRWKLQVQEQRKTVFDRHKMLS

SEQ ID NO: 48 - protein, 199 aa, Mus musculus
MASLWCGNLLRLGSGLSMSCLALSVLLLAQLTGAAKNFEDVRCKCICPPYKENPGHIYNKNISQKDCDCLHVVEPMPVRGPDVEAYCLRCECKYEERSSVTIKVTIIIYLSILGLLLLYMVYLTLVEPILKRRLFGHSQLLQSDDDVGDHQPFANAHDVLARSRSRANVLNKVEYAQQRWKLQVQEQRKSVFDRHVVLS

SEQ ID NO: 49 - protein, 193 aa, Mus musculus
MLRCGLACERCRWILPLLLLSAIAFDIIALAGRGWLQSSNHIQTSSLWWRCFDEGGGSGSYDDGCQSLMEYAWGRAAAATLFCGFIILCICFILSFFALCGPQMLVFLRVIGGLLALAAIFQIISLVIYPVKYTQTFRLHDNPAVNYIYNWAYGFGWAATIILIGCSFFFCCLPNYEDDLLGAAKPRYFYPPA

SEQ ID NO: 50 - protein, 382 aa, Mus musculus
MAGIPGLFILLVLLCVFMQVSPYTVPWKPTWPAYRLPVVLPQSTLNLAKADFDAKAKLEVSSSCGPQCHKGTPLPTYEEAKQYLSYETLYANGSRTETRVGIYILSNGEGRARGRDSEATGRSRRKRQIYGYDGRFSIFGKDFLLNYPFSTSVKLSTGCTGTLVAEKHVLTAAHCIHDGKTYVKGTQKLRVGFLKPKYKDGAEGDNSSSSAMPDKMKFQWIRVKRTHVPKGWIKGNANDIGMDYDYALLELKKPHKRQFMKIGVSPPAKQLPGGRIHFSGYDNDRPGNLVYRFCDVKDETYDLLYQQCDAQPGASGSGVYVRMWKRPQQKWERKIIGIFSGHQWVDMNGSPQDFNVAVRITPLKYAQICYWIKGNYLDCREG

SEQ ID NO: 51 - protein, 269 aa, Mus musculus
MAAAGRRGLLLLFVLWMMVTVILPASGEGGWKQNGLGIAAAVMEEERCTVERRAHITYSEFMQHYAFLKPVILQGLTDNSKFRALCSRENLLASFGDNIVRLSTANTYSYQKVDLPFQEYVEQLLQPQDPASLGNDTLYFFGDNNFTEWASLFQHYSPPPFRLLGTTPAYSFGIAGAGSGVPFHWHGPGFSEVIYGRKRWFLYPPEKTPEFHPNKTTLAWLLEIYPSLALSARPLECTIQAGEVLYFPDRWWHATLNLDTSVFISTFLG

SEQ ID NO: 52 - protein, 253 aa, Mus musculus
MDNRFATAFVIACVLSLISTIYMAASIGTDFWYEYRSPIQENSSDSNKIAWEDFLGDEADEKTYNDVLFRYNGSLGLWRRCITIPKNTHWYAPPERTESFDVVTKCMSFTLNEQFMEKYVDPGNHNSGIDLLRTYLWRCQFLLPFVSLGLMCFGALIGLCACICRSLYPTLATGILHLLAGLCTLGSVSCYVAGIELLHQKVELPKDVSGEFGWSFCLACVSAPLQFMAAALFIWAAHTNRKEYTLMKAYRVA

SEQ ID NO: 53 - protein, 152 aa, Mus musculus
MGGARDVGWVAAGLVLGAGACYCIYRLTRGPRRGVATMRPSRSAEDLTDGSYDDILNAEQLKKLLYLLESTDDPVITEKALVTLGNNAAFSTNQAIIRELGGIPIVGNKINSLNQSIKEKALNALNNLSVNVENQTKIKIYVPQVCEDVFAD

SEQ ID NO: 54 - protein, 192 aa, Mus musculus
MVTPRPAPARGPALLLLLLLATARGQEQDQTTDWRATLKTIRNGIHKIDTYLNAALDLLGGEDGLCQYKCSDGSKPVPRYGYKPSPPNGCGSPLFGVHLNIGIPSLTKCCNQHDRCYETCGKSKNDCDEEFQYCLSKICRDVQKTLGLSQNVQACETTVELLFDSVIHLGCKPYLDSQRAACWCRYEEKTDL

SEQ ID NO: 55 - DNA, 720 bp, Mus musculus
atggctcagttgggagctgttgtggccgtggcttccagtttcttttgtgcatctctcttctcagctgtgcacaagatagaagagggacatattggagtatattacagaggtggtgccctgctgacctccaccagtggcccgggtttccatctcatgctcccgttcatcacatcctataagtctgtacagaccactctccaaactgatgaagtgaagaacgtaccatgtggaaccagtggtggtgtgatgatctactttgacagaattgaagtggtgaacttcctggtcccaaatgcagtgtatgatatagtgaagaactatactgcagactatgacaaggccctcatcttcaacaagatccatcatgagcttaaccagttctgcagcgttcatactcttcaggaagtctatatcgagctgtttgatcaaattgatgaaaacctcaagttggctttgcagcaggacctgacttccatggcccctgggctggttatccaagctgtgcgagtgacaaagcccaatatacctgaggcaatccgcaggaactatgagctgatggaaagcgagaagacgaagcttctcattgcagcccagaagcagaaggtggtggaaaaggaggcagaaacagagaggaagaaggccctcattgaggcagaaaaagtggcacaggttgcagaaatcacctatgggcaaaaggtgatggagaaggagacagagaag

SEQ ID NO: 56 - DNA, 792 bp, Mus musculus
atgatctatattgaccgaatagaagtggttaatatgttggctccttatgcagtgtttgacattgtgaggaactatactgcagactacgacaagactttaatcttcaataaaatccaccatgagctgaaccagttttgcagtgcccacacacttcaagaagtttacatagaattgtttgatcaaatagatgaaaacctgaagcaggccctgcaaaaagatttaaacaccatggccccaggtctcactatccaggctgtgcgtgttacaaaacccaaaatcccagaagccataagaagaaattttgaattaatggaggcagagaagacaaaacttctcatagctgcacagaaacaaaaggtggtggagaaagaagctgagacggagaggaaaagggctgttatagaagcagagaagattgcacaagtagcaaaaattcgatttcaacagaaagtgatggagaaagaaactgaaaaacgcatttctgagattgaagatgctgcgttcctggcccgagagaaggcaaaagcagatgccgagtattacgctgcacacaaatacgccacctcaaacaagcacaaactgaccccagagtatctggagctcaagaaataccaggccattgcctcaaacagtaagatctactttggcagcaacatccccagcatgtttgtggactcctcctgtgctctgaaatactctgatggtaggactgggagagaagactcccttcccccagaggaggcccgtgagccctctggagagagccccatccaaaacaaggagaacgcaggt

SEQ ID NO: 57 - DNA, 549 bp, Mus musculus
atgaagctgctgtgtttggtggctgtggtggggtgcttgctggtgcccccagctcaagccaacaagagctctgaagatatccggtgcaaatgcatctgtccgccttacagaaacatcagcgggcacatttacaaccagaatgtgtctcagaaggactgcaactgcctgcatgtggtggagcccatgccagtgcctggccacgatgtggaagcctactgcctgctctgcgagtgtaggtacgaggagcgtagcaccacaaccatcaaggtcattattgtcatctacctgtctgtggtgggggccctcttactctacatggccttcctgatgctggtggacccgctcatccggaagccagatgcctatactgagcagctgcacaatgaagaggagaatgaggatgctcgcaccatggcaacagccgctgcgtccattggaggaccccgggcaaacactgtcctggagcgggtggaaggcgctcagcagcggtggaagctgcaggtgcaggagcagcggaagacggtcttcgaccgacacaagatgctcagt

SEQ ID NO: 58 - DNA, 597 bp, Mus musculus
atggcgagcctatggtgcggaaacctgctgcggctgggctcggggctcagcatgtcctgcctggcgctgtcggtgctgctgctcgcgcagctgacaggcgccgccaagaattttgaagatgtgagatgtaaatgcatctgccctccctataaagagaatcctgggcacatttataataagaatatatctcagaaagattgtgattgccttcatgtcgtggagcccatgcctgtacggggacctgatgtagaagcatactgtctacgctgtgaatgcaaatacgaagagagaagctctgtcacaatcaaggttaccattataatttatctctctattttgggccttctgcttctgtacatggtatatcttaccttagttgagcccatcctgaagaggcgcctctttggacactcccagctgttgcagagcgatgatgacgttggggatcaccagccttttgcaaatgcccatgatgtgctggcccgctctcgcagccgagccaatgttctaaacaaggtggagtacgctcagcagcgctggaagctccaggtccaggagcagcgaaagtctgtcttcgaccgacacgttgtcctcagc

SEQ ID NO: 59 - DNA, 579 bp, Mus musculus
atgctgcgctgcggcctggcctgcgagcgctgcaggtggatcctgcccctgctgctgctcagcgccatcgccttcgacatcatcgcgctggccggccgcggctggctgcagtctagcaaccacatccagacatcgtcgctttggtggaggtgtttcgacgagggcggcggcagcggctcctacgacgatggctgccagagcctcatggagtacgcatggggacgagcagctgcagccacgcttttctgtggctttatcatcctgtgcatctgcttcattctctcgttcttcgccctgtgtggaccccagatgcttgttttcctgagagtcattggaggcctcctcgcactggctgccatattccagatcatctccctggtaatctaccccgtgaagtacacacagaccttcaggcttcacgataaccctgctgttaattacatctataactgggcctatggcttcggatgggcggccaccatcatcttgattggttgttccttcttcttctgctgcctccccaactacgaggatgaccttttgggggccgccaagcccaggtacttctatcccccagcc

SEQ ID NO: 60 - DNA, 1146 bp, Mus musculus
atggctggaatcccggggctcttcatccttcttgtcctgctctgtgtgttcatgcaggtgagtccctacaccgttccgtggaaacccacatggccggcttatcgcctccctgtagtcttgcctcagtctaccctcaacttagctaaggcagacttcgacgccaaagcgaaattggaggtgtcctcctcatgtggacctcagtgtcacaagggaacaccactgcccacctacgaagaggccaagcagtacctttcctatgaaaccctttatgccaatggcagccgcacagagactcgggtgggcatctacatcctcagcaatggtgaaggcagggcacgaggcagagactcggaggccacagggagatctcgcaggaagaggcagatttatggctacgatggcaggtttagcatttttgggaaggacttcctgctcaattatcctttctcaacatcggtgaagttgtctactggctgcactggcaccctggtggcagagaagcacgtcctcactgctgcccactgcatacacgatgggaaaacctatgtgaaagggacacagaaactccgagtgggcttcctgaagcccaagtataaagatggtgccgaaggggacaacagctcgagctcagccatgccagacaagatgaagtttcagtggatccgcgtgaaacgcacccatgtgcccaaggggtggatcaagggcaatgccaatgacatcggcatggattatgactacgccctgctggaactcaagaaaccccacaaaagacagttcatgaagattggtgtgagtcctccagcgaagcagctcccagggggcaggatccacttctctggttatgacaatgaccggcccggcaatttggtgtaccgcttctgtgatgtcaaagatgagacctacgaccttctctaccagcagtgtgacgcccagcccggggccagtggttcaggggtctatgtgaggatgtggaagagaccacagcagaaatgggaaagaaaaattatcggcatcttttcagggcaccagtgggtggacatgaatggctctccacaggatttcaacgtggcagttagaatcacgcctcttaaatatgcccagatttgctattggattaaaggaaactacctagattgcagggagggg

SEQ ID NO: 61 - DNA, 807 bp, Mus musculus
atggcggcggctgggcggcgcggtctgcttttgctctttgtactatggatgatggtgactgtgattctgcctgcctctggcgaagggggatggaaacagaatgggctgggaattgcagcagcagtaatggaggaggagcgttgcacagtggagcgtcgggcacacatcacgtactccgaattcatgcagcactatgccttcctcaagcccgtcatcttgcaaggactcacggacaactcgaagttccgggccctgtgttcccgggaaaacctgctagcctcgttcggggacaacattgttcgcttgagtacagccaacacctactcctaccagaaagtggacctgcccttccaggaatatgtggaacagctgctgcagccccaggatcctgcatccctaggcaatgacaccctgtacttttttggagacaacaacttcactgagtgggcatccctcttccagcactactctccgccaccattccgtctcctgggaaccacccctgcttacagctttggaattgcaggagctggatctggggtacccttccactggcatgggcctggtttctcagaggttatctatggtcggaagcgctggttcctctaccctcctgagaagacacctgagttccaccctaacaagaccacattggcctggctgctggaaatatacccatctctagccctgtcagcacggcctctagaatgtaccatccaggctggtgaagtactgtattttcctgatcggtggtggcatgccacactcaatctggacaccagtgtcttcatttctaccttccttggc

SEQ ID NO: 62 - DNA, 759 bp, Mus musculus
atggataaccgttttgctactgcgtttgtgattgcttgtgtgcttagtctgatttccaccatctacatggcggcctccataggcacggacttctggtatgagtatcgaagtcccattcaagagaattcaagtgactcgaataaaatcgcctgggaagatttcctcggtgacgaggcggatgagaagacttacaacgatgttctgttccgatacaacggcagcttggggctgtggagacggtgcatcaccatacccaaaaacactcactggtatgcgccaccggaaaggacagagtcatttgatgtggttaccaaatgcatgagtttcacactaaacgagcagttcatggagaagtatgtggaccccggcaaccacaatagcggcatcgacctgcttcgcacctacctgtggcgctgccagttccttttacccttcgtcagcttgggcttgatgtgctttggggcgttgattggcctctgtgcctgtatctgccgcagcctgtatcccaccctcgccactggcattctccatctccttgcaggtctgtgcacactgggctccgtgagttgctatgttgccggcattgaactcttacatcagaaagtagagctgcccaaggatgtatctggagaatttggatggtccttctgcctggcctgcgtctcggctcccttacagttcatggcggccgctctcttcatctgggctgcccacaccaaccggaaagagtacaccttaatgaaggcttatcgtgtggca

SEQ ID NO: 63 - DNA, 456 bp, Mus musculus
atgggtggcgcgcgggacgtgggctgggtggcagcagggctggtcctgggcgccggcgcctgctactgtatctaccggctgactcggggaccgcggcgaggcgtcgcgaccatgcgcccttcgcgatccgcagaagacctaaccgatggctcctatgacgatatcttaaatgcagagcagcttaagaaacttctgtatctgctggagtcaaccgacgatcctgtcattactgaaaaggccttggtcaccttgggaaataatgcagccttctccactaaccaggccattattcgtgagttgggtggtatcccaattgttggaaacaaaatcaactccctgaaccaaagtattaaagagaaagctttaaatgcactgaataacctgagtgtgaatgttgaaaatcaaactaagataaagatatacgtccctcaagtctgtgaggacgtctttgctgac

SEQ ID NO: 64 - protein, 22 aa, Homo sapiens
MKLLSLVAVVGCLLVPPAEANK

SEQ ID NO: 65 - protein, 22 aa, Homo sapiens
MAQLGAVVAVASSFFCASLFSA

SEQ ID NO: 66 - protein, 23 aa, Homo sapiens
MNMTQARVLVAAVVGLVAVLLYA

SEQ ID NO: 67 - protein, 20 aa, Homo sapiens
MKLLSLVAVVGCLLVPPAEA

SEQ ID NO: 68 - protein, 28 aa, Homo sapiens
MATLWGGLLRLGSLLSLSCLALSVLLLA

SEQ ID NO: 69 - protein, 24 aa, Homo sapiens
MIRCGLACERCRWILPLLLLSAIA

SEQ ID NO: 70 - protein, 20 aa, Homo sapiens
MAGIPGLLFLLFFLLCAVGQ

SEQ ID NO: 71 - protein, 23 aa, Homo sapiens
MAPASRLLALWALAAVALPGSGA

SEQ ID NO: 72 - protein, 24 aa, Homo sapiens
MDNRFATAFVIACVLSLISTIYMA

SEQ ID NO: 73 - protein, 25 aa, Homo sapiens
MDNRFATAFVIACVLSLISTIYMAA

SEQ ID NO: 74 - protein, 21 aa, Homo sapiens
MELGCWTQLGLTFLQLLLISS

SEQ ID NO: 75 - protein, 20 aa, Homo sapiens
MGGPRGAGWVAAGLLLGAGA

SEQ ID NO: 76 - protein, 161 aa, Homo sapiens
SSEDIRCKCICPPYRNISGHIYNQNVSQKDCNCLHVVEPMPVPGHDVEAYCLLCECRYEERSTTTIKVIIVIYLSVVGALLLYMAFLMLVDPLIRKPDAYTEQLHNEEENEDARSMAAAAASLGGPRANTVLERVEGAQQRWKLQVQEQRKTVFDRHKMLS

SEQ ID NO: 77 - protein, 317 aa, Homo sapiens
VHKIEEGHIGVYYRGGALLTSTSGPGFHLMLPFITSYKSVQTTLQTDEVKNVPCGTSGGVMIYFDRIEVVNFLVPNAVYDIVKNYTADYDKALIFNKIHHELNQFCSVHTLQEVYIELFDQIDENLKLALQQDLTSMAPGLVIQAVRVTKPNIPEAIRRNYELMESEKTKLLIAAQKQKVVEKEAETERKKALIEAEKVAQVAEITYGQKVMEKETEKKISEIEDAAFLAREKAKADAECYTAMKIAEANKLKLTPEYLQLMKYKAIASNSKIYFGKDIPNMFMDSAGSVSKQFEGLADKLSFGLEDEPLETATKEN

SEQ ID NO: 78 - protein, 325 aa, Homo sapiens
SIHKIEEGHLAVYYRGGALLTSPSGPGYHIMLPFITTFRSVQTTLQTDEVKNVPCGTSGGVMIYIDRIEVVNMLAPYAVFDIVRNYTADYDKTLIFNKIHHELNQFCSAHTLQEVYIELFDQIDENLKQALQKDLNLMAPGLTIQAVRVTKPKIPEAIRRNFELMEAEKTKLLIAAQKQKVVEKEAETERKKAVIEAEKIAQVAKIRFQQKVMEKETEKRISEIEDAAFLAREKAKADAEYYAAHKYATSNKHKLTPEYLELKKYQAIASNSKIYFGSNIPNMFVDSSCALKYSDIRTGRESSLPSKEALEPSGENVIQNKESTG

SEQ ID NO: 79 - protein, 163 aa, Homo sapiens
NKSSEDIRCKCICPPYRNISGHIYNQNVSQKDCNCLHVVEPMPVPGHDVEAYCLLCECRYEERSTTTIKVIIVIYLSVVGALLLYMAFLMLVDPLIRKPDAYTEQLHNEEENEDARSMAAAAASLGGPRANTVLERVEGAQQRWKLQVQEQRKTVFDRHKMLS

SEQ ID NO: 80 - protein, 170 aa, Homo sapiens
QLSDAAKNFEDVRCKCICPPYKENSGHIYNKNISQKDCDCLHVVEPMPVRGPDVEAYCLRCECKYEERSSVTIKVTIIIYLSILGLLLLYMVYLTLVEPILKRRLFGHAQLIQSDDDIGDHQPFANAHDVLARSRSRANVLNKVEYAQQRWKLQVQEQRKSVFDRHVVLS

SEQ ID NO: 81 - protein, 169 aa, Homo sapiens
FDIIALAGRGWLQSSDHGQTSSLWWKCSQEGGGSGSYEEGCQSLMEYAWGRAAAAMLFCGFIILVICFILSFFALCGPQMLVFLRVIGGLLALAAVFQIISLVIYPVKYTQTFTLHANPAVTYIYNWAYGFGWAATIILIGCAFFFCCLPNYEDDLLGNAKPRYFYTSA

SEQ ID NO: 82 - protein, 363 aa, Homo sapiens
VSPYSAPWKPTWPAYRLPVVLPQSTLNLAKPDFGAEAKLEVSSSCGPQCHKGTPLPTYEEAKQYLSYETLYANGSRTETQVGIYILSSSGDGAQHRDSGSSGKSRRKRQIYGYDSRFSIFGKDFLLNYPFSTSVKLSTGCTGTLVAEKHVLTAAHCIHDGKTYVKGTQKLRVGFLKPKFKDGGRGANDSTSAMPEQMKFQWIRVKRTHVPKGWIKGNANDIGMDYDYALLELKKPHKRKFMKIGVSPPAKQLPGGRIHFSGYDNDRPGNLVYRFCDVKDETYDLLYQQCDAQPGASGSGVYVRMWKRQQQKWERKIIGIFSGHQWVDMNGSPQDFNVAVRITPLKYAQICYWIKGNYLDCREG

SEQ ID NO: 83 - protein, 241 aa, Homo sapiens
EGDGGWRPGGPGAVAEEERCTVERRADLTYAEFVQQYAFVRPVILQGLTDNSRFRALCSRDRLLASFGDRVVRLSTANTYSYHKVDLPFQEYVEQLLHPQDPTSLGNDTLYFFGDNNFTEWASLFRHYSPPPFGLLGTAPAYSFGIAGAGSGVPFHWHGPGYSEVIYGRKRWFLYPPEKTPEFHPNKTTLAWLRDTYPALPPSARPLECTIRAGEVLYFPDRWWHATLNLDTSVFISTFLG

SEQ ID NO: 84 - protein, 227 aa, Homo sapiens
ASIGTDFWYEYRSPVQENSSDLNKSIWDEFISDEADEKTYNDALFRYNGTVGLWRRCITIPKNMHWYSPPERTESFDVVTKCVSFTLTEQFMEKFVDPGNHNSGIDLLRTYLWRCQFLLPFVSLGLMCFGALIGLCACICRSLYPTIATGILHLLAGLCTLGSVSCYVAGIELLHQKLELPDNVSGEFGWSFCLACVSAPLQFMASALFIWAAHTNRKEYTLMKAYR

SEQ ID NO: 85 - protein, 226 aa, Homo sapiens
SIGTDFWYEYRSPVQENSSDLNKSIWDEFISDEADEKTYNDALFRYNGTVGLWRRCITIPKNMHWYSPPERTESFDVVTKCVSFTLTEQFMEKFVDPGNHNSGIDLLRTYLWRCQFLLPFVSLGLMCFGALIGLCACICRSLYPTIATGILHLLAGLCTLGSVSCYVAGIELLHQKLELPDNVSGEFGWSFCLACVSAPLQFMASALFIWAAHTNRKEYTLMKAYR

SEQ ID NO: 86 - protein, 699 aa, Homo sapiens
LPREYTVINEACPGAEWNIMCRECCEYDQIECVCPGKREVVGYTIPCCRNEENECDSCLIHPGCTIFENCKSCRNGSWGGTLDDFYVKGFYCAECRAGWYGGDCMRCGQVLRAPKGQILLESYPLNAHCEWTIHAKPGFVIQLRFVMLSLEFDYMCQYDYVEVRDGDNRDGQIIKRVCGNERPAPIQSIGSSLHVLFHSDGSKNFDGFHAIYEEITACSSSPCFHDGTCVLDKAGSYKCACLAGYTGQRCENLLEERNCSDPGGPINGYQKITGGPGLINGRHAKIGTVVSFFCYNSYVLSGNEKRTCQQNGEWSGKQPICIKACREPKISDLVRRRVLPMQVQSRETPLHQLYSAAFSKQKLQSAPTKKPALPFGDLPMGYQHLHTQLQYECISPFYRRLGSSRRTCLRTGKWSGRAPSCIPICGKIENITAPKTQGLRWPWQAAIYRRTSGVHDGSLHKGAWFLVCSGALVNERTVVVAAHCVTDLGKVTMIKTADLKVVLGKFYRDDDRDEKTIQSLQISAIILHPNYDPILLDADIAILKLLDKARISTRVQPICLAASRDLSTSFQESHITVAGWNVLADVRSPGFKNDTLRSGVVSVVDSLLCEEQHEDHGIPVSVTDNMFCASWEPTAPSDICTAETGGIAAVSFPGRASPEPRWHLMGLVSWSYDKTCSHRLSTAFTKVLPFKDWIERNMK

SEQ ID NO: 87 - protein, 323 aa, Homo sapiens
CYCIYRLTRGRRRGDRELGIRSSKSAGALEEGTSEGQLCGRSARPQTGGTWESQWSKTSQPEDLTDGSYDDVLNAEQLQKLLYLLESTEDPVIIERALITLGNNAAFSVNQAIIRELGGIPIVANKINHSNQSIKEKALNALNNLSVNVENQIKIKIYISQVCEDVFSGPLNSAVQLAGLTLLTNMTVTNDHQHMLHSYITDLFQVLLTGNGNTKVQVLKLLLNLSENPAMTEGLLRAQVDSSFLSLYDSHVAKEILLRVLTLFQNIKNCLKIEGHLAVQPTFTEGSLFFLLHGEECAQKIRALVDHHDAEVKEKVVTIIPKI

SEQ ID NO: 88 - protein, 69 aa, Homo sapiens
NKSSEDIRCKCICPPYRNISGHIYNQNVSQKDCNCLHVVEPMPVPGHDVEAYCLLCECRYEERSTTTIK

SEQ ID NO: 89 - protein, 74 aa, Homo sapiens
QLSDAAKNFEDVRCKCICPPYKENSGHIYNKNISQKDCDCLHVVEPMPVRGPDVEAYCLRCECKYEERSSVTIK

SEQ ID NO: 90 - protein, 51 aa, Homo sapiens
FDIIALAGRGWLQSSDHGQTSSLWWKCSQEGGGSGSYEEGCQSLMEYAWGR

SEQ ID NO: 91 - protein, 20 aa, Homo sapiens
VKYTQTFTLHANPAVTYIYN

SEQ ID NO: 92 - protein, 114 aa, Homo sapiens
ASIGTDFWYEYRSPVQENSSDLNKSIWDEFISDEADEKTYNDALFRYNGTVGLWRRCITIPKNMHWYSPPERTESFDVVTKCVSFTLTEQFMEKFVDPGNHNSGIDLLRTYLWR

SEQ ID NO: 93 - protein, 113 aa, Homo sapiens
SIGTDFWYEYRSPVQENSSDLNKSIWDEFISDEADEKTYNDALFRYNGTVGLWRRCITIPKNMHWYSPPERTESFDVVTKCVSFTLTEQFMEKFVDPGNHNSGIDLLRTYLWR

SEQ ID NO: 94 - protein, 23 aa, Homo sapiens
VIIVIYLSVVGALLLYMAFLMLV

SEQ ID NO: 95 - protein, 21 aa, Homo sapiens
VTIIIYLSILGLLLLYMVYLT

SEQ ID NO: 96 - protein, 27 aa, Homo sapiens
AAAAMLFCGFIILVICFILSFFALCGP

SEQ ID NO: 97 - protein, 22 aa, Homo sapiens
VIGGLLALAAVFQIISLVIYPV

SEQ ID NO: 98 - protein, 24 aa, Homo sapiens
WAYGFGWAATIILIGCAFFFCCLP

SEQ ID NO: 99 - protein, 26 aa, Homo sapiens
CQFLLPFVSLGLMCFGALIGLCACIC

SEQ ID NO: 100 - protein, 18 aa, Homo sapiens
LLAGLCTLGSVSCYVAGI

SEQ ID NO: 101 - protein, 26 aa, Homo sapiens
FGWSFCLACVSAPLQFMASALFIWAA

SEQ ID NO: 102 - protein, 71 aa, Homo sapiens
DPLIRKPDAYTEQLHNEEENEDARSMAAAAASLGGPRANTVLERVEGAQQRWKLQVQEQRKTVFDRHKMLS

SEQ ID NO: 103 - protein, 73 aa, Homo sapiens
EPILKRRLFGHAQLIQSDDDIGDHQPFANAHDVLARSRSRANVLNKVEYAQQRWKLQVQEQRKSVFDRHVVLS

SEQ ID NO: 104 - protein, 7 aa, Homo sapiens
QMLVFLR

SEQ ID NO: 105 - protein, 19 aa, Homo sapiens
NYEDDLLGNAKPRYFYTSA

SEQ ID NO: 106 - protein, 13 aa, Homo sapiens
RSLYPTIATGILH

SEQ ID NO: 107 - protein, 16 aa, Homo sapiens
HTNRKEYTLMKAYRVA

SEQ ID NO: 108 - DNA, 916 bp, Mus musculus
  CDS (167)...(913)
gtcgacccacgcgtccgcggacgcgtgggcgcggactgatggcgtcatcgaagcgactggcccggaaggaagtagggtgctgaggggtgtggcggtttctacggttgcacgggggttcggctgtgtacggagcgcctggagggacagcctggatacaggttcactgatggctcagttgggagctgttgtggccgtggcttccagtttcttttgtgcatctctcttctcagctgtgcacaagatagaagagggacatattggagtatattacagaggtggtgccctgctgacctccaccagtggcccgggtttccatctcatgctcccgttcatcacatcctataagtctgtacagaccactctccaaactgatgaagtgaagaacgtaccatgtggaaccagtggtggtgtgatgatctactttgacagaattgaagtggtgaacttcctggtcccaaatgcagtgtatgatatagtgaagaactatactgcagactatgacaaggccctcatcttcaacaagatccatcatgagcttaaccagttctgcagcgttcatactcttcaggaagtctatatcgagctgtttgatcaaattgatgaaaacctcaagttggctttgcagcaggacctgacttccatggcccctgggctggttatccaagctgtgcgagtgacaaagcccaatatacctgaggcaatccgcaggaactatgagctgatggaaagcgagaagacgaagcttctcattgcagcccagaagcagaaggtggtggaaaaggaggcagaaacagagaggaagaaggccctcattgaggcagaaaaagtggcacaggttgcagaaatcacctatgggcaaaaggtgatggagaaggagacagagaagaatgtgaaaagatgtgtagtcctgagttaa

SEQ ID NO: 109 - protein, 249 aa, Mus musculus
MAQLGAVVAVASSFFCASLFSAVHKIEEGHIGVYYRGGALLTSTSGPGFHLMLPFITSYKSVQTTLQTDEVKNVPCGTSGGVMIYFDRIEVVNFLVPNAVYDIVKNYTADYDKALIFNKIHHELNQFCSVHTLQEVYIELFDQIDENLKLALQQDLTSMAPGLVIQAVRVTKPNIPEAIRRNYELMESEKTKLLIAAQKQKVVEKEAETERKKALIEAEKVAQVAEITYGQKVMEKETEKNVKRCVVLS

SEQ ID NO: 110 - DNA, 747 bp, Mus musculus
atggctcagttgggagctgttgtggccgtggcttccagtttcttttgtgcatctctcttctcagctgtgcacaagatagaagagggacatattggagtatattacagaggtggtgccctgctgacctccaccagtggcccgggtttccatctcatgctcccgttcatcacatcctataagtctgtacagaccactctccaaactgatgaagtgaagaacgtaccatgtggaaccagtggtggtgtgatgatctactttgacagaattgaagtggtgaacttcctggtcccaaatgcagtgtatgatatagtgaagaactatactgcagactatgacaaggccctcatcttcaacaagatccatcatgagcttaaccagttctgcagcgttcatactcttcaggaagtctatatcgagctgtttgatcaaattgatgaaaacctcaagttggctttgcagcaggacctgacttccatggcccctgggctggttatccaagctgtgcgagtgacaaagcccaatatacctgaggcaatccgcaggaactatgagctgatggaaagcgagaagacgaagcttctcattgcagcccagaagcagaaggtggtggaaaaggaggcagaaacagagaggaagaaggccctcattgaggcagaaaaagtggcacaggttgcagaaatcacctatgggcaaaaggtgatggagaaggagacagagaagaatgtgaaaagatgtgtagtcctgagt

SEQ ID NO: 111 - DNA, 3087 bp, Mus musculus
  CDS (45)...(1088)
  misc_feature (1)...(3087): n = A,T,C or G
ggaaccccgcgtccggngatgcgtcactgaccggaggaacaaggatgaatatgactcaagcccggcttctggtggctgcagtggtggggttggtggcgatcctcctgtacgcctccatccacaagatcgaagagggacacttggccgtgtactacaggggaggagctttgctaacgagccccagtggaccaggctatcatatcatgttgcctttcattacaacattcagatctgtgcagacaacactacaaacggatgaagttaaaaatgtgccttgtggaacaagtggtggagtcatgatctatattgaccgaatagaagtggttaatatgttggctccttatgcagtgtttgacattgtgaggaactatactgcagactacgacaagactttaatcttcaataaaatccaccatgagctgaaccagttttgcagtgcccacacacttcaagaagtttacatagaattgtttgatcaaatagatgaaaacctgaagcaggccctgcaaaaagatttaaacaccatggccccaggtctcactatccaggctgtgcgtgttacaaaacccaaaatcccagaagccataagaagaaattttgaattaatggaggcagagaagacaaaacttctcatagctgcacagaaacaaaaggtggtggagaaagaagctgagacggagaggaaaagggctgttatagaagcagagaagattgcacaagtagcaaaaattcgatttcaacagaaagtgatggagaaagaaactgaaaaacgcatttctgagattgaagatgctgcgttcctggcccgagagaaggcaaaagcagatgccgagtattacgctgcacacaaatacgccacctcaaacaagcacaaactgaccccagagtatctggagctcaagaaataccaggccattgcctcaaacagtaagatctactttggcagcaacatccccagcatgtttgtggactcctcctgtgctctgaaatactctgatggtaggactgggagagaagactcccttcccccagaggaggcccgtgagccctctggagagagccccatccaaaacaaggagaacgcaggttgatgcaagaggtggaaatgttctcccatatcaagatgcgacccaaggggctaagtgggaacagtggttatgtggactcgtaagattcacagagaatgtgtgctctgttgtgattctcttgtcatagtcctggtttgccagctgactacaggatagacccagctgtctggcactcaaacggtctctgcagccacagttttatcaagtatcctgtatgtgttcctttgtaaaccggtactcatgaatgagggaaagtctgatgctaagatactgcctgcactggaatgtcaaacactatataacaagctgtggtttttaaaagctattgaataatgtttacattggtccctgaggacatgtgtgctcagacattcaagagctaggaggccagagagaagaccttcagaaaacggtaagttaaagaagacaagtgtcatcagacacttgggacccgggctctctttaaagtctagtcccggcattcctccatgtgattgacagccagacctctgggttcccaggaaattatcttccagttgaatgaccatttacttgatacaaattgtacctttctgtttttctagtcaggttggtggcctgcagggacgcgtactttgccacccgaccagaggttcctcgaagatattcccaatcactagtttattgcgttaggagactcagagatatagaaagcagctgaaatttaagggagataaagcctgcactgcaccaaagctacgggtccctgtgtttcctctattcagtgatgtcatcaacctcactgtcccagcccatgtgtgactaaagtgcccggttttagccacagacaactgcttagatgtcacctcttggctgaccaaagctgggacagggctttaaccagacataggagcagtgtgcaattcctgattcactgcacagtattatgtcataattgcaggaattattttttgtttttaaaactggatttggggcacattcattcaccccaacacttctatctaaaggccaaggttctagggctgctatggtcactaacacactgattctccttaaagtaattctcgaagtgtggaacaaagtgaccgagacagcatcctcagtcatctttgtctccttccctgggatgcagataccgaagttgcttttccaactttcgcctccgctaggagatcagaaagaattcttgtgacttcctgggcagccattgaattcattttccatgagaagatgacagagttagcctgtggctataggagatcatgtcatccagacctttttgcccatcacattaactttcctggaatattgtgctgcacaggtagacctgaatctgcccagcttgttgacagctcttgtgtatactgtgttgaagccagacagaaaagtaatggggccacttctgaaacctctcagctgttgatctcacagcagctaaagggttgtgccaaacattttattaagaaagtaaagcccagatttgaatgggggttttccctaggccttatagtatagaggcatttgtaatatggagaaaataatttttctcatttaattatagaaattaccttcaaacagattttgtgttctttggcccttcaaatactggtgttacattgttgctgcagataaatgatgattgtcgtgggatatctggatcactgagctctgtgctttcattcctagagatgtttctcattcccatttagtgaaatgctgttgccccaaagtgatggttgtgggatttcttaccggtcataggccccggtgaggagcagggaagcgccattgtgaaagattaaagaaagcacttccacttgagctccttatggagtgagcttccctgtgcccactcagtgaactaagtctgaccatccttcagggacgttccttttggtaaatatacactgtaatctttaagtctaaatttatatgtgaaagttaactttttttaaaaacctaaataaaattattttcctatcaaaaaaaaaaaaaaaaa

SEQ ID NO: 112 - protein, 348 aa, Mus musculus
MNMTQARLLVAAVVGLVAILLYASIHKIEEGHLAVYYRGGALLTSPSGPGYHIMLPFITTFRSVQTTLQTDEVKNVPCGTSGGVMIYIDRIEVVNMLAPYAVFDIVRNYTADYDKTLIFNKIHHELNQFCSAHTLQEVYIELFDQIDENLKQALQKDLNTMAPGLTIQAVRVTKPKIPEAIRRNFELMEAEKTKLLIAAQKQKVVEKEAETERKRAVIEAEKIAQVAKIRFQQKVMEKETEKRISEIEDAAFLAREKAKADAEYYAAHKYATSNKHKLTPEYLELKKYQAIASNSKIYFGSNIPSMFVDSSCALKYSDGRTGREDSLPPEEAREPSGESPIQNKENAG

SEQ ID NO: 113 - DNA, 1044 bp, Mus musculus
atgaatatgactcaagcccggcttctggtggctgcagtggtggggttggtggcgatcctcctgtacgcctccatccacaagatcgaagagggacacttggccgtgtactacaggggaggagctttgctaacgagccccagtggaccaggctatcatatcatgttgcctttcattacaacattcagatctgtgcagacaacactacaaacggatgaagttaaaaatgtgccttgtggaacaagtggtggagtcatgatctatattgaccgaatagaagtggttaatatgttggctccttatgcagtgtttgacattgtgaggaactatactgcagactacgacaagactttaatcttcaataaaatccaccatgagctgaaccagttttgcagtgcccacacacttcaagaagtttacatagaattgtttgatcaaatagatgaaaacctgaagcaggccctgcaaaaagatttaaacaccatggccccaggtctcactatccaggctgtgcgtgttacaaaacccaaaatcccagaagccataagaagaaattttgaattaatggaggcagagaagacaaaacttctcatagctgcacagaaacaaaaggtggtggagaaagaagctgagacggagaggaaaagggctgttatagaagcagagaagattgcacaagtagcaaaaattcgatttcaacagaaagtgatggagaaagaaactgaaaaacgcatttctgagattgaagatgctgcgttcctggcccgagagaaggcaaaagcagatgccgagtattacgctgcacacaaatacgccacctcaaacaagcacaaactgaccccagagtatctggagctcaagaaataccaggccattgcctcaaacagtaagatctactttggcagcaacatccccagcatgtttgtggactcctcctgtgctctgaaatactctgatggtaggactgggagagaagactcccttcccccagaggaggcccgtgagccctctggagagagccccatccaaaacaaggagaacgcaggt

SEQ ID NO: 114 - DNA, 2883 bp, Mus musculus
  CDS (211)...(924)
gtccaggaaaaagctgcttgcactaggggcatcccgcctgcctggtgaaaggaaccgcagcacacagggtgggagggcttccgattttagcagggcggcttccggaaggcggagctccaaccccatttcctttctctgggctggttctggcccagctgcacctgcgtgtggccctggctcctcggctccctgcagctccgaggcagcagcatgggtggcgcgcgggacgtgggctgggtggcagcagggctggtcctgggcgccggcgcctgctactgtatctaccggctgactcggggaccgcggcgaggcggtcgccgactgcgcccttcgcgatccgcagaagacctaaccgatggctcctatgacgatatcttaaatgcagagcagcttaagaaacttctgtatctgctggagtcaaccgacgatcctgtcattactgaaaaggccttggtcaccttgggaaataatgcagccttctccactaaccaggccattattcgtgagttgggtggtatcccaattgttggaaacaaaatcaactccctgaaccaaagtattaaagagaaagctttaaatgcactgaataacctgagtgtgaatgttgaaaatcaaactaagataaagatatacgtccctcaagtctgtgaggacgtctttgctgaccccctgaactctgcggtgcagctggccggactgaggctgctgacaaacatgacggtcaccaacgactatcagcacctgctcagcggctccgtcgctggcctgttccacctgctgctgctgggaaacggaagcaccaaggtccaggttttgaagctgcttttgaatttgtctgagaattcagccatgacagaaggactactgagtgtccaagtaagtagattacctacccggttcattagtgcacacatacagagattttgacaaatagatctgcaaaggtatgcccaaaaacattcacaggaattatttctgaagatgagtattaagcatattttgttttttaaaacttctctgtggcaccagcagactttccatctctggccactttgcagtatttttctgtcactgcattttaaagtttgttttttttgtgcatgtgtacctcagcatttgctgaaacaactgtactgagtgagtcccctgtgtgggctcggtcctgagcattcagccagcaccagcaagttcttagtgttcccatggaacttaggagaagcaaccatgtaacaaattagcaagactgttgaaaacatgtaacaaaccattgaaacagtccctgtgctctgaagaaggccaggcggtgtgagccgtctgcagaaatcgagccatctgctccgtcctgttaccagaactgtgtgtaagagctaatgctgattgaactaatgtgttcttacaaaaactggatagatcctaaaggggttggtttcccaaatggctacactctggagttccaaagaaatcttagtttttcccctaacaaaacgtcattttcacttgtaacatggaataaaaatgaaacatgtcccttacgcttgcctggagtcagacttttacagtgttaactaatggatgctgttttaaaataggacagtgacgctgtttcctctttcaggtggattcttcattcctttccctttatgacggccaagtagcaaatgagattcttcttcgggctcttacactgtttcagaatataaacaactgcctcaaagtggaaggccggttagctaatcagattccttttgctaaagggtcattgttttttctgttatacggagaagaatgtgcccagaaaatgagagctttagcctgtcatcatgatgtggatgtgaaagagaaagctttagcaataaagccgaaattctgatcggttgctcctatttttatcaaagactcaaacagtaaggcagtcttaagtcagcacacgggagcgtttgcctgcctttaaaaggggtctttcagccgatggagttaaacaataaaagtgagtgagcagctctaatccaacacgatgttcaaaattttagattttggagtagttcagatttggggtttggggattgagtagagtctggaaccttccgaggatgtggatcatttacggggcaaacgtttggttatgatcgtggacagactggccatgctcttcaggactatttgaaggattctagtgctagtgaatgaatatgaggggctgtactgaagatacttgctgaggtatttaatggtttcctgacacgaactgagtggcctgtctctgtacaatcctaactcctgggagcatttgcagttgctcatgagacagcgttaagtgctgagttgaagtctgttactgccacagcaaggaccttgtgcctcaaaccagtgaatactgcaagctcgagtccaccaccaaccctgccatgctgcttgcaagtctgagctcatcgtgagacactgcctgcagcatttctgatcagtaggactgtactcccatttacatggaaagcgttttcttactgcttacccccttgtgtaagatactgcagagcactccaagcttccacccacaggcagacagccctttaaaaaagagtgtcctgataagtccagatggatacatggagaaacatacccatgagatggctgctttgaaagcatgctgggaagcaatgtattagggtcccgtgtcttttttttctctcagtaatgataaatacacttatacatggacagaacatttctagaacgattcagaaaacttctgggactgggactagggtacatagatttctttgtgttcctgtttctaccgtttggatttgtactgagcataaattgtataattttttaataaaaaggaaaaatgcaaggtgtacataaaaaaaaaaaaaaaaaaaa

SEQ ID NO: 115 - protein, 238 aa, Mus musculus
MGGARDVGWVAAGLVLGAGACYCIYRLTRGPRRGGRRLRPSRSAEDLTDGSYDDILNAEQLKKLLYLLESTDDPVITEKALVTLGNNAAFSTNQAIIRELGGIPIVGNKINSLNQSIKEKALNALNNLSVNVENQTKIKIYVPQVCEDVFADPLNSAVQLAGLRLLTNMTVTNDYQHLLSGSVAGLFHLLLLGNGSTKVQVLKLLLNLSENSAMTEGLLSVQVSRLPTRFISAHIQRF

SEQ ID NO: 116 - DNA, 714 bp, Mus musculus
atgggtggcgcgcgggacgtgggctgggtggcagcagggctggtcctgggcgccggcgcctgctactgtatctaccggctgactcggggaccgcggcgaggcggtcgccgactgcgcccttcgcgatccgcagaagacctaaccgatggctcctatgacgatatcttaaatgcagagcagcttaagaaacttctgtatctgctggagtcaaccgacgatcctgtcattactgaaaaggccttggtcaccttgggaaataatgcagccttctccactaaccaggccattattcgtgagttgggtggtatcccaattgttggaaacaaaatcaactccctgaaccaaagtattaaagagaaagctttaaatgcactgaataacctgagtgtgaatgttgaaaatcaaactaagataaagatatacgtccctcaagtctgtgaggacgtctttgctgaccccctgaactctgcggtgcagctggccggactgaggctgctgacaaacatgacggtcaccaacgactatcagcacctgctcagcggctccgtcgctggcctgttccacctgctgctgctgggaaacggaagcaccaaggtccaggttttgaagctgcttttgaatttgtctgagaattcagccatgacagaaggactactgagtgtccaagtaagtagattacctacccggttcattagtgcacacatacagagattt

SEQ ID NO: 117 - DNA, 2823 bp, Mus musculus
  CDS (29)...(2188)
ctcggtaccgacacagcaacgggaaacgatggagctagacagatgggcgcagttggggctggtgttcctgcagctccttctcatctcatcgttgccaagagagtacacggtcattaatgaagcctgtcccggagctgagtggaacatcatgtgtagagaatgttgtgaatatgatcagattgaatgcctctgcccaggaaagaaggaagtggtgggttacaccatcccatgctgcaggaatgaggataatgaatgtgactcctgtctaattcacccaggttgtaccatctttgaaaactgcaagagctgccgcaatggctcctggggcggaactctggatgacttctacgtgaagggattctactgcgcagagtgcagggcaggctggtacggaggagactgcatgcgatgtggccaggttcttcgagcctcaaagggtcagatcttgttggagagctatcccttaaacgctcactgtgaatggactattcatgccagacctgggtttatcatccagttgaggtttggtatgctgagcctagagtttgactacatgtgccaatatgactatgtggaggtccgcgatggggataatagtgacagccctatcatcaagcgtttctgtggcaacgagaggccagctcccatcaggagcactggctcttcactccatgtccttttccattctgatggctccaagaacttcgatggcttccacgctgtctttgaggagatcacagcgtgctcctcatccccttgtttccatgatggcacatgcctccttgacaccactgggtctttcaagtgtgcctgcctggctggctacactgggcagcgctgtgaaaatctacttgaagaaagaaactgctcagaccttggggggccagtcaatgggtacaagaaaatcacagaaggtcctggacttctcaatgagcgccatgtaaaaattggcacggttgtgtctttcttttgtaacggctcatacgttctgagtggcaatgagaaacgaacttgccagcagaatggagagtggtcaggaaagcaacctgtctgcatgaaagcctgccgggaaccgaagatctcagacctggtgagaaggagagtcctttcgatgcaggttcagtcaagggagacaccattacatcagctttattccacggctttcagcaagcagaaattgcaggatgcctctaccaaaaagccagcccttccatttggagacctgccccctggataccaacatctgcacacccaagtccagtatgagtgcatctcgcccttctaccgccgcctgggaagcagcaggaggacatgcctgagaactgggaagtggagtgggcgggccccgtcctgtatcccaatctgtggaaaaatcgagagcactccttctccaaagacccaaggcacccgctggccatggcaggcagccatctaccggaggaccagtggtgtacacgatggtggtctgcacaaaggtgcatggttcttggtctgcagtggtgccctggtgaatgaacggactgtggttgtggctgcccactgtgtgactgagctggggaaggccaccatcatcaagacagcagacctcaaggttgtcttgggaaaattctacagggacgatgatcgggatgagaagagcatccagaatttacgggtttctgctatcattctgcaccccaactatgaccctatcctgcttgacactgacatcgctgttctgaagctcctagacaaagctcgcatcagtacccgtgtccaacccatctgcctggctaccactcgggacctcagcacctctttccaggaatcccacatcactgtggctggctggaacatcctggcagatgtgaggagccctggctttaagaatgataccttacattatggaatggtcagagtggtagacccaatgctttgtgaggaacagcatgaagaccatggcattccagttagtgtcactgacaacatgttctgtgccagcaaagatcccagtaccccttctgacatctgcactgcagagacagggggcatcgctgctttgtccttcccaggccgagcatcccccgagccacgctggcatttggtggggctggtcagctggagctatgacaagacatgtagcaatggcctatccacagccttcacaaaggtgttgccgttcaaagactggattgagagaaacatgaaatgaaccagccacaaggccactgagaagccttttcctagcatccgtctgtacatatgttgtatagaacaatgcgggcctgaagtgtaattttgcccaccatcttggctactgaaaggctcctggtttcagggacttatctcaatagagggtgaacagagtttacttcatcagggaactgtctccctgactgcttgggaatcatctaaaagatgccaggtcttgcaacaactggatttcttcaaagaagaccatgtgactagaaggagaacctcttgctcctgctccactcagagtgatgtgactgtcaatcagtttgggttgagaaggttgatttggggaggcctgggctgcacctggcttctgtcaaagttccaaagaacaaacaacttagactagcccagggcaaaggagattgggtgtggcaccctgtgtaaattgtcacaagattgtctgatcctttccctttccaatcttctgtacacatttcaataaaacaaggtctgctccctgacctaccaaacaaaaaaaaaaaaaaaaaaaaaaaaaaaaaaaaaaatacacatttcaataaaacaaggtctgctccctgacctaccaaacaaaaaaaaaaaaaaaaaaaaaaaaaaaaaaaaaaa

SEQ ID NO: 118 - protein, 720 aa, Mus musculus
MELDRWAQLGLVFLQLLLISSLPREYTVINEACPGAEWNIMCRECCEYDQIECLCPGKKEVVGYTIPCCRNEDNECDSCLIHPGCTIFENCKSCRNGSWGGTLDDFYVKGFYCAECRAGWYGGDCMRCGQVLRASKGQILLESYPLNAHCEWTIHARPGFIIQLRFGMLSLEFDYMCQYDYVEVRDGDNSDSPIIKRFCGNERPAPIRSTGSSLHVLFHSDGSKNFDGFHAVFEEITACSSSPCFHDGTCLLDTTGSFKCACLAGYTGQRCENLLEERNCSDLGGPVNGYKKITEGPGLLNERHVKIGTVVSFFCNGSYVLSGNEKRTCQQNGEWSGKQPVCMKACREPKISDLVRRRVLSMQVQSRETPLHQLYSTAFSKQKLQDASTKKPALPFGDLPPGYQHLHTQVQYECISPFYRRLGSSRRTCLRTGKWSGRAPSCIPICGKIESTPSPKTQGTRWPWQAAIYRRTSGVHDGGLHKGAWFLVCSGALVNERTVVVAAHCVTELGKATIIKTADLKVVLGKFYRDDDRDEKSIQNLRVSAIILHPNYDPILLDTDIAVLKLLDKARISTRVQPICLATTRDLSTSFQESHITVAGWNILADVRSPGFKNDTLHYGMVRVVDPMLCEEQHEDHGIPVSVTDNMFCASKDPSTPSDICTAETGGIAALSFPGRASPEPRWHLVGLVSWSYDKTCSNGLSTAFTKVLPFKDWIERNMK

SEQ ID NO: 119 - DNA, 2160 bp, Mus musculus
atggagctagacagatgggcgcagttggggctggtgttcctgcagctccttctcatctcatcgttgccaagagagtacacggtcattaatgaagcctgtcccggagctgagtggaacatcatgtgtagagaatgttgtgaatatgatcagattgaatgcctctgcccaggaaagaaggaagtggtgggttacaccatcccatgctgcaggaatgaggataatgaatgtgactcctgtctaattcacccaggttgtaccatctttgaaaactgcaagagctgccgcaatggctcctggggcggaactctggatgacttctacgtgaagggattctactgcgcagagtgcagggcaggctggtacggaggagactgcatgcgatgtggccaggttcttcgagcctcaaagggtcagatcttgttggagagctatcccttaaacgctcactgtgaatggactattcatgccagacctgggtttatcatccagttgaggtttggtatgctgagcctagagtttgactacatgtgccaatatgactatgtggaggtccgcgatggggataatagtgacagccctatcatcaagcgtttctgtggcaacgagaggccagctcccatcaggagcactggctcttcactccatgtccttttccattctgatggctccaagaacttcgatggcttccacgctgtctttgaggagatcacagcgtgctcctcatccccttgtttccatgatggcacatgcctccttgacaccactgggtctttcaagtgtgcctgcctggctggctacactgggcagcgctgtgaaaatctacttgaagaaagaaactgctcagaccttggggggccagtcaatgggtacaagaaaatcacagaaggtcctggacttctcaatgagcgccatgtaaaaattggcacggttgtgtctttcttttgtaacggctcatacgttctgagtggcaatgagaaacgaacttgccagcagaatggagagtggtcaggaaagcaacctgtctgcatgaaagcctgccgggaaccgaagatctcagacctggtgagaaggagagtcctttcgatgcaggttcagtcaagggagacaccattacatcagctttattccacggctttcagcaagcagaaattgcaggatgcctctaccaaaaagccagcccttccatttggagacctgccccctggataccaacatctgcacacccaagtccagtatgagtgcatctcgcccttctaccgccgcctgggaagcagcaggaggacatgcctgagaactgggaagtggagtgggcgggccccgtcctgtatcccaatctgtggaaaaatcgagagcactccttctccaaagacccaaggcacccgctggccatggcaggcagccatctaccggaggaccagtggtgtacacgatggtggtctgcacaaaggtgcatggttcttggtctgcagtggtgccctggtgaatgaacggactgtggttgtggctgcccactgtgtgactgagctggggaaggccaccatcatcaagacagcagacctcaaggttgtcttgggaaaattctacagggacgatgatcgggatgagaagagcatccagaatttacgggtttctgctatcattctgcaccccaactatgaccctatcctgcttgacactgacatcgctgttctgaagctcctagacaaagctcgcatcagtacccgtgtccaacccatctgcctggctaccactcgggacctcagcacctctttccaggaatcccacatcactgtggctggctggaacatcctggcagatgtgaggagccctggctttaagaatgataccttacattatggaatggtcagagtggtagacccaatgctttgtgaggaacagcatgaagaccatggcattccagttagtgtcactgacaacatgttctgtgccagcaaagatcccagtaccccttctgacatctgcactgcagagacagggggcatcgctgctttgtccttcccaggccgagcatcccccgagccacgctggcatttggtggggctggtcagctggagctatgacaagacatgtagcaatggcctatccacagccttcacaaaggtgttgccgttcaaagactggattgagagaaacatgaaa

SEQ ID NO: 120 - DNA, 2490 bp, Homo sapiens
  CDS (386)...(1237)
  misc_feature (1)...(2490): n = A,T,C or G
ccacgcgtccggccaggggcgggagggaggaatggttgcttcacgccccgggggaagagacgggaagctcggctctgggttgcgggccccggcgtctccgcgtggggcgcaccgtccgacccgcccctcccggtgtgcagcgccccgcaccgccccgcctcgcctgggagaagccgccgggacgcgccgggctggagtgggcggttataggctttgagctaggccgtttccgggaggcggagctcagaccccatttcctttctccacatccaggtcaggtggcgtttgctgtggcggctaggcccgcgtgcgctggagacctccgcgctggcccccgcgagcctcctgccctggcccggcgctgcggctctgccgcggcggcagcatgggtggcccccggggcgcgggctgggtggcggcgggcctgctgctcggcgcgggcgcctgctactgcatttacaggctgacccggggtcggcggcggggcgaccgcgagctcgggatacgctcttcgaagtccgcaggtgccctggaagaagggacgtcagagggtcanttgtgcgggcgctcggcccggcctcagacnggaggtacctgggagtcacagtggtccaagacctcgcancctgaagacttaactgatggttcatatgatgatgttctaaatgctgaacaacttcagaaactcctttacctgctggagtcaacggaggatcctgtaattattgaaagagctttgattactttgggtaacaatgcagccttttcagttaaccaagctattattcgtgaattgggtggtattccaattgttgcaaacaaaatcaaccattccaaccagagtattaaagagaaagctttaaatgcactaaataacctgagtgtgaatgttgaaaatcaaatcaagataaaggtgcaagttttgaaactgcttttgaatttgnctgaaaatccagccatgacagaaggacttctccgtgcccaagtggattcatcattcctttycctttatgacagccacgtagcaaaggagattcttcttcgagtacttacgctatttcagaatataaagaactgcctcaaaatagaaggccatttagctgtgcagcctactttcactgaaggttcattgtttttcctgttacatggagaagaatgtgcccagaaaataagagctttagttgatcaccatgatgcagaggtgaaggaaaaggttgtaacaataatacccaaaatctgattggtcatatttttccaaagagtaatgcagtctggatataaatgtattttctgtcttccttataaggggattctcccagctgctaaatttaaacagtaaatatcacattttgtcattaacacagctataacttgccgtggttctcagatttattttggactattttgatgccaagtgaatataagagcttgtactgaaaccatttatttctttctattttgctatttgcaaatgcttgttatcttccctacatgaagtggcagtaacctttttcacatttaagctacccttctaccttttgaagtgatttgcagttactcatctgagacagcatcagtatttgactaaatcattgtttcacaactgaatagtcttgttcttttagtagcaatgaaatcctaagctcttgaggccattcacctgccaacctgaccatactgctttcaaaagtcttttctcatcagtagaatctattttggtcacttctagtcaatgaaaaatgtaaacttttaggagagaatgtttcctaggactcacccactccattcaatgttacatataaaatagtgtgatcaatcacaatgtccatctttagacagttggttaaataaattatctggtctttgaaaagaccgtgctgggcgcggtggctcttgcctgtaatcccagcactttgggaggctgaggcgggcagatcacctgagatcgggagtttgagaccaagcctgaccaatatggagaaaccctgtctctactaagaatacaaaattagctgggcatggtggtgcatgcctgtaatcccagctacttgggaggccgaggcaggagaattgcttgaacccgggaggcagaggttgcagtgaggtgagatagcgccattgcactccagcctgggcaacaagagcaaaactctgtctcaaaaaaaaaaaaaaatgatggagctccgaatgtgcttaagtggaaagatatctatgaaatatggtggttttttaaaacacaaaaattatagaatatgggatcccgtgtgtgtgtgtgtgtgtgtgtgtgtgtgtgtgtgtgtgtgtgtgtgtttgaatgaaaaatgcttatgtattgacagaacacttctagaatgatacccaaactcctggagtgggagtggggaatgccttctacgtacacactgttctactgtttgaattttttaatatgagcccaaattgtataatctttttttaataaaggggagaaaaatcaaaaaaaaaaaaaaa

SEQ ID NO: 121 - protein, 284 aa, Homo sapiens
  VARIANT (1)...(284): Xaa = Any Amino Acid
MGGPRGAGWVAAGLLLGAGACYCIYRLTRGRRRGDRELGIRSSKSAGALEEGTSEGXLCGRSARPQTGGTWESQWSKTSXPEDLTDGSYDDVLNAEQLQKLLYLLESTEDPVIIERALITLGNNAAFSVNQAIIRELGGIPIVANKINHSNQSIKEKALNALNNLSVNVENQIKIKVQVLKLLLNLXENPAMTEGLLRAQVDSSFLXLYDSHVAKEILLRVLTLFQNIKNCLKIEGHLAVQPTFTEGSLFFLLHGEECAQKIRALVDHHDAEVKEKVVTIIPKI

SEQ ID NO: 122 - DNA, 852 bp, Homo sapiens
  misc_feature (1)...(852): n = A,T,C or G
atgggtggcccccggggcgcgggctgggtggcggcgggcctgctgctcggcgcgggcgcctgctactgcatttacaggctgacccggggtcggcggcggggcgaccgcgagctcgggatacgctcttcgaagtccgcaggtgccctggaagaagggacgtcagagggtcanttgtgcgggcgctcggcccggcctcagacnggaggtacctgggagtcacagtggtccaagacctcgcancctgaagacttaactgatggttcatatgatgatgttctaaatgctgaacaacttcagaaactcctttacctgctggagtcaacggaggatcctgtaattattgaaagagctttgattactttgggtaacaatgcagccttttcagttaaccaagctattattcgtgaattgggtggtattccaattgttgcaaacaaaatcaaccattccaaccagagtattaaagagaaagctttaaatgcactaaataacctgagtgtgaatgttgaaaatcaaatcaagataaaggtgcaagttttgaaactgcttttgaatttgnctgaaaatccagccatgacagaaggacttctccgtgcccaagtggattcatcattcctttycctttatgacagccacgtagcaaaggagattcttcttcgagtacttacgctatttcagaatataaagaactgcctcaaaatagaaggccatttagctgtgcagcctactttcactgaaggttcattgtttttcctgttacatggagaagaatgtgcccagaaaataagagctttagttgatcaccatgatgcagaggtgaaggaaaaggttgtaacaataatacccaaaatc

SEQ ID NO: 123 - DNA, 2595 bp, Homo sapiens
  CDS (386)...(1342)
  misc_feature (1)...(2595): n = A,T,C or G
ccacgcgtccggccaggggcgggagggaggaatggttgcttcacgccccgggggaagagacgggaagctcggctctgggttgcgggccccggcgtctccgcgtggggcgcaccgtccgacccgcccctcccggtgtgcagcgccccgcaccgccccgcctcgcctgggagaagccgccgggacgcgccgggctggagtgggcggttataggctttgagctaggccgtttccgggaggcggagctcagaccccatttcctttctccacatccaggtcaggtggcgtttgctgtggcggctaggcccgcgtgcgctggagacctccgcgctggcccccgcgagcctcctgccctggcccggcgctgcggctctgccgcggcggcagcatgggtggcccccggggcgcgggctgggtggcggcgggcctgctgctcggcgcgggcgcctgctactgcatttacaggctgacccggggtcggcggcggggcgaccgcgagctcgggatacgctcttcgaagtccgcagaagacttaactgatggttcatatgatgatgttctaaatgctgaacaacttcagaaactcctttacctgctggagtcaacggaggatcctgtaattattgaaagagctttgattactttgggtaacaatgcagccttttcagttaaccaaatccctatgaagttggtcactggcatcacattcgctattattcgtgaattgggtggtattccaattgttgcaaacaaaatcaaccattccaaccagagtattaaagagaaagctttaaatgcactaaataacctgagtgtgaatgttgaaaatcaaatcaagataaagatatacatcagtcaagtatgtgaggatgtcttctctggtcctctgaactctgctgtgcagctggctggactgacattgttgacaaacatgactgttaccaatgaccaccagcacatgcttcacagttacattacagacctgttccaggtgktacttactggaaatggaaacacgaaggtgcaagttttgaaactgcttttgaatttgnctgaaaatccagccatgacagaaggacttctccgtgcccaagtggattcatcattcctttycctttatgacagccacgtagcaaaggagattcttcttcgagtacttacgctatttcagaatataaagaactgcctcaaaatagaaggccatttagctgtgcagcctactttcactgaaggttcattgtttttcctgttacatggagaagaatgtgcccagaaaataagagctttagttgatcaccatgatgcagaggtgaaggaaaaggttgtaacaataatacccaaaatctgattggtcatatttttccaaagagtaatgcagtctggatataaatgtattttctgtcttccttataaggggattctcccagctgctaaatttaaacagtaaatatcacattttgtcattaacacagctataacttgccgtggttctcagatttattttggactattttgatgccaagtgaatataagagcttgtactgaaaccatttatttctttctattttgctatttgcaaatgcttgttatcttccctacatgaagtggcagtaacctttttcacatttaagctacccttctaccttttgaagtgatttgcagttactcatctgagacagcatcagtatttgactaaatcattgtttcacaactgaatagtcttgttcttttagtagcaatgaaatcctaagctcttgaggccattcacctgccaacctgaccatactgctttcaaaagtcttttctcatcagtagaatctattttggtcacttctagtcaatgaaaaatgtaaacttttaggagagaatgtttcctaggactcacccactccattcaatgttacatataaaatagtgtgatcaatcacaatgtccatctttagacagttggttaaataaattatctggtctttgaaaagaccgtgctgggcgcggtggctcttgcctgtaatcccagcactttgggaggctgaggcgggcagatcacctgagatcgggagtttgagaccaagcctgaccaatatggagaaaccctgtctctactaagaatacaaaattagctgggcatggtggtgcatgcctgtaatcccagctacttgggaggccgaggcaggagaattgcttgaacccgggaggcagaggttgcagtgaggtgagatagcgccattgcactccagcctgggcaacaagagcaaaactctgtctcaaaaaaaaaaaaaaatgatggagctccgaatgtgcttaagtggaaagatatctatgaaatatggtggttttttaaaacacaaaaattatagaatatgggatcccgtgtgtgtgtgtgtgtgtgtgtgtgtgtgtgtgtgtgtgtgtgtgtgtttgaatgaaaaatgcttatgtattgacagaacacttctagaatgatacccaaactcctggagtgggagtggggaatgccttctacgtacacactgttctactgtttgaattttttaatatgagcccaaattgtataatctttttttaataaaggggagaaaaatcaaaaaaaaaaaaaaa

SEQ ID NO: 124 - protein, 319 aa, Homo sapiens
  VARIANT (1)...(319): Xaa = Any Amino Acid
MGGPRGAGWVAAGLLLGAGACYCIYRLTRGRRRGDRELGIRSSKSAEDLTDGSYDDVLNAEQLQKLLYLLESTEDPVIIERALITLGNNAAFSVNQIPMKLVTGITFAIIRELGGIPIVANKINHSNQSIKEKALNALNNLSVNVENQIKIKIYISQVCEDVFSGPLNSAVQLAGLTLLTNMTVTNDHQHMLHSYITDLFQVXLTGNGNTKVQVLKLLLNLXENPAMTEGLLRAQVDSSFLXLYDSHVAKEILLRVLTLFQNIKNCLKIEGHLAVQPTFTEGSLFFLLHGEECAQKIRALVDHHDAEVKEKVVTIIPKI

SEQ ID NO: 125 - DNA, 957 bp, Homo sapiens
  misc_feature (1)...(957): n = A,T,C or G
atgggtggcccccggggcgcgggctgggtggcggcgggcctgctgctcggcgcgggcgcctgctactgcatttacaggctgacccggggtcggcggcggggcgaccgcgagctcgggatacgctcttcgaagtccgcagaagacttaactgatggttcatatgatgatgttctaaatgctgaacaacttcagaaactcctttacctgctggagtcaacggaggatcctgtaattattgaaagagctttgattactttgggtaacaatgcagccttttcagttaaccaaatccctatgaagttggtcactggcatcacattcgctattattcgtgaattgggtggtattccaattgttgcaaacaaaatcaaccattccaaccagagtattaaagagaaagctttaaatgcactaaataacctgagtgtgaatgttgaaaatcaaatcaagataaagatatacatcagtcaagtatgtgaggatgtcttctctggtcctctgaactctgctgtgcagctggctggactgacattgttgacaaacatgactgttaccaatgaccaccagcacatgcttcacagttacattacagacctgttccaggtgktacttactggaaatggaaacacgaaggtgcaagttttgaaactgcttttgaatttgnctgaaaatccagccatgacagaaggacttctccgtgcccaagtggattcatcattcctttycctttatgacagccacgtagcaaaggagattcttcttcgagtacttacgctatttcagaatataaagaactgcctcaaaatagaaggccatttagctgtgcagcctactttcactgaaggttcattgtttttcctgttacatggagaagaatgtgcccagaaaataagagctttagttgatcaccatgatgcagaggtgaaggaaaaggttgtaacaataatacccaaaatc

SEQ ID NO: 126 - DNA, 2700 bp, Homo sapiens
  CDS (386)...(1447)
  misc_feature (1)...(2700): n = A,T,C or G
ccacgcgtccggccaggggcgggagggaggaatggttgcttcacgccccgggggaagagacgggaagctcggctctgggttgcgggccccggcgtctccgcgtggggcgcaccgtccgacccgcccctcccggtgtgcagcgccccgcaccgccccgcctcgcctgggagaagccgccgggacgcgccgggctggagtgggcggttataggctttgagctaggccgtttccgggaggcggagctcagaccccatttcctttctccacatccaggtcaggtggcgtttgctgtggcggctaggcccgcgtgcgctggagacctccgcgctggcccccgcgagcctcctgccctggcccggcgctgcggctctgccgcggcggcagcatgggtggcccccggggcgcgggctgggtggcggcgggcctgctgctcggcgcgggcgcctgctactgcatttacaggctgacccggggtcggcggcggggcgaccgcgagctcgggatacgctcttcgaagtccgcaggtgccctggaagaagggacgtcagagggtcanttgtgcgggcgctcggcccggcctcagacnggaggtacctgggagtcacagtggtccaagacctcgcancctgaagacttaactgatggttcatatgatgatgttctaaatgctgaacaacttcagaaactcctttacctgctggagtcaacggaggatcctgtaattattgaaagagctttgattactttgggtaacaatgcagccttttcagttaaccaaatccctatgaagttggtcactggcatcacattcgctattattcgtgaattgggtggtattccaattgttgcaaacaaaatcaaccattccaaccagagtattaaagagaaagctttaaatgcactaaataacctgagtgtgaatgttgaaaatcaaatcaagataaagatatacatcagtcaagtatgtgaggatgtcttctctggtcctctgaactctgctgtgcagctggctggactgacattgttgacaaacatgactgttaccaatgaccaccagcacatgcttcacagttacattacagacctgttccaggtgktacttactggaaatggaaacacgaaggtgcaagttttgaaactgcttttgaatttgnctgaaaatccagccatgacagaaggacttctccgtgcccaagtggattcatcattcctttycctttatgacagccacgtagcaaaggagattcttcttcgagtacttacgctatttcagaatataaagaactgcctcaaaatagaaggccatttagctgtgcagcctactttcactgaaggttcattgtttttcctgttacatggagaagaatgtgcccagaaaataagagctttagttgatcaccatgatgcagaggtgaaggaaaaggttgtaacaataatacccaaaatctgattggtcatatttttccaaagagtaatgcagtctggatataaatgtattttctgtcttccttataaggggattctcccagctgctaaatttaaacagtaaatatcacattttgtcattaacacagctataacttgccgtggttctcagatttattttggactattttgatgccaagtgaatataagagcttgtactgaaaccatttatttctttctattttgctatttgcaaatgcttgttatcttccctacatgaagtggcagtaacctttttcacatttaagctacccttctaccttttgaagtgatttgcagttactcatctgagacagcatcagtatttgactaaatcattgtttcacaactgaatagtcttgttcttttagtagcaatgaaatcctaagctcttgaggccattcacctgccaacctgaccatactgctttcaaaagtcttttctcatcagtagaatctattttggtcacttctagtcaatgaaaaatgtaaacttttaggagagaatgtttcctaggactcacccactccattcaatgttacatataaaatagtgtgatcaatcacaatgtccatctttagacagttggttaaataaattatctggtctttgaaaagaccgtgctgggcgcggtggctcttgcctgtaatcccagcactttgggaggctgaggcgggcagatcacctgagatcgggagtttgagaccaagcctgaccaatatggagaaaccctgtctctactaagaatacaaaattagctgggcatggtggtgcatgcctgtaatcccagctacttgggaggccgaggcaggagaattgcttgaacccgggaggcagaggttgcagtgaggtgagatagcgccattgcactccagcctgggcaacaagagcaaaactctgtctcaaaaaaaaaaaaaaatgatggagctccgaatgtgcttaagtggaaagatatctatgaaatatggtggttttttaaaacacaaaaattatagaatatgggatcccgtgtgtgtgtgtgtgtgtgtgtgtgtgtgtgtgtgtgtgtgtgtgtgtttgaatgaaaaatgcttatgtattgacagaacacttctagaatgatacccaaactcctggagtgggagtggggaatgccttctacgtacacactgttctactgtttgaattttttaatatgagcccaaattgtataatctttttttaataaaggggagaaaaatcaaaaaaaaaaaaaaa

SEQ ID NO: 127 - protein, 354 aa, Homo sapiens
  VARIANT (1)...(354): Xaa = Any Amino Acid
MGGPRGAGWVAAGLLLGAGACYCIYRLTRGRRRGDRELGIRSSKSAGALEEGTSEGXLCGRSARPQTGGTWESQWSKTSXPEDLTDGSYDDVLNAEQLQKLLYLLESTEDPVIIERALITLGNNAAFSVNQIPMKLVTGITFAIIRELGGIPIVANKINHSNQSIKEKALNALNNLSVNVENQIKIKIYISQVCEDVFSGPLNSAVQLAGLTLLTNMTVTNDHQHMLHSYITDLFQVXLTGNGNTKVQVLKLLLNLXENPAMTEGLLRAQVDSSFLXLYDSHVAKEILLRVLTLFQNIKNCLKIEGHLAVQPTFTEGSLFFLLHGEECAQKIRALVDHHDAEVKEKVVTIIPKI

SEQ ID NO: 128 - DNA, 1062 bp, Homo sapiens
  misc_feature (1)...(1062): n = A,T,C or G
atgggtggcccccggggcgcgggctgggtggcggcgggcctgctgctcggcgcgggcgcctgctactgcatttacaggctgacccggggtcggcggcggggcgaccgcgagctcgggatacgctcttcgaagtccgcaggtgccctggaagaagggacgtcagagggtcanttgtgcgggcgctcggcccggcctcagacnggaggtacctgggagtcacagtggtccaagacctcgcancctgaagacttaactgatggttcatatgatgatgttctaaatgctgaacaacttcagaaactcctttacctgctggagtcaacggaggatcctgtaattattgaaagagctttgattactttgggtaacaatgcagccttttcagttaaccaaatccctatgaagttggtcactggcatcacattcgctattattcgtgaattgggtggtattccaattgttgcaaacaaaatcaaccattccaaccagagtattaaagagaaagctttaaatgcactaaataacctgagtgtgaatgttgaaaatcaaatcaagataaagatatacatcagtcaagtatgtgaggatgtcttctctggtcctctgaactctgctgtgcagctggctggactgacattgttgacaaacatgactgttaccaatgaccaccagcacatgcttcacagttacattacagacctgttccaggtgktacttactggaaatggaaacacgaaggtgcaagttttgaaactgcttttgaatttgnctgaaaatccagccatgacagaaggacttctccgtgcccaagtggattcatcattcctttycctttatgacagccacgtagcaaaggagattcttcttcgagtacttacgctatttcagaatataaagaactgcctcaaaatagaaggccatttagctgtgcagcctactttcactgaaggttcattgtttttcctgttacatggagaagaatgtgcccagaaaataagagctttagttgatcaccatgatgcagaggtgaaggaaaaggttgtaacaataatacccaaaatc

SEQ ID NO: 129 - DNA, 2523 bp, Homo sapiens
  CDS (386)...(1270)
  misc_feature (1)...(2523): n = A,T,C or G
ccacgcgtccggccaggggcgggagggaggaatggttgcttcacgccccgggggaagagacgggaagctcggctctgggttgcgggccccggcgtctccgcgtggggcgcaccgtccgacccgcccctcccggtgtgcagcgccccgcaccgccccgcctcgcctgggagaagccgccgggacgcgccgggctggagtgggcggttataggctttgagctaggccgtttccgggaggcggagctcagaccccatttcctttctccacatccaggtcaggtggcgtttgctgtggcggctaggcccgcgtgcgctggagacctccgcgctggcccccgcgagcctcctgccctggcccggcgctgcggctctgccgcggcggcagcatgggtggcccccggggcgcgggctgggtggcggcgggcctgctgctcggcgcgggcgcctgctactgcatttacaggctgacccggggtcggcggcggggcgaccgcgagctcgggatacgctcttcgaagtccgcaggtgccctggaagaagggacgtcagagggtcanttgtgcgggcgctcggcccggcctcagacnggaggtacctgggagtcacagtggtccaagacctcgcancctgaagacttaactgatggttcatatgatgatgttctaaatgctgaacaacttcagaaactcctttacctgctggagtcaacggaggatcctgtaattattgaaagagctttgattactttgggtaacaatgcagccttttcagttaaccaaatccctatgaagttggtcactggcatcacattcgctattattcgtgaattgggtggtattccaattgttgcaaacaaaatcaaccattccaaccagagtattaaagagaaagctttaaatgcactaaataacctgagtgtgaatgttgaaaatcaaatcaagataaaggtgcaagttttgaaactgcttttgaatttgnctgaaaatccagccatgacagaaggacttctccgtgcccaagtggattcatcattcctttycctttatgacagccacgtagcaaaggagattcttcttcgagtacttacgctatttcagaatataaagaactgcctcaaaatagaaggccatttagctgtgcagcctactttcactgaaggttcattgtttttcctgttacatggagaagaatgtgcccagaaaataagagctttagttgatcaccatgatgcagaggtgaaggaaaaggttgtaacaataatacccaaaatctgattggtcatatttttccaaagagtaatgcagtctggatataaatgtattttctgtcttccttataaggggattctcccagctgctaaatttaaacagtaaatatcacattttgtcattaacacagctataacttgccgtggttctcagatttattttggactattttgatgccaagtgaatataagagcttgtactgaaaccatttatttctttctattttgctatttgcaaatgcttgttatcttccctacatgaagtggcagtaacctttttcacatttaagctacccttctaccttttgaagtgatttgcagttactcatctgagacagcatcagtatttgactaaatcattgtttcacaactgaatagtcttgttcttttagtagcaatgaaatcctaagctcttgaggccattcacctgccaacctgaccatactgctttcaaaagtcttttctcatcagtagaatctattttggtcacttctagtcaatgaaaaatgtaaacttttaggagagaatgtttcctaggactcacccactccattcaatgttacatataaaatagtgtgatcaatcacaatgtccatctttagacagttggttaaataaattatctggtctttgaaaagaccgtgctgggcgcggtggctcttgcctgtaatcccagcactttgggaggctgaggcgggcagatcacctgagatcgggagtttgagaccaagcctgaccaatatggagaaaccctgtctctactaagaatacaaaattagctgggcatggtggtgcatgcctgtaatcccagctacttgggaggccgaggcaggagaattgcttgaacccgggaggcagaggttgcagtgaggtgagatagcgccattgcactccagcctgggcaacaagagcaaaactctgtctcaaaaaaaaaaaaaaatgatggagctccgaatgtgcttaagtggaaagatatctatgaaatatggtggttttttaaaacacaaaaattatagaatatgggatcccgtgtgtgtgtgtgtgtgtgtgtgtgtgtgtgtgtgtgtgtgtgtgtgtttgaatgaaaaatgcttatgtattgacagaacacttctagaatgatacccaaactcctggagtgggagtggggaatgccttctacgtacacactgttctactgtttgaattttttaatatgagcccaaattgtataatctttttttaataaaggggagaaaaatcaaaaaaaaaaaaaaa

SEQ ID NO: 130 - protein, 295 aa, Homo sapiens
  VARIANT (1)...(295): Xaa = Any Amino Acid
MGGPRGAGWVAAGLLLGAGACYCIYRLTRGRRRGDRELGIRSSKSAGALEEGTSEGXLCGRSARPQTGGTWESQWSKTSXPEDLTDGSYDDVLNAEQLQKLLYLLESTEDPVIIERALITLGNNAAFSVNQIPMKLVTGITFAIIRELGGIPIVANKINHSNQSIKEKALNALNNLSVNVENQIKIKVQVLKLLLNLXENPAMTEGLLRAQVDSSFLXLYDSHVAKEILLRVLTLFQNIKNCLKIEGHLAVQPTFTEGSLFFLLHGEECAQKIRALVDHHDAEVKEKVVTIIPKI

SEQ ID NO: 131 - DNA, 885 bp, Homo sapiens
  misc_feature (1)...(885): n = A,T,C or G
atgggtggcccccggggcgcgggctgggtggcggcgggcctgctgctcggcgcgggcgcctgctactgcatttacaggctgacccggggtcggcggcggggcgaccgcgagctcgggatacgctcttcgaagtccgcaggtgccctggaagaagggacgtcagagggtcanttgtgcgggcgctcggcccggcctcagacnggaggtacctgggagtcacagtggtccaagacctcgcancctgaagacttaactgatggttcatatgatgatgttctaaatgctgaacaacttcagaaactcctttacctgctggagtcaacggaggatcctgtaattattgaaagagctttgattactttgggtaacaatgcagccttttcagttaaccaaatccctatgaagttggtcactggcatcacattcgctattattcgtgaattgggtggtattccaattgttgcaaacaaaatcaaccattccaaccagagtattaaagagaaagctttaaatgcactaaataacctgagtgtgaatgttgaaaatcaaatcaagataaaggtgcaagttttgaaactgcttttgaatttgnctgaaaatccagccatgacagaaggacttctccgtgcccaagtggattcatcattcctttycctttatgacagccacgtagcaaaggagattcttcttcgagtacttacgctatttcagaatataaagaactgcctcaaaatagaaggccatttagctgtgcagcctactttcactgaaggttcattgtttttcctgttacatggagaagaatgtgcccagaaaataagagctttagttgatcaccatgatgcagaggtgaaggaaaaggttgtaacaataatacccaaaatc

SEQ ID NO: 132 - DNA, 2418 bp, Homo sapiens
  CDS (386)...(1165)
  misc_feature (1)...(2418): n = A,T,C or G
ccacgcgtccggccaggggcgggagggaggaatggttgcttcacgccccgggggaagagacgggaagctcggctctgggttgcgggccccggcgtctccgcgtggggcgcaccgtccgacccgcccctcccggtgtgcagcgccccgcaccgccccgcctcgcctgggagaagccgccgggacgcgccgggctggagtgggcggttataggctttgagctaggccgtttccgggaggcggagctcagaccccatttcctttctccacatccaggtcaggtggcgtttgctgtggcggctaggcccgcgtgcgctggagacctccgcgctggcccccgcgagcctcctgccctggcccggcgctgcggctctgccgcggcggcagcatgggtggcccccggggcgcgggctgggtggcggcgggcctgctgctcggcgcgggcgcctgctactgcatttacaggctgacccggggtcggcggcggggcgaccgcgagctcgggatacgctcttcgaagtccgcagaagacttaactgatggttcatatgatgatgttctaaatgctgaacaacttcagaaactcctttacctgctggagtcaacggaggatcctgtaattattgaaagagctttgattactttgggtaacaatgcagccttttcagttaaccaaatccctatgaagttggtcactggcatcacattcgctattattcgtgaattgggtggtattccaattgttgcaaacaaaatcaaccattccaaccagagtattaaagagaaagctttaaatgcactaaataacctgagtgtgaatgttgaaaatcaaatcaagataaaggtgcaagttttgaaactgcttttgaatttgnctgaaaatccagccatgacagaaggacttctccgtgcccaagtggattcatcattcctttycctttatgacagccacgtagcaaaggagattcttcttcgagtacttacgctatttcagaatataaagaactgcctcaaaatagaaggccatttagctgtgcagcctactttcactgaaggttcattgtttttcctgttacatggagaagaatgtgcccagaaaataagagctttagttgatcaccatgatgcagaggtgaaggaaaaggttgtaacaataatacccaaaatctgattggtcatatttttccaaagagtaatgcagtctggatataaatgtattttctgtcttccttataaggggattctcccagctgctaaatttaaacagtaaatatcacattttgtcattaacacagctataacttgccgtggttctcagatttattttggactattttgatgccaagtgaatataagagcttgtactgaaaccatttatttctttctattttgctatttgcaaatgcttgttatcttccctacatgaagtggcagtaacctttttcacatttaagctacccttctaccttttgaagtgatttgcagttactcatctgagacagcatcagtatttgactaaatcattgtttcacaactgaatagtcttgttcttttagtagcaatgaaatcctaagctcttgaggccattcacctgccaacctgaccatactgctttcaaaagtcttttctcatcagtagaatctattttggtcacttctagtcaatgaaaaatgtaaacttttaggagagaatgtttcctaggactcacccactccattcaatgttacatataaaatagtgtgatcaatcacaatgtccatctttagacagttggttaaataaattatctggtctttgaaaagaccgtgctgggcgcggtggctcttgcctgtaatcccagcactttgggaggctgaggcgggcagatcacctgagatcgggagtttgagaccaagcctgaccaatatggagaaaccctgtctctactaagaatacaaaattagctgggcatggtggtgcatgcctgtaatcccagctacttgggaggccgaggcaggagaattgcttgaacccgggaggcagaggttgcagtgaggtgagatagcgccattgcactccagcctgggcaacaagagcaaaactctgtctcaaaaaaaaaaaaaaatgatggagctccgaatgtgcttaagtggaaagatatctatgaaatatggtggttttttaaaacacaaaaattatagaatatgggatcccgtgtgtgtgtgtgtgtgtgtgtgtgtgtgtgtgtgtgtgtgtgtgtgtttgaatgaaaaatgcttatgtattgacagaacacttctagaatgatacccaaactcctggagtgggagtggggaatgccttctacgtacacactgttctactgtttgaattttttaatatgagcccaaattgtataatctttttttaataaaggggagaaaaatcaaaaaaaaaaaaaaa

SEQ ID NO: 133 - protein, 260 aa, Homo sapiens
  VARIANT (1)...(260): Xaa = Any Amino Acid
MGGPRGAGWVAAGLLLGAGACYCIYRLTRGRRRGDRELGIRSSKSAEDLTDGSYDDVLNAEQLQKLLYLLESTEDPVIIERALITLGNNAAFSVNQIPMKLVTGITFAIIRELGGIPIVANKINHSNQSIKEKALNALNNLSVNVENQIKIKVQVLKLLLNLXENPAMTEGLLRAQVDSSFLXLYDSHVAKEILLRVLTLFQNIKNCLKIEGHLAVQPTFTEGSLFFLLHGEECAQKIRALVDHHDAEVKEKVVTIIPKI

SEQ ID NO: 134 - DNA, 780 bp, Homo sapiens
  misc_feature (1)...(780): n = A,T,C or G
atgggtggcccccggggcgcgggctgggtggcggcgggcctgctgctcggcgcgggcgcctgctactgcatttacaggctgacccggggtcggcggcggggcgaccgcgagctcgggatacgctcttcgaagtccgcagaagacttaactgatggttcatatgatgatgttctaaatgctgaacaacttcagaaactcctttacctgctggagtcaacggaggatcctgtaattattgaaagagctttgattactttgggtaacaatgcagccttttcagttaaccaaatccctatgaagttggtcactggcatcacattcgctattattcgtgaattgggtggtattccaattgttgcaaacaaaatcaaccattccaaccagagtattaaagagaaagctttaaatgcactaaataacctgagtgtgaatgttgaaaatcaaatcaagataaaggtgcaagttttgaaactgcttttgaatttgnctgaaaatccagccatgacagaaggacttctccgtgcccaagtggattcatcattcctttycctttatgacagccacgtagcaaaggagattcttcttcgagtacttacgctatttcagaatataaagaactgcctcaaaatagaaggccatttagctgtgcagcctactttcactgaaggttcattgtttttcctgttacatggagaagaatgtgcccagaaaataagagctttagttgatcaccatgatgcagaggtgaaggaaaaggttgtaacaataatacccaaaatc

SEQ ID NO: 135 - DNA, 2562 bp, Homo sapiens
  CDS (386)...(1309)
  misc_feature (1)...(2562): n = A,T,C or G
ccacgcgtccggccaggggcgggagggaggaatggttgcttcacgccccgggggaagagacgggaagctcggctctgggttgcgggccccggcgtctccgcgtggggcgcaccgtccgacccgcccctcccggtgtgcagcgccccgcaccgccccgcctcgcctgggagaagccgccgggacgcgccgggctggagtgggcggttataggctttgagctaggccgtttccgggaggcggagctcagaccccatttcctttctccacatccaggtcaggtggcgtttgctgtggcggctaggcccgcgtgcgctggagacctccgcgctggcccccgcgagcctcctgccctggcccggcgctgcggctctgccgcggcggcagcatgggtggcccccggggcgcgggctgggtggcggcgggcctgctgctcggcgcgggcgcctgctactgcatttacaggctgacccggggtcggcggcggggcgaccgcgagctcgggatacgctcttcgaagtccgcagaagacttaactgatggttcatatgatgatgttctaaatgctgaacaacttcagaaactcctttacctgctggagtcaacggaggatcctgtaattattgaaagagctttgattactttgggtaacaatgcagccttttcagttaaccaagctattattcgtgaattgggtggtattccaattgttgcaaacaaaatcaaccattccaaccagagtattaaagagaaagctttaaatgcactaaataacctgagtgtgaatgttgaaaatcaaatcaagataaagatatacatcagtcaagtatgtgaggatgtcttctctggtcctctgaactctgctgtgcagctggctggactgacattgttgacaaacatgactgttaccaatgaccaccagcacatgcttcacagttacattacagacctgttccaggtgktacttactggaaatggaaacacgaaggtgcaagttttgaaactgcttttgaatttgnctgaaaatccagccatgacagaaggacttctccgtgcccaagtggattcatcattcctttycctttatgacagccacgtagcaaaggagattcttcttcgagtacttacgctatttcagaatataaagaactgcctcaaaatagaaggccatttagctgtgcagcctactttcactgaaggttcattgtttttcctgttacatggagaagaatgtgcccagaaaataagagctttagttgatcaccatgatgcagaggtgaaggaaaaggttgtaacaataatacccaaaatctgattggtcatatttttccaaagagtaatgcagtctggatataaatgtattttctgtcttccttataaggggattctcccagctgctaaatttaaacagtaaatatcacattttgtcattaacacagctataacttgccgtggttctcagatttattttggactattttgatgccaagtgaatataagagcttgtactgaaaccatttatttctttctattttgctatttgcaaatgcttgttatcttccctacatgaagtggcagtaacctttttcacatttaagctacccttctaccttttgaagtgatttgcagttactcatctgagacagcatcagtatttgactaaatcattgtttcacaactgaatagtcttgttcttttagtagcaatgaaatcctaagctcttgaggccattcacctgccaacctgaccatactgctttcaaaagtcttttctcatcagtagaatctattttggtcacttctagtcaatgaaaaatgtaaacttttaggagagaatgtttcctaggactcacccactccattcaatgttacatataaaatagtgtgatcaatcacaatgtccatctttagacagttggttaaataaattatctggtctttgaaaagaccgtgctgggcgcggtggctcttgcctgtaatcccagcactttgggaggctgaggcgggcagatcacctgagatcgggagtttgagaccaagcctgaccaatatggagaaaccctgtctctactaagaatacaaaattagctgggcatggtggtgcatgcctgtaatcccagctacttgggaggccgaggcaggagaattgcttgaacccgggaggcagaggttgcagtgaggtgagatagcgccattgcactccagcctgggcaacaagagcaaaactctgtctcaaaaaaaaaaaaaaatgatggagctccgaatgtgcttaagtggaaagatatctatgaaatatggtggttttttaaaacacaaaaattatagaatatgggatcccgtgtgtgtgtgtgtgtgtgtgtgtgtgtgtgtgtgtgtgtgtgtgtgtttgaatgaaaaatgcttatgtattgacagaacacttctagaatgatacccaaactcctggagtgggagtggggaatgccttctacgtacacactgttctactgtttgaattttttaatatgagcccaaattgtataatctttttttaataaaggggagaaaaatcaaaaaaaaaaaaaaa

SEQ ID NO: 136 - protein, 308 aa, Homo sapiens
  VARIANT (1)...(308): Xaa = Any Amino Acid
MGGPRGAGWVAAGLLLGAGACYCIYRLTRGRRRGDRELGIRSSKSAEDLTDGSYDDVLNAEQLQKLLYLLESTEDPVIIERALITLGNNAAFSVNQAIIRELGGIPIVANKINHSNQSIKEKALNALNNLSVNVENQIKIKIYISQVCEDVFSGPLNSAVQLAGLTLLTNMTVTNDHQHMLHSYITDLFQVXLTGNGNTKVQVLKLLLNLXENPAMTEGLLRAQVDSSFLXLYDSHVAKEILLRVLTLFQNIKNCLKIEGHLAVQPTFTEGSLFFLLHGEECAQKIRALVDHHDAEVKEKVVTIIPKI

SEQ ID NO: 137 - DNA, 924 bp, Homo sapiens
  misc_feature (1)...(924): n = A,T,C or G
atgggtggcccccggggcgcgggctgggtggcggcgggcctgctgctcggcgcgggcgcctgctactgcatttacaggctgacccggggtcggcggcggggcgaccgcgagctcgggatacgctcttcgaagtccgcagaagacttaactgatggttcatatgatgatgttctaaatgctgaacaacttcagaaactcctttacctgctggagtcaacggaggatcctgtaattattgaaagagctttgattactttgggtaacaatgcagccttttcagttaaccaagctattattcgtgaattgggtggtattccaattgttgcaaacaaaatcaaccattccaaccagagtattaaagagaaagctttaaatgcactaaataacctgagtgtgaatgttgaaaatcaaatcaagataaagatatacatcagtcaagtatgtgaggatgtcttctctggtcctctgaactctgctgtgcagctggctggactgacattgttgacaaacatgactgttaccaatgaccaccagcacatgcttcacagttacattacagacctgttccaggtgktacttactggaaatggaaacacgaaggtgcaagttttgaaactgcttttgaatttgnctgaaaatccagccatgacagaaggacttctccgtgcccaagtggattcatcattcctttycctttatgacagccacgtagcaaaggagattcttcttcgagtacttacgctatttcagaatataaagaactgcctcaaaatagaaggccatttagctgtgcagcctactttcactgaaggttcattgtttttcctgttacatggagaagaatgtgcccagaaaataagagctttagttgatcaccatgatgcagaggtgaaggaaaaggttgtaacaataatacccaaaatc

SEQ ID NO: 138 - protein, 16 aa, Homo sapiens
ELLHQKLELPDNVSGE